(19) United States
(12) Patent Application Publication
(10) Pub. No.: US 2022/0105167 A1
(43) Pub. Date: Apr. 7, 2022

(54) ESCHERICHIA COLI 0157:H7 PROTEINS AND USES THEREOF

(71) Applicants: GLAXOSMITHKLINE BIOLOGICALS SA, Rixensart (BE); Institut National de Recherche pour l'Agriculture, l'Alimentation et I'Environnement, Paris (FR)

(72) Inventors: Mickael DESVAUX, Sauvagnat Sainte Marthe (FR); Mariagrazia PIZZA, Siena (IT); Maricarmen ROJAS-LOPEZ, Boston, MA (US); Roberto ROSINI, Siena (IT)

(21) Appl. No.: 17/251,973

(22) PCT Filed: Jun. 12, 2019
(86) PCT No.: PCT/EP2019/065354

§ 371 Date: Dec. 14, 2020

(30) Foreign Application Priority Data
Jun. 15, 2018 (EP) 18178128.7

(51) Int. Cl. A61K 39/108; C12Q 1/689; C07K 14/245; A61P 31/04

(52) U.S. Cl. CPC A61K 39/0258; A61P 31/04; C07K 14/245; C12Q 1/689

(57) ABSTRACT

The present invention is direct to isolated polypeptides comprising or consisting of: an amino acid sequence selected from the group consisting of SEQ ID NOs: 25, 26, 27, 28, 29, 30, 31, 32, 33, 34, 35, 36, 37, 38, 39, 40, 41, 42, 43, 44, 45, 46, 47 or 48; or (a) a variant and/or fragment of (a), for example: (i) a variant of (a); (ii) a fragment of (a); or (iii) a variant of a fragment of (a); together with corresponding isolated nucleic acid molecules, vectors, host cells, methods of production, vesicles, binding moieties, pharmaceutical compositions, kits, methods of treatment and medical uses.

### SEQUENCE LISTING

The instant application contains an electronically submitted Sequence Listing in ASCII text file format (Name: VB66S11_US_SL.txt; Size: 219,097 bytes; and Date of Creation: Feb. 19, 2021) which is hereby incorporated by reference in its entirety.

### FIELD OF THE INVENTION

The present invention relates to novel enterohaemorrhagic Escherichia coli (EHEC) antigens and their use in treating disease.

### BACKGROUND TO THE INVENTION

Escherichia coli enterohemorrhagic (EHEC) is an etiologic agent for anthropozoonotic diarrheal disease and hemorrhagic colitis. EHEC infections occur mainly in developed countries; the most common O157:H7 serotype and other non-157 serogroups often have been implicated in human outbreaks (Kaper et al., 2004; Moriel et al., 2012; Croxen et al., 2013). The main reservoir of EHEC is ruminants, and anthropozoonotic infection mainly occurs from direct or indirect contamination of food products with animal faeces (Rivas et al., 2016). EHEC strains are characterized for the expression of shiga-like toxin (Stx), this being the hallmark of the pathotype. Some strains also carry the Locus of Enterocyte Effacement (LEE) that encodes the type three secretion system (T3SS) responsible for the attachment and effacing (A/E) lesions on the intestinal microvilli (Kaper et al., 2004).

Symptoms of infection include the development of the hemolytic uremic syndrome (HUS) and later renal failure, due mainly to the Stx (Tarr et al., 2005). Although the use of antibiotic remains a key intervention strategy to treat many bacterial infections, this therapy is not recommended in diseases caused by EHEC (Goldwater and Bettelheim, 2012; Rivas et al., 2016).

Furthermore, the increasing burden of these E. coli diarrheal diseases, the emergence of hybrid strains (genomic plasticity), and the increasing annual cost for the health care systems point out the need to develop effective therapeutic and preventive strategies. Among these, vaccination is a promising strategy to control disease (Croxen and Finlay, 2010; Moriel et al., 2012; Croxen et al., 2013; Rappuoli et al., 2014).

A number of antigen candidates for potential EHEC vaccine use have been identified. Virulence factors expressed as recombinant proteins (such as Shiga-toxin (Stx), Intimin and E. coli secreted protein A (EspA)) and avirulent ghost cells of EHEC O157:H7 have been tested using different routes of immunization and adjuvant combinations in several animal models providing encouraging results (Rojas-Lopez et al., 2018, Front. Microbiol., 2018; 9: 440, doi: 10.3389/fmicb.2018.00440).

To date, no human EHEC vaccine has been brought to market. Hence, there is a continued need to identify novel antigens for vaccine development against infections caused by EHEC.

### DESCRIPTION OF THE INVENTION

Novel antigens have been identified from the EHEC O157:H7 genome.

A first aspect of the invention provides an isolated polypeptide comprising or consisting of:
(a) Group A polypeptides: an amino acid sequence selected from the group consisting of SEQ ID NOs: 25, 26, 27, 28, 29, 30, 31, 32, 33, 34, 35, 36, 37, 38, 39, 40, 41, 42, 43, 44, 45, 46, 47 or 48; or
(b) Group B polypeptides: a variant and/or fragment of (Group A), for example:
   (i) a variant of (Group A);
   (ii) a fragment of (Group A);
   (iii) a variant of a fragment of (Group A).

The term ‘amino acid’ as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g. α,α-disubstituted amino acids, N-alkyl amino acids, etc.) and chemically derivatised amino acids (see below).

Thus, when an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

By “isolated” we mean that the feature (e.g., the polypeptide) of the invention is provided in a context other than that in which it may be found naturally. One of skill in the art would understand that “isolated” means altered “by the hand of man” from its natural state, i.e., if it occurs in nature, it has been changed or removed from its original environment, or both. For example, a polynucleotide or a polypeptide naturally present in a living organism is not “isolated” when in such living organism, but the same polynucleotide or polypeptide separated from the coexisting materials of its natural state is “isolated,” as the term is used in this disclosure. Further, a polynucleotide or polypeptide that is introduced into an organism by transformation, genetic manipulation or by any other recombinant method would be understood to be “isolated” even if it is still present in said organism, which organism may be living or non-living, except where such transformation, genetic manipulation or other recombinant method produces an organism that is otherwise indistinguishable from the naturally-occurring organism.

By ‘variant’ of the polypeptide we include insertions, deletions and/or substitutions, either conservative or non-conservative. In particular, the variant polypeptide may be a non-naturally occurring variant (i.e., does not, or is not known to, occur in nature).

“Sequence identity” can be determined by the Smith Waterman homology search algorithm as implemented in the MPSRCH program (Oxford Molecular), using an affine gap search with parameters gap open penalty=12 and gap extension penalty=1, or by the Needleman-Wunsch global alignment algorithm (see e.g. Rubin (2000) Pediatric. Clin. North Am. 47:269-285), using default parameters (e.g. with Gap opening penalty=10.0, and with Gap extension penalty=0.5, using the EBLOSUM62 scoring matrix). This algorithm is conveniently implemented in the needle tool in the EMBOSS package. Unless specified otherwise, where the application refers to sequence identity to a particular sequence, the identity is intended to be calculated over the entire length of that sequence. Alternatively, percent identity can be determined by methods well known in the art, for example using the LALIGN program (Huang and Miller, Adv. Appl. Math. (1991) 12:337-357, the disclosures of which are incorporated herein by reference) at the ExPASy facility website www.ch.embnet.org/software/LALIGN_form.html using as parameters the global alignment option, scoring matrix BLOSUM62, opening gap penalty −14, extending gap penalty −4. Alternatively, the percent sequence identity between two polypeptides may be determined using suitable computer programs, for example AlignX, Vector NTI Advance 10 (from Invitrogen Corporation) or the GAP program (from the University of Wisconsin Genetic Computing Group).

It will be appreciated that percent identity is calculated in relation to polymers (e.g., polypeptide or polynucleotide) whose sequence has been aligned.

Fragments and variants may be made using the methods of protein engineering and site-directed mutagenesis well known in the art (for example, see Molecular Cloning: a Laboratory Manual, 3rd edition, Sambrook & Russell, 2001, Cold Spring Harbor Laboratory Press, the disclosures of which are incorporated herein by reference).

It will be appreciated by skilled persons that the polypeptide of the invention, or fragment, variant or fusion thereof, may comprise one or more amino acids that are modified or derivatised. Thus, the polypeptide may comprise or consist of a derivative of the amino acid sequences of Group A or of a fragment or variant thereof.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. Thus, by ‘polypeptide’ we include peptidomimetic compounds which exhibit endolysin activity. The term ‘peptidomimetic’ refers to a compound that mimics the conformation and desirable features of a particular polypeptide as a therapeutic agent.

For example, the polypeptides described herein include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, the disclosures of which are incorporated herein by reference. Such retro-inverse peptides, which contain NH—CO bonds instead of CO—NH peptide bonds, are much more resistant to proteolysis. Alternatively, the polypeptide of the invention may be a peptidomimetic compound wherein one or more of the amino acid residues are linked by a -γ(CH2NH)— bond in place of the conventional amide linkage.

It will be appreciated that the polypeptide may conveniently be blocked at its N- or C-terminus so as to help reduce susceptibility to exoproteolytic digestion, e.g., by amidation.

As discussed herein, a variety of uncoded or modified amino acids such as D-amino acids and N-methyl amino acids may be used to modify polypeptides of the invention. In addition, a presumed bioactive conformation may be stabilised by a covalent modification, such as cyclisation or by incorporation of lactam or other types of bridges. Methods of synthesis of cyclic homodetic peptides and cyclic heterodetic peptides, including disulphide, sulphide and alkylene bridges, are disclosed in U.S. Pat. No. 5,643,872. Other examples of cyclisation methods are discussed and disclosed in U.S. Pat. No. 6,008,058, the relevant disclosures in which documents are hereby incorporated by reference. A further approach to the synthesis of cyclic stabilised peptidomimetic compounds is ring-closing metathesis (RCM).

In summary, terminal modifications are useful, as is well known, to reduce susceptibility by proteinase digestion and therefore to prolong the half-life of the peptides in solutions, particularly in biological fluids where proteases may be present. Polypeptide cyclisation is also a useful modification and is preferred because of the stable structures formed by cyclisation and in view of the biological activities observed for cyclic peptides.

Thus, in one embodiment the polypeptide, or fragment, variant, fusion or derivative thereof, is cyclic. However, in a preferred embodiment, the polypeptide, or fragment, variant, fusion or derivative thereof, is linear.

In a further embodiment of the first aspect of the invention, the polypeptide comprises or consists of a fusion of the amino acid sequence of Group A or Group B polypeptides, or of a fragment, variant or derivative thereof.

By ‘fusion’ of a polypeptide we include a polypeptide which is fused to any other polypeptide. For example, the polypeptide may comprise one or more additional amino acids, inserted internally and/or at the N- and/or C-termini of the amino acid sequence of Group A or Group B polypeptides, or of a fragment, variant or derivative thereof.

Thus, as described herein, in one embodiment the polypeptide of the first aspect of the invention comprises a polypeptide of the invention to which is fused an enzymatic domain from a different source (e.g., from a source other than the polypeptide of the first aspect of the invention). Examples of suitable enzymatic domains include: L-alanoyl-D-glutamate endopeptidase; D-glutamyl-m-DAP endopeptidase; interpeptide bridge-specific endopeptidase; N-acetyl-β-D-glucosaminidase (=muramoylhydrolase); N-acetyl-β-D-muramidase (=lysozyme); lytic transglycosylase.

Also N-acetylmuramoyl-L-alanine amidase from other sources could be utilised (see Loessner, 2005, Current Opinion in Microbiology 8: 480-487, the disclosures of which are incorporated herein by reference).

For example, the said polypeptide may be fused to a polypeptide such as glutathione-S-transferase (GST) or 10 protein A in order to facilitate purification of said polypeptide. Examples of such GST fusions are well known to those skilled in the art. Similarly, the said polypeptide may be fused to an oligo-histidine tag such as His6 or to an epitope recognised by an antibody such as the well-known Myc tag epitope. Fusions to any fragment, variant or derivative of said polypeptide are also included in the scope of the invention. It will be appreciated that fusions (or variants or derivatives thereof) which retain desirable properties, e.g., antigenic activity, are preferred. It is also particularly preferred if the fusions are ones which are suitable for use in the methods described herein.

For example, the fusion may comprise a further portion which confers a desirable feature on the said polypeptide of the invention; for example, the portion may be useful in detecting or isolating the polypeptide, promoting cellular uptake of the polypeptide, or directing secretion of the protein from a cell. The portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the polypeptide, as known to those skilled in the art.

It will be appreciated by persons skilled in the art that the polypeptides of the invention also include pharmaceutically acceptable acid or base addition salts of the herein described polypeptides. The acids which are used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically acceptable base addition salts may also be used to produce pharmaceutically acceptable salt forms of the polypeptides. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

The polypeptide, or fragment, variant, fusion or derivative thereof, may also be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of activity loss and that use levels may have to be adjusted upward to compensate. Preferably, the lyophilised (freeze dried) polypeptide loses no more than about 20%, or no more than about 25%, or no more than about 30%, or no more than about 35%, or no more than about 40%, or no more than about 45%, or no more than about 50% of its activity (prior to lyophilisation) when rehydrated.

Alternatively or additionally, the polypeptide of the invention is selected from the group consisting of SEQ ID NOs: 25, 26 and 27. Alternatively or additionally, the polypeptide of the invention is SEQ ID NO: 25.

Alternatively or additionally, a polypeptide of Group B exhibits at least 60% sequence identity to an amino acid sequence listed in (Group A), for example, at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity to an amino acid sequence of Group A;
(i) wherein the at least 60% sequence identity is exhibited over at least 60% of the amino acid sequence listed in (Group A), for example, a contiguous amino acid sequence spanning at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the amino acid sequence of Group A; or
(ii) wherein the at least 60% sequence identity is exhibited over at least 10 contiguous amino acids of the amino acid sequence listed in (Group A), for example, at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 350, 400, 450, 500, 550, 600, 650, 700, 750, 800, 850, 900, 950, 1000, 1500, 2000, 2500, 2600, 2601, 2602, 2603, 2604, 2605, 2606, 2607, 2608, 2609, 2610, 2611, 2612, 2613, 2614, 2615, 2616, 2617, 2618 or 2619 contiguous amino acids of the amino acid sequence of Group A.

Alternatively or additionally, the polypeptide comprises or consists of a fragment comprising at least 10 contiguous amino acids, for example, at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113,114,115, 116,117, 118,119,120,121, 122, 123,124,125, 126,127,128,129,130,131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233 contiguous amino acids, and/or, where present, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 350, 400, 450, 500, 550, 600, 650, 700, 750, 800, 850, 900, 950, 1000, 1500, 2000, 2500 or 2600 contiguous amino acids of an amino acid sequence selected from Group A.

Alternatively or additionally, the polypeptide comprises or consists of a fragment wherein 1, or at least, 1 amino acid, is truncated from the N-terminus with respect to an amino acid sequence listed in Group A, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 amino acids are truncated from the N-terminus with respect to the amino acid sequence listed in Group A

Alternatively or additionally, the polypeptide comprises or consists of a fragment wherein 1, or at least 1 amino acid, is truncated from the C-terminus with respect to the reference sequence, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 amino acids are truncated from the C-terminus with respect to the reference sequence.

Alternatively or additionally, the polypeptide is fused to one or more additional polypeptide, for example:
(a) N-terminal fusion;
(b) C-terminal fusion; or
(c) N-terminal and C-terminal fusions.

Alternatively or additionally, the polypeptide is conjugated to one or more additional moiety, for example:
(a) one or more lipid (to form a lipoprotein);
(b) one or more saccharide or polysaccharide (to form a glycoprotein);
(c) one or more phosphate group (to form a phosphoprotein);
(d) one or more heme group (to form a hemoprotein);
(e) one or more the flavin adenine dinucleotide (FAD) or flavin mononucleotide (FMN) (to form a flavoprotein); and
(f) one or more metal ion cofactor (to form a metalloprotein).

The conjugation may be by means of one or more ionic or covalent bond, but is preferably conjugation by covalent bond.

Conjugation of antigens to carriers and the like is an established procedure for improving immunogenicity, particularly for polysaccharides. For instance, bacterial capsular polysaccharides (CPS) are naturally T cell independent antigens which give rise to an immune response that lacks several important properties. Conjugation to a carrier converts these saccharides to T-cell dependent antigens which can then produce an immunological memory effect, and also elicit a protective immune responses in young children. One known carrier in such conjugates is the ‘OMPC’ outer membrane protein complex, formed from N. meningitidis vesicles (e.g. see EP-0467714), which has been included as the carrier in approved H. influenzae B conjugate vaccines. OMPC has also been used as the carrier in protein conjugates. For example, Wu et al. (PNAS USA 2006; 103(48): 18243-18248) report that conjugation of Pfs25H to OMPC resulted in a Pfs25H-OMPC conjugate vaccine that was >1,000 times more potent in generating anti-Pfs25H ELISA reactivity in mice than a similar dose of Pfs25H alone. Conjugation of OMPC to Pfs25H protein can be achieved by reacting maleimide-activated Pfs25H with thiolated outer membrane proteins within OMPC (see e.g. WO2006/124712).

Polypeptides of the invention are preferably provided in purified or substantially purified form i.e., substantially free from other polypeptides (e.g. free from naturally-occurring polypeptides), particularly from other E. coli or host cell polypeptides, and are generally at least about 50% pure (by weight), for example at least 70%, 80%, 90%, 95%, 96%, 97%, 98% 99%, 99.5%, 99.5% or 100% pure by weight (i.e., less than 50% of a composition is made up of other expressed polypeptides). Thus, the antigens in the compositions are separated from the whole organism with which the antigen molecule is expressed.

Alternatively or additionally, the polypeptide is not naturally occurring. Alternatively or additionally, the polypeptide is recombinant (i.e., expressed from nucleic acid [e.g., a gene] that has been inserted or edited using recombinant nucleic acid technology).

Alternatively or additionally, the polypeptide is a fragment, variant, fusion and/or derivative capable of 10 inducing a specific immune response to an amino acid sequence listed in Group A.

By “specific immune response” to an amino acid sequence or polypeptide listed in Group A, we mean or include the capability to induce an immune response in a subject that generates (e.g., stimulates the release of) antibody capable of binding to an amino acid sequence listed in Group A. It is preferred that the binding 15 moiety is capable of binding in vivo, i.e., under the physiological conditions in which the amino acid sequence or polypeptide exists on or inside of a subject's body. Such binding specificity may be determined by methods well known in the art, such as e.g. ELISA, immunohistochemistry, immunoprecipitation, Western blots and flow cytometry using transfected cells expressing the/a polypeptide of the invention (e.g., any one of SEQ ID NOs: 25-28) (see the accompanying Examples).

In another embodiment, the binding moiety is capable of binding selectively to amino acid sequences or polypeptides of Group A and/or Group B. By ‘capable of binding selectively’ we mean and/or include such binding moieties which bind at least 10-fold more strongly to said polypeptide or amino acid sequence than to another protein (i.e., proteins that are not variants or fragments of the test protein as claimed, or as defined herein); for example at least 50-fold more strongly or at least 100-fold more strongly. The binding moiety may be capable of binding selectively said polypeptide or amino acid sequence under physiological conditions, e.g. in vivo. Suitable methods for measuring relative binding strengths include immunoassays, for example where the binding moiety is an antibody (see Harlow & Lane, “Antibodies: A Laboratory”, Cold Spring Habor Laboratory Press, New York, which is incorporated herein by reference). Alternatively, binding may be assessed using competitive assays or using BIACORE analysis (Biacore International AB, Sweden).

Alternatively or additionally, the immune response is an immune-activating response, for example, a protective immune response. The polypeptide may be capable of eliciting an in vitro protective immune response and/or an in vivo protective immune response when administered to a subject.

In the presence of co-stimulatory signals, T cells differentiate into specific phenotypic subtypes. Several of these subtypes are involved in suppressing or terminating natural inflammatory signals. By “immune-activating response” we mean and/or include that polypeptide induces or is capable of inducing an immune response in a subject that does not result in suppressing or terminating inflammation or inflammatory signals and, preferably, results in the activation or enhancement of inflammation or inflammatory signals (e.g., cytokines).

The in vivo protective immune response may be elicited in a mammal. Alternatively or additionally, the mammal is selected from the group consisting of armadillo (Dasypus novemcinctus), baboon (Papio anubis; Papio cynocephalus), camel (Camelus bactrianus, Camelus dromedarius, Camelus ferus), cat (Felis catus), dog (Canis lupus familiaris), horse (Equus ferus caballus), ferret (Mustela putorius furo), goat (Capra aegagrus hircus), guinea pig (Cavia porcellus), golden hamster (Mesocricetus auratus), kangeroo (Macropus rufus), llama (Lama glama), mouse (Mus musculus), pig (Sus scrofa domesticus), rabbit (Oryctolagus cuniculus), rat (Rattus norvegicus), rhesus macaque (Macaca mulatta), sheep (Ovis aries), non-human primates, and human (Homo sapiens).

Alternatively or additionally, the protective immune response is protective against a disease or condition caused by an organism selected from the group consisting of: bacteria, Gram negative bacteria; proteobacteria, enterobacteriales, enterobacteriaceae (for example, Salmonella, Escherichia (E. albertii, E. coli, E. fergusonii, E. hermannii, E. marmotae, and E. vulneris), Yersinia, Klebsiella, Proteus, Enterobacter, Serratia, and Citrobacter).

Alternatively or additionally, the disease or condition is caused, wholly or in part, by Escherichia co/i, for example, extraintestinal pathogenic E. coli (ExPEC), or intestinal pathogenic E. coli (InPEC).

Alternatively or additionally, the Escherichia col is from a pathotype selected from the group consisting of: (i) enteropathogenic E. coli (EPEC); (ii) enterohemorrhagic E. coli (EHEC); (iii) enterotoxigenic E. coli (ETEC); (iv) enteroaggregative E. coli (EAEC); (v) diffusely adherent E. coli (DAEC); (vi) enteroinvasive E. coli (EIEC); (vii) uropathogenic E. coli (UPEC); (viii) neonatal meningitis E. coli (NMEC); (ix) Shiga Toxin (Stx) producing enteroaggregative E. coli (STEAEC); (x) adherent Invasive E. coli (AIEC); (xi) amoxicillin-resistant E. coli (AREC); (xii) asymptomatic bacteriuria E. coli (ABU); (xiii) Avian pathogenic E. coli (APEC).

Alternatively or additionally, the Escherichia coli is an enterohaemorrhagic E. coli (EHEC) selected from the group consisting of: O157:H7 e.g., EHEC O157:H7 EDL933 strain; EHEC O157:H7 Sakai stain; EHEC 026:H11 (e.g., strain 11368); EHEC 0103:H2 (e.g., strain 12009); and EHEC O111:H− (e.g., strain 11128).

Alternatively or additionally, the Escherichia coli is an enteropathogenic E. coli (EPEC) selected from the group consisting of: 055:H7 (e.g., CB9615); and 0127:H6 (e.g., strain E2348/69).

Alternatively or additionally, the Escherichia coli is an enterotoxigenic E. coli (ETEC) selected from the group consisting of: H10407; E24377A; and Porcine ETEC.

Alternatively or additionally, the Escherichia coli is an adherent Invasive E. coli (AIEC) selected from the group consisting of: LF82; 083:H1 NR G857C; and UM146.

Alternatively or additionally, the Escherichia coli is an enteroaggregative E. coli (EAEC), for example, 042 or 55989.

Alternatively or additionally, the Escherichia coli is a neonatal meningitis E. coli (NMEC) selected from the group consisting of: O7:K1 CE10; 588; and 1H E3034.

Alternatively or additionally, the Escherichia coli is an uropathogenic E. coli (UPEC) selected from the group consisting of: UMN026, CLONEDi14; CLONE Di2; CFT073; IA139; 536; NA114; and UT189.

Alternatively or additionally, the Escherichia coli is AREC SMS-3-5; APEC 01; or ABU 83972.

The acidic capsular polysaccharide (CPS) is a thick, mucous-like, layer of polysaccharide that surrounds some pathogenic E. coli, and is referred to as ‘K-antigen’. There are two separate groups of K-antigen groups, named group I and group II (while a small in-between subset (K3, K10, and K54/K96) has been classified as group III). The former (I) consist of 100 kDa (large) capsular polysaccharides, while the latter (II), associated with extraintestinal diseases, are under 50 kDa in size. Group I K antigens are only found with certain O-antigens (O8, O9, O20, and O101 groups), and are further subdivided on the basis of absence (IA, similar to that of Klebsiella species in structure) or presence (IB) of amino sugars. Some group I K-antigens are attached to the lipid A-core of the lipopolysaccharide (KLPS), in a similar way to 0 antigens (and being structurally identical to O antigens in some instances are only considered as K antigens when co-expressed with another authentic O antigen). Group II K antigens closely resemble those in Gram-positive bacteria and greatly differ in composition and are further subdivided according to their acidic components, generally 20-50% of the CPS chains are bound to phospholipids. In total there are 60 different K antigens that have been recognised.

Alternatively or additionally, the Escherichia coli is a strain with a K antigen selected from the group consisting of K1, K2a/ac, K3, K4, K5, K6, K7 (=K56), K8, K9 (=O104), K10, K11, K12 (K82), K13 (=K20 and =K23), K14, K15, K16, K18a, K18ab (=K22), K19, K24, K26, K27, K28, K29, K30, K31, K34, K37, K39, K40, K41, K42, K43, K44, K45, K46, K47, K49 (046), K50, K51, K52, K53, K54 (=K96), K55, K74, K84, K85ab/ac (=O141), K87 (=O32), K92, K93, K95, K97, K98, K100, K101, K102, K103, KX104, KX105, and KX106).

The outer membrane of an E. coli cell contains millions of lipopolysaccharide (LPS) molecules, which comprise:
O antigen, a polymer of immunogenic repeating oligosaccharides (1-40 units);
Core region of phosphorylated nonrepeating oligosaccharides; and
Lipid A (endotoxin).

The O antigen is used for serotyping E. coli and these O group designations go from O1 to O181, with the exception of some groups which have been historically removed, namely O31, O47, O67, O72, O93 (now K84), O94, and O122; groups 174 to 181 are provisional (O174=OX3 and O175=OX7) or are under investigation (176 to 181 are STEC/VTEC). Additionally subtypes exist for many O groups (e.g. O128ab and O128ac).

A repetitive glycan polymer contained within an LPS is referred to as the O antigen, O polysaccharide, or O side-chain of the bacteria. The O antigen is attached to the core oligosaccharide, and comprises the outermost domain of the LPS molecule. The composition of the O chain varies from strain to strain. For example, there are over 160 different O antigen structures produced by different E. coli strains. O antigen is exposed on the very outer surface of the bacterial cell, and, as a consequence, is a target for recognition by host antibodies.

Alternatively or additionally, the Escherichia coli is a strain with an O antigen selected from the group consisting of O1 A, O1 A1, O1 B, O1 C, O2, O3, O4, O4, O5 ab, O5 ac, O6, O6, O6, O7, O8, O9, O9 a, O10, O11, O12, O13, O15, O16, O16, O16, O17, O18A, O18A1, O18A1A2, O18ab, O18ac, O18B, O18B1, O19ab, O20 ab, O20 ac, O21, O22, O23 A, O24, O25, O26, O27, O28 ab, O28 ac, O29, O30, O32, O33, O34, O35, O36, O37, O38, O39, O40, O41, O42, O43, O44, O45, O45 rel, O46, O48, O49, O50, O51, O52, O53, O54, O55, O55, O56, O58, O59, O60, O61, O62, O63, O64, O65, O66, O68, O69, O70, O71, O73, O73 ab, O74, O75, O76, O77, O78, O79, O80, O81, O82, O83, O84, O85, O86, O86, O86, O87, O88, O89, O90, O91, O92, O95, O96, O97, O98, O99, O100, O101, O102, O103, O104, O105, O106, O107, O108, O109, O110, O111, O112 ab, O112 ac, O113, O114, O115, O116, O117, O118, O119, O120, O121, O123, O124, O125ab, O125ac, O126, O126, O127, O128 ab, O128ab, O128ac, O129, O130, O131, O132, O133, O134, O135, O136, O137, O138, O139, O140, O141, O142, O143, O144, O145, O146, O147, O148, O149, O150, O151, O152, O153, O154, O155, O156, O157, O158 ab, O158 ac, O159, O160, O161, O163, O164, O165, O166, O167, O168, O169, O170, O171, O172, O173, O174 ab, O174 ac, O175, O176, O177, O178, O179, O180, O181, O182, O183, O184, O185, O186 and O187.

The H antigen is a major component of flagella, involved in E. coli movement. It is generally encoded by the fliC gene. There are 53 identified H antigens, numbered from H1 to H56 (H13 and H22 were not E. coli antigens but from Citrobacter freundii, and H50 was found to be the same as H10).

Alternatively or additionally, the Escherichia coli is a strain with a H antigen selected from the group consisting of: H1, H2, H3, H4, H5, H6, H7, H8, H9, H10 (=H50), H11, H12, H14, H15, H16, H17, H18, H19, H20, H21, H23, H24, H25, H26, H27, H28, H29, H30, H31, H32, H33, H34, H35, H36, H37, H38, H39, H40, H41, H42, H43, H44, H45, H46, H47, H48, H49, H50 (=H10), H51, H52, H53, H54, H55 and H56.

Alternatively or additionally, the protective immune response is protective against a disease or condition selected from the group consisting of: gastroenteritis; urinary tract infection; neonatal meningitis; haemorrhagic colitis; and Crohn's disease.

Alternatively or additionally, the protective immune response is protective against one or more symptom of bacterial (e.g., EHEC) infection. Alternatively or additionally, the one or more symptom of bacterial infection is selected from the group consisting of diarrhoea (e.g., non-bloody), abdominal cramps and/or pain, fever, vomiting and haemolytic uremic syndrome (HUS). Alternatively or additionally, the one or more symptom of bacterial infection is prevented, abolished or reduced, for example, reduced by at least by at least 5%, for example, at least 10%, 15%, 20%, 25%, 30%, 35%, 40%, 45%, 50%, 55%, 60%, 65%, 70%, 75%, 80%, 85%, 90%, 95%, 96%, 97%, 98%, 99% or at least 100%. Diarrhoea severity may be measured by evacuation volume and/or frequency. Abdominal cramps and/or pain severity may be measured by frequency and/or by the abdominal pain index (see Van Slyke et al., 1997, Development and validation of the pain response inventory for children, Psychological Assessment, 9:392-405, which is incorporated by reference herein). Vomiting severity may be measured by evacuation volume and/or frequency. HUS severity may be measured by thrombocyte (platelet) count and/or erythrocyte (red blood cell) count (i.e., reduced HUS severity correlates with increased thrombocyte and/or erythrocyte counts).

Alternatively or additionally, the protective immune response is an immune response that results in an increase in the level of one or more serum cytokine; for example, one or more cytokines selected from the group consisting of IL-1α, IL-1β, IL-1RA, IL-18, IL-2, IL-4, IL-7, IL-9, IL-13, IL-15, IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β1, TGF-β2, TGF-β3, Epo, Tpo, Flt-3L, SCF, M-CSF and MSP. The one or more cytokine is preferably a pro-inflammatory cytokines. The one or more cytokine may be measured using any suitable method known to the skilled person, for example, ELISA, or the Bio-Plex 200 system Luminex xMAP technology (Bio-Rad, USA), Meso Scale Discovery multiplex assay (Meso Scale Diagnostics, LLC, USA), TNF-α quantification; or equivalent (e.g., see Thorpe et al., 1992, Detection and measurement of cytokines, Blood Rev. 6(3):133-48, which is incorporated by reference herein).

Alternatively or additionally, the protective immune response is an immune response that results in, or is capable of resulting in bactericidal activity and/or opsonophagocytosis. Serum bactericidal activity may be measured, for example, by the method of Crokaert et al., 1988, Determination of serum bactericidal activity against Escherichia coli by an automated photometric method, J. Clin. Microbiol., 1988 October; 26(10): 2069-2076, or the method of Necchi, Saul and Rondini, 2017, Development of a high-throughput method to evaluate serum bactericidal activity using bacterial ATP measurement as survival readout, PLoS One, 12(2):e0172163, both of which are incorporated by reference herein. Opsonophagocytosis may be measured by the method of Abbanat et al., 2017, Development and Qualification of an Opsonophagocytic Killing Assay To Assess Immunogenicity of a Bioconjugated Escherichia coli Vaccine., Clin. Vaccine. Immunol., 24(12). pii: e00123-17 which is incorporated by reference herein, Phagocytosis assay; Phagolysosome formation assay (e.g., see Ogawa et al., 2017, Activation of IpxR gene through enterohaemorrhagic Escherichia coli virulence regulators mediates lipid A modification to attenuate innate immune response, Cell Microbiol., doi: 10.1111/cmi.12806, which is incorporated by reference herein).

Alternatively or additionally, the bactericidal activity and/or opsonophagocytosis reduces, or is capable of reducing, colony forming units (CFUs):
(a) by at least 1 log, for example, reduced by at least 2, 3, 4, 5, 6, 7, 8, 9 or 10 logs; or
(b) by at least 5%, for example at least 10%, 15%, 20%, 25%, 30%, 35%, 40%, 45%, 50%, 55%, 60%, 65%, 70%, 75%, 80%, 85%, 90%, 95%, 96%, 97%, 98%, 99% or at least 100%.

CFUs can be measured using any suitable means known to the skilled person (e.g., Miller, 1972. In: Experiments in Molecular Genetics. Miller J H, editor. New York: Cold Spring Harbor; Determination of viable cell counts: bacterial growth curves; pp. 31-36).

### Adjuvants

Vaccines and immunogenic compositions of the invention may also comprise an adjuvant in addition to the antigen. Adjuvants are used in vaccines in order to enhance and modulate the immune response to the antigen. The adjuvants described herein may be combined with any of the antigen(s) herein described.

The adjuvant may be any adjuvant known to the skilled person, but adjuvants include (but are not limited to) oil-in-water emulsions (for example MF59 or AS03), liposomes, saponins, TLR2 agonists, TLR3 agonists, TLR4 agonists, TLR5 agonists, TLR6 agonists, TLR7 agonists, TLR8 agonists, TLR9 agonists, aluminium salts, nanoparticles, microparticles, ISCOMS, calcium fluoride and organic compound composites or combinations thereof.

### Oil-in-Water Emulsions

In an embodiment of the present invention, there is provided a vaccine or immunogenic composition for use in the invention comprising an oil-in-water emulsion. Oil-in-water emulsions of the present invention comprise a metabolisable oil and an emulsifying agent. In order for any oil-in-water composition to be suitable for human administration, the oil phase of the emulsion system has to comprise a metabolisable oil. The meaning of the term metabolisable oil is well known in the art. Metabolisable can be defined as “being capable of being transformed by metabolism” (Dorland's Illustrated Medical Dictionary, W. B. Sanders Company, 25th edition, 1974). A particularly suitable metabolisable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in an oil-in-water emulsion of the invention. Squalene is a metabolisable oil by virtue of the fact that it is an intermediate in the biosynthesis of cholesterol (Merck index, 10th Edition, entry no. 8619). In some embodiments, wherein the vaccines or immunogenic compositions of the invention comprise an oil-in-water emulsion, the metabolisable oil is present in the vaccine or in the immunogenic composition in an amount of 0.5% to 10% (v/v) of the total volume of the composition. The oil-in-water emulsion further comprises an emulsifying agent. The emulsifying agent may suitably be polyoxyethylene sorbitan monooleate (POLYSORBATE 80). Further, said emulsifying agent is suitably present in the vaccine or immunogenic composition in an amount of 0.125 to 4% (v/v) of the total volume of the composition. The oil-in-water emulsion may optionally comprise a tocol. Tocols are well known in the art and are described in EP0382271 B1. Suitably, the tocol may be alpha-tocopherol or a derivative thereof such as alpha-tocopherol succinate (also known as vitamin E succinate). Said tocol is suitably present in the adjuvant composition in an amount of 0.25% to 10% (v/v) of the total volume of the immunogenic composition. The oil-in-water emulsion may also optionally comprise sorbitan trioleate (SPAN 85).

The method of producing oil-in-water emulsions is well known to the person skilled in the art. Commonly, the method comprises mixing the oil phase (optionally comprising a tocol) with a surfactant such as a PBS/TWEEN80 solution, followed by homogenisation using a homogenizer; it would be clear to a person skilled in the art that a method comprising passing the mixture twice through a syringe needle is suitable for homogenising small volumes of liquid. Equally, the emulsification process in microfluidiser (e.g., M110S Microfluidics machine, maximum of 50 passes, for a period of 2 minutes at maximum pressure input of 6 bar (output pressure of about 850 bar)) could be adapted by the person skilled in the art to produce smaller or larger volumes of emulsion. The adaptation could be achieved by routine experimentation comprising the measurement of the resultant emulsion until a preparation was achieved with oil droplets of the required diameter.

In an oil-in-water emulsion, the oil and emulsifier should be in an aqueous carrier. The aqueous carrier may be, for example, phosphate buffered saline or citrate.

In particular, the oil-in-water emulsion systems used in the present invention have a small oil droplet size in the sub-micron range. Suitably the droplet sizes will be in the range 120 to 750 nm, more particularly sizes from 120 to 600 nm in diameter. Even more particularly, the oil-in water emulsion contains oil droplets of which at least 70% by intensity are less than 500 nm in diameter, more particular at least 80% by intensity are less than 300 nm in diameter, more particular at least 90% by intensity are in the range of 120 to 200 nm in diameter.

The oil droplet size, i.e. diameter, according to the present invention is given by intensity. There are several ways of determining the diameter of the oil droplet size by intensity. Intensity is measured by use of a sizing instrument, suitably by dynamic light scattering such as the Malvern Zetasizer 4000 or preferably the Malvern Zetasizer 3000HS. A first possibility is to determine the z average diameter ZAD by dynamic light scattering (PCS-Photon correlation spectroscopy); this method additionally gives the polydispersity index (PDI), and both the ZAD and PDI are calculated with the cumulants algorithm. These values do not require the knowledge of the particle refractive index. A second means is to calculate the diameter of the oil droplet by determining the whole particle size distribution by another algorithm, either the Contin, or NNLS, or the automatic “Malvern” one (the default algorithm provided for by the sizing instrument). Most of the time, as the particle refractive index of a complex composition is unknown, only the intensity distribution is taken into consideration, and if necessary the intensity mean originating from this distribution.

### ISCOMs

In some embodiments of the present invention, there are provided vaccines or immunogenic compositions of the invention comprising ISCOMs. ISCOMs are well known in the art (see Kersten & Crommelin, 1995, Biochimica et Biophysica Acta 1241: 117-138). ISCOMs comprise a saponin, cholesterol and phospholipids and form an open-cage-like structure of typically about 40 nm in size. ISCOMs result from the interaction of saponins, cholesterol and further phospholipids. A typical reaction mixture for the preparation of ISCOM is 5 mg/ml saponin and 1 mg/ml each for cholesterol and phospholipid. Phospholipids suitable for use in ISCOMs include, but are not limited, to phosphocholine (didecanoyl-L-α-phosphatidylcholine [DDPC], dilauroylphosphatidylcholine [DLPC], dimyristoylphosphatidylcholine [DMPC], dipalmitoyl phosphatidylcholine [DPPC], Distearoyl phosphatidylcholine [DSPC], Dioleoyl phosphatidylcholine [DOPC], 1-palmitoyl, 2-oleoylphosphatidylcholine [POPC], Dielaidoyl phosphatidylcholine [DEPC]), phosphoglycerol (1,2-Dimyristoyl-sn-glycero-3-phosphoglycerol [DMPG], 1,2-dipalmitoyl-sn-glycero-3-phosphoglycerol [DPPG], 1,2-distearoyl-sn-glycero-3-phosphoglycerol [DSPG], 1-palmitoyl-2-oleoyl-sn-glycero-3-phosphoglycerol [POPG]), phosphatidic acid (1,2-dimyristoyl-sn-glycero-3-phosphatidic acid [DMPA], dipalmitoyl phosphatidic acid [DPPA], distearoyl-phosphatidic acid [DSPA]), phosphoethanolamine (1,2-dimyristoyl-sn-glycero-3-phosphoethanolamine [DMPE], 1,2-Dipalmitoyl-sn-glycero-3-phosphoethanolamine [DPPE], 1,2-distearoyl-sn-glycero-3-phosphoethanolamine DSPE 1,2-Dioleoyl-sn-Glycero-3-Phosphoethanolamine [DOPE]), phoshoserine, polyethylene glycol [PEG] phospholipid (mPEG-phospholipid, polyglycerin-phospholipid, functionalized-phospholipid, terminal activated-phosholipid). In particular embodiments, ISCOMs comprise 1-palmitoyl-2-oleoyl-glycero-3-phosphoethanolamine. In further particular embodiments, highly purified phosphatidylcholine is used and can be selected from the group consisting of: Phosphatidylcholine (from egg), Phosphatidylcholine Hydrogenated (from egg) Phosphatidylcholine (from soy), Phosphatidylcholine Hydrogenated (from soy). In further particular embodiments, ISCOMs comprise phosphatidylethanolamine [POPE] or a derivative thereof. A number of saponins are suitable for use in ISCOMs. The adjuvant and haemolytic activity of individual saponins has been extensively studied in the art. For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit. Rev. Ther. Drug. Carrier Syst., 1996, 12 (1-2): 1-55; and EP0362279 B1. ISCOMs comprising fractions of Quil A have been used in the manufacture of vaccines (EP0109942 B1). These structures have been reported to have adjuvant activity (EP0109942 B1; WO 96/11711). Fractions of QuilA, derivatives of QuilA and/or combinations thereof are suitable saponin preparations for use in ISCOMs. The haemolytic saponins QS21 and QS17 (HPLC purified fractions of Quil A) have been described as potent adjuvants, and the method of their production is disclosed in U.S. Pat. No. 5,057,540 and EP0362279 B1. Also described in these references is the use of QS7 (a non-haemolytic fraction of Quil-A) which acts as a potent adjuvant for systemic vaccines. Use of QS21 is further described in Kensil et al. (1991. J. Immunology vol 146, 431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711 and these are incorporated herein. Other particular QuilA fractions designated QH-A, QH-B, QH-C and a mixture of QH-A and QH-C designated QH-703 are disclosed in WO 96/011711 in the form of ISCOMs and are incorporated herein.

### Microparticles

In some embodiments of the present invention, there is provided a vaccine or immunogenic composition of the invention comprising microparticles. Microparticles, compositions comprising microparticles, and methods of producing microparticles are well known in the art (see Singh et al. [2007 Expert Rev. Vaccines 6(5): 797-808] and WO 98/033487). The term “microparticle” as used herein, refers to a particle of about 10 nm to about 10,000 μm in diameter or length, derived from polymeric materials which have a variety of molecular weights and, in the case of the copolymers such as PLG, a variety of lactide:glycolide ratios. In particular, the microparticles will be of a diameter that permits parenteral administration to a subject without occluding the administrating device and/or the subject's capillaries. Microparticles are also known as microspheres. Microparticle size is readily determined by techniques well known in the art, such as photon correlation spectroscopy, laser diffractometry and/or scanning electron microscopy. Microparticles for use herein will be formed from materials that are sterilizable, non-toxic and biodegradable. Such materials include, without limitation, poly(a-hydroxy acid), polyhydroxybutyric acid, polycaprolactone, polyorthoester, polyanhydride.

### Liposomes

In some embodiments of the present invention, there is provided a vaccine or immunogenic composition of the invention comprising liposomes. The term “liposomes” generally refers to uni- or multilamellar (particularly 2, 3, 4, 5, 6, 7, 8, 9, or 10 lamellar depending on the number of lipid membranes formed) lipid structures enclosing an aqueous interior. Liposomes and liposome formulations are well known in the art. Lipids, which are capable of forming liposomes, include all substances having fatty or fat-like properties. Lipids which can make up the lipids in the liposomes can be selected from the group comprising of glycerides, glycerophospholipides, glycerophosphinolipids, glycerophosphonolipids, sulfolipids, sphingolipids, phospholipids, isoprenolides, steroids, stearines, sterols, archeolipids, synthetic cationic lipids and carbohydrate containing lipids. Liposome size may vary from 30 nm to several μm depending on the phospholipid composition and the method used for their preparation. In particular embodiments of the invention, the liposome size will be in the range of 50 nm to 500 nm, and in further embodiments, 50 nm to 200 nm. Dynamic laser light scattering is a method used to measure the size of liposomes well known to those skilled in the art. The liposomes suitably contain a neutral lipid, for example phosphatidylcholine, which is suitably non-crystalline at room temperature, for example egg yolk phosphatidylcholine, dioleoyl phosphatidylcholine (DOPC) or dilauryl phosphatidylcholine. In a particular embodiment, the liposomes of the present invention contain DOPC. The liposomes may also contain a charged lipid which increases the stability of the liposome-saponin structure for liposomes composed of saturated lipids. In these cases the amount of charged lipid is suitably 1 to 20% (w/w), preferably 5 to 10%. The ratio of sterol to phospholipid is 1 to 50% (mol/mol), suitably 20 to 25% (mol/mol).

### Saponins

In some embodiments of the invention, the vaccine or immunogenic composition of the invention comprises a saponin. A particularly suitable saponin for use in the present invention is Quil A and its derivatives. Quil A is a saponin preparation isolated from the South American tree Quillaja Saponaria Molina and was first described by Dalsgaard et al. in 1974 (“Saponin adjuvants”, Archiv. für die gesamte Virusforschung, Vol. 44, Springer Verlag, Berlin, p 243-254) to have adjuvant activity. Purified fragments of Quil A have been isolated by HPLC which retain adjuvant activity without the toxicity associated with Quil A (EP0362278), for example QS7 and QS21 (also known as QA7 and QA21). QS-21 is a natural saponin derived from the bark of Quillaja saponaria Molina, which induces CD8+ cytotoxic T cells (CTLs), Th1 cells and a predominant IgG2a antibody response and is a particular saponin in the context of the present invention. The saponin adjuvant within the immunogenic compositions of the invention in particular are immunologically active fractions of Quil A, such as QS-7 or QS-21, suitably QS-21. In particular embodiments, the vaccines and/or immunogenic compositions of the invention contain the immunologically active saponin fraction in substantially pure form. In particular, the vaccines or immunogenic compositions of the invention contain 0521 in substantially pure form, that is to say, the QS21 is at least 75%, 80%, 85%, 90% pure, for example at least 95% pure, or at least 98% pure.

In a particular embodiment, Q521 is provided with an exogenous sterol, such as cholesterol for example. Suitable sterols include β-sitosterol, stigmasterol, ergosterol, ergocalciferol and cholesterol. In a further particular embodiment, the adjuvant composition comprises cholesterol as sterol. These sterols are well known in the art, for example cholesterol is disclosed in the Merck Index, 11th Edition, page 341, as a naturally occurring sterol found in animal fat.

In one embodiment, the liposomes of the invention that comprise a saponin suitably contain a neutral lipid, for example phosphatidylcholine, which is suitably non-crystalline at room temperature, for example egg yolk phosphatidylcholine, dioleoyl phosphatidylcholine (DOPC) or dilauryl phosphatidylcholine. The liposomes may also contain a charged lipid which increases the stability of the liposome-Q521 structure for liposomes composed of saturated lipids. In these cases the amount of charged lipid is suitably 1 to 20% (w/w), particularly 5 to 10% (w/w). The ratio of sterol to phospholipid is 1 to 50% (mol/mol), suitably 20 to 25% (mol/mol).

Where the active saponin fraction is QS21, the ratio of Q521:sterol will typically be in the order of 1:100 to 1:1 (w/w), suitably between 1:10 to 1:1 (w/w), and preferably 1:5 to 1:1 (w/w). Suitably, excess sterol is present, the ratio of QS21:sterol being at least 1:2 (w/w). In one embodiment, the ratio of QS21:sterol is 1:5 (w/w). The sterol is suitably cholesterol.

Other useful saponins are derived from the plants Aesculus hippocastanum or Gyophilla struthium. Other saponins which have been described in the literature include Escin, which has been described in the Merck index (12^{th} Edition: entry 3737) as a mixture of saponins occurring in the seed of the horse chestnut tree, Lat: Aesculus hippocastanum. Its isolation is described by chromatography and purification (Fiedler, Arzneimittel-Forsch. 4, 213 (1953)), and by ion-exchange resins (Erbring et al., U.S. Pat. No. 3,238,190). Fractions of Escin have been purified and shown to be biologically active (Yoshikawa et al., 1996, Chem Pharm Bull (Tokyo), 44(8): 1454-1464). Sapoalbin from Gypsophilla struthium (R. Vochten et al., 1968, J. Pharm. Belg. 42: p 213-226) has also been described in relation to ISCOM production for example.

A saponin, such as QS21, can be used at amounts between 1 and 100 μg per human dose of the adjuvant composition. QS21 may be used at a level of about 50 μg, for example between 40 to 60 μg, suitably between 45 to 55 μg or between 49 and 51 μg or 50 μg. In a further embodiment, the human dose of the adjuvant composition comprises QS21 at a level of about 25 μg, for example between 20 to 30 μg, suitably between 21 to 29 μg or between 22 to 28 μg or between 28 and 27 μg or between 24 and 26 μg, or 25 μg.

### TLR4 Agonist

In some embodiments, the vaccine or immunogenic composition of the invention comprises a TLR4 agonist. By “TLR agonist” it is meant a component which is capable of causing a signalling response through a TLR signalling pathway, either as a direct ligand or indirectly through generation of endogenous or exogenous ligand (Sabroe et al, 2003, JI p 1630-5). A TLR4 agonist is capable of causing a signalling response through a TLR-4 signalling pathway. A suitable example of a TLR-4 agonist is a lipopolysaccharide, suitably a non-toxic derivative of lipid A, particularly monophosphoryl lipid A or more particularly 3-Deacylated monophoshoryl lipid A (3D-MPL).

3D-MPL is sold under the name MPL by GlaxoSmithKline Biologicals and is referred throughout the document as MPL or 3D-MPL See, for example, U.S. Pat. Nos. 4,436,727; 4,877,611; 4,866,034 and 4,912,094. 3D-MPL primarily promotes CD4+ T cell responses with an IFN-gamma (Th1) phenotype. 3D-MPL can be produced according to the methods disclosed in GB 2 220 211 A. Chemically, it is a mixture of 3-deacylated monophosphoryl lipid A with 4, 5 or 6 acylated chains. In the compositions of the present invention, small particle 3D-MPL may be used to prepare the aqueous adjuvant composition. Small particle 3D-MPL has a particle size such that it may be sterile-filtered through a 0.22 μm filter. Such preparations are described in WO 94/21292. Preferably, powdered 3D-MPL is used to prepare the aqueous adjuvant compositions of the present invention.

Other TLR-4 agonists which can be used are alkyl glucosaminide phosphates (AGPs) such as those disclosed in WO 98/50399 or U.S. Pat. No. 6,303,347 (processes for preparation of AGPs are also disclosed), suitably RC527 or RC529 or pharmaceutically acceptable salts of AGPs as disclosed in U.S. Pat. No. 6,764,840.

Other suitable TLR-4 agonists are as described in WO 03/011223 and in WO 03/099195, such as compound I, compound II and compound Ill disclosed on pages 4-5 of WO 03/011223 or on pages 3 to 4 of WO 03/099195 and in particular those compounds disclosed in WO 03/011223, as ER803022, ER803058, ER803732, ER804053, ER804057m ER804058, ER804059, ER804442, ER804680 and ER804764. For example, one suitable TLR-4 agonist is ER804057.

A TLR-4 agonist, such as a lipopolysaccharide, such as 3D-MPL, can be used at amounts between 1 and 100 μg per human dose of the adjuvant composition. 3D-MPL may be used at a level of about 50 μg. for example between 40 to 60 μg, suitably between 45 to 55 μg or between 49 to 51 μg or 50 μg per human dose. In a further embodiment, the human dose of the adjuvant composition comprises 3D-MPL at a level of about 25 μg, for example between 20 to 30 μg, suitably between 21 to 29 μg or between 22 to 28 μg or between 28 to 27 μg or between 24 to 26 μg, or 25 μg.

Synthetic derivatives of lipid A are known and thought to be TLR 4 agonists including, but not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)
OM294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO 99/64301 and WO 00/0462)
OM197 MP-Ac DP (3S-,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127).

Other suitable TLR-4 ligands, capable of causing a signalling response through TLR-4 (Sabroe et al, JI 2003 p 1630-5) are, for example, lipopolysaccharide from gram-negative bacteria and its derivatives, or fragments thereof, in particular a non-toxic derivative of LPS (such as 3D-MPL). Other suitable TLR agonist are: heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2, muramyl dipeptide (MDP) or F protein of respiratory syncytial virus (RSV). In one embodiment, the TLR agonist is HSP 60, 70 or 90.

### TLR Agonists

Rather than a TLR4 agonist, other natural or synthetic agonists of TLR molecules may be used in vaccines or immunogenic composition of the invention. These include, but are not limited to, agonists for TLR2, TLR3, TLR5, TLR6,TLR7,TLR8 and TLR9.

In one embodiment of the present invention, a TLR agonist is used that is capable of causing a signalling response through TLR-1 (Sabroe et al, JI 2003 p 1630-5). Suitably, the TLR agonist capable of causing a signalling response through TLR-1 is selected from: Tri-acylated lipopeptides (LPs); phenol-soluble modulin; Mycobacterium tuberculosis LP; S-(2,3-bis(palmitoyloxy)-(2-RS)-propyl)-N-palmitoyl-(R)-Cys-(S)-Ser-(S)-Lys(4)-OH, trihydrochloride (Pam3Cys) LP which mimics the acetylated amino terminus of a bacterial lipoprotein and OspA LP from Borrelia burgdorferi.

In a further embodiment, a TLR agonist is used that is capable of causing a signalling response through TLR-2 (Sabroe et al, JI 2003 p 1630-5). Suitably, the TLR agonist capable of causing a signalling response through TLR-2 is one or more of a lipoprotein, a peptidoglycan, a bacterial lipopeptide from M. tuberculosis, B. burgdorferi, T. pallidum, peptidoglycans from species including Staphylococcus aureus, lipoteichoic acids, mannuronic acids, Neisseria porins, bacterial fimbriae, Yersinia virulence factors, CMV virions, measles haemagglutinin, and zymosan from yeast.

In a further embodiment, a TLR agonist is used that is capable of causing a signalling response through TLR-3 (Sabroe et al, JI 2003 p 1630-5). Suitably, the TLR agonist capable of causing a signalling response through TLR-3 is double stranded RNA (dsRNA), or polyinosinic-polycytidylic acid (Poly IC), a molecular nucleic acid pattern associated with viral infection.

In an alternative embodiment, a TLR agonist is used that is capable of causing a signalling response through TLR-5 (Sabroe et al, JI 2003 p 1630-5). Suitably, the TLR agonist capable of causing a signalling response through TLR-5 is bacterial flagellin. Said TLR-5 agonist may be flagellin or may be a fragment of flagellin which retains TLR-5 agonist activity. The flagellin can include a polypeptide selected from the group consisting of H. pylori, S. typhimurium, V. cholera, S. marcesens, S. flexneri, T. pallidum, L. pneumophilia, B. burgdorferei; C. difficile, R. meliloti, A. tumefaciens; R. lupine; B. clarridgeiae, P. mirabilis, B. subtilus, L. moncytogenes, P. aeruginoa and E. coli.

In a particular embodiment, the flagellin is selected from the group consisting of S. typhimurium flagellin B (Genbank Accession number AF045151), a fragment of S. typhimurium flagellin B, E. coli FliC. (Genbank Accession number AB028476); fragment of E. coli FliC; S. typhimurium flagellin FliC (ATCC14028) and a fragment of S. typhimurium flagellin FliC

In a further particular embodiment, said TLR-5 agonist is a truncated flagellin, as described in WO 09/156405 i.e. one in which the hypervariable domain has been deleted. In one aspect of this embodiment, said TLR-5 agonist is selected from the group consisting of: FliC_{Δ174.400}; FliC_{Δ161.405} and FliC_{Δ138-405}.

In a further particular embodiment, said TLR-5 agonist is a flagellin, as described in WO 09/128950.

In a further embodiment, a TLR agonist is used that is capable of causing a signalling response through TLR-6 (Sabroe et al, JI 2003 p 1630-5). Suitably, the TLR agonist capable of causing a signalling response through TLR-6 is mycobacterial lipoprotein, di-acylated LP, and phenol-soluble modulin. Further TLR6 agonists are described in WO 03/043572.

In a further embodiment, a TLR agonist is used that is capable of causing a signalling response through TLR-7 (Sabroe et al, JI 2003 p 1630-5). Suitably, the TLR agonist capable of causing a signalling response through TLR-7 is a single stranded RNA (ssRNA), loxoribine, a guanosine analogue at positions N7 and C8, or an imidazoquinoline compound, or derivative thereof. In a particular embodiment, the TLR agonist is imiquimod. Further TLR7 agonists are described in WO 02/085905.

In a further embodiment, a TLR agonist is used that is capable of causing a signalling response through TLR-8 (Sabroe et al, JI 2003 p 1630-5). Suitably, the TLR agonist capable of causing a signalling response through TLR-8 is a single stranded RNA (ssRNA), an imidazoquinoline molecule with anti-viral activity, for example resiquimod (R848); resiquimod is also capable of recognition by TLR-7. Other TLR-8 agonists which may be used include those described in WO 04/071459.

In a further embodiment, a TLR agonist is used that is capable of causing a signalling response, such as one that comprises a CpG motif. The term “immunostimulatory oligonucleotide” is used herein to mean an oligonucleotide that is capable of activating a component of the immune system. In one embodiment, the immunostimulatory oligonucleotide comprises one or more unmethylated cytosine-guanosine (CpG) motifs. In a further embodiment, the immunostimulatory oligonucleotide comprises one or more unmethylated thymidine-guanosine (TG) motif or may be T-rich. By T-rich, it is meant that the nucleotide composition of the oligonucleotide comprises greater than 50, 60, 70 or 80% thymidine. In one embodiment, the oligonucleotide is not an immunostimulatory oligonucleotide and does not comprise an unmethylated CpG motif. In a further embodiment the immunostimulatory oligonucleotide is not T-rich and/or does not comprise an unmethylated TG motif.

The oligonucleotide may be modified in order to improve in vitro and/or in vivo stability. For example, in one embodiment, the oligonucleotides are modified so as to comprise a phosphorothioate backbone, i.e. internucleotide linkages. Other suitable modifications including diphosphorothioate, phosphoroamidate and methylphosphonate modifications as well as alternative internucleotide linkages to oligonucleotides are well known to those skilled in the art and are encompassed by the invention.

In another embodiment, the vaccines or immunogenic compositions of the invention further comprise an immunostimulant selected from the group consisting of: a TLR-1 agonist, a TLR-2 agonist, TLR-3 agonist, a TLR-4 agonist, a TLR-5 agonist, a TLR-6 agonist, a TLR-7 agonist, a TLR-8 agonist, TLR-9 agonist, or a combination thereof.

### Calcium Composites

In some embodiments, the vaccine or immunogenic composition of the invention comprises a calcium fluoride composite, the composite comprising Ca, F, and Z. “Z” as used herein refers to an organic molecule. As used herein, a “composite” is a material that exists as a solid when dry, and that is insoluble, or poorly soluble, in pure water. In some aspects, Z comprises a functional group that forms an anion when ionized. Such functional groups include without limitation one or more functional groups selected from the group consisting of: hydroxyl, hydroxylate, hydroxo, oxo, N-hydroxylate, hydroaxamate, N-oxide, bicarbonate, carbonate, carboxylate, fatty acid, thiolate, organic phosphate, dihydrogenophosphate, monohydrogenophosphate, monoesters of phosphoric acid, diesters of phosphoric acid, esters of phospholipid, phosphorothioate, sulphates, hydrogen sulphates, enolate, ascorbate, phosphoascorbate, phenolate, and imine-olates.

In some aspects, the calcium fluoride composites herein described comprise Z, where Z is an anionic organic molecule possessing an affinity for calcium and forming a water insoluble composite with calcium and fluoride. In further aspects, the calcium fluoride composites herein described comprise Z, where Z may be categorized as comprising a member of a chemical category selected from the group consisting of: hydroxyl, hydroxylates, hydroxo, oxo, N-hydroxylate, hydroaxamate, N-oxide, bicarbonates, carbonates, carboxylates and dicarboxylate, salts of carboxylic-acids, salts of QS21, extract of bark of Quillaja saponaria, extract of immunological active saponine, salts of satured or unsaturated fatty acid, salts of oleic acid, salts of amino-acids, thiolates, thiolactate, salt of thiol-compounds, salts of cysteine, salts of N-acetyl-cysteine, L-2-Oxo-4-thiazolidinecarboxylate, phosphates, dihydrogenophosphates, monohydrogenophosphate, salts of phosphoric-acids, monoesters of phosphoric acids and their salts, diesters of phosphoric acids and their salts, esters of 3-O-desacyl-4′-monophophoryl lipid A, esters of 3D-MLA, MPL, esters of phospholipids, DOPC, dioleolyphosphatidic derivatives, phosphates from CpG motifs, phosphorothioates from CpG family, sulphates, hydrogen sulphates, salts of sulphuric acids, enolates, ascorbates, phosphoascorbate, phenolate, α-tocopherol, imine-olates, cytosine, methyl-cytosine, uracyl, thymine, barbituric acid, hypoxanthine, inosine, guanine, guanosine, 8-oxo-adenine, xanthine, uric acid, pteroic acid, pteroylglutamic acid, folic acid, riboflavin, and lumiflavin. In further aspects, the calcium fluoride composites herein described comprise Z, where Z is selected from the group consisting of: N-acetyl cysteine; thiolactate; adipate; carbonate; folic acid; glutathione; and uric acid. In some aspects, the calcium fluoride composites herein comprise Z, where Z is selected from the group consisting of: N-acetyl cysteine; adipate; carbonate; and folic acid. In further aspects, the calcium fluoride composites herein comprise Z, where Z is N-acetyl cysteine, and the composite comprises between 51% Ca, 48% F, no more than 1% N-acetyl cysteine (w/w) and 37% Ca, 26% F, and 37% N-acetyl cysteine (w/w). In further aspects, the calcium fluoride composites herein comprise Z, where Z is thiolactate, and the composite comprises between 51% Ca, 48% F, no more than 1% thiolactate (w/w) and 42% Ca, 30% F, 28% thiolactate (w/w). In further aspects, the calcium fluoride composites herein comprise Z, where Z is adipate, and the composite comprises between 51% Ca, 48% F, no more than 1% adipate (w/w) and 38% Ca, 27% F, 35% adipate (w/w). In further aspects, the calcium fluoride composites herein comprise Z, where Z is carbonate, and the composite comprises between 51% Ca, 48% F, no more than 1% carbonate (w/w) and 48% Ca, 34% F, 18% carbonate (w/w). In further aspects, the calcium fluoride composites herein comprise Z, where Z is folic acid, and the composite comprises between 51% Ca, 48% F, no more than 1% folic acid (w/w) and 22% Ca, 16% F, 62% folic acid (w/w). In further aspects, the calcium fluoride composites herein comprise Z, where Z is glutathione, and the composite comprises between 51% Ca, 48% F, no more than 1% glutathione (w/w) and 28% Ca, 20% F, 52% glutathione (w/w). In further aspects, the calcium fluoride composites herein comprise Z, where Z is uric acid, and the composite comprises between 51% Ca, 48% F, and no more than 1% uric acid (w/w) and 36% Ca, 26% F, and 38% uric acid (w/w).

### Aluminium Salts

In one embodiment, the vaccine or immunogenic composition of the invention comprises an aluminium salt. Suitable aluminium salt adjuvants are well known to the skilled person and include but are not limited to aluminium phosphate, aluminium hydroxide or a combination thereof. Suitable aluminium salt adjuvants include but are not limited to REHYDRAGEL HS, ALHYDROGEL 85, REHYDRAGEL PM, REHYDRAGEL AB, REHYDRAGEL HPA, REHYDRAGEL LV, ALHYDROGEL or a combination thereof.

In particular, the aluminium salts may have a protein adsorption capacity of between 2.5 and 3.5, 2.6 and 3.4, 2.7 and 3.3 or 2.9 and 3.2, 2.5 and 3.7, 2.6 and 3.6, 2.7 and 3.5, or 2.8 and 3.4 protein (BSA)/ml aluminium salt. In a particular embodiment of the invention, the aluminium salt has a protein adsorption capacity of between 2.9 and 3.2 mg BSA/mg aluminium salt. Protein adsorption capacity of the aluminium salt can be measured by any means known to the skilled person. The protein adsorption capacity of the aluminium salt may be measured using the method as described in Example 1 of WO 12/136823 (which utilises BSA) or variations thereof.

Aluminium salts described herein (i.e. having the protein adsorption capacity described herein) may have a crystal size of between 2.8 and 5.7 nm as measured by X-ray diffraction, for example 2.9 to 5.6 nm, 2.8 to 3.5 nm, 2.9 to 3.4 nm or 3.4 to 5.6 nm or 3.3 and 5.7 nm as measured by X-ray diffraction. X-ray diffraction is well known to the skilled person. In a particular embodiment of the invention the crystal size is measured using the method described in Example 1 of WO 12/136823 or variations thereof.

The polypeptide(s) and/or nucleic acid(s) described herein may be administered to a subject by any route of administration, for example, orally, nasally, sublingually, intravenously, intramuscularly, intradermally (e.g. a skin patch with microprojections) or transdermally (e.g. an ointment or cream).

A second aspect of the invention provides an isolated nucleic acid molecule comprising or consisting of:
(Group C) a nucleic acid sequence selected from the group consisting of selected from the group consisting of SEQ ID NO: 1, 2, 3, 4, 5, 6, 7, 8, 9, 10, 11, 12, 13, 14, 15, 16, 17, 18, 19, 20, 21, 22, 23 or 24; or
(Group D) a fragment, variant and/or fusion of (Group C).

Alternatively or additionally, wherein the nucleic acid sequence of Group C is selected from the group consisting of SEQ ID NOs: 1, 2 and 3, for example, the nucleic acid sequence may be SEQ ID NO: 1.

Alternatively or additionally, the nucleic acid of Group D exhibits at least 60% sequence identity to a nucleic acid sequence listed in Group C, for example, at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence listed in (Group C); and
(I) wherein the at least 60% sequence identity is exhibited over at least 60% of the nucleic acid sequence listed in (Group C), for example, a contiguous amino acid sequence spanning at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the nucleic acid sequence listed in (Group C); or
(II) wherein the at least 60% sequence identity is exhibited over at least 30 contiguous nucleic acids of the nucleic acid sequence listed in (Group C), for example, at least 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, or 7500 contiguous amino acids of the nucleic acid sequence listed in (Group C).

Alternatively or additionally, the nucleic acid encodes a polypeptide according to the first aspect of the invention.

The terms “polynucleotide” and “oligonucleotide” are used interchangeably herein, and refer to a heteropolymer of nucleotides or the sequence of these nucleotides. These phrases also refer to DNA or RNA of genomic or synthetic origin which may be single-stranded or double-stranded and may represent the sense or the antisense strand, to peptide nucleic acid (PNA) or to any DNA-like or RNA-like material. In the sequences herein A is adenine, C is cytosine, T is thymine, G is guanine and N is A, C, G or T (U). Where the polynucleotide is RNA, the T (thymine) in the sequences provided herein is substituted with U (uracil). Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

A third aspect of the invention provides a vector comprising a nucleic acid molecule as defined in the second aspect of the invention.

Suitable prokaryotic vector plasmids are: pUC18, pUC19, pBR322 and pBR329 available from Biorad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA), and pBAD-A (Thermo Fisher).

A suitable mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems (La Jolla, Calif. 92037, USA). Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

Methods well known to those skilled in the art can be used to construct expression vectors containing the coding sequence and, for example appropriate transcriptional or translational controls. One such method involves ligation via homopolymer tails. Homopolymer polydA (or polydC) tails are added to exposed 3′ OH groups on the DNA fragment to be cloned by terminal deoxynucleotidyl transferases. The fragment is then capable of annealing to the polydT (or polydG) tails added to the ends of a linearised plasmid vector. Gaps left following annealing can be filled by DNA polymerase and the free ends joined by DNA ligase.

Another method involves ligation via cohesive ends. Compatible cohesive ends can be generated on the DNA fragment and vector by the action of suitable restriction enzymes. These ends will rapidly anneal through complementary base pairing and remaining nicks can be closed by the action of DNA ligase.

A further method uses synthetic molecules called linkers and adaptors. DNA fragments with blunt ends are generated by bacteriophage T4 DNA polymerase or E. coli DNA polymerase I which remove protruding 3′ termini and fill in recessed 3′ ends. Synthetic linkers, pieces of blunt-ended double-stranded DNA which contain recognition sequences for defined restriction enzymes, can be ligated to blunt-ended DNA fragments by T4 DNA ligase. They are subsequently digested with appropriate restriction enzymes to create cohesive ends and ligated to an expression vector with compatible termini. Adaptors are also chemically synthesised DNA fragments which contain one blunt end used for ligation but which also possess one preformed cohesive end.

Synthetic linkers containing a variety of restriction endonuclease sites are commercially available from a number of sources including International Biotechnologies Inc, New Haven, Conn., USA.

A desirable way to modify the DNA encoding the polypeptide of the invention is to use the polymerase chain reaction as disclosed by Saiki et al (1988) Science 239, 487-491. In this method the DNA to be enzymatically amplified is flanked by two specific oligonucleotide primers which themselves become incorporated into the amplified DNA. The said specific primers may contain restriction endonuclease recognition sites which can be used for cloning into expression vectors using methods known in the art.

### Self-Replicating RNA

Nucleic acid immunisation may be achieved by delivering a self-replicating RNA (or self-amplifying RNA) encapsulated within and/or adsorbed to a small particle. The RNA encodes a polypeptide antigen of interest, and the particle may deliver this RNA by mimicking the delivery function of a natural virus. After in vivo administration of the particles, RNA is released from the particles and is translated inside a cell to provide the antigen in situ.

Any of the polypeptide antigens described herein as suitable to be included in the vaccines or immunogenic compositions in accordance with the invention may be expressed in the form of a self-replicating RNA molecule encoding said antigen, as described in WO 12/006376 which is incorporated herein by reference. Accordingly, in particular embodiments where antigens in the vaccines or immunogenic compositions for use in the invention are polypeptides, such polypeptides are encoded by a self-replicating RNA. In such cases, said self-replicating RNA is suitably coupled with a delivery system, in particular lipid-based delivery systems, such as a cationic nanoemulsion (CNE), or a liposome. Suitably, when the lipid-based system is a CNE the self-replicating RNA is adsorbed to the outer surface of the CNE, while when said lipid-based system is a liposome the self-amplifyfing RNA is encapsulated into the liposome.

By “self-replicating RNA molecule” (or “self-amplifying RNA”), it is meant that, when delivered to a vertebrate cell, even without any proteins, the molecule leads to the production of multiple daughter RNAs by transcription from itself as explained in WO 12/006376, which ultimately results into the expression of the encoded antigen, becoming a major polypeptide of the cells.

One suitable system for achieving self-replication in this manner is to use an alphavirus-based replicon, as further described in WO 12/006376. Suitably, said replicon encodes (i) a RNA-dependent RNA polymerase which can transcribe RNA from the self-replicating RNA molecule and (ii) an antigen of interest. The polymerase can be an alphavirus replicase e.g. comprising one or more alphavirus proteins nsP1, nsP2, nsP3 and nsP4. Appropriate features of self-replicating RNA molecules and methods for preparing them are also described in WO 12/006376.

In some embodiments, the vaccines or immunogenic compositions for use in the present invention comprise a liposome and a self-replicating RNA encoding any of the polypeptide antigens herein described encapsulated into the liposome. In further embodiments, the vaccines or immunogenic compositions for use in the present invention comprise a CNE and a self-replicating RNA encoding any of the polypeptide antigens herein described adsorbed to the outer surface of the CNE. In particular embodiments, the self-replicating RNA molecule encodes polypeptide antigens derived from the group consisting of: HCMV, RSV and HIV.

Exemplary CNE for use in the present invention, as well as methods for their preparation are disclosed in WO 12/006380 which is incorporated herein by reference.

Various amphiphilic lipids can form bilayers in an aqueous environment to encapsulate a RNA-containing aqueous core as a liposome. These lipids can have an anionic, cationic or zwitterionic hydrophilic head group. Some phospholipids are anionic whereas others are zwitterionic and others are cationic. Suitable classes of phospholipids include, but are not limited to, phosphatidylethanolamines, phosphatidylcholines, phosphatidylserines, and phosphatidyl-glycerols. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,N dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Further useful cationic lipids are described in WO 15/095340, for example the lipids as claimed in any of claims 1 to 8 of WO 15/095340 incorporated herein by reference. Zwitterionic lipids include, but are not limited to, acyl zwitterionic lipids and ether zwitterionic lipids. Examples of useful zwitterionic lipids are DPPC, DOPC and dodecylphosphocholine. Uposomal particles of the invention can be formed from a single lipid or from a mixture of lipids. A mixture may comprise (i) a mixture of anionic lipids, (ii) a mixture of cationic lipids, (iii) a mixture of zwitterionic lipids, (iv) a mixture of anionic lipids and cationic lipids, (v) a mixture of anionic lipids and zwitterionic lipids, (vi) a mixture of zwitterionic lipids and cationic lipids or (vii) a mixture of anionic lipids, cationic lipids and zwitterionic lipids. Where a mixture of lipids is used, not all of the component lipids in the mixture need to be amphiphilic e.g. one or more amphiphilic lipids can be mixed with cholesterol. The hydrophilic portion of a lipid can be PEGylated (i.e. modified by covalent attachment of a polyethylene glycol). This modification can increase stability and prevent non-specific adsorption of the liposomes. Liposomal particles are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core; SUVs typically have a diameter 550 nm, and LUVs have a diameter>50 nm. Liposomal particles useful in this aspect of the invention are ideally LUVs with a diameter in the range of 50-220 nm. Techniques for preparing suitable liposomes are well known in the art. One useful method is described in Jeffs et al. (Pharmaceutical Research, 2005, 22(3): 362-372) and involves mixing (i) an ethanolic solution of the lipids (ii) an aqueous solution of the nucleic acid and (iii) buffer, followed by mixing, equilibration, dilution and purification.

### Viral Vectors

Alternatively, nucleic acid immunisation may be achieved by using a replicating or replication-defective 5 vector, such as a viral vector. Numerous viral vectors suitable for introducing nucleic acids encoding antigens of interest into a subject are known in the art, and include both DNA and RNA viruses. Suitable examples are for instance: adenovirus vectors (replication or replication deficient), pox virus vectors, including vaccinia virus vectors, such as modified vaccinia Ankara virus (MVA), NYVAC, avipox vectors, canarypox (ALVAC) and fowl pox virus (FPV), Alphavirus vectors (such as Sindbis virus, Semlike Forest virus, Ross River virus, and Venezuelan equine encephalitis virus) and chimeras and replicons thereof, herpes virus vectors (e.g. cytomegalovirus-derived vectors), arena virus vectors, such as lymphocytic choriomeningitis virus (LCMV) retrovirus, lentivirus, viral like particles, and many others. In one embodiment, the polypeptide antigen in the vaccines or immunogenic compositions for use in the present invention is encoded by an adenoviral vector. In particular embodiments, the polypeptide antigen encoded by an adenoviral vector derives from HIV, Malaria, Ebola or RSV. The production and use of adenovirus vectors are well known to those of ordinary skill in the art. Suitable examples of disclosure of the design, production and use of adenovirus vectors can be found, for instance, in WO 05/071093, and WO 10/086189 which are incorporated herein by reference. Adenoviral vectors for use in the present invention may be derived from a range of mammalian hosts. Adenoviral vectors may be derived from a human adenovirus. Examples of such human-derived adenoviruses are Ad1, Ad2, Ad4, Ad5, Ad6, Ad11, Ad24, Ad34, Ad35, particularly Ad5, Ad11 and Ad35.

Alternatively, adenoviral vectors may be derived from a non-human primate adenovirus e.g. a chimpanzee adenovirus, such as one selected from serotypes ChAd3, ChAd63, ChAd83, ChAd155, Pan5, Pan6, Pan7 and Pan9. Specifically, the virus may be a non-human adenovirus, such as a simian adenovirus and in particular a chimpanzee adenovirus such as ChAd155, Pan 5, 6, 7 or 9. Examples of such strains are described in WO 03/000283 which is incorporated herein by reference and are available from the American Type Culture Collection, 10801 University Boulevard, Manassas, Va. 20110-2209, and other sources. Desirable chimpanzee adenovirus strains include Pan 5 [ATCC VR-591], Pan 6 [ATCC VR-592], and Pan 7 [ATCC VR-593].

The adenoviral vectors for use in the present invention may be derived from replication-defective adenovirus, for example, comprising a functional E1 deletion. Adenoviral vectors for use in the present invention include PanAd3 (WO 10/086189) and ChAd155 (GB1510357.5). In some embodiments, the antigen of the vaccines or immunogenic compositions for use in the invention is recombinantly expressed in the adenoviral vector ChAd155. The adenoviral vectors can be produced on any suitable cell line in which the virus is capable of replication. Without limitation, such a cell line may be HeLa [ATCC Accession No. CCL 2], A549 [ATCC Accession No. CCL 185], HEK 293, KB [CCL 17], Detroit [e.g., Detroit 510, CCL 72] and WI-38 [CCL 75] cells, among others.

A fourth aspect of the invention provides host cell comprising a nucleic acid molecule defined the second 5 aspect or a vector as defined in the third aspect of the invention.

Host cells that have been transformed by the nucleotide sequences or recombinant DNA of the invention are then cultured for a sufficient time and under appropriate conditions known to those skilled in the art in view of the teachings disclosed herein to permit the expression of the polypeptide, which can then be recovered.

The polypeptide of the invention can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Many expression systems are known, including (but not limited to) systems employing: bacteria (eg. E. coli and Bacillus subtilis) transformed with, for example, recombinant bacteriophage, plasmid or cosmid DNA expression vectors; yeasts (eg. Saccharomyces cerevisiae) transformed with, for example, yeast expression vectors; insect cell systems transformed with, for example, viral expression vectors (eg. baculovirus); plant cell systems transfected with, for example viral or bacterial expression vectors; animal cell systems transfected with, for example, adenovirus expression vectors.

The vectors can include a prokaryotic replicon, such as the Col E1 ori, for propagation in a prokaryote, even if the vector is to be used for expression in other, non-prokaryotic cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a nucleotide sequence or DNA segment of the present invention.

As used herein, the term “subject” means all animals including humans (e.g., mammals as defined herein). Examples of subjects include humans, non-human primates, cows, dogs, cats, goats, sheep, and pigs. The term “patient” means a subject having a disorder in need of treatment.

A fifth aspect of the invention provides a method for producing a polypeptide according to the first aspect of the invention comprising or consisting of the steps of culturing a population of host cells according to the fourth aspect of the invention under conditions in which the peptide is expressed, and isolating the peptide therefrom.

Alternatively or additionally, the polypeptide of the first aspect of the invention may be produced by in vitro translation, for example, using a nucleic acid of the first aspect of the invention or a vector of the third aspect of the invention.

In addition to the available technologies, new vaccine development strategies have been explored. These innovations ideally serve to make vaccine production simpler, more cost effective, improve antigen presentation and immune response (MacLennan and Saul, 2014). For instance, the outer membrane vesicles are one of these systems employed in the vaccine development. Naturally, Gram-negative bacteria release native outer membrane vesicles (NOMV) that are rich in outer membrane lipids, outer membrane and periplasmic proteins, representing antigens in a natural conformation (Ellis and Kuehn, 2010). Therefore, NOMV based vaccines have been largely employed against the organism from they are recovered (Nieves et al., 2014; Petersen et al., 2014) or to express and deliver heterologous antigens (Bartolini et al., 2013; Daleke-Schermerhorn et al., 2014; Fantappie et al., 2014). However, in native conditions, blebs are recovered in small quantity, thus, E. coli strains can be genetically modified by deletion the tolR gene to enhance the level of vesicle production (Bernadac et al., 1998). This system has been successfully used for expression of properly folded membrane associated recombinant antigens and to induce functional immune responses (Bartolini et al., 2013). Recently, this antigen delivery approach, also known as Generalized Modules for Membrane Antigens (GMMA), has been successfully implemented and largely used (Berlanda Scorza et al., 2012; Gerke et al., 2015; De Benedetto et al., 2017).

A sixth aspect of the invention provides a vesicle comprising one or more polypeptide as defined in the first aspect of the invention.

Alternatively or additionally, the vesicle is derived from the membrane of a cell, for example, a Gram positive bacteria membrane vesicle or a Gram negative bacteria outer membrane vesicle (OMV).

OMVs are naturally-occurring membrane vesicles that form spontaneously during bacterial growth and are released into culture medium. They can be obtained e.g. by culturing bacteria in broth culture medium, separating whole cells from the smaller OMVs in the broth culture medium (e.g. by filtration or by low-speed centrifugation to pellet only the cells and not the smaller vesicles), and then collecting the OMVs from the cell-depleted medium (e.g. by filtration, by differential precipitation or aggregation, by high-speed centrifugation to pellet the vesicles). Strains for use in production of OMVs can generally be selected on the basis of the amount of OMVs produced in culture. The present OMVs are characterised by the fact of being collected and isolated following a detergent-free procedure. Preferably, the present OMV are released into the fermentation broth and are purified using a centrifugation and subsequent filtration step (for a general reference see e.g. Clin Vaccine Immunol. 2016 April; 23(4): 304-314). Still preferably, the present OMV are released into the fermentation broth and are purified using the following two consecutive Tangential Flow Filtration (TFF) steps: (i) a microfiltration in which the culture supernatant containing the OMV is separated from the bacteria, and (ii) an ultrafiltration in which the OMV are separated from soluble proteins (for a general reference see e.g. PLoS One. 2015; 10(8): e0134478). The thus obtained OMV can then directly be used within the present invention without additional purification/isolation steps. The presently considered OMVs have a preferred size distribution comprised from 20 to 250 nm, measured by Dynamic Light Scattering DLS technique.

According to some embodiments, the OMVs are prepared from wild-type bacteria or from bacteria which have been genetically manipulated generally to increase immunogenicity (e.g. to hyper-express immunogens), to reduce toxicity, to inhibit capsular saccharide synthesis, to down-regulate immunodominant antigen expression, and the like. They also may be prepared from hyperblebbing strains. The OMVs of the invention may also express exogenous proteins on their surface and they may be endotoxin-depleted.

Preferably, the OMVs to be used in the present invention are produced from genetically-modified bacterial strains that are mutated to enhance vesicles production, and optionally also to remove or modify antigens (e.g. lipid A) and/or to over-express homologous antigens or antigens from other organisms. Said preferred OMV are also known as Generalized Modules of Membrane Antigens (GMMA) as e.g. described in PLoS One. 2015; 10(8): e0134478.

Enhanced spontaneous generation of vesicles can be achieved, for example, by targeted deletion of proteins involved in maintenance of membrane integrity. It has been observed that the outer surface of OMVs substantially corresponds to the outer surface of the bacterium from which they are derived, preserving the membrane antigens (including e.g. lipopolysaccharides, lipooligosaccharides and lipoproteins) in the context of the membrane. Advantageously, the OMVs used in the invention (unlike detergent-extracted dOMVs) retain these outer membrane components in their native conformation and correct orientation, better preserving immunogenicity against the bacterial strain from which they are derived.

Generally, the OMVs for use in the present invention may be prepared from any suitable bacterium, where preferred bacteria include, but are not limited to: Neisseria (e.g. in particular N. meningitidis of any serogroup including A, B, C, Y, X or W135, or from a non-pathogenic Neisseria), Salmonella enterica serovars (such as Salmonella Paratyphi, Salmonella Enteritidis, Salmonella Typhi or Salmonella Typhimurium), Haemophilus influenzae (e.g. non-typable H. influenzae), Vibrio cholerae, Bordetella pertussis, Mycobacterium smegmatis, Mycobacterium bovis BCG, Escherichia coli, Bacteroides (including Porphyromonas), Pseudomonas aeruginosa, Helicobacter pylori, Brucella melitensis Campylobacter jejuni, Actinobacillus actinomycetemcomitans, Xenorhabdus nematophilus, Moraxella catarrhalis, or Borrelia burgdorferi.

Particularly preferred bacteria are selected from at least one of: S. sonnei, S. flexneri, Salmonella bacterium, and meningococcus, particularly meningococcus serogroup B.

As far as Salmonella bacterium is concerned, a particularly preferred strain is selected from: Salmonella Typhimurium, Salmonella Enteritidis and Salmonella Paratyphi A.

Meningococcus bacteria OMVs are also preferred. Such vesicles can be prepared from any meningococcal strain. The vesicles are preferably prepared from a serogroup B strain, but it is also suitable to prepare them from serogroups other than B, such as one of: A, C, W135, Y or X, according to procedures known in the art.

The strain may be of any serotype (e.g. 1, 2a, 2b, 4, 14, 15, 16, etc.), any serosubtype (e.g. P1.4), and any immunotype (e.g. L1; 12; L3; L3,7; L3,7,9; L10; etc.). The meningococci may be from any suitable lineage, including hyperinvasive and hypervirulent lineages, preferably any of the following seven hypervirulent lineages: subgroup I; subgroup III; subgroup IV-1; ET-5 complex; ET-37 complex; A4 cluster; lineage 3. Most preferably, OMVs are prepared from the strain NZ98/254, or another strain with the P1.4 PorA serosubtype.

In another embodiment, bacteria for preparing OMVs useful for the invention may be mutant strains which have been manipulated e.g. to enhance vesicles production, to express one or more desired antigen(s), and/or to knockout or modify an undesired gene (e.g. one which encodes a toxin or which encodes an enzyme involved in generating a toxic product, such as endotoxin).

In this direction, other preferred OMVs for the invention are produced by a Salmonella bacterium, particularly a S. Typhimurium (also known as Salmonella enterica serovar Typhimurium) which does not express a functional TolR protein.

Where the vesicles are prepared from E. coli or Salmonella the bacterium may express no more than 4 of TolA, TolB, TolQ TolR and Pal proteins. Thus at least one protein from the natural five-protein Tol-Pal system may be absent, resulting in a bacterium which, during growth in culture medium, releases greater quantities of outer membrane vesicles into the medium than the same bacterium expressing all 5 Tol-Pal proteins. Preferably TolR is not expressed, but the other four proteins may be expressed (i.e. a ΔTolR strain).

In preferred embodiments, at least one of the five Tol-Pal proteins in E. coli or Salmonella is removed e.g. by deletion or inactivation of the gene encoding the protein. Thus the bacterium may express 0, 1, 2, 3 or 4 of TolA, TolB, TolQ, TolR and Pal proteins. Removal of one of the five proteins can suffice, in which case the bacterium expresses only 4 of these proteins. Preferably the TolR protein is removed e.g. by inactivation of a starting strain's tolR gene. Thus a preferred bacterium may be tolA+tolB+tolQ+TolR−Pal+.

In some embodiments, the bacterium expresses all five Tol-Pal proteins, but at least one is mutated to cause hyperblebbing. For instance, the TolA, TolQ, TolR and/or Pal protein may be mutated such that the protein retains its membrane localisation but its interactions with other members of the Tol-Pal system are disrupted. The bacterium will thus retain TolA, TolQ and TolR as transmembrane proteins in the inner membrane, and Pal protein as a periplasm-facing lipoprotein in the outer membrane, but at least one of the TolA, TolQ, TolR and/or Pal proteins is mutated and not fully functional.

In addition other mutations may also be present e.g. to give lipopolysaccharide O antigen (OAg) deficient strains, such as DgalU, DgalE or DwbaP in E. coli or Salmonella strains.

In one further preferred embodiment, a meningococcus does not express a functional MltA protein. Knockout of MltA (the membrane-bound lytic transglycosylase, also known as GNA33) in meningococcus provides bacteria which spontaneously release large amounts of OMVs into culture medium, from which they can be readily purified. For instance, the vesicles can be purified using the two stage size filtration process, comprising: (i) a first filtration step in which vesicles are separated from the bacteria based on their different sizes, with the vesicles passing into the filtrate; and (ii) a second filtration step in which the vesicles are retained in the retentate.

In the present invention, it is preferred that —OAg is present on the OMVs because it has been observed (e.g. OMVs from Salmonella) that, the presence of the —OAg on the surface of said OMVs is advantageous in providing a bivalent vaccine, as the —OAg can act as a protective antigen. Some preferred strains have penta- or tetra-acylated less toxic LPS, which includes attached —OAg, after the mutation of msbB, htrB, ddg and/or PagP (see e.g. Rossi O et al, Clin Vaccine Immunol. 2016 Apr. 4; 23(4):304-14 and Rossi O et al, J Biol Chem. 2014 Sep. 5; 289(36):24922-35).

Where the vesicles are prepared from Neisseria, the strain has preferably a modified fur gene. According to this embodiment, mutant Neisseria are engineered to reduce or switch off expression of at least one gene involved in rendering toxic the lipid A portion of LPS, in particular of IpxI1 gene. In this way, the resulting OMVs present a reduced toxicity with respect to the wild type strain, since the conversion of acylated lipid A in a less acylated form.

Similarly, preferred mutant Neisseria for the invention are engineered to reduce or switch off expression of at least one gene involved in the capsular saccharide synthesis or export, in particular of synX and/or ctrA genes. In this way, the resulting OMVs may present a cross protection versus different serotypes, particularly appreciated by the skilled in the art.

In preferred embodiments a strain may include one or more of the knockout and/or hyper-expression mutations disclosed e.g. in Fukusawa et al. (1999), Vaccine 17:2951-2958. For instance, following the therein guidance and nomenclature, useful genes for down-regulation and/or knockout include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB; (b) CtrA, CtrB, CtrC, CtrD, FrpB, GalE, HtrB/MsbB, LbpA, LbpB, LpxK, Opa, Opc, PhoP, PilC, PmrE, PmrF, SiaA, SiaB, SiaC, SiaD, TbpA, and/orTbpB; (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB; or (d) CtrA, CtrB, CtrD, FrpB, OpA, OpC, PilC, PorB, SiaD, SynA, SynB, SynX and/or SynC.

Alternatively or additionally, the vesicle is a Gram negative bacteria outer membrane vesicle (OMV) of a bacterium defined in the first aspect of the invention.

Alternatively or additionally, the polypeptide is:
(a) homologous to the cell; or
(b) heterologous to the cell.

Alternatively or additionally, the polypeptide is:
(a) located in the lumen of the vesicle (for example, where the vesicle is a cell, in the cytoplasm);
(b) located in the/a inter-membrane space of the vesicle (for example, where the vesicle is a cell, the periplasm]); and/or
(c) located on the outer surface of the outer membrane (for example, where the vesicle is a cell, the outer membrane).

Alternatively or additionally, the polypeptide is expressed by the cell.

Alternatively or additionally, the polypeptide is chemically conjugated to the vesicle (for example, to a lipid, protein or polysaccharide component of the vesicle membrane). Hence, the invention may comprise vesicle conjugates prepared by a process comprising the steps of:
i) reacting at least a vesicle surface protein or lipopolysaccharide residue with the first terminal portion of a divalent Linker to obtain a vesicle-linker derivative; and
ii) connecting the vesicle-linker derivative of (i) to one or more polypeptide or nucleic acid of the invention via the second terminal portion of the divalent Linker, thus obtaining a vesicle-linker-antigen.

The linker may be a divalent homobifunctional linker, i.e. having the same first and second terminal functionalities or a divalent heterobifunctional linker, i.e. having different terminal functionalities.

A seventh aspect of the invention provides a binding moiety capable of specifically binding to one or more polypeptide defined in the first aspect or a nucleic acid defined in the second aspect of the invention.

As used herein, the term “binding moiety” includes a region or regions of the agent of the invention capable of reversibly and/or irreversibly associating with a region or regions of another molecule or molecules by covalent and/or ionic interaction.

Alternatively or additionally, the binding moiety comprises an antibody or an antigen-binding fragment thereof, or a variant, fusion or derivative of said antibody or an antigen-binding fragment, or a fusion of a said variant or derivative thereof, which retains the binding specificity for a polypeptide of the invention (e.g., any one of SEQ ID Nos: 25-48).

By “antibody” we include substantially intact antibody molecules, as well as chimaeric antibodies, humanised antibodies, human antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, and antigen binding fragments and derivatives of the same.

For example, the antibody or antigen-binding fragment, or variant, fusion or derivative thereof, may comprise, consist or consist essentially of an intact antibody. By “consist essentially of” we mean that the antibody or antigen-binding fragment, variant, fusion or derivative thereof consists of a portion of an intact antibody sufficient to retain binding specificity for a polypeptide of the invention (e.g., any one of SEQ ID NOs: 25-48).

The term ‘antibody’ also includes all classes of antibodies, including IgG, IgA, IgM, IgD and IgE. Thus, the antibody may be an IgG molecule, such as an IgG1, IgG2, IgG3, or IgG4 molecule.

Preferably, the antibody is an IgG antibody, for example, an IgG2 or IgG4 antibody.

In a preferred embodiment, the antibody, antigen-binding fragment, variant, fusion or derivative thereof is in an isolated and/or purified form.

In one embodiment, the antibody is a non-naturally occurring antibody. Where the antibody is a naturally occurring antibody, it is provided in an isolated form (i.e. separated from the milieu in which it is found in nature).

It will be appreciated by persons skilled in the art that the binding specificity of an antibody or antigen binding fragment thereof is conferred by the presence of Complementarity Determining Regions (CDRs) within the variable regions of the constituent heavy and light chains.

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent-parented antibody (Morrison et al (1984) Proc. Natl. Acad. Sci. USA 81, 6851-6855).

Antigenic specificity is conferred by variable domains and is independent of the constant domains, as known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

As used herein, the term “antigen-binding fragment” refers to a functional fragment of an antibody that is capable of binding to a polypeptide of the invention (e.g., any one of SEQ ID NOs: 25-48).

Exemplary antigen-binding fragments of the invention may be selected from the group consisting of Fv fragments (e.g. single chain Fv and disulphide-bonded Fv), and Fab-like fragments (e.g. Fab fragments, Fab′ fragments and F(ab)₂ fragments).

In a preferred embodiment, the antigen-binding fragment is an scFv.

The advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli or yeast, thus allowing the facile production of large amounts of the said fragments.

Also included within the scope of the invention are modified versions of antibodies and antigen-binding fragments thereof, e.g., modified by the covalent attachment of polyethylene glycol or other suitable polymer.

Methods of generating antibodies and antibody fragments are well known in the art. For example, antibodies may be generated via any one of several methods which employ induction of in vivo production of antibody molecules, screening of immunoglobulin libraries (Orlandi et al, 1989. Proc. Natl. Acad. Sci. U.S.A. 86:3833-3837; Winter et al., 1991, Nature 349:293-299) or generation of monoclonal antibody molecules by cell lines in culture. These include, but are not limited to, the hybridoma technique, the human B-cell hybridoma technique, and the Epstein-Barr virus (EBV)-hybridoma technique (Kohler et al., 1975. Nature 256:4950497; Kozbor et al., 1985. J. Immunol. Methods 81:31-42; Cote et al., 1983. Proc. Natl. Acad. Sci. USA 80:2026-2030; Cole et al., 1984. Mol. Cell. Biol. 62:109-120).

The antibody or antigen-binding fragment or derivative thereof may be produced by recombinant means.

Preferably, the antibody is a monoclonal antibody.

Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982), which are incorporated herein by reference.

Antibody fragments can also be obtained using methods well known in the art (see, for example, Harlow & Lane, 1988, “Antibodies: A Laboratory Manual”, Cold Spring Harbor Laboratory, New York, which is incorporated herein by reference). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods, or by cell free in vitro expression.

As defined herein, the binding moiety may be a variant, fusion or derivative thereof of an antibody or antigen-binding fragment, provided such variants, fusions and derivatives retain binding specificity for a polypeptide of the invention (e.g., any one of SEQ ID NOs: 25-48).

Variants may be made using the methods of protein engineering and site-directed mutagenesis well known in the art using the recombinant polynucleotides (see example, see Molecular Cloning: a Laboratory Manual, 3rd edition, Sambrook & Russell, 2001, Cold Spring Harbor Laboratory Press, which is incorporated herein by reference).

In other preferred embodiments, the antibody variant may be a single-domain antibody, such as a nanobody. Such antibodies are known to exist in camelids (Curr. Opin. Pharmacol., 8, (2008), 600-608) and sharks (e.g. IgNAR; Curr. Opin. Pharmacol., 8, (2008), 600-608). Other preferred antibody variants include isolated heavy-chain variable (V_{H}) regions or isolated light-chain (V_{L}) regions, for example from human antibodies (Curr. Opin. Pharmacol., 8, (2008), 600-608), and iMabs (WO 03/050283).

In a preferred embodiment, the invention provides an agent wherein the antibody, antigen-binding fragment, variant, fusion or derivative thereof is capable of competing for binding to a polypeptide of the invention (e.g., any one of SEQ ID NOs: 25-48) with an antibody molecule as defined herein, or a variant, fusion or derivative of said antibody or antigen-binding fragment, or a fusion of a said variant or derivative thereof, which retains the binding specificity for a polypeptide of the invention (e.g., any one of SEQ ID NOs: 25-48).

By “capable of competing” for binding to a polypeptide of the invention (e.g., any one of SEQ ID NOs: 24-48) with an antibody molecule as defined herein (or a variant, fusion or derivative of said antibody or antigen-binding fragment, or a fusion of a said variant or derivative thereof, which retains the binding specificity for a polypeptide of the invention (e.g., any one of SEQ ID NOs: 25-48)) we mean that the tested antibody, antigen-binding fragment, variant, fusion or derivative thereof is capable of inhibiting or otherwise interfering, at least in part, with the binding of an antibody molecule as defined herein (or a variant, fusion or derivative of said antibody or antigen-binding fragment, or a fusion of a said variant or derivative thereof).

For example, the antibody or antigen-binding fragment, variant, fusion or derivative thereof, or fusion of a said variant or derivative thereof, may be capable of inhibiting the binding of an antibody molecule defined herein by at least 10%, for example at least 20%, 30%, 40%, 50%, 60%, 70%, 80%, 90%, 35% or even by 100%.

Competitive binding may be determined by methods well known to those skilled in the art, such as ELISA (as described herein) and/or SPR (as described in the accompanying Examples).

In a “Sandwich ELISA”, a suitable amount of, e.g. a polyclonal antibody, such as 10 μg/ml of a polyclonal rabbit antibody, directed against the/a polypeptide of the invention (e.g., any one of SEQ ID NOs: 25-48) is used as a capture antibody when coated in a 96-well plate, such as e.g. MAXISORP NUNC. Coating is done according to standard procedures known in the art. Wells may be blocked for 1h with 3% bovine serum albumin (BSA) in tris-buffered saline-TWEEN 20 (TBS-T) at e.g. room temperature. Cell extract from HEK-cells stably transfected with an expression vector expressing the/a polypeptide of the invention (e.g., any one of SEQ ID NOs: 25-48), or from cells in which a/the polypeptide of the invention (e.g., any one of SEQ ID NOs: 25-48), is then diluted in assay buffer (e.g. TBS-T supplemented with 0.1% BSA, 1 mM MgCl2 and 10 μM CaCl₂). Suitable amount of diluted cell extract, such as e.g. 50 μl is then added per well and incubated to allow binding to the coated antibody, e.g. for 1h at room temperature. Plates are then washed three times with TBS-T. Primary antibody conjugated with biotin is then added in a suitable amount, such as e.g. at 2 μg/ml in assay buffer. Plates are then incubated for a sufficient time to allow binding of primary and control antibody where possible, e.g. for 1h at room temperature followed by three washes in TBS-T. In case where biotinylated primary antibodies are used, Streptavidin-HRP antibody (DAKO) may be used and diluted accordingly in assay buffer (1:5000), added to the wells. Plates are then incubated enough for the streptavidin-biotin complex to form, e.g. for 1h at room temperature. Following washing, e.g. three times with TBS-T and the plates are developed with peroxidase substrate (e.g. OPD SigmaFast, Sigma). The absorbance of the colorimetric change is determined at suitable wavelength, in this case 490 nm.

If primary antibodies are not conjugated, the same ELISA can instead be incubated with a secondary antibody against the human IgG4 directly conjugated with HRP, e.g. mouse anti-human IgG4-HRP, from e.g. Serotec, or, if not conjugated, followed by a HRP-conjugated anti-mouse antibody from e.g. DAKO. Plates are then washed and developed as outlined herein.

Further methods include reversing the sandwich ELISA outlined herein, and instead using the/a antibody against the/a polypeptide of the invention (e.g., any one of SEQ ID NOs: 25-48) as a capture antibody, as will appreciated by those in the art.

These ELISA assays can be used to evaluate epitope-modifying or blocking antibodies. Additional methods suitable for identifying competing antibodies are disclosed in Antibodies: A Laboratory Manual, Harlow & Lane, which is incorporated herein by reference (for example, see pages 567 to 569, 574 to 576, 583 and 590 to 612, 1988, CSHL, NY, ISBN 0-87969-314-2).

In a preferred embodiment, the invention provides an agent wherein the antibody, antigen-binding fragment, variant, fusion or derivative thereof is capable of binding to the same epitope as an antibody molecule as defined herein.

In an alternative embodiment, the invention provides an agent wherein the antibody, antigen-binding fragment, variant, fusion or derivative thereof is capable of binding to an epitope distinct from that to which an antibody molecule as defined herein.

As used herein, the term “epitope refers to a site of a molecule to which an antibody binds, i.e. a molecular region of an antigen. An epitope may be a linear epitope, which is determined by e.g. the amino acid sequence, i.e. the primary structure, or a three-dimensional epitope, defined by the secondary structure, e.g. folding of a peptide chain into beta sheet or alpha helical, or by the tertiary structure, e.g. way which helices or sheets are folded or arranged to give a three-dimensional structure, of an antigen.

Alternatively or additionally, the binding moiety is selected from the group consisting of: antibodies; antigen-binding fragments; and antibody mimetics. Alternatively or additionally, the binding moiety is an antibody. Alternatively or additionally, the antibody is polyclonal or monoclonal. Alternatively or additionally, the binding moiety is an antigen-binding fragment selected from the group consisting of: Fab (fragment antigen binding); F(ab′)₂; Fab′; scFv (single chain variable fragment); di-scFv; sdAb (single domain antibody/domain antibody); trifunctional antibody; chemically-linked F(ab′)₂; and BiTE (bi-specific T-cell engager). Alternatively or additionally, the antibody or antigen binding fragment thereof is an antigen binding fragment selected from the group consisting of affibodies molecules; affilins; affimers; affitins; alphabodies; anticalins; avimers; DARPins; fynomers; kunitz domain peptides; monobodies and nanoCLAMPs.

An eighth aspect of the invention provides a pharmaceutical composition comprising a polypeptide defined in the first aspect, a nucleic acid molecule defined in the second aspect, a vector as defined in the third aspect, a host cell as defined in the fourth aspect and/or a binding moiety as defined in the fifth aspect of the invention.

A ninth aspect of the invention provides a kit comprising or consisting of a polypeptide defined in the first aspect, a nucleic acid molecule defined in the second aspect, a vector as defined in the third aspect, a host cell as defined in the fourth aspect and/or a binding moiety as defined in the fifth aspect and/or a pharmaceutical composition as defined in the eighth aspect of the invention; and (optionally) instructions for use.

The antigen(s) and adjuvants may be combined extemporaneously, at the time of delivery. Thus, the invention provides kits including the antigen(s) and adjuvant ready for mixing. The kits allow the antigen(s) and adjuvant to be kept separately until the time of use.

The components may be physically separate from each other within a kit, and this separation can be achieved in various ways. For instance, the two components may be in two separate containers, such as vials. The contents of the two vials can then be mixed e.g. by removing the contents of one vial and adding them to the other vial, or by separately removing the contents of both vials and mixing them in a third container (for example vial). One or more of the kit components may be in a syringe.

The kit components may be in aqueous form. In some embodiments, a component such as the antigen(s) is in dry form (e.g., in a lyophilised form), with the other component being in aqueous form. The two components can be mixed in order to reactivate the dry component and give an aqueous composition for administration to a subject or patient. A lyophilised component will typically be located within a vial rather than a syringe.

Dried components may include stabilizers such as lactose, sucrose or mannitol, as well as mixtures thereof e.g. lactose/sucrose mixtures, sucrose/mannitol mixtures, etc. One possible arrangement uses an aqueous adjuvant component in a pre-filled syringe and a lyophilised antigen component in a vial.

A tenth aspect of the invention provides a polypeptide as defined in the first aspect, a nucleic acid molecule as defined in the second aspect, a vector as defined in the third aspect, a host cell as defined in the fourth aspect and/or a binding moiety as defined in the fifth aspect and/or a pharmaceutical composition as defined in the eighth aspect of the invention, for use in medicine.

An eleventh aspect of the invention provides a polypeptide as defined in the first aspect, a nucleic acid molecule as defined in the second aspect, a vector as defined in the third aspect, a host cell as defined in the fourth aspect and/or a binding moiety as defined in the fifth aspect and/or a pharmaceutical composition as defined in the eighth aspect of the invention, for use in preventing or treating bacterial infection and/or symptoms thereof.

Alternatively or additionally, the bacterial infection is, wholly or in part, infection with one or more bacterium defined in the first aspect of the invention.

Alternatively or additionally, the use comprises the consecutive or concurrent use of:
(a) a polypeptide defined in the first aspect;
(b) a nucleic acid molecule defined in the second aspect;
(c) a vector as defined in the third aspect;
(d) a vesicle as defined in the fourth aspect;
(e) a host cell as defined in the fifth aspect;
(f) a binding moiety as defined in the sixth aspect; and/or
(g) a pharmaceutical composition as defined in the eighth aspect of the invention.

Alternatively or additionally, the use comprises the consecutive or concurrent use of a binding moiety as defined in the sixth aspect of the invention with:
(a) a nucleic acid molecule of the second aspect and/or a vector as defined the third aspect of the invention; and/or
(b) a binding moiety as defined in the sixth aspect and/or a vesicle as defined in the fourth aspect of the invention.

An twelfth aspect of the invention provides the use of a polypeptide as defined in the first aspect, a nucleic acid molecule as defined in the second aspect, a vector as defined in the third aspect, a host cell as defined in the fourth aspect and/or a binding moiety as defined in the fifth aspect and/or a pharmaceutical composition as defined in the eighth aspect of the invention, in preventing or treating bacterial infection and/or symptoms thereof in a subject or patient, for example, wherein the bacterial infection is, wholly or in part, infection with one or more bacterium defined in the first aspect.

A thirteenth aspect of the invention provides the use of a polypeptide as defined in the first aspect, a nucleic acid molecule as defined in the second aspect, a vector as defined in the third aspect, a host cell as defined in the fourth aspect and/or a binding moiety as defined in the fifth aspect and/or a pharmaceutical composition as defined in the eighth aspect of the invention, in the manufacture of a medicament for preventing or treating bacterial infection and/or symptoms thereof, for example, wherein the bacterial infection is, wholly or in part, infection with one or more bacterium defined in the first aspect.

A fourteenth aspect of the invention provides a method for preventing or treating bacterial infection and/or symptoms thereof comprising or consisting of administering to a subject or patient an effective amount of a polypeptide as defined in the first aspect of the invention, a nucleic acid molecule as defined in the second aspect, a vector as defined in the third aspect, a host cell as defined in the fourth aspect and/or a binding moiety as defined in the fifth aspect and/or a pharmaceutical composition as defined in the eighth aspect of the invention, for example, wherein the bacterial infection is, wholly or in part, infection with one or more bacterium defined in the first aspect.

A fifteenth aspect of the invention provides the use of a nucleic acid molecule as defined in the second aspect of the invention, or a binding moiety as defined in the fifth aspect, for detecting the presence of bacteria, for example, wherein the bacteria are one or more bacterium defined in the first aspect of the invention. Alternatively or additionally, the detection is in vitro and/or in vivo.

Sequences of the antigen candidates are as follows:

| Antigen Candidate | Polynucleotide | Polypeptide |
|---|---|---|
| MC001 | SEQ ID NO: 1 | SEQ ID NO: 25 |
| MC007 | SEQ ID NO: 2 | SEQ ID NO: 26 |
| MC020 | SEQ ID NO: 3 | SEQ ID NO: 27 |
| MC002 | SEQ ID NO: 4 | SEQ ID NO: 28 |
| MC003 | SEQ ID NO: 5 | SEQ ID NO: 29 |
| MC004 | SEQ ID NO: 6 | SEQ ID NO: 30 |
| MC005 | SEQ ID NO: 7 | SEQ ID NO: 31 |
| MC006 | SEQ ID NO: 8 | SEQ ID NO: 32 |
| MC008 | SEQ ID NO: 9 | SEQ ID NO: 33 |
| MC009 | SEQ ID NO: 10 | SEQ ID NO: 34 |
| MC010 | SEQ ID NO: 11 | SEQ ID NO: 35 |
| MC011 | SEQ ID NO: 12 | SEQ ID NO: 36 |
| MC012 | SEQ ID NO: 13 | SEQ ID NO: 37 |
| MC013 | SEQ ID NO: 14 | SEQ ID NO: 38 |
| MC014 | SEQ ID NO: 15 | SEQ ID NO: 39 |
| MC015 | SEQ ID NO: 16 | SEQ ID NO: 40 |
| MC016 | SEQ ID NO: 17 | SEQ 10 NO: 41 |
| MC017 | SEQ ID NO: 18 | SEQ ID NO: 42 |
| MC018 | SEQ ID NO: 19 | SEQ ID NO: 43 |
| MC019 | SEQ ID NO: 20 | SEQ ID NO: 44 |
| MC021 | SEQ ID NO: 21 | SEQ ID NO: 45 |
| MC022 | SEQ ID NO: 22 | SEQ ID NO: 46 |
| MC023 | SEQ ID NO: 23 | SEQ ID NO: 47 |
| MC024 | SEQ ID NO: 24 | SEQ ID NO: 48 |

Preferred, non-limiting examples which embody certain aspects of the invention will now be described, with reference to the following figures:

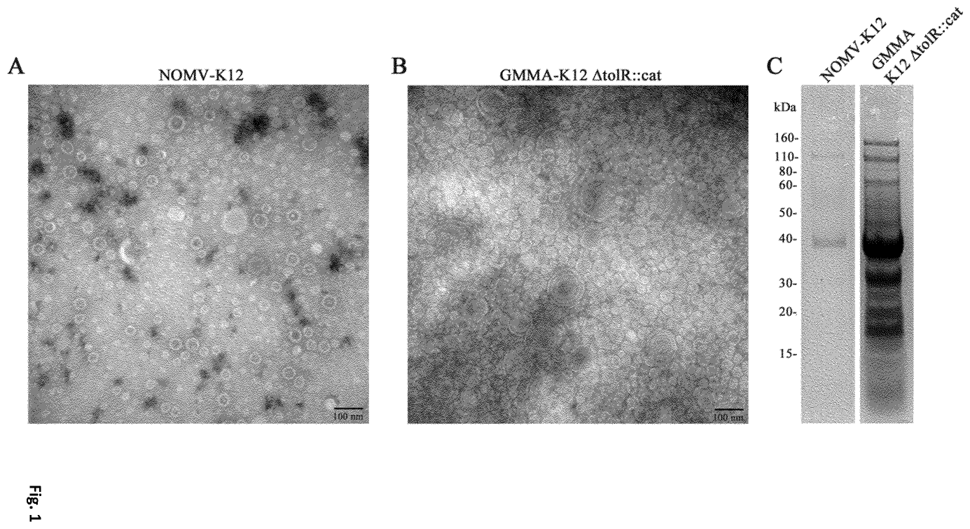

FIG. 1. E. coli K12 engineering to generate Generalized Modules for Membrane Antigens (GMMA). NOMV and GMMA were isolated by ultracentrifugation from supernatants of E. coli K12 WT and K12 ΔtolR::cat (A) Negative staining of native NOMV released from a wild-type E. coli K12 observed by transmission electron microscopy (TEM). NOMV from K12 WT strain appeared as closed spherical particles and homogeneous in shape with a size ranging from 20 to 100 nm. (B) Negative staining of GMMA produced by the K12 tolR::cat (K12tolR::cat) strain analyzed by TEM GMMA from tolR mutant were released in higher amount (size ranging from 20 to 200 nm), and some of them showed an atypical shape characterized by more than one membrane layer (magnification 120,000×). (C) SDS Page (4-12% bis-tris polyacrylamide) of membrane vesicles (NOMV and GMMA) purified from 75 ml of culture supernatants. Total protein content was quantified and 50 ug of GMMA obtained from K12 ΔtolR::cat sample was loaded into the gel. An equivalent volumetric amount of 15 NOMV from K12 WT obtained from 75 mL of supernatant was loaded and separated by SDS-PAGE. The tolR mutant showed an extensive protein profile in the supernatant compared to wild type. SDS-PAGE comparison of these preparations showed that the tolR mutant was able to yield 25-fold more vesicles than wild-type strain in terms of total protein content.

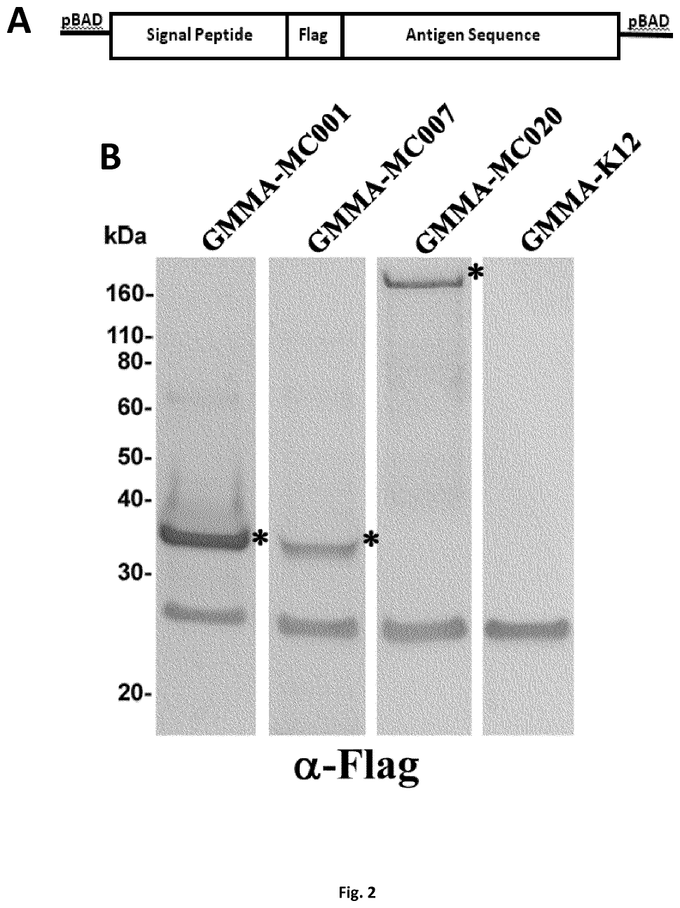

FIG. 2. Antigen delivery into GMMA. (A) Scheme of the antigens cloned into pBAD with flag-tag. The three selected candidates were cloned into a pBAD vector using their own signal peptide. A flag-tag was inserted after the signal peptide of each protein. (B) Western blot of MC001, MC007 and MC020 independently fused to a flag-tag. Each construct was independently transformed in the K12tolR::cat mutant and induced with arabinose. The western blots show the expression of the flag-fused antigens (*) on GMMA recovered from these 16h cultures, in comparison to the GMMA-K12.

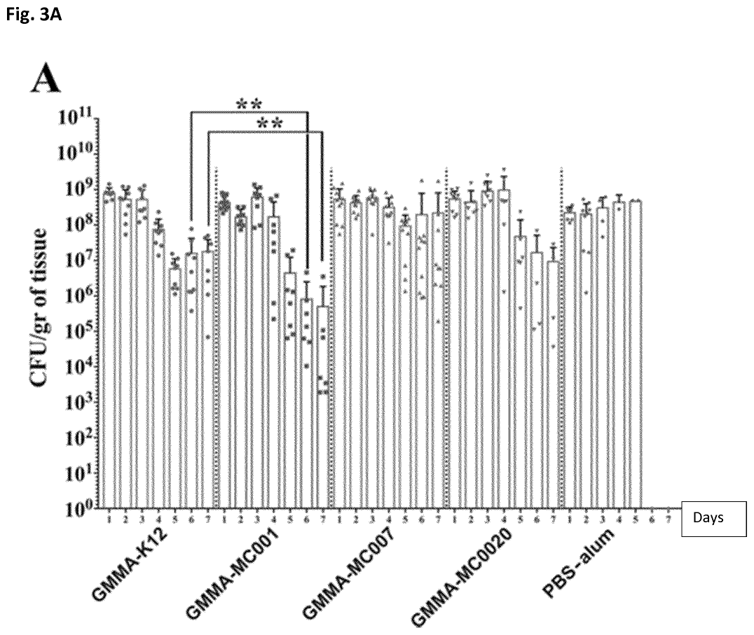

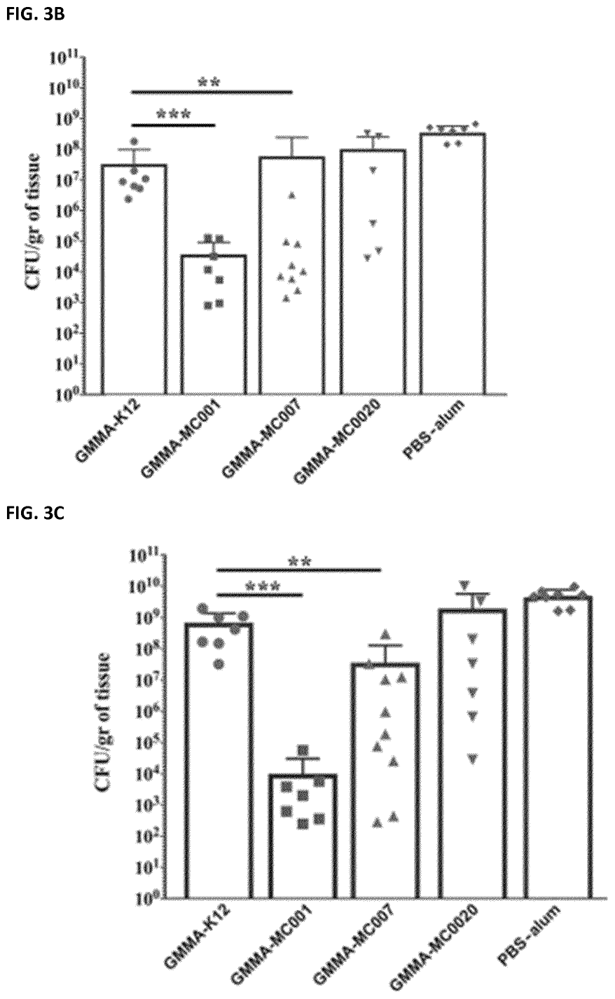

FIG. 3. Vaccination with GMMA-MC001 reduces bacteria colonization feces, colon and caecum. (A) Graphic representation of bacterial counts in feces from challenged mice at Days 1-7 post-challenge. The mice immunized with GMMA-MC001 showed significant reduction in bacterial shedding at day 6 (≈2-logs, **P value=0.0033) and day 7 (≈4-logs reduction, **P value=0.0037) compared to the mice immunized with GMMA-K12. (B) The bacterial colonization in colon was reduced in mice immunized with GMMA-MC001 (≈3-logs, ***P=0.0003) and GMMA-MC007 (≈2-logs, **P=0.0068) compared to the mice immunized with GMMA-K12. (C) As for bacteria counts in caecum at day-7 post-challenge a significant reduction of bacteria was observed in mice immunized with GMMA-MC001 (=5-logs, ***P=0.0006) and GMMA-MC007 (≈3-logs, **P=0.0012), compared to mice immunized with GMMA-K12. The plots represent individual average and standard error of the mean (SEM) of individual mice. Significant differences in colonization were calculated by Mann-Whitney.

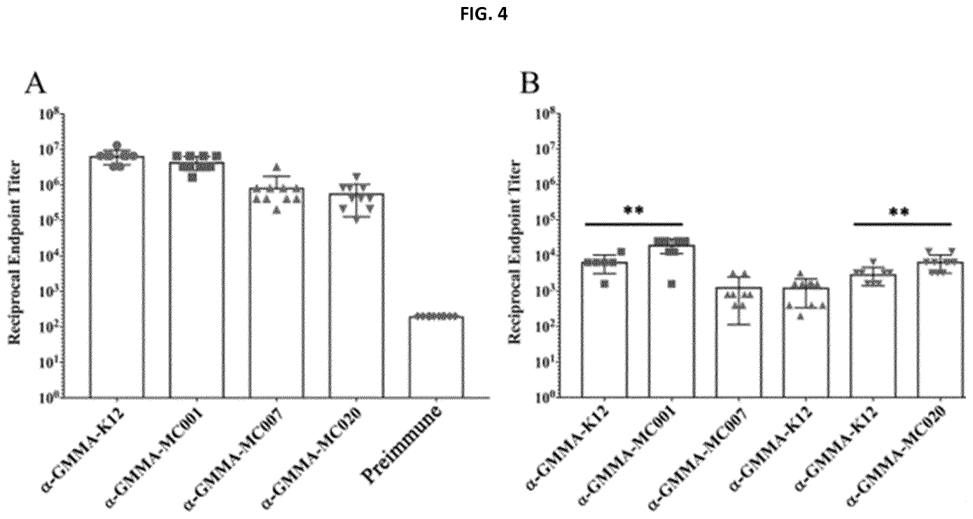

FIG. 4. Specific total IgG response after immunization. Serum was collected from vaccinated animals previous the first immunization and 2 weeks post third immunization. Specific IgG antibody relative titers of GMMA over expressing the antigens or recombinant protein were measured by ELISA assays. Data are shown as mean of each group. (A) The GMMA vaccines were able to trigger the immune response, compared to pre-immune sera/PBS-alum group. However, the total IgG showed no significant difference among the GMMA groups. (B) The further test of specific IgG against the recombinant proteins form of each antigen, showed a significant difference of GMMA-MC001 (**P=0.0076) and GMMA-020 (**P=0.0075) against GMMA-K12 group. The endpoint titer of a sample is defined as the reciprocal of the highest dilution that has a reading above the cut-off. (C) The vaccines candidates were also able to generate specific antibodies against the antigens. By western blot assays, using recombinant proteins forms of each antigens as target, allowed the recognition only of the MC001 and MC020 recombinant proteins challenged with GMMA-MC001 and GMMA-MC020 sera respectively.

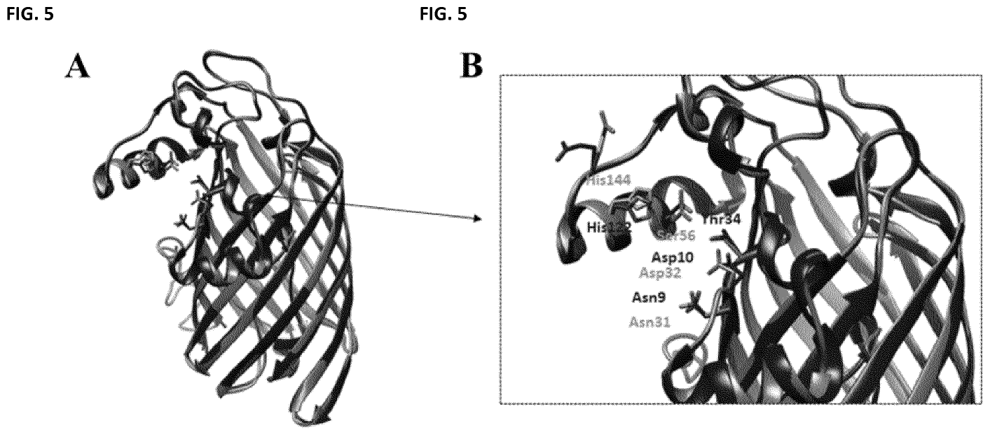

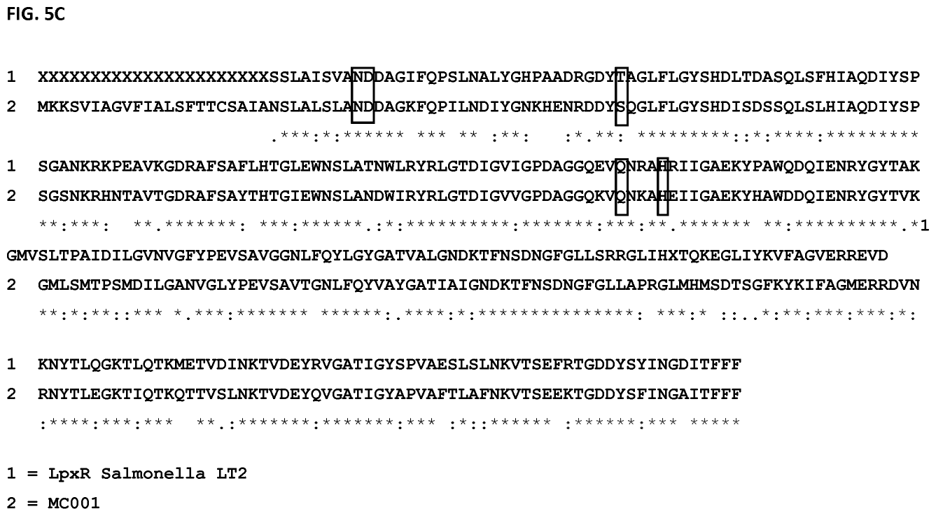

FIG. 5. MC001 comparative protein modeling. (A) Structural model of MC001 obtained with SWISS-MODEL (depicted in green) using LpxR from Salmonella (3FID) as template (depicted in blue). (B) A detail of FIG. 9a show the conservation of the active site residues. (C) Sequence alignment between MC001 and LpxR from Salmonella in which the residues of the active site are boxed.

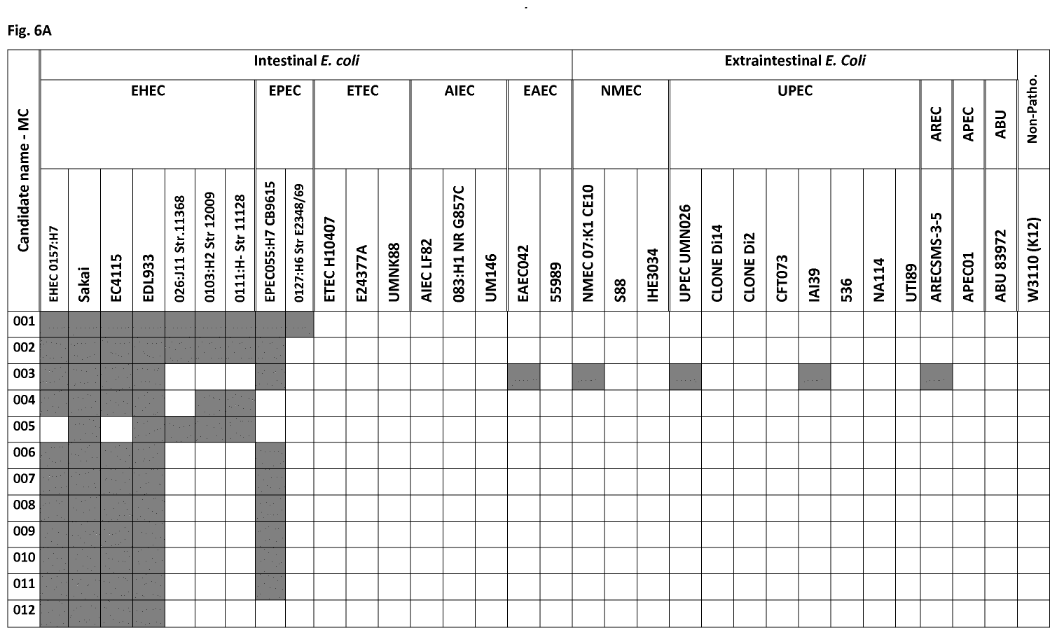

FIG. 6. The selected candidates are mainly present and conserved among EHEC strains. The gene distribution analysis (by BLASTP) shows the presence/absence of the antigens in a panel of 47 E. coli complete genomes. Black cells represent a sequence identity>=80% with a query coverage>=90%, white cells represent gene absence or presence with a sequence identity<80% and query coverage below 90%. FIG. 6A provides analysis for antigen candidates MC001-MC012; FIG. 6B provides analysis for antigen candidates MC013-MC024.

### EXAMPLES

### Introduction

Enterohemorrhagic E. col (EHEC) are a major cause of large outbreaks mainly affecting developed countries. For instance, from 1982 to 2002, a total of 350 E. coli 0157 outbreaks were reported in the United States. EHEC infection causes diarrheal disease often associated with clinical complications like hemorrhagic colitis and hemolytic uremic syndrome (HUS). Antibiotic based therapy is discouraged due to their potential undesirable effect in releasing shiga-toxin from the bacteria. No licensed vaccine specific for human use against EHEC is currently available. In this study, candidate antigens were identified from the EHEC O157:H7 genome and used with the GMMA antigen delivery system to provide new potential vaccine candidates. In particular, one of the candidate antigens (MC001) was able to reduce intestinal bacterial colonization in a mouse model; to our knowledge this study is the first report describing a lipid A deacylase enzyme (LpxR) as an antigen candidate.

### Materials and Methods

### Bacterial Strains and Culture Conditions

All bacterial strains were routinely grown in Luria Bertani (LB) media with antibiotic selection pressure if needed, at 37° C. The E. coli MACH1-T1R (Thermofisher) strain was used for cloning while BL21 DE3 (NEB) strain was used to express and purify the antigens as recombinant proteins. EHEC O157:H7 strain EDL933, is the prototype pathotype used in this study for antigen identification. The strain EHEC O157:H7 86-24 was used in the animal challenging experiments.

### Antigen Candidate Selection

In silico antigen candidate identification was performed as follows. The 5675 CDS (coding DNA sequences) of EHEC O157:H7 EDL933 strain annotated genome (GeneBank sequence CP008957.1) were analyzed by PSORT software (Yu et al., 2010) to predict the cellular localization. TMHMM (http://www.cbs.dtu.dk/services/TMHMM/, (Krogh et al., 2001) was used for prediction of transmembrane regions in putative proteins.

RNA-Seq mapping and reads per kilobase per million mapped reads (RPKM) (cutoff>10) calculation was performed using Geneious R9 software (http://www.geneious.com, (Kearse et al., 2012). Distribution and sequence variability analysis into 47 E. coli complete annotated genomes was performed BLASTP (Altschul et al., 1997) using a cutoff of 290% of query coverage and a 280% of sequence protein identity %). Only antigens present in more than 5 intestinal pathogenic E. coli strains were selected. Presence of antigen candidates in non-pathogenic E. coli K12 and/or BL21 strains was used as exclusion criterion.

Cloning and recombinant protein production of antigen candidates. All candidate antigens were cloned and expressed as His-tagged fusion proteins without the predicted signal sequence. Prediction of the signal peptide was performed by Signal P (Nielsen, 2017). All fragments were amplified by PCR using primers listed in Table S4, using genomic DNA of E. coli EHEC O157:H7 EDL933 strain. The PCR amplicons were cloned into a pET-15b plasmid (Novagen, EMD Millipore) with a His-tag in the carboxyl-terminus by the polymerase incomplete primer extension (PIPE) method (Klock and Lesley, 2009) or the NEBUILDER HiFi DNA Assembly Master Mix (NEB). Plasmids were transformed in BL21-DE3 (NEB). Briefly, E. coli BL21(DE3) harboring pET-15b constructs were grown in ENPRESSO (BioSilta) following the manufacturer suggestions and using Isopropyl β-D-1-thiogalactopyranoside (IPTG) 0.01M (Sigma-Aldrich) for induction. After biomass collection, cultures were lysed by sonication. The suspension obtained was centrifuged and the supernatant passed through a Ni-NTAAgarose chelating column (Qiagen). Proteins were eluted using an immidazol concentration gradient. In the case of insoluble proteins, the purification-immidazol buffer contained urea 6 M. Protein concentrations were measured by PIERCE BCA Protein Assay Kit (Thermofisher).

### Construction of TolR Mutant

The tolR mutant in a E. coli K12 was constructed by allelic maker exchange using the Lambda red system (Datsenko and Wanner, 2000). The tolR was interrupted with a chloramphenicol resistance cassette (cat). Briefly, the cat cassette was amplified using forward and reverse primers with ≈70-nucleotides tail homologous to the flanking region of tolR, Table S4. The PCR product was purified and used to transform E. coli K12 recipient cells (carrying the plasmid expressing the recombinase E, pKD46) as described previously (Datsenko and Wanner, 2000). The deletion of the tolR gene was confirmed by PCR genomic DNA amplification using primers specifically annealing to the genes upstream (tolQ) and downstream (tolA) to tolR.

### NOMV and GMMA Production

For NOMV isolation the E. coli K12 MC4100 WT strain was grown at 37° C. in liquid Luria-Bertani medium. For GMMA production E. coli K12 ΔtolR::cat was grown at 37° C. in liquid Luria-Bertani (LB) medium containing 10 chloramphenicol (20 μg/ml) as previously described (Fantappie et al., 2014; Rossi et al., 2016). Briefly, 75 ml of media were inoculated with E. coli K12 WT or ΔtolR::cat and grown at 37° C., 150 rpm overnight (=16 hrs.). To recover the supernatants cultures were centrifuged for 30 min at 8,000 g and 0.22 μm filtered. These media were ultracentrifuged using propylene ultracentrifuge tubes (Beckman Coulter) at 32,000 rpm for 2h at 4° C. Pellets were washed with phosphate-buffered saline (PBS). Pellets were resuspended in 2 ml of PBS followed by 0.22-μm filtration. Vesicles were stored at 4° C. To determine the total protein content present in these preparations a quantification was performed by DC protein assay (Bio-Rad) based on the Lowry assay (Rossi et al., 2015)

### Negative-Staining Transmission Electron Microscopy

A drop of 10 μL of GMMA or NOMV suspension was placed on copper formvar/carbon-coated grids and adsorbed for 2 minutes. Grids were then washed with few drops of distilled water and blotted with a Whatman filter paper. For negative staining, grids were treated with Uranyless EM stain (Delta Microscopy with Chromalys France) for 1 minute, air-dried and viewed through transmission electron microscope Hitachi H-7650 at 80 kV. Electron micrographs were recorded at a nominal magnification of 120,000×.

### Over-Expression of Antigens in GMMA

To over express the MC001, MC007 and MC020 candidates in GMMA, the corresponding coding sequence including their own signal peptide was cloned into a pBAD-A plasmid (Thermofisher) using the NEBUILDER HiFi DNA Assembly Master Mix (NEB). Also, a Flag-tag sequence (DYKDDDDK (SEQ ID NO: 116)) was introduced between the signal peptide of each protein and the rest of their sequence. The generated constructs (pBAD-MC001F, pBAD-MC007F, pBAD-MC020F), were transformed into the E. coli K12 ΔtolR::cat mutant and induced with arabinose (0.01% in final concentration). The generated GMMA were named GMMA-MC001, GMMA-MC007 and GMMA-MC020. The GMMA not expressing any antigen and obtained by transforming the empty pBAD-A plasmid were named GMMA-K12.

### Mice Immunization and Colonization Model

Five-weeks old BALB/c mice (Janvier) (10 mice per group) were immunized using either (a) PBS-alum hydroxide as adjuvant (ALHYDROGEL 2%, Invivogen), (b) GMMA-expressing the MC001 candidate (GMMA-MC001) plus adjuvant, (c) GMMA expressing the MC007 candidate (GMMA-MC007) plus adjuvant, (d) GMMA expressing the MC020 candidate (GMMA-MC020) plus adjuvant, or GMMA-K12 (GMMA not expressing any candidate) plus adjuvant. All GMMA preparations were formulated using 2 mg/ml alum hydroxide as adjuvant. Animals were immunized (Day 1) by intraperitoneal injections (i.p.) with 10 μg of GMMA plus alum hydroxide, and at day 21 and day 35 with 5 μg of GMMA plus alum hydroxide. Blood was collected from all the mice prior immunization and two weeks after the third dose. The challenge experiment was performed at day-49 and using the EHEC O157:H7 strain 86-24. Mice were treated with streptomycin 24-hours prior infection. Also, animals received cimetidine 2 hours before infection via i.p. The animals were infected with 5×10⁹ CFU via gavage. Animal monitoring was performed on a daily basis including weight, signs and symptoms surveillance. Mice displaying signals of illness and losing more than the 15% of the total weight were euthanized, collecting the colon and colon organs. Fecal pellets were collected every day from day 1 to day 7 post-infection. At day 7 the remaining mice were euthanized and their colon and colon organs were collected. This animal model was adapted from models previously reported (Mohawk and O'Brien, 2011; Garcia-Angulo et al., 2013; Garcia-Angulo et al., 2014). All animal experiments were reviewed and approved by the Auvergne Committee for Animal Experimentation C2EA (Agreement No 6065-2016071216144325V2).

### Enzyme-Linked Immunosorbent Assay (ELISA)

Ninety-six well Maxisorp plates (Nunc, Thermo Fisher Scientific) were coated with 1 mg/mi of GMMA preparations antigens or 1 mg/ml of recombinant protein in PBS overnight (O/N) at 4 QC. Next day, plates were washed 3 times with T-PBS (0.05% Tween 20 in PBS, pH 7.4) and blocked with 100 μl 2% BSA (Sigma-Aldrich) for 1 hour at 37° C. Every incubation step was followed by triple T-PBS wash. Serum samples were initially diluted 1:200 in 2% BSA in T-PBS, transferred to coated-blocked plates and serially 2-fold diluted followed by 2-hours incubation at 37 QC. Then 100 μl/well of 1:2,000 diluted alkaline phosphatase-conjugated goat anti-mouse IgG (H+L) (Southern Biotech) were added and incubated for 2 hours at 37 QC. Bound alkaline phosphatase was visualized by adding SIGMAFAST p-Nitrophenyl phosphate (Sigma-Aldrich) After 30 minutes at room temperature, plates were analyzed at 405 nm in a microplate spectrophotometer. The endpoint titer of a sample is defined as the reciprocal of the highest dilution that has a reading above the cut-off (Frey, 1998).

### Western Blotting

Western blots were carried out on whole cell extracts (wce), recombinant proteins or GMMA preparations. SDS page was performed in MES buffer (Thermofisher) and transferred to iBlot 2 nitrocellulose stacks (iBlot system, Thermofisher). To visualize transferred proteins, the membranes were stained with ponceau red. Then, membranes were blocked with 10% (w/v) blotting-grade blocker (Bio-Rad) in T-PBS. The membranes were later incubated with the respective mouse polyclonal antisera in a 1:1000 dilution in T-PBS-3% blocker 1 hr. at room temperature. Membranes were washed three times with T-PBS and then incubated with goat anti-mouse horseradish peroxidase-conjugated IgG (Dako antibodies) diluted (1:2000) in T-PBS-3% blocker. Colorimetric staining was performed using Opti-4CN Substrate Kit (Bio-Rad) following manufacturer instructions. To detect the FLAG-tag monoclonal ANTI-FLAG M2 secondary antibody was used (Sigma Aldrich).

### Comparative Structural Modelling

Structural models of MC001 have been obtained by employing three different approaches: the threading/ab initio modelling method implemented in the I-TASSER pipeline (Roy et al., 2010), the membrane proteins-specific approach of MEMOIR (Ebejer et al., 2013) and the homology modelling method of SWISS-MODEL (Biasini et al., 2014). The search for suitable modelling templates has been carried out with PSI-BLAST (Position-Specific Iterated BLAST) (Altschul et al., 1997) sequence similarity search against the Protein Data Bank using the amino acid sequence of MC001 as a bait. While MEMOIR does not provide a proper quality assessment of the models, in the case of I-TASSER and SWISS-MODEL, the quality of the final models has been assessed through the parameters C-score and QMEAN4 (Benkert et al., 2009), respectively. The C-score is a confidence score calculated based on the reliability of threading template alignments and the convergence parameters of the structure assembly simulations. C-score values typically range between −5 and 2, higher values characterizing high confidence models and vice-versa. The QMEAN4 score is a linear combination of four statistical potential terms and is typically in the range 0-1, with higher values characterizing better quality models. MC001 models are characterized by a C-score of −5 and a QMEAN4 score of 0.74.

### Statistical Analysis.

All of the statistical analyses were done using GraphPad Prism 7 software. Mann-Whitney (unpaired and non-parametric) and Student t test with threshold of P=<0.05 were used to analyse the data of the bacterial counts from the mouse colonization model and for the IgG antibody response.

### Results

### Identification of Antigen Candidates

Potential antigens in EHEC O157:H7 EDL933 strain were identified by first analysing the putative cellular localization of the 5675 CDS from the annotated genome of EHEC O157:H7 EDL933 strain using the PSORT algorithm. We focused mainly on chromosome-encoded proteins predicted to be exported, surface associated proteins, outer-membrane-associated proteins, and proteins with an unknown localization. The selection criteria also included proteins greater than 200 amino acids and with less than 3 transmembrane repeats determined by the TMHMM algorithm. As result of this analysis, 329 potential antigen candidates were identified (Table S1). Next, RNA-Seq data available in NCBI Sequence Read Archive (SRA) was used to identify genes that were expressed at transcriptional level. These RNA-Seq dataset were previously generated using EHEC EDL933 strain grown in LB, LB with antibiotics, LB-agar media and cattle faeces (Landstorfer et al., 2014). Reads mapping on EHEC EDL933 resulted in 68 genes showing an absolute index number of ≥10 RPKM (reads per kilobase per million mapped reads) in at least one of the four growth conditions analysed (Table S2). Another selection criterion was based on gene variability and distribution analysis of these 68 EHEC EDL933 potential antigens on 47 complete genomes to select those present (query coverage: ≥290%) and conserved (sequence identity≥80%) in more than 5 different intestinal pathogenic E. coli strains. In addition, presence of antigen candidates in non-pathogenic E. coli such as K12 was used as exclusion criterion (FIG. 6). This in silico selection led to the identification of 24 potential antigens which were cloned, expressed and purified as recombinant His-tagged fusion proteins in E. coli. Polynucleotide and polypeptide sequences of these 24 antigen candidates are provided in Table 55. Of these, 12 were successfully purified as soluble and 12 as insoluble proteins (Table S3).

The recombinant proteins were then used to immunize mice to produce polyclonal antibodies. These antibodies were subsequently tested in Western Blot analysis to assess the expression level of the corresponding potential candidate in the homologous EHEC O157:H7 strain, leading to the identification of 17 expressed proteins in standard laboratory growth conditions (Table 53). Finally, among these expressed proteins and as proof of concept, we focused on three potential antigens satisfying all the criteria mentioned herein. In particular, these three antigen candidates included an outer membrane protein (MC001), a putative aminopeptidase (MC007) and an autotransporter belonging to the AIDA family (MC020) (Table 1).

TABLE 1
Features of the three selected antigen candidates

| | | | | | | Purified |
|---|---|---|---|---|---|---|
| Selected | Protein | Annotation | Localization | Pfam | Signal- | recombinant |
| Candidates | ID | feature | prediction | Domain | Localization | protein |
| MC001 | AIG67060.1 | Putative outer | Extracellular | DUF2219 | Non- | Insoluble |
| | | membrane | | | Cytoplasmic | |
| | | protein | | | | |
| MC007 | AIG66424.1 | Putative | Unknown | Unknown | Non- | Soluble |
| | | aminopeptidase | | | Cytoplasmic | |
| MC020 | AIG69974.1 | Pertactine | Extracellular | AIDA, | Unknown | Soluble |
| | | precursor | | Pertactine | | |

### Antigen Delivery in Generalized Modules for Membrane Antigens (GMMA)

To express the three selected candidates in GMMA, we first generated an overblebbing E. coli K12 by mutating the tolR gene (K12 ΔtolR::cat). NOMV and GMMA were then isolated by ultracentrifugation from supernatants of E. coli K12 WT and the K12 ΔtolR::cat. The native OMV (NOMV) released from the wild-type E. coli K12 and GMMA preparation produced by the tolR mutant were observed by transmission electronic microscopy (TEM). This analysis showed that NOMV from K12 WT strain appeared as closed spherical particles and were homogeneous in shape with a size ranging from 20 to 100 nm (FIG. 1A). On the other hand, GMMA from tolR mutant were released in higher amount (size ranging from 20 to 200 nm), and some of these GMMA showed an atypical shape characterized by more than one membrane layer (magnification 120,000×)(FIG. 1B). SDS-PAGE (4-12% bis-tris polyacrylamide) comparison of membrane vesicles (NONV and GMMA) purified from 75 ml of culture supernatants showed that the tolR mutant yielded 25-fold more vesicles than wild-type strain in terms of total protein content (FIG. 1C). Total protein content was quantified and 50 ug of GMMA obtained from K12 ΔtolR::cat sample was loaded into the gel. An equivalent volumetric amount of NOMV from K12 WT obtained from 75 mL of supernatant was loaded and separated by SDS-PAGE. The tolR mutant showed an extensive protein profile in the supernatant compared to wild type.

In order to express the three antigen candidates (MC001, MC007 and MC0021) in GMMA, each of their coding sequences was cloned in pBAD plasmid and the FLAG tag was inserted after their own signal peptide sequence (FIG. 2A). The generated constructs (pBAD-MC001F, pBAD-MC007F, pBAD-MC020F), were each independently transformed in the K12tolR::cat mutant and induced with arabinose. To test whether these plasmids were expressing the antigen candidates and incorporated into GMMA, we performed a western blot on GMMA recovered from 16h cultures using the anti-FLAG antibody. As shown in FIG. 2B all the three antigens were specifically recognized by the anti-Flag. These results indicate that the selected antigen candidates were expressed in GMMA and preparations of these vesicles can be used as an antigen delivery system.

Immunization with MC001-GMMA Reduces EHEC Intestinal Bacterial Colonization in Mice

To test the ability of the selected candidate antigens to prevent or reduce bacterial infection, an intestinal colonization model was setup using BALB/c mice. The EHEC O157:H7 86-24 strain was used for its ability to maintain stable intestinal bacterial colonization for 7-days post-infection using 5×10⁹ CFU (data not shown). Groups of ten mice were immunized with GMMA over-expressing the candidate antigens or with empty GMMA-K12 via intraperitoneal delivery at day 1, 21 and 35. At day 49, mice were infected with EHEC O157:H7 86-24 strain, via gavage. Fecal samples were collected in a daily basis for performing bacterial counts. GMMA-MC001 immunized mice showed a ≈3-log reduction (P=0.0001) in fecal bacteria number compared to PBS-alum immunized mice at day 5 after infection, while a ≈2-log and ≈4-log reduction (P value=0.0033 and 0.0037) was obtained in comparison to empty GMMA-K12 immunized groups at day 6 and 7 respectively (FIG. 3A). For ethical reasons, at day 5 most of the PBS-alum immunized mice were euthanized due their weight loss (>15% of initial body weight). By contrast, mice immunized with GMMA-MC007 and GMMA-MC020 preparations did not show significant reduction in fecal bacterial shedding in comparison to PBS-alum and GMMA-K12 immunized mice (FIG. 3A).

At day 7 post infection, colon and caecum tissues were collected from all mice groups and bacterial count was performed. The number of bacteria in colon and caecum tissues was significantly reduced (3-logs and 5-logs, P=0.0003 and 0.0006 respectively) in mice immunized with GMMA-MC001 in comparison to mice immunized with GMMA-K12, while GMMA-MC007 immunized mice showed a reduction of =2-logs and %3-logs for colon and caecum respectively (P=0.0068 and 0.0012) (FIGS. 3B and 3C) compared to the mice immunized with GMMA-K12.

Immunization with GMMA Expressing Antigen Candidates Induces Specific Antigen Antibody Response

To assess the immune response induced by GMMA immunization per se and the possible contribution in raising specific immune response against the three antigen candidates (MC001, MC007 and MC020) expressed in GMMA, serum antibody levels were determined by ELISA.

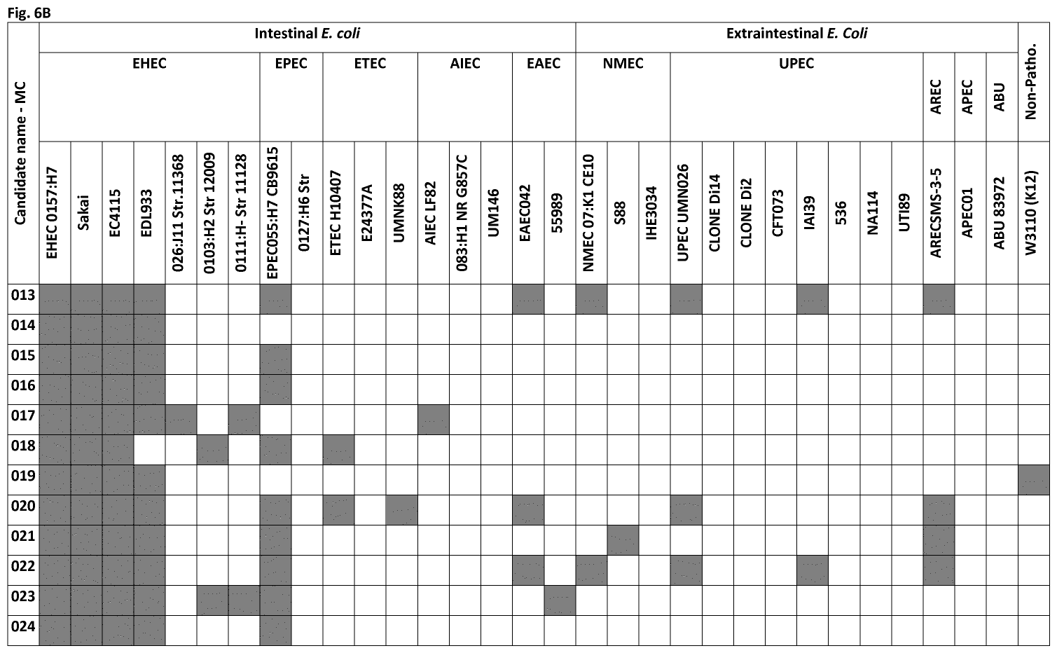

To measure the total level of immunoglobulins G (IgG), serum samples were collected from the mice before the first immunization (preimmune sera) and two weeks after the third immunization, before challenging mice. Microtiter plates coated with purified preparations of GMMA-K12 and each of the three GMMA carrying a antigen candidate showed higher total IgG levels in all the immunized groups versus the preimmune sera. A non-significant difference was found among the four GMMA immunized groups (FIG. 5A). To test whether there was an induction of a specific immune response attributable to the antigens expressed in GMMA, we perform ELISA assays using microtitre plates coated with the MC001, MC007 and MC020 recombinant proteins. A significant increase in antibody response was found for GMMA-MC001 (P=0.0076) and GMMA-MC020 (P=0.0075) sera in comparison to GMMA-K12 sera (FIG. 58). To confirm that the immunization with GMMA expressing these antigen candidates was able to generate antigen specific antibodies, we performed western blot assays, using MC001, MC020 or MC007 recombinant proteins as target. Only MC001 and MC020 recombinant proteins were detected using GMMA-MC001 and GMMA-MC020 sera respectively, while MC007 was not recognized by GMMA-MC007 serum.

MC001 is Homologous to Salmonella typhimurium Lipid A Deacylase (LpxR)

To obtain more insights about the structural features of the MC001 antigen candidate an in-silico analysis was performed. In order to find proteins with known structure and significant sequence similarity with MC001, its protein sequence was used to run a PSI-BLAST search against the Protein Data Bank (PDB) (Berman et al., 2000). This search retrieved as first hit the sequence of the Salmonella typhimurium lipid A deacylase (LpxR). Furthermore, a PSI-BLAST search over the non-redundant protein sequences database revealed a high sequence similarity also with LpxR from Vibrio cholerae, Yersinia enterocolitica and Helicobacter pylori. Structural MC001 models using the LpxR structure as a template (PDB code: 3FID (Rutten et al., 2009) were built by I-TASSER, MEMOIR and SWISS-MODEL software. All three generated models that were in agreement with each other and showing a pairwise Cα root mean square deviation being in all cases lower than 0.5 Å.

FIG. 5A provides a representative MC001 structural model obtained with the SWISS-MODEL The MC001 model was composed of a 12-stranded β-barrel in which the β-strands were arranged in an antiparallel fashion in a structure that is quite common in porins and other cell membrane proteins. The high homology between MC001 and Salmonella typhimurium LpxR was confirmed by the presence in the active site of six conserved residues essential for Ca+2 binding and LpxR catalytic activity: (Salmonella/E. coli) Asn (9/31), Asp (10/32), Thr/Ser (34/56), His (122/144), Gln (118/140) and Glu (128/150) (FIG. 5B). Sequence comparison revealed that MC001 has the same length of the LpxR Salmonella orthologue (319 amino acids) and shares approximately 74% sequence identity and 93.73% sequence similarity (74% of identity with 93% query coverage) (FIG. 5C).

### Discussion

In this study, we identified 329 potential antigen candidates starting from the 5675 CDS of the EHEC O157:H7 EDL933 annotated genome. Further, to determine whether these candidates are expressed during infection or in vitro thus having a higher potential to be immunogenic we took into account the expression at the RNA level. Exploiting previously generated RNA-Seq dataset (Landstorfer et al., 2014) we identified antigens expressed in at least one out of four analyzed conditions, and selected potential antigens possibly present and conserved in more than a single intestinal pathotype. Following these criteria, we selected 24 candidate antigens; 17 of these 24 were found to be expressed also at the protein level in standard laboratory growth condition. Among this antigen panel, 12 proteins were predicted to be outer membrane associated proteins, three with extracellular localization and nine unknown.

On the basis of these observations we used GMMA to express and deliver these candidate antigens (Berlanda Scorza et al., 2012; Bartolini et al., 2013; Daleke-Schermerhorn et al., 2014; Fantappie et al., 2014); GMMA is a system that has been successfully used for expression of properly folded membrane associated recombinant antigens and to induce functional immune responses (Bartolini et al., 2013). Also, GMMA can be a useful delivery system because the presence of native proteins on the membrane surface can act as self-adjuvants, helping to elicit an immune response (Kaparakis-Liaskos and Ferrero, 2015). However, in native conditions, blebs are recovered in small quantity and as consequence E. coli strains may be genetically modified by deletion of the tolR gene to enhance the level of vesicle production (Bernadac et al., 1998; Berlanda Scorza et al., 2012). In a first attempt, we introduced the tolR mutation in EHEC in which Stx was deleted to avoid possible release of toxins into the vesicles. Once these purified blebs were used to immunize mice we observed sick animals, showing bristled hair, lethargic behavior and lose of weight (data not shown). For this reason, we constructed a tolR mutant in an avirulent E. coli K12 which was able to release higher amounts of blebs in comparison to the wild-type K12. A small fraction of these vesicles when observed in TEM showed an atypical shape characterized by more than one membrane layer (double-bilayer) a characteristic observed by Perez-Crutz et al., (Perez-Cruz et al., 2013; Perez-Cruz et al., 2016). In the present study, we used GMMA for antigen expression and delivery. As proof of concept we selected three out of the 24 candidate antigens, including an outer membrane protein (MC001), a putative aminopeptidase (MC007) and an autotransporter (MC020) for expression in GMMA. Two out of the three selected candidate antigens (MC001 and MC020) showed typical features of membrane associated proteins, while for the MC007 the putative cellular localization was unknown. In addition, the MC001 candidate when purified as recombinant protein was obtained as insoluble form. Thus, we reasoned that the expression and delivery of these antigen candidates in GMMA would increase their antigenic potential since they could be presented in their native conformation. Our data showed that all the three candidates expressed by GMMA were specifically recognized by the anti-Flag antibody.

For testing the ability of the selected candidate antigens to prevent or reduce bacterial infection, an intestinal colonization model was setup using BALB/c mice. This animal model was adapted from the previous animal models of infection for EHEC and Garcia-Angulo and colleagues (Mohawk and O'Brien, 2011; Garcia-Angulo et al., 2013). Although mice did not develop the symptoms associated with diarrheal disease as observed in humans, these murine models of E. coli O157:H7 infection, based on streptomycin-treated Balb/c mice, are promising for EHEC colonization and candidate vaccine testing (Mohawk and O'Brien, 2011; Garcia-Angulo et al., 2013). Our data indicated that a stable EHEC intestinal bacterial colonization for 7-days post-infection using 5×10⁹ CFU was maintained in the animals, and immunization with GMMA-K12 did not result in toxicity. We showed that intraperitoneal immunization using MC001 was able to significantly reduce EHEC colonization in mice feces (day 6 and 7), colon and caecum tissues (day 7), in comparison to immunization with empty GMMA-K12. For MC007 a less significant reduction was observed in colon and caecum tissues.

Moreover, the immune response of the treated mice not only showed high titers of total IgG in the GMMA-MC001 vaccinated group, but also IgG specific to MC001. In fact, MC001 recombinant protein was recognized by GMMA-MC001 serum indicating this antigen candidate was associated with membrane vesicles and also easily accessible to immune system.

In Western Blot, MC020 recombinant protein was detected by its respective GMMMA serum, and in agreement with the ELISA results showing higher IgG titer in comparison with GMMA-K12; this was not the case for MC007.

As MC001 was indicated by these studies to be a promising antigen candidate, we used bioinformatic pipelines to predict molecular model by homology. BLAST analysis showed that MC001 was homologous to the Salmonella typhimurium lipid A deacylase (LpxR) and shared similarity also with LpxR from Vibrio cholerae, Yersinia enterocolitica and Helicobacter pylori. Structural MC001 model using the Salmonella typhimurium LpxR as a template revealed a structure composed of a 12-stranded β-barrel in which the β-strands were arranged in an antiparallel fashion. The high homology between MC001 and Salmonella typhimurium LpxR was also confirmed by the presence in the active site of six conserved residues essential for Ca+2 binding and LpxR catalytic activity. In addition, we showed that MC001 was present and conserved among different EHEC strains as Sakai, 026:H11, 0103:H2 and EPEC genomes.

Recently, it has been reported that LpxR can play an important role in pathogenesis by removing the 3′-acyloxyacyl group of lipid A (the hydrophobic anchor of lipopolysaccharide, LPS). This modification increases the ability of Salmonella Typhimurium to evade the innate immune response and promotes survival within macrophages (Kawasaki et al., 2012; Petrone et al., 2014). More recently, a role of LpxR from EHEC Sakai strain was shown in the innate immune response evasion. Experiments with an EHEC O157:H7 IpxR mutant strain, reduced the lipid A deacylation and showed an increased inflammatory and phagocytic responses. These effects were attributed to the augmented NF-κB signaling and phosphorylated p38 mitogen protein kinase (MPK), both via TLR4 response. In contrast, LpxR-positive strains, able to modify the lipid A, were capable of attenuating these immune responses. This study also showed the prevalence of LpxR in other LEE positive pathotypes, as EPEC, and as we did in this study (FIG. 6). Furthermore, LpxR seems to be under the positive regulation of Ler and Pch-LEE-positive transcriptional regulators- and H—NS—global-negative regulator. Ler and H—NS seem to directly regulate the LpxR expression (as many of both LEE- and non-LEE-encoded genes). In addition, Pch and expression of LEE-virulence genes are involved also in the modulation of LPxR effect on lipid A, more directly by activating these virulence genes for colonization and in the phagocytic regulation response. In this regard, targeting a specific antibody response toward LpxR could potentially avoid the LPS modification and subsequent immune evasion. However, further studies are ongoing to characterize this protein and its role in EHEC pathogenesis and immune modulation in the host.

The present studies on the EHEC O157:H7 genome combined with the GMMA antigen delivery system led us to identify new potential antigen candidates. In particular, one of them (MC001) was able to reduce intestinal bacterial colonization and to our knowledge this study was the first report describing a lipid A deacylase enzyme (LpxR) as an antigen candidate.

### REFERENCES

Altschul, S. F., Madden, T. L., Schaffer, A. A., Zhang, J., Zhang, Z., Miller, W., et al. (1997). Gapped BLAST and PSI-BLAST: a new generation of protein database search programs. Nucleic Acids Res 25(17), 3389-3402.
Bartolini, E., Ianni, E., Frigimelica, E., Petracca, R., Galli, G., Berlanda Scorza, F., et al. (2013). Recombinant outer membrane vesicles carrying Chlamydia muridarum HtrA induce antibodies that neutralize chlamydial infection in vitro. J Extracell Vesicles 2. doi: 10.3402/jev.v2i0.20181.
Benkert, P., Kunzli, M., and Schwede, T. (2009). QMEAN server for protein model quality estimation. Nucleic Acids Res 37 (Web Server issue), W510-514. doi: 10.1093/nar/gkp322.
Berlanda Scorza, F., Colucci, A. M., Maggiore, L., Sanzone, S., Rossi, O., Ferlenghi, I., et al. (2012). High yield production process for Shigella outer membrane particles. PLoS One 7(6), e35616. doi: 10.1371/journal.pone.0035616.
Berman, H. M., Westbrook, J., Feng, Z., Gilliland, G., Bhat, T. N., Weissig, H., et al. (2000). The Protein Data Bank. Nucleic Acids Res 28(1), 235-242.
Bernadac, A., Gavioli, M., Lazzaroni, J. C., Raina, S., and Lloubes, R. (1998). Escherichia coli tol-pal mutants form outer membrane vesicles. J Bacterial 180(18), 4872-4878.
Biasini, M., Bienert, S., Waterhouse, A., Arnold, K., Studer, G., Schmidt, T., et al. (2014). SWISS-MODEL: modelling protein tertiary and quaternary structure using evolutionary information. Nucleic Acids Res 42 (Web Server issue), W252-258. doi: 10.1093/nar/gku340.
Croxen, M. A., and Finlay, B. B. (2010). Molecular mechanisms of Escherichia coli pathogenicity. Nat Rev Microbial 8(1), 26-38. doi: 10.1038/nrmicro2265.
Croxen, M. A., Law, R. J., Scholz, R., Keeney, K. M., Wlodarska, M., and Finlay, B. B. (2013). Recent advances in understanding enteric pathogenic Escherichia coli. Clin Microbial Rev 26(4), 822-880. doi: 10.1128/CMR.00022-13.
Daleke-Schermerhorn, M. H., Felix, T., Soprova, Z., Ten Hagen-Jongman, C. M., Vikstrom, D., Majlessi, L, et al. (2014). Decoration of outer membrane vesicles with multiple antigens by using an autotransporter approach. Appl Environ Microbial 80(18), 5854-5865. doi: 10.1128/AEM.01941-14.
Datsenko, K. A., and Wanner, B. L. (2000). One-step inactivation of chromosomal genes in Escherichia coli K-12 using PCR products. Proc Natl Acad Sci USA 97(12), 6640-6645. doi: 10.1073/pnas.120163297.
De Benedetto, G., Alfini, R., Cescutti, P., Caboni, M., Lanzilao, L, Necchi, F., et al. (2017). Characterization of O-antigen delivered by Generalized Modules for Membrane Antigens (GMMA) vaccine candidates against nontyphoidal Salmonella. Vaccine 35(3), 419-426. doi: 10.1016/j.vaccine.2016.11.089.
Ebejer, J. P., Hill, J. R., Kelm, S., Shi, J., and Deane, C. M. (2013). Memoir: template-based structure prediction for membrane proteins. Nucleic Acids Res 41 (Web Server issue), W379-383. doi: 10.1093/nar/gkt331.
Ellis, T. N., and Kuehn, M. J. (2010). Virulence and immunomodulatory roles of bacterial outer membrane vesicles. Microbial Mol Biol Rev 74(1), 81-94. doi: 10.1128/MMBR.00031-09.
Fantappie, L, de Santis, M., Chiarot, E., Carboni, F., Bensi, G., Jousson, O., et al. (2014). Antibody-mediated immunity induced by engineered Escherichia coli OMVs carrying heterologous antigens in their lumen. J Extracell Vesicles 3. doi: 10.3402/jev.v3.24015.
Garcia-Angulo, V. A., Kalita, A., Kalita, M., Lozano, L., and Torres, A. G. (2014). Comparative genomics and immunoinformatics approach for the identification of vaccine candidates for enterohemorrhagic Escherichia coli O157:H7. Infect Immun 82(5), 2016-2026. doi: 10.1128/IAI.01437-13.
Garcia-Angulo, V. A., Kalita, A., and Torres, A. G. (2013). Advances in the development of enterohemorrhagic Escherichia coli vaccines using murine models of infection. Vaccine 31(32), 3229-3235. doi: 10.1016/j.vaccine.2013.05.013.
Gerke, C., Colucci, A. M., Giannelli, C., Sanzone, S., Vitali, C. G., Sollai, L., et al. (2015). Production of a Shigella sonnei Vaccine Based on Generalized Modules for Membrane Antigens (GMMA), 1790GAHB. PLoS One 10(8), e0134478. doi: 10.1371/journal.pone.0134478.
Goldwater, P. N., and Bettelheim, K. A. (2012). Treatment of enterohemorrhagic Escherichia coli (EHEC) infection and hemolytic uremic syndrome (HUS). BMC Med 10, 12. doi: 10.1186/1741-7015-10-12.
Kaparakis-Liaskos, M., and Ferrero, R. L. (2015). Immune modulation by bacterial outer membrane vesicles. Nat Rev Immunol 15(6), 375-387. doi: 10.1038/nri3837.
Kaper, J. B., Nataro, J. P., and Mobley, H. L (2004). Pathogenic Escherichia coli. Nat Rev Microbiol 2(2), 123-140. doi: 10.1038/nrmicro818.
Kawasaki, K., Teramoto, M., Tatsui, R., and Amamoto, S. (2012). Lipid A 3′-O-deacylation by Salmonella outer membrane enzyme LpxR modulates the ability of lipid A to stimulate Toll-like receptor 4. Biochem Biophys Res Commun 428(3), 343-347. doi: 10.1016/j.bbrc.2012.10.054.
Kearse, M., Moir, R., Wilson, A., Stones-Havas, S., Cheung, M., Sturrock, S., et al. (2012). Geneious Basic: an integrated and extendable desktop software platform for the organization and analysis of sequence data. Bioinformatics 28(12), 1647-1649. doi: 10.1093/bioinformatics/bts199.
Klock, H. E., and Lesley, S. A. (2009). The Polymerase Incomplete Primer Extension (PIPE) method applied to high-throughput cloning and site-directed mutagenesis. Methods Mol Biol 498, 91-103. doi: 10.1007/978-1-59745-196-3_6.
Krogh, A., Larsson, B., von Heijne, G., and Sonnhammer, E. L. (2001). Predicting transmembrane protein topology with a hidden Markov model: application to complete genomes. J Mol Biol 305(3), 567-580. doi: 10.1006/jmbi.2000.4315.
Landstorfer, R., Simon, S., Schober, S., Keim, D., Scherer, S., and Neuhaus, K. (2014). Comparison of strand-specific transcriptomes of enterohemorrhagic Escherichia coli O157:H7 EDL933 (EHEC) under eleven different environmental conditions including radish sprouts and cattle feces. BMC Genomics 15, 353. doi: 10.1186/1471-2164-15-353.
MacLennan, C. A., and Saul, A. (2014). Vaccines against poverty. Proc Natl Acad Sci USA 111(34), 12307-12312. doi: 10.1073/pnas.1400473111.
Maione, D., Margarit, I., Rinaudo, C. D., Masignani, V., Mora, M., Scarselli, M., et al. (2005). Identification of a universal Group B streptococcus vaccine by multiple genome screen. Science 309(5731), 148-150. doi: 10.1126/science.1109869.
Mohawk, K. L, and O'Brien, A. D. (2011). Mouse models of Escherichia coli O157:H7 infection and shiga toxin injection. J Biomed Biotechnol 2011, 258185. doi: 10.1155/2011/258185.
Mora, M., Donati, C., Medini, D., Covacci, A., and Rappuoli, R. (2006). Microbial genomes and vaccine design: refinements to the classical reverse vaccinology approach. Curr Opin Microbial 9(5), 532-536. doi: 10.1016/j.mib.2006.07.003.
Mora, M., Veggi, D., Santini, L., Pizza, M., and Rappuoli, R. (2003). Reverse vaccinology. Drug Discov Today 8(10),459-464.
Moriel, D. G., Bertoldi, I., Spagnuolo, A., Marchi, S., Rosini, R., Nesta, B., et al. (2010). Identification of protective and broadly conserved vaccine antigens from the genome of extraintestinal pathogenic Escherichia coli. Proc Natl Acad Sci USA 107(20), 9072-9077. doi: 10.1073/pnas.0915077107.
Moriel, D. G., Rosini, R., Seib, K. L, Serino, L, Pizza, M., and Rappuoli, R. (2012). Escherichia coli: great diversity around a common core. MBio 3(3). doi: 10.1128/mBio.00118-12.
Nesta, B., Valeri, M., Spagnuolo, A., Rosini, R., Mora, M., Donato, P., et al. (2014). SslE elicits functional antibodies that impair in vitro mucinase activity and in vivo colonization by both intestinal and extraintestinal Escherichia coli strains. PLoS Pathog 10(5), e1004124. doi: 10.1371/journal.ppat.1004124.
Nielsen, H. (2017). Predicting Secretory Proteins with SignalP. Methods Mol Biol 1611, 59-73. doi: 10.1007/978-1-4939-7015-5_6.
Nieves, W., Petersen, H., Judy, B. M., Blumentritt, C. A., Russell-Lodrigue, K., Roy, C. J., et al. (2014). A Burkholderia pseudomallei outer membrane vesicle vaccine provides protection against lethal sepsis. Clin Vaccine Immunol 21(5), 747-754. doi: 10.1128/CVI.00119-14.
Perez-Cruz, C., Canas, M. A., Gimenez, R., Badia, J., Mercade, E., Baldoma, L, et al. (2016). Membrane Vesicles Released by a hypervesiculating Escherichia coli Nissle 1917 tolR Mutant Are Highly Heterogeneous and Show Reduced Capacity for Epithelial Cell Interaction and Entry. PLoS One 11(12), e0169186. doi: 10.1371/journal.pone.0169186.
Perez-Cruz, C., Carrion, O., Delgado, L., Martinez, G., Lopez-Iglesias, C., and Mercade, E. (2013). New type of outer membrane vesicle produced by the Gram-negative bacterium Shewanella vesiculosa M7T: implications for DNA content. Appl Environ Microbial 79(6), 1874-1881. doi: 10.1128/AEM.03657-12.
Petersen, H., Nieves, W., Russell-Lodrigue, K., Roy, C. J., and Morici, L. A. (2014). Evaluation of a Burkholderia pseudomallei Outer Membrane Vesicle Vaccine in Nonhuman Primates. Procedia Vaccinol 8, 38-42. doi: 10.1016/j.provac.2014.07.007.
Petrone, B. L., Stringer, A. M., and Wade, J. T. (2014). Identification of HilD-regulated genes in Salmonella enterica serovar Typhimurium. J Bacteriol 196(5), 1094-1101. doi: 10.1128/JB.01449-13.
Pizza, M., Scarlato, V., Masignani, V., Giuliani, M. M., Arico, B., Comanducci, M., et al. (2000). Identification of vaccine candidates against serogroup B meningococcus by whole-genome sequencing. Science 287(5459), 1816-1820.
Rappuoli, R. (2000). Reverse vaccinology. Curr Opin Microbial 3(5), 445-450.
Rappuoli, R., Pizza, M., Del Giudice, G., and De Gregorio, E. (2014). Vaccines, new opportunities for a new society. Proc Natl Acad Sci USA 111(34), 12288-12293. doi: 10.1073/pnas.1402981111.
Rivas, M., Chinen, I., and Guth, B. E. C. (2016). “Enterohemorrhagic (Shiga Toxin-Producing) Escherichia coli,” in Escherichia coli in the Americas, ed. A. G. Torres. (Cham: Springer International Publishing), 97-123.
Rossi, O., Caboni, M., Negrea, A., Necchi, F., Alfini, R., Micoli, F., et al. (2016). Toll-Like Receptor Activation by Generalized Modules for Membrane Antigens from Lipid A Mutants of Salmonella enterica Serovars Typhimurium and Enteritidis. Clin Vaccine Immunol 23(4), 304-314. doi: 10.1128/CVI.00023-16.
Rossi, O., Maggiore, L, Necchi, F., Koeberling, O., MacLennan, C. A., Saul, A., et al. (2015). Comparison of Colorimetric Assays with Quantitative Amino Acid Analysis for Protein Quantification of Generalized Modules for Membrane Antigens (GMMA). Molecular Biotechnology 57(1), 84-93. doi: 10.1007/s12033-014-9804-7.
Roy, A., Kucukural, A., and Zhang, Y. (2010). I-TASSER: a unified platform for automated protein structure and function prediction. Nat Protoc 5(4), 725-738. doi: 10.1038/nprot.2010.5.
Rutten, L., Mannie, J. P., Stead, C. M., Raetz, C. R., Reynolds, C. M., Bonvin, A. M., et al. (2009). Active-site architecture and catalytic mechanism of the lipid A deacylase LpxR of Salmonella typhimurium. Proc Natl Acad Sci USA 106(6), 1960-1964. doi: 10.1073/pnas.0813064106.
Tapia, D., Ross, B. N., Kalita, A., Kalita, M., Hatcher, C. L, Muruato, L. A., et al. (2016). From In silico Protein Epitope Density Prediction to Testing Escherichia coli O157:H7 Vaccine Candidates in a Murine Model of Colonization. Front Cell Infect Microbiol 6, 94. doi: 10.3389/fcimb.2016.00094.
Tarr, P. I., Gordon, C. A., and Chandler, W. L. (2005). Shiga-toxin-producing Escherichia coli and haemolytic uraemic syndrome. Lancet 365(9464), 1073-1086. doi: 10.1016/50140-6736(05)71144-2.
Yu, N. Y., Wagner, J. R., Laird, M. R., Melli, G., Rey, S., Lo, R., et al. (2010). PSORTb 3.0: improved protein subcellular localization prediction with refined localization subcategories and predictive capabilities for all prokaryotes. Bioinformatics 26(13), 1608-1615. doi: 10.1093/bioinformatics/btq249.

### Numbered Embodiments

1. An isolated polypeptide comprising or consisting of:
   (a) an amino acid sequence selected from the group consisting of SEQ ID NOs: 25, 26, 27, 28, 29, 30, 31, 32, 33, 34, 35, 36, 37, 38, 39, 40, 41, 42, 43, 44, 45, 46, 47 or 48; or
   (b) a variant and/or fragment of (a), for example:
      (i) a variant of (a);
      (ii) a fragment of (a);
      (iii) a variant of a fragment of (a).
2. The isolated polypeptide according to embodiment 1, wherein (a) is selected from the group consisting of SEQ ID NOs: 25, 26 and 27.
3. The isolated polypeptide according to any preceding embodiment, wherein (a) is SEQ ID NO: 25.
4. The isolated polypeptide according to any preceding embodiment, wherein (b) exhibits at least 60% sequence identity to an amino acid sequence listed in (a), for example, at least 61%,62%, 63%, 64%,65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity to an amino acid sequence listed in (a);
   (i) wherein the at least 60% sequence identity is exhibited over at least 60% of the amino acid sequence listed in (a), for example, a contiguous amino acid sequence spanning at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the amino acid sequence listed in (a); or
   (ii) wherein the at least 60% sequence identity is exhibited over at least 10 contiguous amino acids of the amino acid sequence listed in (a), for example, at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 350, 400, 450, 500, 550, 600, 650, 700, 750, 800, 850, 900, 950, 1000, 1500, 2000, 2500, 2600, 2601, 2602, 2603, 2604, 2605, 2606, 2607, 2608, 2609, 2610, 2611, 2612, 2613, 2614, 2615, 2616, 2617, 2618 or 2619 contiguous amino acids of the amino acid sequence listed in (a).
5. The isolated polypeptide according to any preceding embodiment, wherein the polypeptide comprises or consists of a fragment comprising at least 10 contiguous amino acids, for example, at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233 contiguous amino acids, and/or, where present, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 350, 400, 450, 500, 550, 600, 650, 700, 750, 800, 850, 900, 950, 1000, 1500, 2000, 2500 or 2600 contiguous amino acids.
6. The isolated polypeptide according to any preceding embodiment, wherein the polypeptide comprises or consists of a fragment wherein 1, or at least, 1 amino acid, is truncated from the N-terminus with respect to an amino acid sequence listed in (a), for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 amino acids are truncated from the N-terminus with respect to the amino acid sequence listed in (a).
7. The isolated polypeptide according to any preceding embodiment, wherein the polypeptide comprises or consists of a fragment wherein 1, or at least 1 amino acid, is truncated from the C-terminus with respect to the reference sequence, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 amino acids are truncated from the C-terminus with respect to the reference sequence.
8. The isolated polypeptide according to any preceding embodiment, wherein the polypeptide is fused to one or more additional polypeptide, for example:
   (a) N-terminal fusion;
   (b) C-terminal fusion; or
   (c) N-terminal and C-terminal fusion.
9. The isolated polypeptide according to any preceding embodiment, wherein the polypeptide is conjugated to one or more additional moiety, for example:
   (a) one or more lipid (to form a lipoprotein);
   (b) one or more saccharide or polysaccharide (to form a glycoprotein);
   (c) one or more phosphate group (to form a phosphoprotein);
   (d) one or more heme group (to form a hemoprotein);
   (e) one or more the flavin adenine dinucleotide (FAD) or flavin mononucleotide (FMN) (to form a flavoprotein); and
   (f) one or more metal ion cofactor (to form a metalloprotein).
10. The isolated polypeptide according to any preceding embodiment, wherein the polypeptide is substantially purified.
11. The isolated polypeptide according to any preceding embodiment, wherein the polypeptide is not naturally occurring.
12. The isolated polypeptide according to any preceding embodiment, wherein the polypeptide is recombinant.
13. The isolated polypeptide according to embodiment 1 wherein the polypeptide is a fragment, variant, fusion and/or derivative capable of inducing a specific immune response to an amino acid sequence listed in (a).
14. The isolated polypeptide according to embodiment 1 wherein the polypeptide is a fragment, variant, fusion and/or derivative capable of inducing an immune response to an amino acid sequence listed in (a).
15. The isolated polypeptide according to embodiment 2, wherein the immune response is an immune activating response.
16. The isolated polypeptide according to embodiment 2, wherein the immune response is a protective immune response.
17. The isolated polypeptide according to embodiment 3, wherein the polypeptide is capable of eliciting an in vitro protective immune response.
18. The isolated polypeptide according to embodiment 3, wherein the polypeptide is capable of eliciting an in vivo protective immune response.
19. The isolated polypeptide according to embodiment 5, wherein the polypeptide is capable of eliciting an in vivo protective immune response in a mammal.
20. The isolated polypeptide according to embodiment 6, wherein the mammal is selected from the group consisting of armadillo (dasypus novemcinctus), baboon (Papio anubis; Papio cynocephalus); camel (Camelus bactrianus, Camelus dromedarius, Camelus ferus) cat (Felis catus), dog (Canis lupus familiaris), horse (Equus ferus caballus), ferret (Mustela putorius furo), goat (Capra aegagrus hircus), guinea pig (Cavia porrellus), golden hamster (Mesocricetus auratus), kangeroo (Macropus rufus), llama (Lama glama), mouse (Mus musculus), pig (Sus scrofa domesticus), rabbit (Oryctolagus cuniculus), rat (Rattus norvegicus), rhesus macaque (Macaca mulatta), sheep (Ovis aries), non-human primate, and human (Homo sapiens).
21. The isolated polypeptide according to embodiment 18, wherein the protective immune response is protective against a disease or condition caused, wholly or in part, by an organism selected from the group consisting of: bacteria, Gram negative bacteria; proteobacteria, enterobacteriales, enterobacteriaceae (for example, Salmonella, Escherichia [E. alberti, E. coli, E. fergusonii, E. hermannii, E. marmotae, and E. vulneris], Yersinia, Klebsiella, Proteus, Enterobacter, Serratia, and Citrobacter).
22. The isolated polypeptide according to embodiment 21, wherein disease or condition caused, wholly or in part, by Escherichia coli, for example, extraintestinal pathogenic E. coli(ExPEC), or intestinal pathogenic E. coli (InPEC).
23. The isolated polypeptide according to embodiment 22, wherein the Escherichia coli is from a pathotype selected from the group consisting of: (i) enteropathogenic E. coli (EPEC); (ii) enterohemorrhagic E. coli (EHEC); (iii) enterotoxigenic E. coli (ETEC); (iv) enteroaggregative E. coli (EAEC); (v) diffusely adherent E. coli (DAEC); (vi) enteroinvasive E. coll (EIEC); (vii) uropathogenic E. coli (UPEC); (viii) neonatal meningitis E. coli (NMEC); (ix) Shiga Toxin (Stx) producing enteroaggregative E. coli(STEAEC); (x) adherent Invasive E. coli (AIEC); (xi) amoxicillin-resistant E. coli (AREC); (xii) asymptomatic bacteriuria E. coli (ABU); (xiii) Avian pathogenic E. coli (APEC).
24. The isolated polypeptide according to embodiment 22 or 23, wherein the Escherichia coli is an enterohaemorrhagic E. coli (EHEC) selected from the group consisting of: O157:H7 e.g., EHEC O157:H7 EDL933 strain; EHEC O157:H7 Sakai stain; EHEC 026:H11 (e.g., strain 11368); EHEC 0103:H2 (e.g., strain 12009); and EHEC O111:H− (e.g., strain 11128).
25. The isolated polypeptide according to embodiment 22 or 23, wherein the Escherichia coli is an enteropathogenic E. coli (EPEC) selected from the group consisting of: 055:H7 (e.g., CB9615); and 0127:H6 (e.g., strain E2348/69).
26. The isolated polypeptide according to embodiment 22 or 23, wherein the Escherichia coli is an enterotoxigenic E. coli(ETEC) selected from the group consisting of: H10407; E24377A; and Porcine ETEC.
27. The isolated polypeptide according to embodiment 22 or 23, wherein the Escherichia coli is an adherent Invasive E. coli (AIEC) selected from the group consisting of: LF82; 083:H1 NR G857C; and UM146.
28. The isolated polypeptide according to embodiment 22 or 23, wherein the Escherichia coli is an enteroaggregative E. coli (EAEC) selected from the group consisting of: 042, and 55989.
29. The isolated polypeptide according to embodiment 22 or 23, wherein the Escherichia coli is a neonatal meningitis E. coli (NMEC) selected from the group consisting of: 07:K1 CE10, 588, and 1H E3034.
30. The isolated polypeptide according to embodiment 22 or 23, wherein the Escherichia coli is an uropathogenic E. coli (UPEC) selected from the group consisting of: UMN026, CLONEDi14; CLONE Di2; CFT073; IA139; 536; NA114; and UTI89.
31. The isolated polypeptide according to embodiment 22 or 23, wherein the Escherichia coli is AREC SMS-3-5; APEC 01; or ABU 83972.
32. The isolated polypeptide according to embodiment 22, wherein the Escherichia coli is a strain with a K antigen selected from the group consisting of K1, K2a/ac, K3, K4, K5, K6, K7 (=K56), K8, K9 (=O104), K10, K11, K12 (K82), K13 (=K20 and =K23), K14, K15, K16, K18a, K18ab (=K22), K19,K24, K26, K27, K28, K29, K130, K31, K34, K37, K39, K40, K41,K42, K43, K44, K45, K46, K47, K49 (046), K50, K51, K52, K53, K54 (=K96), K55, K74, K84, K85ab/ac (=O141), K87 (=O32), K92, K93, K95, K97, K98, K100, K101, K102, K103, KX104, KX105, and KX106).
33. The isolated polypeptide according to embodiment 22 or 32, wherein the Escherichia coli is a strain with an O antigen selected from the group consisting of O1 A, O1 A1, O1 B, O1 C, O2, O3, O4, O4, O5 ab, O5 ac, O6, O6, O6, O7, O8, O9, O9 a, O10, O11, O12, O13, O15, O16, O16, O16, O17, O18 A, O18 A1, O18 A1A2, O18 ab, O18 ac, O18 B, O18 B1, O19 ab, O20 ab, O20 ac, O21, O22, O23 A, O24, O25, O26, O27, O28 ab, O28 ac, O29, O30, O32, O33, O34, O35, O36, O37, O38, O39, O40, O41, O42, O43, O44, O45, O45 rel, O46, O48, O49, O50, O51, O52, O53, O54, O55, O55, O56, O58, O59, O60, O61, O62, O63, O64, O65, O66, O68, O69, O70, O71, O73, O73 ab, O74, O75, O76, O77, O78, O79, O80, O81, O82, O83, O84, O85, O86, O86, O86, O87, O88, O89, O90, O91, O92, O95, O96, O97, O98, O99, O100, O101, O102, O103, O104, O105, O106, O107, O108, O109, O110, O111, O112 ab, O112 ac, O113, O114, O115, O116, O117, O118, O119, O120, O121, O123, O124, O125ab, O125ac, O126, O126, O127, O128ab, O128 ab, O128 ac, O129, O130, O131, O132, O133, O134, O135, O136, O137, O138, O139, O140, O141, O142, O143, O144, O145, O146, O147, O148, O149, O150, O151, O152, O153, O154, O155, O156, O157, O158 ab, O158 ac, O159, O160, O161, O163, O164, O165, O166, O167, O168, O169, O170, O171, O172, O173, O174 ab, O174 ac, O175, O176, O177, O178, O179, O180, O181, O182, O183, O184, O185, O186 and O187.
34. The isolated polypeptide according to embodiment 22, 32 or 33, wherein the Escherichia coli is a strain with a H antigen selected from the group consisting of: H1, H2, H3, H4, H5, H6, H7, H8, H9, H10 (=H50), H11, H12, H13, H14, H15, H16, H17, H18, H19, H20, H21, H22, H23, H24, H25, H26, H27, H28, H29, H30, H31, H32, H33, H34, H35, H36, H37, H38, H39, H40, H41, H42, H43, H44, H45, H46, H47, H48, H49, H50 (=H10), H51, H52, H53, H54, H55 and H56.
35. The isolated polypeptide according to any one of embodiments 16-20, wherein the protective immune response is protective against a disease or condition selected from the group consisting of: gastroenteritis; haemolytic uremic syndrome (HUS), urinary tract infection; neonatal meningitis; haemorrhagic colitis; and Crohn's disease.
36. The isolated polypeptide according to any preceding embodiment, wherein the protective immune response is an immune response that results in increased seral cytokine levels; for example, cytokines selected from the group consisting of IL-1α, IL-1β, IL-IRA, IL-18, IL-2, IL-4, IL-7, IL-9, IL-13, IL-15, IL-3, IL-5, GM-CSF, IL-6, IL-11, G-C5F, IL-12, LIF, O5M, IL-10, IL-20, IL-14, IL-16, IL-17, IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β1, TGF-β2, TGF-β3, Epo, Tpo, Flt-3L, SCF, M-CSF and MSP.
37. The isolated polypeptide according to any preceding embodiment, wherein the protective immune response is an immune response is an immune response that results in bactericidal activity and/or opsonophagocytosis.
38. An isolated nucleic acid molecule comprising or consisting of:
   (A) a nucleic acid sequence selected from the group consisting of selected from the group consisting of SEQ ID NO: 1, 2, 3, 4, 5, 6, 7, 8, 9, 10, 11, 12, 13, 14, 15, 16, 17, 18, 19, 20, 21, 22, 23 or 24; or
   (B) a fragment, variant and/or fusion of (A).
39. The isolated nucleic acid according to embodiment 38, wherein (A) is selected from the group consisting of SEQ ID NOs: 1, 2 and 3.
40. The isolated nucleic acid according to embodiment 38 or 39, wherein (A) is SEQ ID NO: 1.
41. The isolated nucleic acid according to embodiment 38, wherein (B) exhibits at least 60% sequence identity to a nucleic acid sequence listed in (A), for example, at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleic acid sequence listed in (a); and
   (I) wherein the at least 60% sequence identity is exhibited over at least 60% of the nucleic acid sequence listed in (a), for example, a contiguous amino acid sequence spanning at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the nucleic acid sequence listed in (A); or
   (II) wherein the at least 60% sequence identity is exhibited over at least 30 contiguous nucleic acids of the nucleic acid sequence listed in (A), for example, at least 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 350, 400, 450, 500, 550,600,650, 700, 750, 800, 850,900, 950, 1000, 1500, 2000, 2500, 3000, 3500, 4000, 4500, 5000, 5500,6000,6500,7000, or 7500 contiguous amino acids of the nucleic acid sequence listed in (A).
42. The isolated nucleic acid according to any one of embodiments 38-41, wherein the nucleic acid encodes a polypeptide according to embodiments 1-37.
43. A vector comprising a nucleic acid molecule defined in any one of embodiments 38-42.
44. A host cell comprising a nucleic acid molecule defined in any one of embodiments 38-42 or a vector as defined in embodiment 43.
45. A method for producing a polypeptide according to any one of embodiments 1-37: comprising or consisting of the steps of culturing a population of host cells according to embodiment 44 under conditions in which the peptide is expressed, and isolating the peptide therefrom.
46. A vesicle comprising one or more polypeptide defined in any one of embodiments 1-37.
47. The vesicle according to embodiment 46, wherein the vesicle is derived from the membrane of a cell, for example, a Gram positive bacteria membrane vesicle or a Gram negative bacteria outer membrane vesicle (OMV).
48. The vesicle according to embodiment 46, wherein the vesicle is a Gram negative bacteria outer membrane vesicle (OMV) of a bacterium defined in any one of embodiments 21-34.
49. The vesicle according to any one of embodiments 46-48, wherein the polypeptide is:
   (a) homologous to the cell; or
   (b) heterologous to the cell.
50. The vesicle according to any one of embodiments 45-49, wherein the polypeptide is:
   (a) located in the lumen of the vesicle (for example, where the vesicle is a cell, in the cytoplasm);
   (b) located in the/a inter-membrane space of the vesicle (for example, where the vesicle is a cell, the periplasm); and/or
   (c) located on the outer surface of the outer membrane (for example, where the vesicle is a cell, the outer membrane).
51. The vesicle according to any one of embodiments 46-50, wherein the polypeptide expressed by the cell.
52. The vesicle according to any one of embodiments 46-50, wherein the polypeptide is chemically conjugated to the vesicle (for example, to a lipid, protein or polysaccharide component of the vesicle membrane).
53. A binding moiety capable of specifically binding to one or more polypeptide defined in any one of embodiments 1-37.
54. The binding moiety according to embodiment 53, wherein the binding moiety is selected from the group consisting of: antibodies; antigen-binding fragments; and antibody mimetics.
55. The binding moiety according to embodiment 53 or 54, wherein the binding moiety is an antibody.
56. The binding moiety according to embodiment 55, wherein the antibody is polyclonal or monoclonal.
57. The binding moiety according to embodiment 55, wherein the binding moiety is an antigen-binding fragment selected from the group consisting of: Fab (fragment antigen binding); F(ab′)2; Fab′; scFv (single chain variable fragment); di-scFv; sdAb (single domain antibody/domain antibody); trifunctional antibody; chemically-linked F(ab′)₂; and BiTE (bi-specific T-cell engager).
58. The binding moiety according to embodiment 57, wherein the antibody or antigen binding fragment thereof is an antigen binding fragment selected from the group consisting of affibodies molecules; affilins; affimers; affitins; alphabodies; anticalins; avimers; DARPins; fynomers; kunitz domain peptides; monobodies and nanoCLAMPs.
59. A pharmaceutical composition comprising a polypeptide defined in any one of embodiments 1-37, a nucleic acid molecule defined in any one of embodiments 38-42, a vector as defined in embodiment 43, a host cell as defined in embodiment 46, a vesicle as defined in any one of embodiments 46-52 and/or a binding moiety as defined in any one of embodiments 53-58.
60. A kit comprising or consisting of a polypeptide defined in any one of embodiments 1-37, a nucleic acid molecule defined in any one of embodiments 38-42, a vector as defined in embodiment 43, a host cell as defined in embodiment 44, a vesicle as defined in any one of embodiments 46-52, a binding moiety as defined in any one of embodiments 53-58 and/or a pharmaceutical composition as defined in embodiment 59; and (optionally) instructions for use.
61. A polypeptide defined in any one of embodiments 1-37, a nucleic acid molecule defined in any one of embodiments 38-42, a vector as defined in embodiment 43, a host cell as defined in embodiment 44, a vesicle as defined in any one of embodiments 46-52, a binding moiety as defined in any one of embodiments 53-58 and/or a pharmaceutical composition as defined in embodiment 59, for use in medicine.
62. A polypeptide defined in any one of embodiments 1-37, a nucleic acid molecule defined in any one of embodiments 38-42, a vector as defined in embodiment 43, a host cell as defined in embodiment 44, a vesicle as defined in any one of embodiments 46-52, a binding moiety as defined in any one of embodiments 53-58 and/or a pharmaceutical composition as defined in embodiment 59, for use in preventing or treating bacterial infection and/or symptoms thereof.
63. The polypeptide, nucleic acid molecule, vector, vesicle, host cell, binding moiety and/or pharmaceutical composition for use according to embodiment 62, wherein the bacterial infection is, wholly or in part, infection with one or more bacterium defined in any one of embodiments 21-34.
64. The polypeptide, nucleic acid molecule, vector, vesicle, host cell, binding moiety and/or pharmaceutical composition for use according to embodiment 63, wherein the use comprises the consecutive or concurrent use of:
   (a) a polypeptide defined in embodiments 1-37;
   (b) a nucleic acid molecule defined in embodiments 38-42;
   (c) a vector as defined in embodiment 43;
   (d) a vesicle as defined in any one of embodiments 46-52;
   (e) a host cell as defined in embodiment 44;
   (f) a binding moiety as defined in any one of embodiments 53-58; and/or
   (g) a pharmaceutical composition as defined in embodiment 59.
65. The polypeptide, nucleic acid molecule, vector, vesicle, host cell, binding moiety and/or pharmaceutical composition for use according to embodiment 64, wherein the use comprises the consecutive or concurrent use of a binding moiety as defined in any one of embodiments 53-58 with:
   (a) a nucleic acid molecule defined in any one of embodiments 37-42 and/or a vector as defined in embodiment 43; and/or
   (b) a binding moiety as defined in any one of embodiments 53-58 and/or a vesicle as defined in any one of embodiments 46-52.
66. The use of polypeptide defined in any one of embodiments 1-37, a nucleic acid molecule defined in any one of embodiments 38-42, a vector as defined in embodiment 43, a host cell as defined in embodiment 44, a vesicle as defined in any one of embodiments 46-52, a binding moiety as defined in any one of embodiments 53-58 and/or a pharmaceutical composition as defined in embodiment 59, in preventing or treating bacterial infection and/or symptoms thereof, for example, wherein the bacterial infection is, wholly or in part, infection with one or more bacterium defined in any one of embodiments 21-34.
67. The use of polypeptide defined in any one of embodiments 1-37, a nucleic acid molecule defined in any one of embodiments 38-42, a vector as defined in embodiment 43, a host cell as defined in embodiment 44, a vesicle as defined in any one of embodiments 46-52, a binding moiety as defined in any one of embodiments 53-58 and/or a pharmaceutical composition as defined in embodiment 59, in the manufacture of a medicament for preventing or treating bacterial infection and/or symptoms thereof, for example, wherein the bacterial infection is, wholly or in part, infection with one or more bacterium defined in any one of embodiments 21-34.
68. A method for preventing or treating bacterial infection and/or symptoms thereof comprising or consisting of administering to a subject an effective amount of a polypeptide defined in any one of embodiments 1-37, a nucleic acid molecule defined in any one of embodiments 38-42, a vector as defined in embodiment 43, a host cell as defined in embodiment 44, a vesicle as defined in any one of embodiments 46-52, a binding moiety as defined in any one of embodiments 53-58 and/or a pharmaceutical composition as defined in embodiment 59, for example, wherein the bacterial infection is, wholly or in part, infection with one or more bacterium defined in any one of embodiments 21-34.
69. The use of a nucleic acid molecule defined in any one of embodiments 38-42, or a binding moiety as defined in any one of embodiments 53-58, for detecting the presence of bacteria, for example, wherein the bacteria are one or more bacterium defined in any one of embodiments 21-34.
70. The use according to embodiment 69, wherein the detection is in vitro and/or in vivo.
71. A polypeptide, nucleic acid molecule, vector, vesicle, host cell, binding moiety, pharmaceutical composition, use or method as described in specification and figures herein.

TABLE S1
Antigen candidates selected by Psort analysis

| | | Length | PSORT | PSORT | Mod |
|---|---|---|---|---|---|
| Protein ID | Functional annotation | (bp) | Local.¹ | score | HMM² |
| AIG68144.1 | Ferric siderophore transport system, | 720 | U | 2 | 1 |
| | periplasmicbinding protein TonB CDS | | | | |
| AIG70214.1 | Type III secretion outermembrane pore | 1704 | OM | 10 | 1 |
| | forming protein (YscC, MxiD, HrcC, InvG) | | | | |
| | CDS | | | | |
| AIG71811.1 | adherence and invasion outermembrane | 5037 | OM | 9.95 | 0 |
| | protein (Inv, enhances Peyer's patches | | | | |
| | colonization) CDS | | | | |
| AIG66227.1 | Putative outer membrane protein CDS | 2451 | OM | 10 | 0 |
| AIG66265.1 | Outer membrane protein Imp, required for | 2322 | OM | 10 | 0 |
| | envelope biogenesis CDS | | | | |
| AIG66267.1 | hypothetical protein CDS | 753 | U | 2 | 0 |
| AIG66308.1 | Secretion monitor precursor CDS | 588 | U | 6.49 | 1 |
| AIG66347.1 | Fimbrial protein Yad like protein CDS | 1110 | U | 2 | 1 |
| AIG66348.1 | Fimbrial protein YadK CDS | 591 | U | 2 | 0 |
| AIG66349.1 | Fimbrial protein YadL CDS | 606 | U | 2.5 | 1 |
| AIG66351.1 | Outer membrane usher protein HtrE CDS | 2601 | OM | 10 | 0 |
| AIG66353.1 | Fimbrial protein YadN CDS | 597 | Ex | 9.65 | 1 |
| AIG66363.1 | Ferric hydroxamate outer membrane | 2244 | OM | 10 | 0 |
| | receptor FhuA CDS | | | | |
| AIG66390.1 | Outer membrane protein assembly factor | 2433 | OM | 10 | 1 |
| | YaeT precursor CDS | | | | |
| AIG66406.1 | Copper homeostasis protein CutF precursor | 711 | OM | 9.92 | 0 |
| | CDS | | | | |
| AIG66407.1 | putative lipoprotein yaeF precursor CDS | 825 | U | 2.5 | 0 |
| AIG66409.1 | hypothetical protein CDS | 708 | U | 2 | 0 |
| AIG66417.1 | hypothetical protein CDS | 801 | U | 2 | 0 |
| AIG66424.1 | putative aminopeptidase CDS | 801 | U | 2.5 | 1 |
| AIG66428.1 | hypothetical protein CDS | 1410 | U | 2 | 1 |
| AIG66446.1 | core protein CDS | 4215 | U | 2 | 0 |
| AIG66447.1 | hypothetical protein CDS | 612 | U | 2 | 1 |
| AIG66450.1 | core protein CDS | 1761 | Ex | 9.52 | 0 |
| AIG66460.1 | putative exported protein CDS | 741 | U | 2.5 | 1 |
| AIG66464.1 | putative lipoprotein yafL precursor CDS | 774 | U | 4.9 | 1 |
| AIG66480.1 | Outer membrane pore protein E precursor | 1056 | OM | 10 | 1 |
| | CDS | | | | |
| AIG66490.1 | putative tail fiber protein CDS | 795 | U | 2 | 0 |
| AIG66511.1 | Zinc binding domain protein CDS | 2334 | U | 2 | 0 |
| AIG66518.1 | hypothetical protein CDS | 1137 | U | 2.5 | 1 |
| AIG66526.1 | CFA/I fimbrial chaperone CDS | 711 | U | 2.5 | 1 |
| AIG66527.1 | CFA/I fimbrial minor adhesin CDS | 1644 | Ex | 9.64 | 1 |
| AIG66528.1 | CFA/I fimbrial subunit C usher protein CDS | 2526 | U | 4.69 | 2 |
| AIG66528.1 | Putative adhesin CDS | 4254 | OM | 10 | 0 |
| AIG66545.1 | AidA-I adhesin-like protein CDS | 4050 | U | 5.87 | 0 |
| AIG66568.1 | hypothetical protein CDS | 864 | U | 2 | 0 |
| AIG66574.1 | Putative flagellin structural protein CDS | 2943 | OM | 10 | 0 |
| AIG66636.1 | hypothetical protein CDS | 624 | U | 2 | 0 |
| AIG66640.1 | putative lipoprotein CDS | 1095 | U | 2.5 | 0 |
| AIG66656.1 | Protein YkiA CDS | 2166 | U | 2 | 0 |
| AIG66683.1 | Nucleoside-specific channel-forming | 885 | OM | 10 | 0 |
| | protein Tsx precursor CDS | | | | |
| AIG66684.1 | putative lipoprotein yajI CDS | 540 | U | 2 | 0 |
| AIG66769.1 | Large repetitive protein CDS | 4386 | U | 6.04 | 1 |
| AIG66781.1 | Putative stomatin/prohibitin-family | 918 | U | 2 | U |
| | membrane protease subunit YbbK | | | | |
| AIG66819.1 | Oligopeptide ABC transporter, periplasmic | 1701 | U | 2 | 0 |
| | oligopeptide-binding protein OppA CDS | | | | |
| AIG66819.1 | Outer membrane usher protein SfmD CDS | 2610 | OM | 10 | 2 |
| AIG66854.1 | Agglutination protein CDS | 1356 | OM | 10 | 1 |
| AIG66854.1 | TonB-dependent receptor CDS | 2241 | OM | 10 | 0 |
| AIG66909.1 | Rare lipoprotein A precursor CDS | 1089 | Ex | 9.65 | 0 |
| AIG66920.1 | hypothetical protein CDS | 588 | Ex | 9.71 | 0 |
| AIG66922.1 | hypothetical protein CDS | 708 | U | 2 | 0 |
| AIG66972.1 | core protein CDS | 4200 | U | 2 | 0 |
| AIG66973.1 | orf, hypothetical protein CDS | 543 | U | 2 | 0 |
| AIG66984.1 | hypothetical protein CDS | 1062 | U | 2.5 | 0 |
| AIG66987.1 | putative fimbrial-like protein ygiL precursor | 567 | Ex | 9.72 | 0 |
| | CDS | | | | |
| AIG67020.1 | TolA protein CDS | 1185 | U | 2 | 2 |
| AIG67060.1 | Putative outer membrane protein CDS | 960 | U | 2.5 | 1 |
| AIG67099.1 | Biotin synthesis protein BioC CDS | 756 | U | 2 | 0 |
| AIG67129.1 | Ferrichrome-iron receptor CDS | 2283 | OM | 10 | 0 |
| AIG67212.1 | Virulence factor VirK CDS | 993 | U | 2 | 0 |
| AIG67245.1 | Urease accessory protein UreF CDS | 675 | U | 2 | 0 |
| AIG67247.1 | Per-activated serine protease autotransporter | 126 | Ex | 9.71 | U |
| | enterotoxin EspC | | | | |
| AIG67280.1 | Colicin I receptor precursor CDS | 2010 | OM | 10 | 0 |
| AIG67308.1 | putative hemolysin activator protein CDS | 1065 | OM | 8.86 | 1 |
| AIG67367.1 | Putative metalloprotease yggG | 789 | U | 2 | U |
| AIG67384.1 | exported protein CDS | 549 | U | 2.5 | 1 |
| AIG67387.1 | Outer membrane protein F precursor CDS | 1089 | OM | 10 | 1 |
| AIG67397.1 | type 1 fimbriae major subunit FimA CDS | 540 | Ex | 10 | 0 |
| AIG67400.1 | type 1 fimbriae anchoring protein FimD | 1893 | OM | 10 | 0 |
| | CDS | | | | |
| AIG67407.1 | hypothetical protein CDS | 543 | U | 2 | 0 |
| AIG67412.1 | Paraquat-inducible protein B CDS | 1641 | U | 2 | 1 |
| AIG67413.1 | Paraquat-inducible protein B CDS | 564 | U | 2 | 0 |
| AIG67419.1 | Outer membrane protein A precursor CDS | 1065 | OM | 10 | 0 |
| AIG67426.1 | UPF0319 protein YccT precursor CDS | 663 | U | 2.5 | 1 |
| AIG67448.1 | hypothetical protein CDS | 558 | U | 2 | 0 |
| AIG67453.1 | hypothetical protein CDS | 774 | U | 2 | 0 |
| AIG67464.1 | Hypothetical protein CDS | 1851 | U | 2 | 0 |
| AIG67503.1 | hypothetical protein CDS | 753 | Ex | 9.71 | 0 |
| AIG67516.1 | Putative polysaccharide export protein | 1140 | OM | 9.92 | 1 |
| | YccZ precursor CDS | | | | |
| AIG67517.1 | Putative outer membrane lipoprotein YmcA | 2097 | OM | 9.52 | 0 |
| | CDS | | | | |
| AIG67577.1 | Shiga-like toxin II subunit A precursor CDS | 960 | U | 2.5 | 1 |
| AIG67607.1 | hypothetical protein CDS | 618 | U | 2 | 0 |
| AIG67611.1 | hypothetical protein CDS | 657 | U | 2 | 0 |
| AIG67633.1 | hypothetical protein CDS | 1344 | Ex | 9.64 | 0 |
| AIG67644.1 | Biofilm PGA synthesis deacetylase PgaB | 2019 | U | 2 | 1 |
| | CDS | | | | |
| AIG67645.1 | Biofilm PGA outer membrane secretin | 2424 | U | 2.5 | 0 |
| | PgaA CDS | | | | |
| AIG67652.1 | Putative exported protein precursor CDS | 1086 | U | 2 | 0 |
| AIG67653.1 | outer membrane fimbrial usher protein CDS | 2523 | OM | 10 | 0 |
| AIG67658.1 | Hemolysin CDS | 3813 | OM | 9.95 | 0 |
| AIG67671.1 | hypothetical protein CDS | 789 | U | 2 | 0 |
| AIG67672.1 | hypothetical protein CDS | 1389 | U | 2 | 0 |
| AIG67699.1 | Per-activated serine protease autotransporter | 126 | Ex | 9.71 | U |
| | enterotoxin EspC | | | | |
| AIG67732.1 | TonB-dependent receptor CDS | 2010 | OM | 10 | 0 |
| AIG67760.1 | putative hemolysin activator protein CDS | 1065 | OM | 8.86 | 1 |
| AIG67820.1 | Protein yceI precursor CDS | 576 | U | 2.5 | 1 |
| AIG67827.1 | Putative lipoprotein yceB precursor CDS | 561 | U | 2 | 0 |
| AIG67831.1 | Protein of unknown function YceH CDS | 648 | U | 2 | 0 |
| AIG67832.1 | Virulence factor MviM CDS | 924 | U | 2 | 0 |
| AIG67840.1 | Flagellar basal-body rod modification | 696 | Ex | 10 | 0 |
| | protein FlgD CDS | | | | |
| AIG67841.1 | Flagellar hook protein FlgE CDS | 1206 | Ex | 10 | 0 |
| AIG67843.1 | Flagellar basal-body rod protein FlgG CDS | 783 | Ex | 10 | 0 |
| AIG67844.1 | Flagellar L-ring protein FlgH CDS | 699 | OM | 10 | 1 |
| AIG67847.1 | Flagellar hook-associated protein FlgK CDS | 1644 | Ex | 9.96 | 0 |
| AIG67848.1 | Flagellar hook-associated protein FlgL CDS | 954 | Ex | 10 | 0 |
| AIG67862.1 | YceG like protein CDS | 1023 | U | 6.49 | 1 |
| AIG67867.1 | Putative OMR family iron-siderophore | 2190 | OM | 10 | 0 |
| | receptor precursor CDS | | | | |
| AIG67871.1 | Lipoprotein YcfM, part of a salvage | 642 | U | 2 | 0 |
| | pathway of unknown substrate CDS | | | | |
| AIG67876.1 | Putative exported protein CDS | 540 | U | 2.5 | 1 |
| AIG67905.1 | hypothetical protein CDS | 588 | U | 2 | 0 |
| AIG67914.1 | Hypothetical protein CDS | 1938 | U | 2 | 0 |
| AIG67929.1 | hypothetical protein CDS | 1938 | U | 2 | 0 |
| AIG67963.1 | hypothetical protein CDS | 744 | U | 2 | 0 |
| AIG68016.1 | hypothetical protein CDS | 1602 | U | 2 | 0 |
| AIG68033.1 | Attachment invasion locus protein precursor | 600 | OM | 10 | 1 |
| | CDS | | | | |
| AIG68048.1 | Protease VII (Omptin) precursor CDS | 954 | OM | 10 | 0 |
| AIG68053.1 | Pertactin precursor CDS | 705 | U | 7 | 0 |
| AIG68064.1 | Hemolysin E, chromosomal CDS | 1056 | Ex | 10 | 0 |
| AIG68081.1 | Putative TonB dependent outer membrane | 1971 | OM | 10 | 0 |
| | receptor CDS | | | | |
| AIG68093.1 | Putative adhesion and penetration protein | 1644 | Ex | 9.65 | 0 |
| | CDS | | | | |
| AIG68114.1 | Invasin CDS | 1431 | OM | 10 | 0 |
| AIG68125.1 | UPF0028 protein YchK CDS | 945 | Ex | 8.89 | 0 |
| AIG68148.1 | Outer membrane protein W precursor CDS | 639 | OM | 10 | 0 |
| AIG68163.1 | hypothetical protein CDS | 582 | U | 2 | 0 |
| AIG68165.1 | Putative intestinal colonization factor | 747 | U | 2 | 0 |
| | encoded by prophage CP-933O CDS | | | | |
| AIG68173.1 | Hypothetical protein CDS | 1854 | U | 2 | 0 |
| AIG68183.1 | hypothetical protein CDS | 1938 | U | 2 | 0 |
| AIG68190.1 | putative major tail subunit CDS | 786 | Ex | 9.64 | 0 |
| AIG68238.1 | Hypothetical protein CDS | 1851 | U | 2 | 0 |
| AIG68271.1 | Attachment invasion locus protein precursor | 600 | OM | 10 | 1 |
| | CDS | | | | |
| AIG68309.1 | Autoinducer 2 (AI-2) aldolase LsrF CDS | 876 | U | 2 | 0 |
| AIG68316.1 | hypothetical protein CDS | 3894 | U | 5.87 | 0 |
| AIG68317.1 | hypothetical protein CDS | 1401 | OM | 9.83 | 0 |
| AIG68320.1 | type 1 fimbriae major subunit FimA CDS | 564 | Ex | 10 | 1 |
| AIG68322.1 | type 1 fimbriae anchoring protein FimD | 2223 | OM | 10 | 0 |
| | CDS | | | | |
| AIG68325.1 | mannose-specific adhesin FimH CDS | 915 | U | 2 | 1 |
| AIG68333.1 | hypothetical protein CDS | 2373 | OM | 9.49 | 0 |
| AIG68337.1 | redicted glycoside hydrolase CDS | 1320 | U | 2 | 0 |
| AIG68357.1 | Outer membrane porin protein NmpC | 1101 | OM | 10 | 1 |
| | precursor CDS | | | | |
| AIG68359.1 | hypothetical protein CDS | 669 | U | 2 | 0 |
| AIG68376.1 | internalin, putative CDS | 1260 | U | 2 | 0 |
| AIG68383.1 | hypothetical protein CDS | 1062 | U | 2.5 | 0 |
| AIG68385.1 | core protein CDS | 4203 | U | 5.48 | 0 |
| AIG68385.1 | putative tonB-dependent receptor yncD | 2019 | OM | 10 | 0 |
| | precursor CDS | | | | |
| AIG68406.1 | putative membrane lipoprotein clustered | 669 | U | 2 | 0 |
| | with tellurite resistance proteins TehA/TehB | | | | |
| | CDS | | | | |
| AIG68433.1 | putative BigA-like protein CDS | 2460 | U | 6.26 | 0 |
| AIG68444.1 | Outer membrane protein N precursor CDS | 753 | OM | 10 | 0 |
| AIG68484.1 | porin, autotransporter (AT) family CDS | 3036 | Ex | 9.65 | 2 |
| AIG68484.1 | Hypothetical protein CDS | 1854 | U | 2 | 0 |
| AIG68547.1 | Outer membrane protein G precursor CDS | 906 | OM | 10 | 0 |
| AIG68579.1 | RND efflux system, outer membrane | 1374 | OM | 9.98 | 0 |
| | lipoprotein CmeC CDS | | | | |
| AIG68638.1 | Attachment invasion locus protein precursor | 600 | OM | 10 | 1 |
| | CDS | | | | |
| AIG68679.1 | Hypothetical protein CDS | 1851 | U | 2 | 0 |
| AIG68688.1 | hypothetical protein CDS | 585 | U | 2 | 0 |
| AIG68723.1 | Putative protease ydgD | 822 | U | 2.5 | U |
| AIG68730.1 | Protein ydgH precursor CDS | 945 | U | 2.5 | 0 |
| AIG68784.1 | Putative lipoprotein CDS | 816 | U | 6.49 | 0 |
| AIG68794.1 | putative enzyme CDS | 1257 | U | 7 | 0 |
| AIG68797.1 | hypothetical protein CDS | 813 | U | 2 | 0 |
| AIG68812.1 | Iron-sulfur cluster assembly protein SufB | 1488 | U | 2 | 0 |
| | CDS | | | | |
| AIG68838.1 | hypothetical protein CDS | 714 | U | 2 | 0 |
| AIG68851.1 | hypothetical protein CDS | 1899 | U | 2 | 0 |
| AIG68853.1 | Putative outer membrane protein CDS | 759 | U | 2.5 | 0 |
| AIG68861.1 | hypothetical protein CDS | 768 | U | 2 | 0 |
| AIG68883.1 | Hypothetical protein YdjY CDS | 678 | U | 2.5 | 0 |
| AIG68886.1 | ABC transporter, periplasmic substrate- | 1167 | U | 2.5 | 0 |
| | binding protein YnjB CDS | | | | |
| AIG68897.1 | Protein ydjA CDS | 552 | U | 2 | 1 |
| AIG68899.1 | putative lipoprotein CDS | 711 | U | 2 | 1 |
| AIG68918.1 | MltA-interacting protein MipA CDS | 747 | OM | 10 | 0 |
| AIG68945.1 | Starvation lipoprotein Slp-like protein CDS | 582 | OM | 9.92 | 1 |
| AIG68946.1 | Inactive metal-dependent proteases like | 696 | U | 2 | U |
| | protein, putative molecular chaperone | | | | |
| AIG68978.1 | Paraquat-inducible protein B CDS | 2640 | U | 2 | 2 |
| AIG68989.1 | Protease II | 2061 | U | 5.41 | U |
| AIG69001.1 | Cell wall endopeptidase, family M23/M37 | 1323 | U | 2 | 1 |
| | CDS | | | | |
| AIG69007.1 | hypothetical protein CDS | 603 | U | 2 | 0 |
| AIG69057.1 | putative membrane protein CDS | 651 | U | 2 | 1 |
| AIG69095.1 | Flagellar biosynthesis protein FliZ CDS | 552 | U | 2 | 0 |
| AIG69098.1 | Flagellar biosynthesis protein FliC CDS | 1758 | Ex | 9.96 | 0 |
| AIG69099.1 | Flagellar hook-associated protein FliD CDS | 1398 | Ex | 10 | 0 |
| AIG69108.1 | invasion plasmid antigen CDS | 1140 | Ex | 8.89 | 0 |
| AIG69110.1 | invasion plasmid antigen CDS | 1140 | Ex | 8.89 | 0 |
| AIG69118.1 | Flagellar hook-length control protein FliK | 1128 | Ex | 10 | 0 |
| | CDS | | | | |
| AIG69140.1 | Outer membrane protein N precursor CDS | 645 | OM | 10 | 1 |
| AIG69141.1 | Outer membrane protein N precursor CDS | 576 | OM | 10 | 0 |
| AIG69149.1 | putative zinc-binding lipoprotein ZinT CDS | 651 | U | 2.5 | 0 |
| AIG69153.1 | hypothetical protein CDS | 711 | U | 2 | 0 |
| AIG69158.1 | Attachment invasion locus protein precursor | 600 | OM | 10 | 1 |
| | CDS | | | | |
| AIG69189.1 | Hypothetical protein CDS | 1851 | U | 2 | 0 |
| AIG69198.1 | hypothetical protein CDS | 564 | U | 2 | 0 |
| AIG69216.1 | adherence and invasion outermembrane | 7863 | OM | 9.95 | 0 |
| | protein (Inv, enhances Peyer's patches | | | | |
| | colonization) CDS | | | | |
| AIG69305.1 | AsmA protein CDS | 1854 | U | 2.5 | 1 |
| AIG69312.1 | Putative chaperonin CDS | 1941 | U | 2 | 2 |
| AIG69314.1 | hypothetical protein CDS | 660 | U | 2 | 0 |
| AIG69346.1 | Uncharacterized protein YehA precursor | 1035 | U | 2 | 0 |
| | CDS | | | | |
| AIG69347.1 | Fimbriae usher protein StcC CDS | 2481 | OM | 10 | 0 |
| AIG69349.1 | Putative fimbrial-like protein CDS | 543 | U | 2.5 | 1 |
| AIG69361.1 | hypothetical protein CDS | 1530 | U | 2 | 0 |
| AIG69362.1 | hypothetical protein CDS | 732 | U | 2 | 0 |
| AIG69377.1 | Attachment invasion locus protein precursor | 600 | OM | 10 | 1 |
| | CDS | | | | |
| AIG69390.1 | Minor tail protein Z CDS | 624 | U | 2 | 0 |
| AIG69393.1 | Prophage Clp protease-like protein | 1041 | U | | U |
| AIG69411.1 | Shiga toxin A-chain precursor CDS | 948 | U | 2.5 | 1 |
| AIG69429.1 | putative superinfection exclusion protein | 555 | U | 2 | 2 |
| | CDS | | | | |
| AIG69485.1 | Colicin I receptor precursor CDS | 1884 | OM | 10 | 0 |
| AIG69508.1 | Lipoprotein spr precursor CDS | 567 | U | 2.5 | 0 |
| AIG69510.1 | ABC transporter, periplasmic substrate- | 1815 | U | 6.58 | 0 |
| | binding protein CDS | | | | |
| AIG69523.1 | Putative ATP-binding component of a | 2592 | Ex | 9.46 | 0 |
| | transport system CDS | | | | |
| AIG69547.1 | Outer membrane protein C precursor CDS | 1104 | OM | 10 | 1 |
| AIG69552.1 | hypothetical protein CDS | 777 | U | 2 | 0 |
| AIG60553.1 | Putative membrane protein CDS | 4515 | U | 4.69 | 0 |
| AIG60558.1 | Type V secretory pathway, adhesin AidA | 3705 | OM | 9.83 | 0 |
| | CDS | | | | |
| AIG69581.1 | Polymyxin resistance protein PmrG CDS | 603 | U | 2.5 | 1 |
| AIG69644.1 | DedD protein CDS | 663 | U | 2 | 1 |
| AIG69664.1 | Uncharacterized protein YadU in stf | 843 | U | 2 | 0 |
| | fimbrial cluster CDS | | | | |
| AIG69669.1 | Fimbriae usher protein StfC CDS | 2583 | OM | 10 | 0 |
| AIG69676.1 | Long-chain fatty acid transport protein CDS | 1341 | OM | 10 | 0 |
| AIG69679.1 | Lipoprotein CDS | 756 | OM | 9.92 | 2 |
| AIG69706.1 | hypothetical protein CDS | 636 | U | 2.5 | 0 |
| AIG69727.1 | putative virulence protein CDS | 1149 | U | 2 | 0 |
| AIG60790.1 | YpfJ protein, zinc metalloprotease | 864 | U | 2 | U |
| | superfamily | | | | |
| AIG69792.1 | Outer membrane protein NlpB, lipoprotein | 1035 | OM | 9.93 | 0 |
| | component of the protein assembly complex | | | | |
| | (forms a complex with YaeT, YfiO, and | | | | |
| | YfgL) CDS | | | | |
| AIG69809.1 | Exported zinc metalloprotease YfgC | 1464 | U | 2.5 | U |
| | precursor | | | | |
| AIG69829.1 | Outer membrane protein YfgL, lipoprotein | 1179 | OM | 9.92 | 0 |
| | component of the protein assembly complex | | | | |
| | (forms a complex with YaeT, YfiO, and | | | | |
| | NlpB) CDS | | | | |
| AIG69833.1 | putative membrane protein CDS | 1014 | U | 2 | 1 |
| AIG69842.1 | Thiosulfate sulfurtransferase, rhodanese | 846 | U | 5.41 | 1 |
| | CDS | | | | |
| AIG69880.1 | putative alpha helix protein CDS | 636 | U | 2 | 0 |
| AIG69913.1 | hypothetical protein CDS | 564 | U | 2 | 0 |
| AIC69916.1 | hypothetical protein CDS | 732 | U | 2 | 0 |
| AIC69918.1 | putative component of the lipoprotein | 738 | OM | 10 | 0 |
| | assembly complex (forms a complex with | | | | |
| | YaeT, YfgL, and NlpB) CDS | | | | |
| AIG69931.1 | Signal recognition particle, subunit Ffh | 1362 | U | 5.6 | 0 |
| | SRP54 CDS | | | | |
| AIG69958.1 | Hypothetical protein CDS | 1854 | U | 2 | 0 |
| AIG69974.1 | Pertactin precursor CDS | 4308 | U | 5.87 | 0 |
| AIG70043.1 | Coenzyme F420 hydrogenase maturation | 471 | U | | U |
| | protease | | | | |
| AIG70069.1 | Lipoprotein NlpD CDS | 1140 | OM | 9.93 | 0 |
| AIC70199.1 | Type III secretion bridge between inner and | 735 | OM | 9.92 | 1 |
| | outermembrane lipoprotein | | | | |
| | (YscJ, HrcJ, EscJ, PscJ) CDS | | | | |
| AIG70202.1 | Type III secretion protein EprH CDS | 735 | U | 2 | 1 |
| AIG70217.1 | Uncharacterized protein YgeP CDS | 1026 | U | 2 | 0 |
| AIG70219.1 | putative lipoprotein YgeR precursor CDS | 756 | OM | 9.93 | 1 |
| AIG70290.1 | Uridine kinase family protein CDS | 714 | U | 2 | 0 |
| AIG70298.1 | Putative metalloprotease yggG | 759 | U | 2 | U |
| AIG70300.1 | hypothetical protein CDS | 732 | U | 2.5 | 0 |
| AIG70312.1. | UPF0301 protein YqgE CDS | 564 | U | 2 | 0 |
| AIG70323.1 | Uncharacterized protein YggN CDS | 720 | U | 2.5 | 0 |
| AIG70398.1 | Modulator of drug activity B CDS | 582 | U | 2 | 0 |
| AIG70405.1 | Ferrichrome-iron receptor CDS | 2142 | OM | 10 | 0 |
| AIG70411.1 | Type I secretion outer membrane protein, | 1482 | OM | 10 | 0 |
| | TolC precursor CDS | | | | |
| AIG70414.1 | Uncharacterized protein ygiD CDS | 816 | U | 2 | 0 |
| AIG70516.1 | type 1 fimbriae anchoring protein FimD | 2592 | OM | 10 | 0 |
| | CDS | | | | |
| AIG70532.1 | Putative lipid carrier protein CDS | 525 | U | 2 | 0 |
| AIG70535.1 | Putative protease | 879 | U | 2 | U |
| AIG70568.1 | putative ABC transporter, auxiliary | 636 | U | 2.5 | 0 |
| | component YrbC CDS | | | | |
| AIG70569.1 | putative ABC transporter, periplasmic | 552 | U | 2.5 | 1 |
| | component YrbD CDS | | | | |
| AIG70575.1 | Uncharacterized protein YrbK clustered | 576 | U | 2 | 1 |
| | with lipopolysaccharide transporters CDS | | | | |
| AIG70604.1 | Outer membrane stress sensor protease | 1368 | P | | U |
| | DegQ, serine protease | | | | |
| AIG70605.1 | Outer membrane stress sensor protease | 1068 | P | | U |
| | DegS | | | | |
| AIG70619.1 | Rod shape-determining protein MreC CDS | 1104 | U | 2 | 1 |
| AIG70742.1 | Type IV pilus biogenesis protein PilM CDS | 795 | U | 2 | 0 |
| AIG70779.1 | hypothetical protein CDS | 993 | U | 2.5 | 0 |
| AIG70798.1 | hypothetical protein CDS | 912 | U | 2 | 0 |
| AIG70844.1 | Putative transmembrane protein CDS | 606 | U | 2.5 | 0 |
| AIG70880.1 | hypothetical protein CDS | 753 | U | 2 | 0 |
| AIG70893.1 | TonB-dependent hemin, ferrichrome | 1983 | OM | 10 | 0 |
| | receptor CDS | | | | |
| AIG70922.1 | Uncharacterized protein YhjG CDS | 2061 | U | 2 | 2 |
| AIG70925.1 | Protein YhjJ, putative peptidase CDS | 1497 | U | 2.5 | 0 |
| AIG70935.1 | hypothetical protein CDS | 1560 | U | 2 | 0 |
| AIG70950.1 | Putative fimbrial protein CDS | 1056 | Ex | 9.65 | 1 |
| AIG70952.1 | Long polar fimbria protein A precursor | 525 | Ex | 9.72 | 0 |
| | CDS | | | | |
| AIG70954.1 | Putative lipase CDS | 699 | U | 7 | 1 |
| AIG70958.1 | Outer membrane protein A precursor CDS | 660 | OM | 10 | 3 |
| AIG70960.1 | putative exported protein CDS | 711 | U | 2.5 | 0 |
| AIG70976.1 | BAX protein CDS | 573 | U | 2 | 0 |
| AIG70983.1 | Putative outer membrane protein yiaT | 741 | OM | 10 | 1 |
| | precursor CDS | | | | |
| AIG70995.1 | core protein CDS | 4230 | U | 2 | 0 |
| AIG71007.1 | hypothetical protein CDS | 4767 | OM | 9.95 | 2 |
| AIG71034.1 | Lipopolysaccharide heptosyltransferase III | 1023 | U | 2 | 0 |
| | CDS | | | | |
| AIG71060.1 | Putative exported protein CDS | 1710 | U | 2 | 1 |
| AIG71083.1 | Secreted protein EspB CDS | 939 | Ex | 10 | 1 |
| AIG71084.1 | Secreted protein EspD CDS | 1125 | Ex | 10 | 3 |
| AIG71087.1 | Type III secretion system EscD protein | 1221 | U | 2 | 1 |
| | CDS | | | | |
| AIG71088.1 | Intimin CDS | 2805 | OM | 10 | 2 |
| AIG71090.1 | translocated intimin receptor Tir CDS | 1677 | Ex | 10 | 2 |
| AIG71096.1 | SepQ CDS | 918 | U | 2 | 0 |
| AIG71102.1 | Type III secretion bridge between inner and | 573 | OM | 9.93 | 0 |
| | outermembrane lipoprotein | | | | |
| | (YscJ, HrcJ, EscJ, PscJ) CDS | | | | |
| AIG71104.1 | Type III secretion outermembrane pore | 1539 | OM | 10 | 0 |
| | forming protein (YscC, MxiD, HrcC, InvG) | | | | |
| | CDS | | | | |
| AIG71117.1 | ROrf2 CDS | 1146 | Ex | 10 | 0 |
| AIG71137.1 | hypothetical protein CDS | 624 | U | 2 | 0 |
| AIG71158.1 | Uncharacterized protein YidR CDS | 1251 | U | 2 | 0 |
| AIG71159.1 | Uncharacterized protein YidS CDS | 1083 | U | 2 | 0 |
| AIG71177.1 | hypothetical protein CDS | 750 | U | 2 | 0 |
| AIG71181.1 | hypothetical protein CDS | 2409 | U | 5.48 | 0 |
| AIG71182.1 | hypothetical protein CDS | 672 | U | 2 | 0 |
| AIG71192.1 | Putative fimbrial protein CDS | 1083 | Ex | 10 | 0 |
| AIG71193.1 | Putative fimbrial protein CDS | 1071 | Ex | 9.65 | 0 |
| AIG71194.1 | type 1 fimbriae anchoring protein FimD | 2535 | OM | 9.93 | 1 |
| | CDS | | | | |
| AIG71272.1 | hypothetical protein CDS | 903 | U | 2.5 | 0 |
| AIG71273.1 | hypothetical protein CDS | 765 | U | 2.5 | 0 |
| AIG71277.1 | hypothetical protein CDS | 849 | OM | 9.49 | 0 |
| AIG71291.1 | Putative carboxymethylenebutenolidase | 816 | U | 2 | 0 |
| | CDS | | | | |
| AIG71333.1 | Outer membrane sugar transport protein | 693 | OM | 9.93 | 1 |
| | YshA CDS | | | | |
| AIG71349.1 | hypothetical protein CDS | 915 | U | 2 | 1 |
| AIG71360.1 | hypothetical protein CDS | 1056 | U | 4.9 | 0 |
| AIG71365.1 | Putative glycoporin CDS | 1395 | U | 2.5 | 0 |
| AIG71378.1 | hypothetical protein CDS | 675 | U | 2 | 0 |
| AIG71388.1 | Putative uncharacterized protein YiiQ CDS | 600 | U | 2.5 | 1 |
| AIG71408.1 | core protein CDS | 4185 | U | 2 | 0 |
| AIG71411.1 | hypothetical protein CDS | 609 | U | 2.5 | 0 |
| AIG71419.1 | hypothetical protein CDS | 618 | U | 2.5 | 0 |
| AIG71443.1 | Outer membrane vitamin B12 receptor BtuB | 1845 | OM | 10 | 0 |
| | CDS | | | | |
| AIG71471.1 | hypothetical protein CDS | 636 | U | 2 | 0 |
| AIG71532.1 | NMN phosphatase CDS | 714 | U | 2.5 | 1 |
| AIG71558.1 | Putative exported protein CDS | 690 | U | 2.5 | 0 |
| AIG71638.1 | Outer membrane lipoprotein BLc CDS | 534 | OM | 10 | 1 |
| AIG71658.1 | MflK protein CDS | 1260 | U | 5.48 | 1 |
| AIG71669.1 | hypothetical protein CDS | 639 | U | 2 | 0 |
| AIG71674.1 | YjfP protein CDS | 750 | U | 2 | 0 |
| AIG71687.1 | Uncharacterized protein yjfZ CDS | 795 | U | 2 | 0 |
| AIG71690.1 | putative virulence protein CDS | 1149 | U | 2 | 0 |
| AIG71691.1 | Putative cell envelope opacity-associated | 639 | U | 2 | 0 |
| | protein A CDS | | | | |
| AIG71701.1 | Protein ytfJ precursor CDS | 555 | U | 4.9 | 0 |
| AIG71707.1 | Uncharacterized protein YtfM precursor | 1734 | OM | 10 | 0 |
| | CDS | | | | |
| AIG71708.1 | Uncharacterized protein YtfN CDS | 3780 | U | 4.72 | 1 |
| AIG71752.1 | hypothetical protein CDS | 1503 | U | 2 | 0 |
| AIG71763.1 | hypothetical protein CDS | 774 | U | 2.5 | 0 |
| AIG71781.1 | hypothetical protein CDS | 981 | U | 2 | 0 |
| AIG71788.1 | type 1 fimbriae major subunit FimA CDS | 549 | Ex | 10 | 1 |
| AIG71791.1 | type 1 fimbriae anchoring protein FimD | 2637 | OM | 10 | 0 |
| | CDS | | | | |
| AIG71794.1 | mannose-specific adhesin FimH CDS | 903 | U | 4.65 | 2 |
| AIG71800.1 | Uncharacterized protein YjiC CDS | 831 | U | 2 | 0 |
| AIG71813.1 | hypothetical protein CDS | 843 | U | 4.9 | 1 |
| AIG71857.1 | hypothetical protein CDS | 1074 | U | 2 | 0 |
| AIG68360.1 | internalin, putative_EDL933_2172 | 1260 | U | 2 | 0 |

¹PSORT Localization, where Ex = Extracellular, OM = Outer Membrane, P = Periplasmic, U = Unknown
²Number of internal helices, where U = unknown

TABLE S2
Vaccine candidates expressed at transcriptional level (cutoff >10 RPKM)

| | | | | Expression (absolute value RPKM) | | | |
|---|---|---|---|---|---|---|---|
| Protein | Locus | Functional | PSORT | RPKM | RPKM | RPKM (LB | RPKM (LB |
| ID | Tag | annotation | score | (LB) | (LB agar) | antibiotics) | Feces) |
| AIG66267.1 | EDL933_0059 | hypothetical protein CDS | 2 | 32.7 | 65.6 | 75.0 | 0.0 |
| AIG66347.1 | EDL933_0139 | Fimbrial protein Yad like | 2 | 192.8 | 327.0 | 101.7 | 347.5 |
| | | protein CDS | | | | | |
| AIG66348.1 | EDL933_0140 | Fimbrial protein YadK CDS | 2 | 32.2 | 37.6 | 0.0 | 171.7 |
| AIG66349.1 | EDL933_0141 | Fimbrial protein YadL CDS | 2.5 | 44.4 | 48.9 | 0.0 | 134.0 |
| AIG66424.1 | EDL933_0216 | putative aminopeptidase CDS | 2.5 | 9.8 | 18.5 | 105.7 | 0.0 |
| AIG66447.1 | EDL933_0241 | hypothetical protein CDS | 2 | 223.3 | 250.2 | 288.3 | 779.5 |
| AIG66490.1 | EDL933_0284 | putative tail fiber protein CDS | 2 | 12 | 0.0 | 1305.2 | 51.1 |
| AIG66511.1 | EDL933_0305 | Zinc binding domain protein CDS | 2 | 11 | 10.6 | 48.4 | 30.4 |
| AIG66518.1 | EDL933_0312 | hypothetical protein CDS | 2.5 | 24.6 | 0.0 | 24.8 | 0.0 |
| AIG66656.1 | EDL933_0452 | Protein YkiA CDS | 2 | 8.3 | 6.8 | 0.0 | 28.1 |
| AIG66781.1 | EDL933_0577 | Putative stomatin/prohibitin- | 2 | 45.2 | 225.9 | 0.0 | 619.2 |
| | | family membrane protease | | | | | |
| | | subunit YbbK | | | | | |
| AIG66972.1 | EDL933_0770 | core protein CDS | 2 | 6.8 | 12.9 | 6.7 | 4.8 |
| AIG66984.1 | EDL933_0782 | hypothetical protein CDS | 2.5 | 21.6 | 18.6 | 13.3 | 38.2 |
| AIG66987.1 | EDL933_0786 | putative fimbrial-like | 9.72 | 41.5 | 17.4 | 24.9 | 143.2 |
| | | protein ygiL precursor | | | | | |
| | | CDS | | | | | |
| AIG67020.1 | EDL933_0819 | TolA protein CDS | 2 | 454.8 | 283.4 | 405.1 | 325.5 |
| AIG67060.1 | EDL933_0859 | Putative outer membrane | 2.5 | 45.5 | 30.9 | 338.2 | 126.9 |
| | | protein CDS | | | | | |
| AIG67245.1 | EDL933_1046 | Urease accessory protein | 2 | 23.2 | 0.0 | 0.0 | 0.0 |
| | | UreF CDS | | | | | |
| AIG67308.1 | EDL933_1109 | putative hemolysin activator | 8.86 | 0 | 0.0 | 13.3 | 19.1 |
| | | protein CDS | | | | | |
| AIG67453.1 | EDL933_1258 | hypothetical protein CDS | 2 | 68 | 51.0 | 383.0 | 131.1 |
| AIG67464.1 | EDL933_1259 | Hypothetical protein CDS | 2 | 24.9 | 27.4 | 78.4 | 0.0 |
| AIG67503.1 | EDL933_1308 | hypothetical protein CDS | 9.71 | 35.7 | 45.9 | 0.0 | 53.9 |
| AIG67577.1 | EDL933_1383 | Shiga-like toxin II subunit | 2.5 | 372.9 | 262.4 | 566.2 | 42.3 |
| | | A precursor CDS | | | | | |
| AIG67652.1 | EDL933_1458 | Putative exported protein | 2 | 17.5 | 9.1 | 13.0 | 37.4 |
| | | precursor CDS | | | | | |
| AIG67653.1 | EDL933_1459 | outer membrane fimbrial | 10 | 11.5 | 7.8 | 0.0 | 72.4 |
| | | usher protein CDS | | | | | |
| AIG67671.1 | EDL933_1477 | hypothetical protein CDS | 2 | 2.8 | 0.0 | 0.0 | 25.7 |
| AIG67672.1 | EDL933_1478 | hypothetical protein CDS | 2 | 2.4 | 0.0 | 0.0 | 21.9 |
| AIG67760.1 | EDL933_1570 | putative hemolysin | 8.86 | 3.2 | 4.6 | 13.3 | 76.2 |
| | | activator protein CDS | | | | | |
| AIG67905.1 | EDL933_1717 | hypothetical protein CDS | 2 | 32.4 | 8.4 | 144.1 | 138.1 |
| AIG67914.1 | EDL933_1726 | Hypothetical protein CDS | 2 | 3.5 | 0.0 | 0.0 | 10.5 |
| AIG67963.1 | EDL933_1775 | hypothetical protein CDS | 2 | 484.9 | 491.2 | 1537.0 | 2182.8 |
| AIG68016.1 | EDL933_1828 | hypothetical protein CDS | 2 | 3.8 | 0.0 | 8.8 | 12.7 |
| AIG68165.1 | EDL933_1977 | Putative intestinal colonization | 2 | 30 | 171.9 | 0.0 | 135.9 |
| | | factor encoded by prophage | | | | | |
| | | CP-933O CDS | | | | | |
| AIG68173.1 | EDL933_1985 | Hypothetical protein CDS | 2 | 70.7 | 40.0 | 7.6 | 10.9 |
| AIG68238.1 | EDL933_2050 | Hypothetical protein CDS | 2 | 53.3 | 26.7 | 45.8 | 0.0 |
| AIG68357.1 | EDL933_2169 | Outer membrane porin | 10 | 16.3 | 22.4 | 0.0 | 110.6 |
| | | protein NmpC precursor | | | | | |
| | | CDS | | | | | |
| AIG68359.1 | EDL933_2171 | hypothetical protein CDS | 2 | 1.7 | 14.8 | 0.0 | 30.3 |
| AIG68376.1 | EDL933_2189 | internalin, putative CDS | 2 | 6.3 | 2.3 | 10.1 | 19.3 |
| AIG68484.1 | EDL933_2297 | Hypothetical protein CDS | 2 | 8.5 | 8.0 | 30.5 | 10.9 |
| AIG68679.1 | EDL933_2505 | Hypothetical protein CDS | 2 | 27.2 | 13.3 | 15.3 | 21.9 |
| AIG68688.1 | EDL933_2514 | hypothetical protein CDS | 2 | 76.6 | 33.8 | 0.0 | 242.9 |
| AIG69057.1 | EDL933_2888 | putative membrane protein CDS | 2 | 606.7 | 933.1 | 401.2 | 1262.9 |
| AIG69153.1 | EDL933_2986 | hypothetical protein CDS | 2 | 108.7 | 83.4 | 119.1 | 142.8 |
| AIG69189.1 | EDL933_3022 | Hypothetical protein CDS | 2 | 18.8 | 10.7 | 30.5 | 0.0 |
| AIG69198.1 | EDL933_3031 | hypothetical protein CDS | 2 | 37.7 | 78.8 | 25.0 | 180.0 |
| AIG69346.1 | EDL933_3180 | Uncharacterized protein | 2 | 14.1 | 0.0 | 0.0 | 19.6 |
| | | YehA precursor CDS | | | | | |
| AIG69349.1 | EDL933_3183 | Putative fimbrial-like | 2.5 | 88.7 | 582.1 | 208.0 | 37.4 |
| | | protein CDS | | | | | |
| AIG69390.1 | EDL933_3224 | Minor tail protein Z CDS | 2 | 3.6 | 0.0 | 22.6 | 0.0 |
| AIG69393.1 | EDL933_3227 | Prophage Clp protease- | | 2.2 | 0.0 | 13.6 | 0.0 |
| | | like protein | | | | | |
| AIG69411.1 | EDL933_3245 | Shiga toxin A-chain | 2.5 | 654.7 | 1586.3 | 871.2 | 235.5 |
| | | precursor CDS | | | | | |
| AIG69664.1 | EDL933_3500 | Uncharacterized protein | 2 | 33.2 | 93.7 | 50.2 | 72.2 |
| | | YadU in stf fimbrial | | | | | |
| | | cluster CDS | | | | | |
| AIG69958.1 | EDL933_3799 | Hypothetical protein CDS | 2 | 31.4 | 16.0 | 22.8 | 21.9 |
| AIG70298.1 | EDL933_4145 | Putative metalloprotease yggG | 2 | 519.6 | 390.4 | 93.0 | 320.9 |
| AIG70798.1 | EDL933_4653 | hypothetical protein CDS | 2 | 3.7 | 0.0 | 15.5 | 22.3 |
| AIG70844.1 | EDL933_4699 | Putative transmembrane | 2.5 | 10.2 | 0.0 | 0.0 | 0.0 |
| | | protein CDS | | | | | |
| AIG71087.1 | EDL933_4946 | Type III secretion system | 2 | 55.1 | 44.5 | 46.2 | 83.1 |
| | | EscD protein CDS | | | | | |
| AIG71096.1 | EDL933_4955 | SepQ CDS | 2 | 223.3 | 102.2 | 399.8 | 309.6 |
| AIG71137.1 | EDL933_4996 | hypothetical protein CDS | 2 | 19.8 | 39.6 | 22.6 | 130.1 |
| AIG71181.1 | EDL933_5041 | hypothetical protein CDS | 5.48 | 5.6 | 0.0 | 111.3 | 16.9 |
| AIG71182.1 | EDL933_5042 | hypothetical protein CDS | 2 | 0 | 14.7 | 84.0 | 151.0 |
| AIG71349.1 | EDL933_5210 | hypothetical protein CDS | 2 | 6.1 | 16.2 | 0.0 | 0.0 |
| AIG71763.1 | EDL933_5624 | hypothetical protein CDS | 2.5 | 34.7 | 82.9 | 145.9 | 26.2 |
| AIG71811.1 | EDL933_5672 | adherence and invasion | 9.95 | 17.3 | 23.5 | 11.2 | 64.5 |
| | | outermembrane protein | | | | | |
| | | (Inv, enhances Peyer's | | | | | |
| | | patches colonization) CDS | | | | | |
| AIG66227.1 | EDL933_0019 | Putative outer membrane | 10.00 | 3.2 | 10.1 | 0.0 | 8.3 |
| | | protein EDL933 0019 | | | | | |
| AIG68053.1 | EDL933_1865 | Pertactin precursor | 7.00 | 36.6 | 42.0 | 40.0 | 28.8 |
| AIG68360.1 | EDL933_2172 | internalin, putative | 2.00 | 8 | 7.8 | 11.2 | 145.0 |
| AIG69216.1 | EDL933_3049 | adherence and invasion | 9.95 | 5.8 | 12.6 | 10.8 | 10.3 |
| | | outermembrane protein | | | | | |
| | | (Inv, enhances Peyer's | | | | | |
| | | patches colonization) | | | | | |
| AIG69974.1 | EDL933_3815 | Pertactin precursor | 5.87 | 12 | 21.8 | 3.3 | 47.1 |
| AIG68899.1 | EDL933_2727 | Putative lipoprotein | 2 | 11 | 6.9 | 0 | 0 |

TABLE S3
Features of the 24 antigen candidates expressed as recombinant proteins

| | | | | | | | | Expression (absolute value RPKM) | | | | |
|---|---|---|---|---|---|---|---|---|---|---|---|---|
| | Protein | | Functional | | | | Pfam | | LB | LB | | |
| No. | ID | Desig | annotation | Sol. | kDa | Localization | domain | LB | Agar | Antibiotics | Feces | WB |
| 1 | AIG67060.1 | MC001 | Putative outer | I | 35 | OM | DUF2219 | 45.5 | 30.9 | 338.2 | 126.9 | ++ |
| | | | membrane protein | | | | | | | | | |
| 2 | AIG68165.1 | MC002 | Putative intestinal | S | 27 | EC | SBP bac 11 | 30 | 171.9 | 0 | 135.9 | ++ |
| | | | colonization factor | | | | | | | | | |
| | | | encoded by prophage | | | | | | | | | |
| | | | CP-933O | | | | | | | | | |
| 3 | AIG68357.1 | MC003 | Outer membrane porin | I | 41 | OM | Porin 1 | 16.13 | 22.4 | 0 | 110.6 | + |
| | | | protein NmpC | | | | | | | | | |
| | | | precursor | | | | | | | | | |
| 4 | AIG67577.1 | MC004 | Shiga-like toxin | I | 36 | EC | RIP | 372 | 262.4 | 566.2 | 42.3 | + |
| | | | II subunit A | | | | | | | | | |
| | | | precursor | | | | | | | | | |
| 5 | AIG69411.1 | MC005 | Shiga toxin | S | 35 | EC | RIP | 654.7 | 1586.3 | 871.2 | 235.5 | − |
| | | | A-chain precursor | | | | | | | | | |
| 6 | AIG66347.1 | MC006 | Fimbrial protein Yad | S | 40 | OM | Fimbrial | 192.8 | 327 | 101.7 | 347.5 | − |
| | | | like protein | | | | | | | | | |
| 7 | AIG66424.1 | MC007 | putative aminopeptidase | S | 30 | Unknown | unknown | 9.8 | 18.5 | 105.7 | 0 | ++ |
| 8 | AIG66984.1 | MC008 | hypothetical protein | I | 40 | Fimbrial | Fimbrial | 21.6 | 18.6 | 13.3 | 38.2 | + |
| 9 | AIG67652.1 | MC009 | Putative exported | I | 38 | OM | Fimbrial | 17.5 | 9.1 | 13 | 37.4 | ++ |
| | | | protein precursor | | | | | | | | | |
| 10 | AIG67671.1 | MC010 | hypothetical protein | I | 31 | Unknown | DUF1329 | 2.8 | 0 | 0 | 25.7 | + |
| 11 | AIG67672.1 | MC011 | hypothetical protein | I | 51 | OM | unknown | 2.4 | 0 | 0 | 21.9 | + |
| 12 | AIG68053.1 | MC012 | Pertactin precursor | I | 25 | OM | unknown | 36.6 | 42 | 40 | 28.8 | + |
| 13 | AIG68360.1 | MC013 | internalin, putative | S | 48 | Unknown | unknown | 8 | 7.8 | 11.2 | 145 | + |
| 14 | AIG68899.1 | MC014 | putative lipoprotein | S | 25 | OM | SIMPL | 11 | 6.9 | 0 | 0 | − |
| 15 | AIG69664.1 | MC015 | Uncharacterized protein | I | 31 | Unknown | DUF2544 | 33.2 | 93.7 | 50.2 | 72.2 | − |
| | | | YadU in | | | | | | | | | |
| | | | stf fimbrial cluster | | | | | | | | | |
| 16 | AIG70798.1 | MC016 | hypothetical protein | I | 34 | Unknown | DUF4225 | 3.7 | 0 | 15.5 | 22.3 | − |
| 17 | AIG67308.1 | MC017 | putative hemolysin | I | 37 | OM | Potra 2 | 0 | 0 | 13.3 | 19.1 | + |
| | | | activator protein | | | | | | | | | |
| | | | 100% ID | | | | | | | | | |
| 18 | AIG66972.1 | MC018 | core protein | S | 159 | Unknown | unknown | 6.8 | 12.9 | 6.7 | 4.8 | + |
| 19 | AIG69216.1 | MC019 | adherence and invasion | S | 275 | OM | Invasin | 5.8 | 12.6 | 10.8 | 10.3 | − |
| | | | outermembrane protein | | | | | | | | | |
| | | | (Inv, enhances Peyer's | | | | | | | | | |
| | | | patches colonization) | | | | | | | | | |
| 20 | AIG69974.1 | MC020 | Pertactin precursor | S | 148 | OM | AIDA/Pertectin | 12 | 21.8 | 3.3 | 47.1 | ++ |
| 21 | AIG71811.1 | MC021 | adherence and invasion | S | 181 | OM | DUF3442 | 17.3 | 23.5 | 11.2 | 64.5 | − |
| | | | outer membrane protein | | | | | | | | | |
| | | | (Inv, enhances Peyer's | | | | | | | | | |
| | | | patches colonization) | | | | | | | | | |
| 22 | AIG71181.1 | MC022 | hypothetical protein | S | 93 | Unknown | unknown | 5.6 | 0 | 111.3 | 16.9 | + |
| 23 | AIG66227.1 | MC023 | Putative outer | S | 92 | OM | PapC Usher | 3.2 | 10.1 | 0 | 8.3 | + |
| | | | membrane | | | | | | | | | |
| | | | protein | | | | | | | | | |
| 24 | AIG66656.1 | MC024 | Protein YkiA | I | 83 | Unknown | DUF2773 | 8.3 | 6.8 | 0 | 28.1 | + |

S = soluble;
I = insoluble
OM = Outer membrane;
EC = extracellular

able S4. Primers

| | | | Notes (ID, |
|---|---|---|---|
| Primer | Sequence | Product | purpose) |
| MCRL-34Fw | CTGTACTTCCAGGGCTTTCAACCAATACTTAATGAT | MC001 | AIG67060.01 |
| | [SEQ ID NO: 49] | | |
| MCRL-34Rv | AATTAAGTCGCGTTAAAAAAAGAAGGTGATTGC | | |
| | [SEQ ID NO: 50] | | |
| MCRL-24Fw | CTGTACTTCCAGGGCGATATCAATCTGTATGGTCC | MC002 | AIG68165.1 |
| | [SEQ ID NO: 51] | | |
| MCRL-24Rv | AATTAAGTCGCGTTAAGTGCCTTTCCTGGT | | |
| | [SEQ ID NO: 52] | | |
| MCRL-40Fw | CTGTACTTCCAGGGCGACCTTTATGGCAAGG | MC003 | AIG68357.1 |
| | [SEQ ID NO: 53] | | |
| MCRL-40Rv | AATTAAGTCGCGTTAGAACTGATAGGTAATGCC | | |
| | [SEQ ID NO: 54] | | |
| MCRL-35Fw | CTGTACTTCCAGGGCGAGTTTACGATAGACTTTTCG | MC004 | AIG67577.1 |
| | [SEQ ID NO: 55] | | |
| MCRL-35Rv | AATTAAGTCGCGTTATTTACCCGTTGTATATAAAAAC | | |
| | [SEQ ID NO: 56] | | |
| MCRL-32Fw | CTGTACTTCCAGGGCACGTATGTAGATTCGCTG | MC005 | AIG69411.1 |
| | [SEQ ID NO: 57] | | |
| MCRL-32Rv | AATTAAGTCGCGTTAACTGCTAATAGTTCTGCG | | |
| | [SEQ ID NO: 58] | | |
| MCRL-41Fw | CTGTACTTCCAGGGCATGAAGATAAGCTCTACT | MC006 | IAG66347.1 |
| | [SEQ ID NO: 59] | | |
| MCRL-41Rv | AATTAAGTCGCGTTATTCGTAGGTAAAGGA | | |
| | [SEQ ID NO: 60] | | |
| MCRL-28Fw | CTGTACTTCCAGGGCCAGCAACTGACAGACAA | MC007 | AIG66424.1 |
| | [SEQ ID NO: 61] | | |
| MCRL-28Rv | AATTAAGTCGCGTTACTGGAATCGACTCACC | | |
| | [SEQ ID NO: 62] | | |
| MCRL-36Fw | CTGTACTTCCAGGGCAACTGCTATTTTGGTACC | MC008 | AIG66984.1 |
| | [SEQ ID NO: 63] | | |
| MCRL-36Rv | AATTAAGTCGCGTTAGTTGTAGTTAATTTTGAAAAG | | |
| | [SEQ ID NO: 64] | | |
| MCRL-38Fw | CTGTACTTCCAGGGCATGTGGGAATGTGATG | MC009 | AIG67652.1 |
| | [SEQ ID NO: 65] | | |
| MCRL-38Rv | AATTAAGTCGCGTTATTGCATTTTCACCAA | | |
| | [SEQ ID NO: 66] | | |
| MCRL-26Fw | CTGTACTTCCAGGGCACGCTGACGGTAACGG | MC010 | AIG67671.1 |
| | [SEQ ID NO: 67] | | |
| MCRL-26Rv | AATTAAGTCGCGTTATTTCGCACCTCGCTG | | |
| | [SEQ ID NO: 68] | | |
| MCRL-43Fw | CTGTACTTCCAGGGCACGCTTTACGAGCAG | MC011 | AIG67672.1 |
| | [SEQ ID NO: 69] | | |
| MCRL-43Rv | AATTAAGTCGCGTTATAGATAGTATTTAAAGCCGT | | |
| | [SEQ ID NO: 70] | | |
| MCRL-22Fw | CTGTACTTCCAGGGCGTGGGGATCGACAGC | MC012 | AIG68053.1 |
| | [SEQ ID NO: 71] | | |
| MCRL-22Rv | AATTAAGTCGCGTTAGAACGACCAGTTCACAC | | |
| | [SEQ ID NO: 72] | | |
| MCRL-42Fw | CTGTACTTCCAGGGCATGAAATTCCCTTCA | MC013 | AIG68360.1 |
| | [SEQ ID NO: 73] | | |
| MCRL-42Rv | AATTAAGTCGCGTTAGTGATAAAAAGGCCA | | |
| | [SEQ ID NO: 74] | | |
| MCRL-23Fw | CTGTACTTCCAGGGCGGTTATTTTGTTGGCG | MC014 | AIG68899.1 |
| | [SEQ ID NO: 75] | | |
| MCRL-23Rv | AATTAAGTCGCGTTAATCCTGCAACGCATA | | |
| | [SEQ ID NO: 76] | | |
| MCRL-29Fw | CTGTACTTCCAGGGCATTTATTATGCGATGAAA | MC015 | AIG69664.1 |
| | [SEQ ID NO: 77] | | |
| MCRL-29Rv | AATTAAGTCGCGTTACAGTTCATTCAGTACATACTG | | |
| | [SEQ ID NO: 78] | | |
| MCRL-30Fw | CTGTACTTCCAGGGCATGGGTACAGCAGCTATA | MC016 | AIG70798.1 |
| | [SEQ ID NO: 79] | | |
| MCRL-30Rv | AATTAAGTCGCGTTATTGTATTCCTGCACCA | | |
| | [SEQ ID NO: 80] | | |
| MCRL-37Fw | CTGTACTTCCAGGGCGATGTCCGGCGTAGC | MC017 | AIG67308.1 |
| | [SEQ ID NO: 81] | | |
| MCRL-37Rv | AATTAAGTCGCGTTACTGCTTTTTACAACCATTC | | |
| | [SEQ ID NO: 82] | | |
| MCRL-48Fw | CTGTACTTCCAGGGCATGAGCGGAAAACCG | MC018 | AIG66972.1 |
| | [SEQ ID NO: 83] | | |
| MCRL-48Rv | AATTAAGTCGCGTTATTTTCTTATTCCTCTCGATG | | |
| | [SEQ ID NO: 84] | | |
| MCRL-52Fw | CTGTACTTCCAGGGCATGGCTGCGGCAGCA | MC019 | AIG69216.1 |
| | [SEQ ID NO: 85] | | |
| MCRL-52Rv | AATTAAGTCGCGTTACGCAATATTGACGAT | | |
| | [SEQ ID NO: 86] | | |
| MCRL-49Fw | CTGTACTTCCAGGGCGTGGGGCAGTCTAAT | MC020 | AIG69974.1 |
| | [SEQ ID NO: 87] | | |
| MCRL-49Rv | AATTAAGTCGCGTTAACTCGCTTTCATTATGTT | | |
| | [SEQ ID NO: 88] | | |
| MCRL-51Fw | CTGTACTTCCAGGGCGTGCCTTACACGCTTGGT | MC021 | AIG71811.1 |
| | [SEQ ID NO: 89] | | |
| MCRL-51Rv | AATTAAGTCGCGTTAAAGTGATTTACGGCAGGC | | |
| | [SEQ ID NO: 90] | | |
| MCRL-45Fw | CTGTACTTCCAGGGCATGGTCGCTAAATTAAAAC | MC022 | AIG71181.1 |
| | [SEQ ID NO: 91] | | |
| MCRL-45Rv | AATTAAGTCGCGTTAAGCCTGGGTTATATTAAC | | |
| | [SEQ ID NO: 92] | | |
| MCRL-45Fw | CTGTACTTCCAGGGCATGGTCGCTAAATTAAAAC | MC023 | AIG66227.1 |
| | [SEQ ID NO: 93] | | |
| MCRL-45Rv | AATTAAGTCGCGTTAAGCCTGGGTTATATTAAC | | |
| | [SEQ ID NO: 94] | | |
| MCRL-46Fw | CTGTACTTCCAGGGCAGTTATGGCCGATTT | MC024 | AIG66656.1 |
| | [SEQ ID NO: 95] | | |
| MCRL-46Rv | AATTAAGTCGCGTTATGTAAACTGCACATAAGA | | |
| | [SEQ ID NO: 96] | | |
| pET-TEV-Fw | TAACGCGACTTAATTCTAGCATAACC | pET15 | pET-15 |
| | [SEQ ID NO: 97] | | expression |
| pET-REV-Rv | GCCCTGGAAGTACAGGTTTTC | | vector |
| | [SEQ ID NO: 98] | | |
| TolR-Cat-Fw | TGGAGGTCGATTTGCCAGACGCTACTGAATCACAGGCGGTGAGCAGTAACGATA | Cat-TolR | tolR mutant |
| | ATCCGCCAGTGATTGTTGACATATGAATATCCTCCTTAGTTCCTATTCC | | construction |
| | [SEQ ID NO: 99] | | |
| TolR-Cat-Rv | CCTTGAAACGGCTGGACACTTCCGCCACCACCTGCTCTGGTGGTAAACGCTCCA | | |
| | GGCGATCTTTCTCAACCAGTGTAGGCTGGAGCTGCTTCGAAG | | |
| | [SEQ ID NO: 100] | | |
| GMMA- | CAGGAGGAATTAACCATGAAAAAAAGTGTCATCGCTGGC | MC001 with | Antigen |
| MC001Fw | [SEQ ID NO: 101] | signal | expression on |
| GMMA- | ATGATGATGATGATGATGAAAAAAGAAGGTGATTGCTCC | sequence | GMMA |
| MC001Rv- | [SEQ ID NO: 102] | | |
| NONhis | | | |
| GMMA- | CAGGAGGAATTAACCATGGGGGCAGTCTAATAATACCAC | MC007 with | Antigen |
| MC007Fw | [SEQ ID NO: 103] | signal | expression on |
| GMMA- | ATGATGATGATGATGATGGAACGACCAGTTCACACCAGC | sequence | GMMA |
| MC007Rv- | [SEQ ID NO: 104] | | |
| NONhis | | | |
| GMMA- | CAGGAGGAATTAACCATGGGGGCAGTCTAATAATACCACC | MC020 with | Antigen |
| MC020Fw | [SEQ ID NO: 105] | signal | expression on |
| GMMA- | ATGATGATGATGATGATGGAACGACCAGTTCACACCAG | sequence | GMMA |
| MC020Rv- | [SEQ ID NO: 106] | | |
| NONhis | | | |
| MC001tagFw | GATTACAAAGACGATGATGACAAGGATTACAAAGACGATGATGACAAGAACAGC | MC001 with | Antigen |
| | CTTGCATTATCATT | signal | expression on |
| | [SEQ ID NO: 107] | sequence | GMMA with |
| MC001tagRv | CTTGTCATCATCGTCTTTGTAATCCTTGTCATCATCGTCTTTGTAATCCGCGAT | and Flag | Flag Tag |
| | AGCTGAACACGTGG | Tag | |
| | [SEQ ID NO: 108] | | |
| MC007tagFw | GATTACAAAGACGATGATGACAAGGATTACAAAGACGATGATGACAAGTCTGAA | MC007 with | Antigen |
| | TCTTCTATTGATGA | signal | expression on |
| | (SEQ ID NO: 109] | sequence | GMMA with |
| MC007tagRv | CTTGTCATCATCGTCTTTGTAATCCTTGTCATCATCGTCTTTGTAATCGGCACA | and Flag | Flag Tag |
| | TCCTGCAAGCAGCG | Tag | |
| | [SEQ ID NO: 110] | | |
| MC020tagFw | GATTACAAAGACGATGATGACAAGGATTACAAAGACGATGATGACAAGGCCTTC | MC020 with | Antigen |
| | ACTCCTGATGTTAT | signal | expression on |
| | [SEQ ID NO: 111] | sequence | GMMA with |
| MC020tagRv | CTTGTCATCATCGTCTTTGTAATCCTTGTCATCATCGTCTTTGTAATCAGCCATC | and Flag | Flag Tag |
| | CCGGGCGGGGCAT | Tag | |
| | [SEQ ID NO: 112] | | |
| pBADRv | CATGGTTAATTCCTCCTGTTAGCCC | pBAD | Expression |
| | [SEQ ID NO: 113] | Vector | of Ag in |
| pBADFw | TGAGTTTAAACGGTCTCCAGCTTGG | without | GMMA |
| NonHIS | [SEQ ID NO: 114] | His-Tag | |

TABLE 5
Nucleotide and Protein Sequences of the 24 Antigen Candidates

| | | Sequence |
|---|---|---|
| Name | Sequence | Length |
| MC001 | ATGAAAAAAAGTGTCATCGCTGGCGTCTTTATTGCTCTGTCATTTACCACGTGTTCAGCTATCGCGAAG | 960 |
| | AGCCTTGCATTATCATTAGCAAATGATGATGCAGGGAAGTTTCAACCAATACTTAATGATATTTATGGC | |
| | AATAAACATGAAAACAGAGATGATTACTCACAAGGCTTATTTCTGGGATATAGCCACGATATCTCAGAC | |
| | TCGAGCCAATTATCTCTCCATATTGCGCAAGATATTTACTCTCCATCAGGCAGTAATAAAAGACACAAC | |
| | ACAGCTGTAACTGGAGACAGAGCTTTTAGTGCATACACTCACACTGGTATTGAATGGAACTCCCTTGCG | |
| | AATGACTGGATTCGCTATCGATTAGGTACTGACATAGGTGTTGTTGGCCCCGACGCAGGCGGTCAGAAA | |
| | GTACAAAATAAAGCTCATGAGATTATTGGGGCAGAAAAATATCATGCATGGGATGATCAAATAGAGAAT | |
| | CGCTACGGTTATACTGTAAAAGGGATGCTATCCATGACACCAAGTATGGATATTTTAGGTGCTAATGTT | |
| | GGATTATACCCTGAAGTTTCTGCTGTTACTGGAAACTTATTTCAATATGTAGCATATGGCGCAACCATT | |
| | GCCATTGGTAATGATAAAACCTTCAATTCGGATAATGGCTTTGGTCTGCTGGCTCCCCGTGGTTTAATG | |
| | CATATGTCCGATACAAGCGGATTCAAATACAAGATTTTTGCAGGTATGGAAAGACGAGATGTCAATCGC | |
| | AACTATACTCTCGAAGGAAAAACAATACAGACGAAACAAACAACAGTATCGCTAAACAAAACTGTTGAT | |
| | GAATATCAAGTTGGCGCAACAATTGGGTATGCACCTGTAGCCTTCACACTAGCATTTAATAAAGTAACA | |
| | TCAGAATTCAAGACAGGGGATGACTATTCATTTATAAATGGAGCAATCACCTTCTTTTTTTAA | |
| | [SEQ ID NO: 1] | |
| MC001 | MKKSVIAGVFIALSFTTCSAIANSLALSLANDDAGKFQPILNDIYGNKHENRDDYSQGLFLGYSHDISD | 319 |
| prot | SSQLSLHIAQDIYSPSGSNKRHNTAVTGDRAFSAYTHTGIEWNSLANDWIRYRLGTDIGVVGPDAGGQK | |
| | VQNKAHEIIGAEKYHAWDDQIENRYGYTVKGMLSMTPSMDILGANVGLYPEVSAVTGNLFQYVAYGATI | |
| | AIGNDKTFNSDNGFGLLAPRGLMHMSDTSGFKYKIFAGMERRDVNRNYTLEGKTIQTKQTTVSLNKTVD | |
| | EYQVGATIGYAPVAFTLAFSKVTSEFKTGDDYSFINGAITFFF | |
| | [SEQ ID NO: 25] | |
| MC007 | ATGAATTCAAAAAAGCTTTGTTGCATATGTGTGTTATTCTCGCTGCTTGCAGGATGTGCCTCTGAATCT | 801 |
| | TCTATTGATGAAAAGAAGAAAAAAGCTCAAGTCACACAAAGTAATATTAATAAAAACACTCCCCAGCAA | |
| | CTGACAGACAAAGATTTATTCGGTAATGAAACCACTCTGGCCGTATCCGAAGAGGATATTCAAGCAGCG | |
| | CTTGATGGAGATGAGTTCCGCGTTCCCCTTAATTCTCCTGTAATACTTGTTCAATCCGGCAACCGCGCA | |
| | CCGGAAACCATTATGCAGGAGGAGATGCGAAAATATTATACTGTTTCCACATTCTCTGGTATCCCGGAC | |
| | AGGCAAAAGCCTCTGACTTGTAACAAAAACAAAGATAAAAATGAGAACGAGGATGTTGCCCGTGCTGAG | |
| | AATATGAACTGGATGGAGGCACTGCGTTTTGTGGCTGCAAAAGGACATCAGAAAGCGATTATTGTTTAC | |
| | CAGGATATGTTGCAGACAGGAAAATATGACTCTGCGCTGAAATCAACAGTATGGTCCGACTATAAAAAT | |
| | GAAAAACTCACTGACGCTATCTCCCTGCGCTACCTGGTACGTTTCACGCTGGTGGATGTGGCAACAGGT | |
| | GAGTGGGCTACCTGGTCGCCGGTGAATTATGAATCGAGAGTAATTTTTCCGCCGGCTGGAATAACAAAA | |
| | ACGAGCAATAAAGAACTATCAAATGACCATGTTACGGAAGCGCAACTGTTTAATTTGAAACAAAAAACA | |
| | TATGTATCGATGGTGAAAGACTTGGTGAGTCGATTCCAGTAA | |
| | [SEQ ID NO: 2] | |
| MC007 | MNSKKLCCICVLFSLLAGCASESSIDEKKKKAQVTQSNINKNTPQQLTDKDLFGNETTLAVSEEDIQAA | 266 |
| pro& | LDGDEFRVPLNSPVILVQSGNRAPSTIMQEEMRKYYTVSTFSGIPDRQKPLTCNKNKDKNENEDVARAE | |
| | NMNWMQALRFVAAKGHQKAIIVYQDMLQTGKYDSALKSTVWSDYKNEKLTDAISLRYLVRFTLVDVATG | |
| | EWATWSPVNYESRVIFFPAGITKTSNKELSNDHVTEAQLFNLKQKTYVSMVKDLVSRFQ | |
| | [SEQ ID NO: 26] | |
| MC020 | ATGAACAGGACCAGTCCCTATTATTGTCGTGGCTCAGTACTTTCCTTATTGATATCTGCCTTGATATAT | 4587 |
| | GCCCCGCCCGGGATGGCTGCCTTCACTCCTGATGTTATTGGTGTGGTAAACGATGAGACTGTAGATGGC | |
| | AGCCAACGAGTAGATGAACGAGGTACAACAAATAACACTCATATTATCAACCATGGCCAGCAGAATGTT | |
| | TATGGCGGGGTATCTAATGGAAGTCTTATTGAATCTGGTGGATATCAAGATGTAGGAAGGCATAACAAT | |
| | TATGTGGGGCAGTCTAATAATACCACCATTAACGGGGGCAGACAGTCAATTCATGACGGGGGTATTTCC | |
| | ACAGGTACGATAATCGAGAGTGGCAATCAGGACGTTTATAAAGGGGGTATCAGCAATGGAACGACAATT | |
| | AAGGGCGGTGCTTCACGCGTAGAGGGAGGGAGTGCGAATGGAACACTCATTGATGGTGGTAGCCAGATA | |
| | GTAAAAGTTCAAGGGCATGCTGATGGTACAACGATAAATAAGTCTGGCTCTCAGGACGTAGTACAAGGA | |
| | AGTCTGGCAACGAACACAACCATAAATGGTGGTCGACAGTATGTTGAACAGAGCACAGTAGAAACAACC | |
| | ACCATCAAAAATGGCGGTGAGCAAAGAGTATATGAGAGCCGTGCGCTGGACACGACGATTGAAGGCGGA | |
| | ACTCAGTCTCTGAATAGTAAGTCAACGGCAAAAAATACTCAGATCTATTCTGGTGGTACGCAAATTATT | |
| | GATAACACCAGCTCCTCGGATGTTATTGAAGTTTATTCCGGTGGCGTGCTTGATGTTAGTGGTGGTACG | |
| | GCAACAAATGTTACCCAGCACGATGGTGCAATTTTAAAAACTAACACTAACGGTACGACGGTGAGCGGT | |
| | ACGAATAGTGAAGGTGCATTCTCCATCCACAATCACGTGGCAGACAATGTGTTGCTGGAAAACGGTGGT | |
| | CATTTAGACATAAACGCATATGGTTCGGCAAACAAGACGATTATTAAAGATAAAGGAACAATGTCAGTT | |
| | TTAACCAATGCTAAAGCTGATGCGACCCGAATAGATAATGGCGGGGTTATGGATGTTGGAGGAAACGCG | |
| | ACAAATACCATAATTAATGGTGGCACACAGAATATTAATAATTATGGCATAGCCACAGGCACCAATATC | |
| | AACAGCGGAACGCAAAATATCAAAAGCGGCGGGAAAGCTGACACAACAATTATATCCTCCGGGAGCCGG | |
| | CAGGTTGTTGAGAAAGATGGTACGGCAATTGGCAGCAATATTAGCGCCGGAGGCTCGCTGATTGTCTAT | |
| | ACCGGCGGTATTGCACATGGGGTTAACCAGGAGACGGGCAGTGCTTTAGTTGCCAACACGGGTGCAGGG | |
| | ACTGATATCGAAGGATACAACAAGCTCTCTCACTTCACTATTACCGGAGGGGAGGCTAATTATGTTGTG | |
| | CTGGAAAATACCGGCGAACTGACGGTAGTGGCTAAAACCTCGGCGAAAAATACTACCATTGATGCTGGC | |
| | GGTAAGCTGATTGTCCAGAAGGAGGCTAAAACAGATAGCACCAGACTTAATAATGGCGGCGTTCTGGAG | |
| | GTTCAGGACGGTGGTGAGGCTAAGCATGTTGAGCAACAATCCGGCGGCGCATTAATTGCTTCCACGACC | |
| | TCCGGAACACTTATCGAAGGAACCAACAGTTATGGTGATGCTTTCTACATCAGGAATTCAGAAGCTAAA | |
| | AATGTAGTGCTGGAAAACGCTGGCTCATTAACAGTCGTCACTGGTTCCCGGGCAGTTGACACGATTATT | |
| | AATGCCAACGGCAAAATGGATGTTTATGGAAAAGATGTTGGCACTGTACTCAATAGTGCTGGCACCCAA | |
| | ACAATATATGCCAGTGCCACTTCTGATAAAGCAAATATCAAAGGTGGCAAGCAAACGGTATATGGTTTA | |
| | GCCACTGAAGCAAATATCGAAAGTGGTGAACAAATTGTTGATGGTGGGTCAACAGAGAAAACACACATC | |
| | AATGGTGGCACGCAAACCGTTCAGAATTATGGTAAGGCGATCAATACCGATATCGTCTCTGGCCTACAA | |
| | CAAATTATGGCAAACGGGACAGCGGAAGGTTCCATTATTAATGGCGGTTCACAGATAGTTAATGAGGGC | |
| | GGTCTGGCTGAAAACTCGGTGCTTAATGATGGCGGCACACTCGATGTGCGGGAGAAAGGCAGCGCAACG | |
| | GGGATACAGCAGAGTAGCCAGGGCGCGTTGGTTGCAACCACCAGGGCGACGCGGGTCACAGGAACACGC | |
| | GCGGATGGCGTCGCGTTCAGCATCGAGCAGGGTGCGGCGAACAATATCCTGCTGGCAAATGGCGGAGTG | |
| | TTAACCGTGGAGTCAGACACCTCTTCTGACAAAACACAGGTCAATACGGGCGGACGGGAGATCGTCAAA | |
| | ACAAAAGCCACTGCGACAGGCACGACGCTCACCGGCGGTGAACAAATTGTCGAGGGTGTGGCGAATGAG | |
| | ACAACAATTAACGACGGCGGAATACAAACAGTTTCAGCTAACGGAGAGGCAATAAAAAGAACGATCAAT | |
| | GAAGGCGGTACGCTGACAGTCAACGATAATGGGAAAGCGACAGATATCGTCCAGAACAGCGGTGCCGCT | |
| | CTCCAGACGAGCACGGCTAACGGTATTGAAATCAGCGGTACTCACCAGTACGGCACTTTTTCCATTTCC | |
| | GGCAATTTAGCGACCAATATGTTGCTGGAAAATGGCGGTAATTTATTGGTATTAGCAGGTACCGAAGCT | |
| | CGCGACTCCACGGTTGGCAAGGGGGGGGCAATGCAAAACCAGGGTCAGGACTCCGCCACAAAGGTTAAC | |
| | TCTGGTGGGCAATATACCCTTGGGCGGTCAAAAGATGAGTTTCAGGCTCTGGCCCGGGCAGAAGATCTC | |
| | CAGGTTGCTGGCGGGACAGCAATCGTCTACGCAGGTACGCTGGCGGATGCATCGGTCAGTGGCGCGACA | |
| | GGAAGCCTGTCGTTAATGACGCCACGGGATAATGTTACGCCAGTTAAACTCGAAGGGGCGATCCGGATT | |
| | ACCGATAGCGCGACATTAACTATCGGCAATGGCGTTGATACGACGCTTGCCGACCTGACGGCTGCCAGC | |
| | CGGGGCAGTGTCTGGCTTAACAGCAATAATTCCTGTGCAGGCACCAGCAACTGCGAGTATAGAGTAAAC | |
| | AGTTTGCTACTTAACGACGGTAATGTTTATTTATCAGCACAAACAGCAGCGCCTGCCACAACTAACGGT | |
| | ATATACAATACGCTGACAACCAATGAACTTTCCGGTAGCGGTAATTTCTACCTGCATACCAACGTTGCA | |
| | GGCTCTCGGGGCGATCAACTGGTCGTCAACAACAACGCGACTGGTAATTTTAAAATCTTTGTTCAGGAT | |
| | ACCGGCGTCAGTCCTCAGTCTGACGACGCGATGACGCTGGTGAAAACAGGGGGAGGGGATGCTTCGTTT | |
| | TCGCTGGGCAATACTGGCGGTTTCGTTGATGTTGGGACCTATGAGTATGTCCTGAAAAGCGATGGCAAC | |
| | AGCAACTGGAACCTGACCAATGATGTCAAACCCAACCCGGATCCCAACCCAAATCCCAACGCAAATCCG | |
| | AAGCCGGATCCAAAACCAGACCCAAAACCGGATCCGAAACCAGACCCGACTCCCGAGCCAACGCCGACA | |
| | CCCGTTCCGGAGAAACGCATCACGCCTTCTACCGCAGCCGTACTCAATATGGCAGCAACATTACCGTTG | |
| | GTATTTGATGCTGAGCTAAACAGTATTCGCGAGCGGTTGAACATAATGAAAGCGAGTCCACACAACAAT | |
| | AATGTCTGGGGGGCGACGTATAACACCCGTAATAATGTCACCACCGATGCGGGGGCCGGGTTTGAGCAG | |
| | ACGCTGACCGGAATGACAGTGGGGATCGACAGCCCTAATGATATTCCTGAGGGGATTGCGACGCTGGGC | |
| | GCTTTTATGGGTTATTCCCATTCACATATCGGTTTTGATCGCGGAGGACATGGCAGTGTGGGCAGTTAT | |
| | TCTCTGGGCGGCTATGCCAGTTGGGAACATGAAAGTGGTTTCTATCTGGACGGTGTCGTGAAGCTGAAC | |
| | CGTTTTGAAAGTAACGTAGCCGGTAAAATGAGCAGCGGTGGAGCCGCCAATGGCAGTTACCACAGCAAC | |
| | GGGCTGGGCGGTCACATTGAAACCGGGATGCGATTTACCGATGGTAACTGGAACCTGACGCCGTATGCA | |
| | TCGTTAACGGGGTTCACCGCTGATAACCCCGAATATCATTTATCCAATGGCATGGAATCGAAATCAGTC | |
| | GATACCCGCAGTATATATCGTGAACTGGGCGCAACGCTGAGTTACAACATGCGTCTGGGGAACGGTATG | |
| | GAAATTGAGCCGTGGCTGAAGGCGGCTGTGCGCAAAGAATTTGTCGATGATAACCGGGTGAAGGTGAAT | |
| | AATGACGGTAATTTCGTCAATGATTTGTCGGGCAGACGTGGAATATACCAGGCAGGTATTAAAGCCTCA | |
| | TTCAGCAGTACGTTAAGCGGGCATCTTGGGGTGGGGTATAGCCATGGTGCCGGTGTGGAATCGCCGTGG | |
| | AACGCGGTAGCTGGTGTGAACTGGTCGTTCTGA | |
| | [SEQ ID NO: 3] | |
| MC020 | MNRTSPYYCRRSVLSLLISALIYAPPGMAAFTPDVIGVVNDETVDGSQRVDERGTTNNTHIINHGQQNV | 1528 |
| prot | YGGVSNGSLIESGGYQDVGRHNNYVGQSNNTTINGGRQSIHDGGISTGTIIESGNQDVYKGGISNGTTI | |
| | KGGASRVEGGSANGTLIDGGSQIVKVQGHADGTTINKSGSQDVVQGSLATNTTINGGRQYVEQSTVETT | |
| | TIKNGGEQRVYESRALDTTIEGGTQSLNSKSTAKNTQIYSGGTQIIDNTSSSDVIEVYSGGVLDVSGGT | |
| | ATNVTQHDGAILKTNTNGTTVSGTNSEGAFSIHNHVADNVLLENGGHLDINAYGSANKTIIKDKGTMSV | |
| | LTNAKADATRIDNGGVMDVAGNATNTIINGGTQNINNYGIATGTNINSGTQNIKSGGKADTTIISSGSR | |
| | QVVEKDGTAIGSNISAGGSLIVYTGGIAHGVNQETGSALVANTGAGTDIEGYNKLSHFTITGGEANYVV | |
| | LENTGELTVVAKTSAKNTTIDAGGKLIVQKEAKTDSTRLNNGGVLEVQDGGEAKHVEQQSGGALIASTT | |
| | SGTLIEGTNSYGDAFYIRNSEAKNVVLENAGSLTVVTGSRAVDTIINANGKMDVYGKDVGTVLNSAGTQ | |
| | TIYASATSDKANIKGGKQTVYGLATEANIESGEQIVDGGSTEKTHINGGTQTVQNYGKAINTDIVSGLQ | |
| | QIMANGTAEGSIINGGSQIVNEGGLAENSVLNDGGTLDVREKGSATGIQQSSQGALVATTRATRVTGTR | |
| | ADGVAFSIEQGAANNILLANGGVLTVESDTSSDKTQVNTGGREIVKTKATATGTTLTGGEQIVEGVANE | |
| | TTINDGGIQTVSANGEAIKTTINEGGTLTVNDNGKATDIVQNSGAALQTSTANGIEISGTHQYGTFSIS | |
| | GNLATNMLLENGGNLLVLAGTEARDSTVGKGGAMQNQGQDSATKVNSGGQYTLGRSKDEFQALARAEDL | |
| | QVAGGTAIVYAGTLADASVSGATGSLSLMTPRDNVTPVKLEGAIRITDSATLTIGNGVDTTLADLTAAS | |
| | RGSVWLNSNNSCAGTSNCEYRVNSLLLNDGNVYLSAQTAAPATTNGIYNTLTTNELSGSGNFYLHTNVA | |
| | GSRGDQLVVNNNATGNFKIFVQDTGVSPQSDDAMTLVKTGGGDASFSLGNTGGFVDLGTYEYVLKSDGN | |
| | SNWNLTNDVKPNPDPNPNPNPNPKPDPKPDPKPDPKPDPTPEPTPTPVPEKRITPSTAAVLNMAATLPL | |
| | VFDAELNSIRERLNIMKASPHNNNVWGATYNTRNNVTTDAGAGFEQTLTGMTVGIDSPNDIPEGIATLG | |
| | AFMGYSHSHIGFDRGGHGSVGSYSLGGYASWEHESGFYLDGVVKLNRFESNVAGKMSSGGAANGSYHSN | |
| | GLGGHIETGMRFTDGNWNLTPYASLTGFTADNPEYHLSNGMESKSVDTRSIYRELGATLSYNMRLGNGM | |
| | EIEPWLKAAVRKEFVDDNRVKVNNDGNFVNDLSGRRGIYQAGIKASFSSTLSGHLGVGYSHGAGVESPW | |
| | NAVAGVNWSF | |
| | [SEQ ID NO: 27] | |
| MC002 | ATGGCAGGTTTTTTAATATTCCTGTCTTCTGCTGCTTATGCTGATATCAATCTGTATGGTCCTGGTGGC | 747 |
| | CCGCATACAGCCTTGCTTGATGCAGCCAAACTTTATGCCGAAAAAACAGGTATTATAGTGAACGTTCAT | |
| | TACGGCCCACAGAACAAATGGAATGAAGATGCCAAAAAAAATGCAGATATCTTGTTTGGCGCATCAGAA | |
| | CAATCTGCTCTGGCTATCATTCGGGACCATAAAGACAGCTTCAGTGAAAAAGATATTCAGCCTCTTTAT | |
| | CTGCGAAAAAGTATTTTACTGGTAAAGAAAGGTAATCCTAAAAATATCCGGAGTATTGACGACCTGACC | |
| | AGACCTGGGATTGGCGTAATTGTTAATGATGGTGGTGGTACCAGTAATACATCAGGCACTGGCGTCTGG | |
| | GAAGATATTGCCGGACGTAAAGGGAATATAGAAACTGTCGCCGCAATCCGAAAAAATATTATTTTATAT | |
| | GCGCCGAATAGCGGAACTGCACGTAAGGCTCTTGAGAATCAGCCTGGAGCAGATGTCTGGATAACCTGG | |
| | GCTGACTGGGCAGCCAGTAATCCAGAAATTGGTGATGTCGTGGAAATAGCGCCAGACTACGTGATATGG | |
| | CGTGATATGAACATTACAGTACGTCAGGATGCAAATGATGAAACCCGTCGATTTGCAGAATGGCTACAA | |
| | ACCGATGAAGCGGCGCCTGCATTCAAAAAATATGGCTGGACCAGGAAAGGCACTTGA | |
| | [SEQ ID NO: 4] | |
| MC002 | MAGFLIFLSSAAYADINLYGPGGPHTALLDAAKLYAEKTGIIVNVHYGPQNKWNEDAKKNADILFGASE | 248 |
| prot | QSALAIIRDHKDSFSEKDIQPLYLRKSILLVKKGNPKNIRSIDDLTRPGIGVIVNDGGGTSNTSGTGVW | |
| | EDIAGRKGNIETVAAIRKNIILYAPNSGTARKALENQPGADVWITWADWAASNPEIGDVVEIAPDYVIW | |
| | RDMNITVRQDANDETRRFAEWLQTDEAAPAFKKYGWTRKGT | |
| | [SEQ ID NO: 28] | |
| MC003 | ATGAAATTAAAAATAGTTGCGGTGGTTGTAACTGGTTTGTTAGCTGCGAACGTAGCACACGCTGCCGAA | 1101 |
| | GTCTATAACAAGGATGGTAATAAACTCGACCTTTATGGCAAGGTTACCGCTCTACGTTATTTTACTGAT | |
| | GATAAGCGTGACGATGGTGATAAAACTTATGCCCGTCTCGGCTTTAAAGGAGAAACGCAAATCAATGAT | |
| | CAAATGATTGGTTTTGGTCACTGGGAATATGATTTTAAAGGCTATAACGATGAAGCCAACGGCTCGCGC | |
| | GACAACAAGACCCGTCTGGCCTATGCTGGTTTAAAAATTAGTGAATTTGGCTCTCTGGACTATGGCCGT | |
| | AACTACGGTGTCGGCTATGACATTGGTTCATGGACTGATATGTTGCCAGAATTTGGTGGCGATACCTGG | |
| | AGTCAGAAAGATGTCTTCATGACATACGGTACCACCGGTGTAGCAACCTATCGCAACTACGATTTCTTT | |
| | GGCTTAATTGAAGGGCTGAACTTTGCCGCGCAATATCAAGGCAAAAATGAACGTACTGACAACAGTCAT | |
| | CTTTATGGTGCTGACTACACGCGTGCCAACGGTGACGGTTTCGGTATCTCCTCAACTTATGTTTATGAT | |
| | GGCTTTGGTATCGGAGCGGTGTATACCAAATCCGATCGGACAAATGCGCAGGAAAGAGCCGCTGCTAAT | |
| | CCTCTCAATGCCTCCGGTAAGAATGCAGAACTGTGGGCTACAGGTATAAAATATGATGCCAACAACATC | |
| | TACTTTGCAGCTAATTACGCTGAAACATTAAACATGACCACCTATGGCGATGGTTATATTTCTAACAAA | |
| | GCACAAAGTTTTGAAGTGGTGGCGCAATATCAATTCGACTTCGGCTTGCGCCCCTCACTCGCTTACCTG | |
| | AAATCGAAAGGCATAGATCTGGGCCGCTACGGCGATCAGGACATGATTGAGTATATCGACGTTGGTGCG | |
| | ACGTATTTCTTCAACAAAAATATGTCGACCTATGTTGATTATAAAATCAACCTGATTGATGAAAGCGAC | |
| | TTTACCCGTGCCGTAGATATTCGCACCGATAACATCGTCGCTACGGGCATTACCTATCAGTTCTAA | |
| | [SEQ ID NO: 5] | |
| MC003 | MKLKIVAVVVTGLLAANVAHAAEVYNKDGNKLDLYGKVTALRYFTDDKRDDGDKTYARLGFKGETQIND | 366 |
| prot | QMIGFGHWEYDFKGYNDEANGSRDNKTRLAYAGLKISEFGSLDYGRNYGVGYDIGSWTDMLPEFGGDTW | |
| | SQKDVFMTYRTTGVATYRNYDFFGLIEGLNFAAQYQGKNERTDNSHLYGADYTRANGDGFGISSTYVYD | |
| | GFGIGAVYTKSDRTNAQERAAANPLNASGKNAELWATGIKYDANNIYFAANYAETLNMTTYGDGYISNK | |
| | AQSFEVVAQYQFDFGLRPSLAYLKSKGIDLGRYGDQDMIEYIDVGATYFFNKNMSTYVDYKINLIDESD | |
| | FTRAVDIRTDNIVATGITYQF | |
| | [SEQ ID NO: 29] | |
| MC004 | ATGAAGTGTATATTATTTAAATGGGTACTGTGCCTGTTACTGGGTTTTTCTTCGGTATCCTATTCCCGG | 960 |
| | GAGTTTACGATAGACTTTTCGACCCAACAAAGTTATGTCTCTTCGTTAAATAGTATACGGACAGAGATA | |
| | TCGACCCCTCTTGAACATATATCTCAGGGGACCACATCGGTGTCTGTTATTAACCACACCGCACCGGGC | |
| | AGTTATTTTGCTGTGGATATACGAGGGCTTGATGTCTATCAGGCGCGTTTTGACCATCTTCGTCTGATT | |
| | ATTGAGCAAAATAATTTATATGTGGCCGGGTTCGTTAATACGGCAACAAATACTTTCTACCGTTTTTCA | |
| | GATTTTACACATATATCAGTGCCCGGTGTGACAACGGTTTCCATGACAACGGACAGCAGTTATACCACT | |
| | CTGCAACGTGTCGCAGCGCTGGAACGTTCCGGAATGCAAATCAGTCGTCACTCACTGGTTTCATCATAT | |
| | CTGGCGTTAATGGAGTTCAGTGGTAATACAATGACCAGAGATGCATCCAGAGCAGTTCTGCGTTTTGTC | |
| | ACTGTCACAGCAGAAGCCTTACGCTTCAGGCAGATACAGAGAGAATTTCGTCAGGCACTGTCTGAAACT GCTCCTGTGTATACGATGACGCCGGGAGACGTGGACCTCACTCTGAACTGGGGGCGAATCAGCAATGTG | |
| | CTTCCGGAGTATCGGGGAGAGGATGGTGTCAGAGTGGGGAGAATATCCTTTAATAATATATCAGCGATA | |
| | CTGGGGACTGTGGCCGTTATACTGAATTGCCATCATCAGGGGGCGCGTTCTGTTCGCGCCGTGAATGAA | |
| | GAGAGTCAACCAGAATGTCAGATAACTGGCGACAGGCCTGTTATAAAAATAAACAATACATTATGGGAA | |
| | AGTAATACAGCTGCAGCGTTTCTGAACAGAAAGTCACAGTTTTTATATACAACGGGTAAATAA | |
| | [SEQ ID NO: 6] | |
| MC004 | MKCILFKWVLCLLLGFSSVSYSREFTIDFSTQQSYVSSLNSIRTEISTPLEHISQGTTSVSVINHTPPG | 319 |
| prot | SYFAVDIRGLDVYQARFDHLRLIIEQNNLYVAGFVNTATNTFYRFSDFTHISVPGVTTVSMTTDSSYTT | |
| | LQRVAALERSGMQISRHSLVSSYLALMEFSGNTMTRDASRAVLRFVTVTAEALRFRQIQREFRQALSET | |
| | APVYTMTPGDVDLTLNWGRISNVLPEYRGEDGVRVGRISFNNISAILGTVAVILNCHHQGARSVRAVNE | |
| | ESQPECQITGDRPVIKINNTLWESNTAAAFLNRKSQFLYTTGK | |
| | [SEQ ID NO: 30] | |
| MC005 | ATGAAAATAATTATTTTTAGAGTGCTAACTTTTTTCTTTGTTATCTTTTCAGTTAATGTGGTTGCGAAG | 948 |
| | GAATTTACCTTAGACTTCTCGACTGCAAAGACGTATGTAGATTCGCTGAATGTCATTCGCTCTGCAATA | |
| | GGTACTCCATTACAGACTATTTCATCAGGAGGTAGGTCTTTACTGATGATTGATAGTGGCACAGGGGAT | |
| | AATTTGTTTGCAGTTGATGTCAGAGGGATAGATCCAGAGGAAGGGCGGTTTAATAATCTACGGCTTATT | |
| | GTTGAACGAAATAATTTATATGTGACAGGATTTGTTAACAGGACAAATAATGTTTTTTATCGCTTTGCT | |
| | GATTTTTCACATGTTACCTTTCCAGGTACAACAGCGGTTACATTGTCTGGTGACAGTAGCTATACCACG | |
| | TTACAGCGTGTTGGAGGGATCAGTCGTACGGGGATGCAGATAAATCGCCATTCGTTGACTACTTCTTAT | |
| | CTGGATTTAATGTCGCATAGTGGAACCTCACTGACGCAGTCTGTGGCAAGAGCGATGTTACGGTTTGTT | |
| | ACTGTGACAGCTGAAGCTTTACGTTTTCGGCAAATACAGAGGGGATTTCGTACAACACTGGATGATCTC | |
| | AGTGGGCGTTCTTATGTAATGACTGCTGAAGATGTTGATCTTACATTGAACTGGGGAAGGTTGAGTAGT | |
| | GTCCTGCCTGATTATCATGGACAAGACTCTGTTCGTGTAGGAAGAATTTCTTTTGGAAGCATTAATGCA | |
| | ATTCTGGGAAGCGTGGCATTAATACTGAATTGTCATCATCATGCATCGCGAGTTGCCAGAATGGCATCT | |
| | GATGAGTTTCCTTCTATGTGTCCGGCAGATGGAAGAGTCCGTGGGATTACGCACAATAAAATATTGTGG | |
| | GATTCATCCACTCTGGGGGCAATTCTGATGCGCAGAACTATTAGCAGTTGA | |
| | [SEQ ID NO: 7] | |
| MC005 | MKIIIFRVLTFFFVIFSVNVVAKEFTLDFSTAKTYVDSLNVIRSAIGTPLQTISSGGTSLLMIDSGTGD | 315 |
| prot | NLFAVDVRGIDPEEGRFNNLRLIVERNNLYVTGFVNRTNNVFYRFADFSHVTFPGTTAVTLSGDSSYTT | |
| | LQRVAGISRTGMQINRHSLTTSYLDLMSHSGTSLTQSVARAMLRFVTVTAEALRFRQIQRGFRTTLDDL | |
| | SGRSYVMTAEDVDLTLNWGRLSSVLPDYHGQDSVRVGRISFGSINAILGSVALILNCHHHASRVARMAS | |
| | DEFPSMCPADGRVRGITHNKILWDSSTLGAILMRRTISS | |
| | [SEQ ID NO: 31] | |
| MC006 | ATGCAAAGGAAAGGCAATAAACTGTTGATTCAGTTATGCAGTGTGATACTGCTATTTTTTACCACATCC | 1110 |
| | TGGTATGCATTGGCGAATGAATGTTATATAGAGAGAAATGCTGAAGGGGATTATCACATGAAGATAAGC | |
| | TCTACTCAGCTTAGTCTGGCGTCACAAATGGTCGAGGTTCCGACAGAAATAGCCGAAGCTACATGGGAT | |
| | GTAAATATTCAACTAAGAGGCGATGCCATAGGGTGTAAATCTCTTGGGGATAGTAAGGCAGTTCACTTT | |
| | CTTAATACAGCTGACCCAAGTTTAATATCCACGTACACCACAACGAATGGCGCAGCGTTATTAAAAACA | |
| | ACTGTTCCAGGCATTGTGTATTCTGTCGAGTTATTATGCCTTAGTTGTGGTGCCGCAGATGAACTTGAT | |
| | TTATGGCTACCTGCACAAAGTGGCGCAGATAACTTCATACCAAGCACCCAGACGAAATGGGCCTATGAG | |
| | TACAGTGATCAAAGTTGGTATTTACGTTTTCGCTTATTCATAACTCCTGAATTTAAACCCAAGAATGGT | |
| | GTTTCCAGCGGAACAACGATAGCAGGAAAGATTGCGTCATGGTATATAGGTACCAATGACCAGCCGTGG | |
| | ATCAACTTTTACATTGACAATGACTCTTTAAAGTTTTTCGTCGATGAACCGACCTGTGCAACAGTTGCC | |
| | CTGGCACAAGATCAGGGCAACGTCAGTGGCAATCAGGTAACGCTTGGGAACAGCTATGTTTCGGAAGTG | |
| | AAAAATGGGCTTACGCGGGAAATCCCTTTTTCTATCCGTGCTGAATACTGTTATGCCAGTAAAATTACG | |
| | GTTAAGTTGAAAGCGGCAAATAAACCCAGCGATGCCACACTGGTGGGTAAAACGACTGGCTCGGCTTCA | |
| | GGCGTGGCTGTAAAAGTAAATTCAACTTATGACAATAGCAAAGTATTGTTAAAAGCAGATGGTAGCAAC | |
| | ACGGTTGACTACAACTTCGCCGCCTGGTCAAACAACCTGCTGTTTTTACCTTTTACGGCGCAGCTGGTA | |
| | CCGGATGGTAGCGGTAATGCTGTCGGTGTTGGAACATTTTCAGGTAACGCGACCTTCTCCTTTACCTAC | |
| | GAATAA | |
| | [SEQ ID NO: 8] | |
| MC006 | MQRKGNKLLIQLCSVILLFFTTSWYALANECYIERNAEGDYHMKISSTQLSLASQMVEVPTEIAEATWD | 369 |
| prot | VNIQLRGDAIGCKSLGDSKAVHFLNTADPSLISTYTTTNGAALLKTTVPGIVYSVELLCLSCGAADELD | |
| | LWLPAQSGADNFIPSTQTKWAYEYSDQSWYLRFRLFITPEFKPKNGVSSGTTIAGKIASWYIGTNDQPW | |
| | INFYIDNDSLKFFVDEPTCATVALAQDQGNVSGNQVTLGNSYVSEVKNGLTREIPFSIRAEYCYASKIT | |
| | VKLKAANKPSDATLVGKTTGSASGVAVKVNSTYDNSKVLLKADGSNTVDYNFAAWSNNLLFLPFTAQLV | |
| | PDGSGNAVGVGTFSGNATFSFTYE | |
| | [SEQ ID NO: 32] | |
| MC008 | ATGAGATTCGCCAAAGGCGTGTTGCTCGCCATCTGTCTGATTTTTTTGCCGCTAAAAGCGGCGCTGGCG | 1062 |
| | CTGAACTGCTATTTTGGTACCGCAAACGGCGCTGTAGAAAAATCGGAAGCTATTATGCCCTTCGCCGTT | |
| | CCGGCCAATTCCAAACCCGGCGATAAAATCTGGGAATCTGACGATATAAAAATTCCGGTCTACTGTGAC | |
| | AACAACACAAACGGTAATTTTGAAAGCGAGCACGTCTACGCCTGGGTAAACCCTTACCCCGGTATTCAG | |
| | GATCCCTATTATCAATTGGGCGTAACCTATGAAGGCGTGGACTATGACGCAAGCCTTGGGAAAAGCCGC | |
| | ATCGAGACGAATCAGTGCATCGACAGTAAGAATATTGATATTTAGACCCCGGAGCAGATCATTGCGATG | |
| | GGCTGGCAAAATAAACTTTGCTCCGGCGATGCAACCGTTATGCACAAATCGCGGACGTTTGTGGCGCGT | |
| | ATGCGGCTTTACGTTAAAGTTCGCGCCATGCCGCCCCATGATTATCAGAGCAAACTCAGCGATTACATC | |
| | GTCGTACAGTTTGATGGCGCAGGCAGCGTGAACGAAGACCCTACCGCGAAGAATCTGAAATATCACATT | |
| | ACCGGTCTGGAAAATATCCGCGTGCTGGATTGCAGCGTCAACTTTGCTATCTCACCTGAAACCCAGGTC | |
| | GTTGATTTTGGCCGATTTAACGTGCTGGATATTCGTCGCCATACGATGTCGCAGCAATTTAAAATAACT | |
| | ACAACCAAATCACAAAACGACCAGTGTACCGACGGTTTTAAAGTCAGCTCTTCGTTTTATACCGATGAA | |
| | ACCCTTATCGACGAAGATAAATCACTGCTCATTGGCAATGGCTTGAAGCTTCGATTGTTGGATGAAAAT | |
| | GCCTCACCCTATACTTTCAACAAATACAGTGAGTATGCCGATTTTACCAGCGACCTGTTAGTGTATGAA | |
| | AAAAGCTACACGGCAGAATTGTCATCCACGCCGGGTACGCCCATCGACGTTGGTCCCTTTGATACAGTG | |
| | GTACTTTTCAAAATTAACTACAACTGA | |
| | [SEQ ID NO: 9] | |
| MC008 | MRFAKGVLLAICLIFLPLKAALALNCYFGTANGAVEKSEAIMPFAVPANSKPGDKIWESDDIKIPVYCD | 353 |
| prot | NNTNGNFESEHVYAWVNPYPGIQDPYYQLGVTYEGVDYDASLGKSRIDTNQCIDSKNIDIYTPEQIIAM | |
| | GWQNKLCSGDPTVMHKSRTFVARMRLYVKVRAMPPHDYQSKLSDYIVVQFDGAGSVNEDPTAKNLKYHI | |
| | TGLENIRVLDCSVNFAISPETQVVDFGRFNVLDIRRHTMSQQFKITTTKSQNDQCTDGFKVSSSFYTDE | |
| | TLIDEDKSLLIGNGLKLRLLDENASPYTFNKYSEYADFTSDLLVYEKTYTAELSSTPGTPIDVGPFDTV | |
| | VLFKINYN | |
| | [SEQ ID NO: 33] | |
| MC009 | TTGTGGGAATGTGATGCAACAGATTTGCCAAACATCTATTTTCTGGTCGCTACCAATGGCGACGACCGC | 1086 |
| | GTGGGGGGCTTTTACGATGGAGGCGGGCCTGATGGTCTGAGTGATGTCTATGCCACCTGGTTGGCTTTT | |
| | GTCGGTCTCAAGCAGACCATGGCGGGCGTGACGCTTGGTCGTTACTGGAAGAAAGTGCCCATCACCAGT | |
| | TATGCCACTCAGGGAACTAAAATCCAGATTCGCTTACAGGATATCCCTCCTCTTCATGCTGAGCTTTAT | |
| | CGCATCAGTACGCTACCTGATACATCAGCAACAACAAGTTGGTGCGGTAATAATAATACAGATAGTAGT | |
| | GGAGTCGGATTCGCAAAACCTTCCGGTACAATCTATAACTGTGTTCAGCCCAATGCCTATATTCAGCTT | |
| | TCCGGTACCAGCGGCATTTTATTTGGTCATGATGAGCCCGGCGAAGATAGTTCTGTTCATTGGGATTTC | |
| | TGGGGTGCTGATAATGGTTTTGGTTACGGAATGCGTTCGGCCAATCGACTCTACAACAATGCCACCTGC | |
| | GTTGCCCGCAGCGCCACGCCGTTAGTATTGCTGCCGACAATTGCAGAAGCACAACTGAATGCGGGCATG | |
| | GAAAGTACCGGTAATTTTAATGTCCGCGTCGAGTGTAGTAACTCGGTTCAATCAGGGATTAGCGATACT | |
| | CAGACAGCATTAGGAATCCAGGTGTCTGAAGGTGCATATACAGCGGCGCAAAAACTGGGGATTATCAAT | |
| | AGCAACGGCGGCGTCAGCGCCCTGGTCTCTGATAATTATGACGCAGCAGAGATGGCAAAGGGCGTTGGG | |
| | ATCTACATTTCTAACAGTGCTCACCCCGATACGGCGATGACGCTGGTTGGTCAACCGGGCATCGCGAAG | |
| | TTAACCCCCGGAGGAAATGCAGCGGGGTGGTATCCTGTATTTGAAGGGGCAACATTAGAAGGTGCGACT | |
| | CACCCCGGATACTCCAGCTATAGTTACTCTTTTATCGCCCGGTTGAAGAAACTGCCAAATCAGACAGTC | |
| | AGTGCGGGAAAAGTGCGGGCAACGGCTTATATATTGGTGAAAATGCAATGA | |
| | [SEQ ID NO: 10] | |
| MC009 | MWECDATDLPNIYFLVATNGDDRVGGFYDAGGPDGLSDVYATWFAFVGLKQTMAGVTLGRYWKKVPITS | 361 |
| prot | YATQGTKIQIRLQDIPPLHAELYRISTLPDTSATTSWCGNNNTDSSGVGFAKPSGTIYNCNQPNAYIQL | |
| | SGTSGILFGHDEPGEDSSVHWDFWGADNGFGYGMRSANRLYNNATCVARSATPLVLLPTIAEAQLNAGM | |
| | ESTGNFNVRVECSNSVQSGISDTQTALGIQVSEGAYTAAQKLGIINSNGGVSALVSDNYDAAEMAKGVG | |
| | IYISNSAHPDTAMTLVGQPGIAKLTPGGNAAGWYPVFEGATLEGATHPGYSSYSYSFIARLKKLPNQTV | |
| | SAGKVRATAYILVKMQ | |
| | [SEQ ID NO: 34] | |
| MC010 | ATGAATATGGCTTTTTACGGCAAGTGGTTTGCTTGCCTGTGGCTCGCTACGTCGTGCGTTCAGGCGGCG | 789 |
| | AGCACAGATAATAAGGCGCTGGAGATAATTCGTCGCGCTGACGAAATTCGTTCGCCGAATAAACCGTTT | |
| | CGTTAGACGCTGACGGTAACGGAATATAAAGCGGGCGCAACCCAGCCAGAGAATAAACAGGTCCTCGAT | |
| | ATTTCCATGCGCTTTATGAAACCGCAGGGTAATGAAAAAGCGGATGCACGCTCGCTGGTGCGTTTTATT | |
| | TATCCGCCGCGAGATAAAGGAAAAATCATGCTTTCCGACTGGTACGATTTATGGTTTTACACGCCGGAA | |
| | CTGCGACGTCCTATGCCTATTTCACGGCAACAAAGATTAATTGGGCAAATCTCTAATGGCGATGTCAT7 | |
| | GTCACTAACTTTGAATATGCCTATGACTCGACCCTGATGGGGGAAGTCACGTGTGCTGAAAAGCAGTGT | |
| | TACAAGCTGGCGCTGGTGCGTAAATCGGCGGATATCACCTGGCCGAAGGTTATCTATTACGTTGAAAAA | |
| | GACGGTGATAATCGCCCGTGGAAGGCGGCCTATTATTCGCAGGATGACCAGTTAATAAAAGAAGTGTTG | |
| | TATCAGGATTTCCAGCCGGTGTTGGGGAAAACGCGACCCATGAAAATAACCGTTACCGATGTTCGTCAC | |
| | GGCAATAACTATTCAGTGATGGAGTACAGCGATGTTCGTCTGGAATCGCTGCCGGAGTTTCATTTCACC | |
| | AAAGAGTATATTCAGCGAGGTGCGAAATGA | |
| | [SEQ ID NO: 11] | |
| MC010 | MNMAFYGKWFACLWLATSCVQAASTDNKALEIIRRADEIRSPNKPFRYTLTVTEYKAGATQPENKQVLD | 262 |
| prot | ISMRFMKPQGNEKADARSLVRFIYPPRDKGKIMLSDWYDLWFYTPELRRPMPISRQQRLIGQISNGDVI | |
| | VTNFEYAYDSTLMGEVTCAEKQCYKLALVRKSADITWPKVIYYVEKDGDNRPWKAAYYDQDDQLIKEVL | |
| | YQDFQPVLGKTRPMKITVTDVRHGNNYSVMEYSDVRLESLPEFHFTKEYIQRGAK | |
| | [SEQ ID NO: 35] | |
| MC011 | ATGAAACGTACGCTGAAGATCTCTTCGTTGCTGTGTGTTGCGCTGCCGTTGACGGTACAGGCGGATTGC | 1389 |
| | CTGTCAGGTGACGAGGTGGCACAAAATAGCGATGTGACGCTTTACGAGCAGGTGTCTTATGTCAATAAA | |
| | CAGGCTTCGGCCTGGCAGATTGCAGGCAAAAATCCCTACACCCGTCACAACGGTTATCAGGAGGCGGGG | |
| | ATTGGCATCAATAGCGGTTGTTCGATAATCGACAATACGCTGGACCTTAAGCTCAATCTGTATGGAATA | |
| | AACGAATATGCGCTTAAACCCGCAGGGAAATTCGAAACGGATGATAGCCGCACGCGTGCGTTAATTAAC | |
| | CGCCTCAGTCTGGTCTATAGCGCCTCGGACAGCGTTCAATTTGAAGCAGGCAAATTTGCCGCGCCGTCA | |
| | GGAATGTTCTTTTTGCGTTCGCCTTCTGATTTGCAAACGCATTATTACACCGGATTCCAGTCGACGCGT | |
| | TTGCACGATCCCAAAATGACATCGGCGTATCAGGCGTCATCGTGGGGCGCAAAAATGAGCGTGGATACA | |
| | CGCGATTATGCGTTTTCGGCGAGTGTCATACCGAAGTTAGCCACCATCGACAAACGCTATGTGACGTCT | |
| | GGCAACTGGTCGGCTAATCAGCAGGGAAACAGCGACGAAGCCTATCTGCTGAGCTTCAGCGATCATCGC | |
| | TTTGGTGAGCACACGCCGACAGTCAATGTGCGGCTCGGACCATCGCCGTCACTCGCGCTGTCTGACAGT | |
| | TATCACTACACCCCGCAGCTTACGCTGAATGTCGACGCAGCCTACCACCGCTCCCAGCAATGGCGGCAT | |
| | CTTTCCCACCGTGAGACGGCGCAGGTCGAGGAATATCAGTTTCCGGATTCGCTCTATGAAACGAAAGAT | |
| | GAAAGCGGCGTAGAGCTGGCGCTGGGTGGGGAGTACACCAGTGATAATTTCAGCGTGTTTGGCGTTGAG | |
| | TATTACTTTCAGAGCGAGGGTTATTCGCGCGCTGAACAGCGTCAGCAGCGCGAGTTAATTGACTTCCTC | |
| | AATACCACAACCGGCTATGCGCCACTGGATCAGGCATTTGATTCTTACAAATACCTGATGGCTTCAGAA | |
| | ATCAGCAATACCGCCAATCAGGGGATGCTGCAGGGTAAGCACTATCTGAACGCCTGGGCCAGTCTGCCG | |
| | CTGGCGGGAGAGTCGACGCTGCAGCCTTCGCTGGTGGTGAATCTCGTTGATGGCAGCACGTTGCTGGGC | |
| | CTGCATTATTCCACGCCGCTCAGCGCTATAAGTAACCAGCTTGAAGCCTATGCCGGTGGCTATAGCGCG | |
| | CTGGGAAGTCGGTACTCCGAATTTGCGCTTTTTGGCGACACGTTAGGCCTTTATTTGGGCTTTAAATAC | |
| | TATCTATAA | |
| | [SEQ ID NO: 12] | |
| MC011 | MKRTLKISSLLCVALPLTVQADCLSGDEVAQNSDVTLYEQVSYVNKQASAWQIAGKNPYTRHNGYQEAG | 462 |
| prot | IGINSGCSIIDNTLDLKLNLYGINEYALKPAGKFETDDSRTRALINRLSLVYSASDSVQFEAGKFAAPS | |
| | FMFFLRSPSDLQTHYYTGFQSTRLHDPKMTSAYQASSWGAKMSVDTRDYAFSASVIPKLATIDKRYVTS | |
| | GNWSANQQGNSDEAYLLSFSDHRFGEHTPTVNVRLGPSPSLALSDSYHYTPQLTLNVDAAYHRSQQWRH | |
| | LSHRETAQVEEYQFPDSLYETKDESGVELALGGQYTSDNFSVFGVEYYFQSEGYSRAEQRQQRELIDFL | |
| | NTTTGYAPLDQAFDSYKYLMASEISNTANQGMLQGKHYLNAWASLPLAGESTLQPSLVVNLVDGSTLLG | |
| | LHYSTPLSAISNQLEAYAGGYSALGSRYSEFALFGDTLGLYLGFKYYL | |
| | [SEQ ID NO: 36] | |
| MC012 | GTGGGGATCGACAGCCGTAATGATATTCCTGAGGGGATTGCGACGCTGGGCGCTTTTATGGGTTATTCC | 705 |
| | CATTCACATATCGGTTTTGATCGTGGAGGACATGGCAGTGTGGACAGTTATTCTCTGGGCGGCTATGCC | |
| | AGTTGGGAACATGAAAGTGGTTTCTATCTGGACGGTGTCGTGAAGCTGAACCGTTTTGAAAGTAACGTA | |
| | GCCGGTAAAATGAGCAGCGGTGGAGCCGCCAATGGCAGTTACCATAGCAACGGGCTGGGCGGTCACATT | |
| | GAAACCGGGATGCGATTTACCGATGGTAACTGGAACCTGACGCCGTATGCCTCGTTAACGGGGTTCACC | |
| | GCTGATAACCCCGAATATCATTTATCCAATGGCATGGAATCGAAATCAGTCGATACCCGCAGTATATAT | |
| | CGTGAACTGGGTGCAACGCTGAGTTACAACATGCGTCTGGGGAACGGTATGGAAGTTGAGCCGTGGCTG | |
| | AAGGCGGCTGTGCGCAAAGAATTTGTCGATGATAACCGGGTGAAAGTGAATAGTGACGGTAATTTCGTC | |
| | AATGATTTGTCGGGCAGACGTGGAATATACCAGGCAGGTATTAAAGCCTCATTCAGCAGTACGTTAAGC | |
| | GGGCATCTTGGGGTGGGGTATAGCAACGGTGCTGGTATGGAATCCCCGTGGAACGCGGTGGCTGGTGTG | |
| | AACTGGTCGTTCTGA | |
| | [SEQ ID NO: 13] | |
| MC012 | MGIDSRNDIPEGIATLGAFMGYSHSHIGFDRGGHGSVDSYSLGGYASWEHESGFYLDGVVKLNRFESNV | 234 |
| prot | AGKMSSGGAANGSYHSNGLGGHIETGMRFTDGNWNLTPYASLTGFTADNPEYHLSNGMESKSVDTRSIY | |
| | RELGATLSYNMRLGNGMEVEPWLKAAVRKEFVDDNRVKVNSDGNFVNDLSGRRGIYQAGIKASFSSTLS | |
| | GHLGVGYSNGAGMESPWNAVAGVNWSF | |
| | [SEQ ID NO: 37] | |
| MC013 | ATGAAATTCCCTTCAATATTTAACAAAATAAAACCACAATCCATACAGCAACATCCAGAAAAAAATCAA | 1260 |
| | CTTAACTGGATGCTCGAATTAAATAAATGGAAAGAAGAACGTATACTTACAGGTGAAATCCATCGTCCG | |
| | GAATGTCGAAACGAAGCCGCTAAAAGGATAAACTGTGCTTTTTTGTCGAAACAGAATGACATTGATTTA | |
| | TCAGGACTTAATTTATCTACTCAACCACCAGGGCTGCAAAACTTCACCTCTATCAATCTTGATAATAAC | |
| | CAACTCACACATTTTGATGCAACCAACTACGATAGACTCGTAAAACTTAGTCTGAATAGTAACACTCTT | |
| | GAGTCAATAAATATTCATCAAGGCAGAAATGTAAGCATTACACATATATCTATGAATAATAATTGTCTC | |
| | AGAAATATTGATATAGATAGGCTTTCATCAATTACTTATTTTAGTGCGGCACATAATAAACTAGAGTTT | |
| | GTGCAATTAGAATCTTGCGAATGGCTGCAATACCTGAATCTCAGCCATAATCAATTAACTGATATTGTT | |
| | ACAGGAAATAAAGAAGAACTCTTACTGCTGGATCTATCCCATAATAAACTAGCAAGTTTACACAATGCC | |
| | TTATTTCCCAACTTAAATACGTTACTTATCAACAACAACTTGCTTTCTGAAATTAAAATGTTTTATAGC | |
| | AACTTCTGCAAAGTTCAGACATTAAACGCTGCTAACAATCAGTTGGAAAAAATAAACCTTCATTTCCTG | |
| | ACTTATCTTTCATCTATCAAAAGTTTAAGGCTGGACAATAATAAAATAACTCGCATTGATACTGAGAAC | |
| | ACATCCGATATTAGAAGTTTATTCCCCATAATAAAGAAGAGCGAAAGCTTAAATTTTTTAAATATTTCT | |
| | GGCGAGAACAATTGCCCTACTATCCAGCTCATGTTATTTAATTTGTTTTCCCCAGCACTTAAGCTTAAT | |
| | ACTGGCCTGGCAATTCTTTCGCCTGGTGCATTTGAAGATCACTCTGACGGATTAGATGTGGATAACGAA | |
| | TTGTTTCACTATACTATTAATAAAGCATATACCCCATATAATATACATACTTATAAAACAGAAGAAGTT | |
| | GTAAACCAGAGGAATATAAAAATTAAAAATATGACCTTAGATGAAATAAACAATACTTATTGTAATAAC | |
| | GATTATTACAATGAGGCAATAAGAGAGGAACCGATAGACTTTCTGGACAGATCGTTTTCCTCCAGCTCA | |
| | TGGCCTTTTTATCACTAA | |
| | [SEQ ID NO: 14] | |
| MC013 | MKFPSIFNKIKPQSIQQHPEKNQLNWMLELNKWKEERILTGEIHRPECRNEAAKRINCAFLSKQNDIDL | 419 |
| prot | SGLNLSTQPPGLQNFTSINLDNNQLTHFDATNYDRLVKLSLNSNTLESINIHQGRNVSITHISMGGGCL | |
| | RNIDIDRLSSITYFSAAHNKLEFVQLESCEWLQYLNLSHNQLTDIVTGNKEELLLLDLSHNKLASLHNA | |
| | LFPNLNTLLINNNLLSEIKMFYSNFCKVQTLNAANNQLEKINLHFLTYLSSIKSLRLDNNKITRIDTEN | |
| | TSDIRSLFPIIKKSESLNFLNISGENNCPTIQLMLFNLFSPALKLNTGLAILSPGAFEDHSDGLDVDNE | |
| | LFHYTINKAYTPYNIHTYKTEEVVNQRNIKIKNMTLDEINNTYCNNDYYNEAIREEPIDFLDRSFSSSS | |
| | WPFYH | |
| | [SEQ ID NO: 38] | |
| MC014 | GTGAACAAGGCCCCCTCTTTAATTGCCGCTATCGTCCTCGGACTGGGGATTAGCGCCTGTGGTTATTTT | 711 |
| | GTTGGCGATGGTGTTAAACATTTAAAAACCAACAACCGTTATGTCAATGTGCGCGGGCTTTCTGAAAAA | |
| | GAAGTTCGCGCAGATACGGCGGAATTAAGAATTGCGATTAACTTTAAGGGTAATGTTCCCGGCGAACTG | |
| | TTCCCGAAACTGGAAGAAGCGCAGAAAAAAATTGTTGCCGAACTCAATGCTCAGGGGATTAACGAGAAA | |
| | GAGATAATTCTGGGCCAATGGACAAGCAAGCGAACAGATTCCTTTTATCTGAAAGATGATCCCACAATG | |
| | CCACGCTATAACGCAGACGGGTCAGTGACGATAAAAACCCATAATGTTGCTGCTGTCGAAAAAGTCGTC | |
| | GCGAAATTAAACGAACTGCAAGTCGCTACGGATGGCGCTATCGCTGAGAGCAAAGTCGCCTATCGCTTT | |
| | AATGGTATTGGCGCACTGCGCGCTGAGATGATTGCTGCCGCCACCAAAGATGCACGCAACGCCGCACTA | |
| | CAATTTGCAACGGACTCAGGTAGCCAGGTGGGGTCAATTAGCGATGCCTCACAAGGCGTATTCCAGATC | |
| | TTTGCCAGTGGCAGTGATGAAGATGATCCTACGGCGATTAATAAAACGGTACGCGTAGTCACGACCGTA | |
| | ACCTATGCGTTGCAGGATTAA | |
| | [SEQ ID NO: 15] | |
| MC014 | MNKAPSLIAAIVLGLGISACGYFVGDGVKHLKTNNRYVNVRGLSEKEVRADTAELTIAINFKGNVPGEL | 236 |
| prot | FPKLEEAQKKIVAELNAQGINEKEIILGQWTSKRTDSFYLKDDPTMPRYNADGSVTIKTHNVAAVEKVV | |
| | AKLNELQVATDGAIAESKVAYRFNGIGALRAEMIAAATKDARNAALQFATDSGSQVGSISDASQGVFQI | |
| | FASGSDEDDPTAINKTVRVVTTVTYALQD | |
| | [SEQ ID NO: 39] | |
| MC015 | ATGAAAAAATGGACGATATTTTTGACAAGTCTGATATTACTTGCTCTCAGTTTAGAAACACCAAAGTGC | 843 |
| | TATGCCGGTGATAAACTCATGAGTGCAAGCTTTTCCAGTACTAAAATTTATTATGCGATGAAAAACGTT | |
| | ACTGCTTCCGGCAGTTTATATTTCTATGTCACGGTAGTGACGCCGGGAGAAGTAAGCTACGGACAATAC | |
| | AACTCCAATGCAAGAAAAGGCGACACCCTCAAGTTAATATCCTGGAGCGGATCTGGCCCGGCACCGACA | |
| | CTGGTTTTGACTGATTATAGACGTACGGACACCTCAAATTGCCCTGGTATCAATACTCGTGTCTTTGTC | |
| | TGTGCATATATGACTTTTAACGTCACTGTAGAATCTGATAATTATGGTTGTCCGTGGATTGCCTCATTT | |
| | TATTGAGTATCTGAGGCATTTGGATTTGGTAGGTATACAAGCCGAACAGTTCATGATTCTATTTGGCCG | |
| | ACAATTCCCGTCGCTTCGTTTGATATATCCTGGAGTGATAATTATGTTAGCCATAATAAAGCGCTGCGA | |
| | TTGCAATCCGATGGTTCGACAATCACGACTACGCTTTCGACCTACCTGATGGAAAGCGGCAAACTCTGC | |
| | GATGGCAGTATTTTTGATTCCAGAGGGGCATATTGTCGTGCTGTTTCTGACTTATTAACCTTTACCTCG | |
| | TATGGTTGTGATAATGCAAAGGTGACAGTCACCCCCTCACGTCAACCATTGACGGATAGAAAGCTACAC | |
| | GACATTGTGGTACAGGTGAATACCAGTAGCCGTGAGCCAATTGATTCTACATGCCGTTTCCAGTATGTA | |
| | CTGAATGAACTGTAG | |
| | [SEQ ID NO: 16] | |
| MC015 | MKKWTIFLTSLILLALSLETPKCYAGDKLMSASFSSTKIYYAMKNVTASGSLYFYVTVVTPGEVSYGQY | 280 |
| prot | NSNARKGDTLKLISWSGSGPAPTLVLTDYRRTDTSNCPGINTRVFVCAYMTFNVTVESDNYGCPWIASF | |
| | YSVSEAFGFGTYTSPTVHDSICPTIPVASFDISWSDNYVSHNKALRLQSDGSTITTTLSTYLMESGKLC | |
| | DGSIFDSRGAYCRAVSDLLTFTSYGCDNAKVTVTPSRQPLTDRKLHDIVVQVNTSSREPIDSTCRFQYV | |
| | LNEL | |
| | [SEQ ID NO: 40] | |
| MC016 | ATGGGTACAGCAGCTATAAGTCATTTACGATATGATCTTAACAAATATGCCCTTTCTTTACGCAAAACC | 912 |
| | GCAACACTTGCTTCAACATTTTTTATTGAGTCCCCATTAGTTAGATTCGAGTATCTGCAAGAAATAGAA | |
| | AATACTATTAATGATATTACTCACAGATTCAATTCTTCATATGACATCAATGAAAAAGCACGACTTATC | |
| | AATGAATTAAAAATGGAGTCAGAAACAGCAAGAAAAGAATATCAACTTTTCAGACAAGGAAATTATGAT | |
| | AAATATATCACTACTGATATTTTCGAAGAACATGGTTTGATAAAATATGTGAACTTAAGTTTGGATATT | |
| | GTTGCATCTGTAGGAGAGGTCGTAGGCGGGGTTGGCGCGTTGAAATTTGGTAAGGTAGTGCATTCAAAC | |
| | CGAATAAAAGGTATAGGCGTCACATTAGTTGCTCATGGGGCGAATAATTTTTATGAAAGCTTATCACCT | |
| | TTATTTTTTAACGAGTATGATTCAGGTCCAATACGTGAACTCTACAGAGTAATAGCAAAAAAAATGGGA | |
| | GGTGATATTAATTCTGCTGATTATGCATACAGCATCGTGGACTTTTCGATTACTGCATATGGTGGATAT | |
| | TCAGGCATGAAAATCGTACCTAAGTATAATAGATTGATTCGTCCTTCATTAGGAAATAGACCTGGTACA | |
| | GGACGATTATTTCATTACACATCAGTTGATTTTAAAAATAAATTCTCATTAAAACCTACCCCTTTAAAA | |
| | ATCATACAAATTAGTTCTACTGTAAAAAAATTCAAGGTTACATTTTATGATGAAAATACAAATTTGAGG | |
| | ATAAAACATGGCCTCAATAATCATATGCCACATCAATTATTAATGAATATATCAGGAACTACAACTGGT | |
| | GCAGGAATACAATAA | |
| | [SEQ ID NO: 17] | |
| MC016 | MGTAAISHLRYDLNKYALSLRKTATLASTFFIESPLVRFEYLQEIENTINDITHRFNSSYDINEKARLI | 303 |
| prot | NELKMESETARKEYQLFRQGNYDKYITTDIFEEHGLIKYVNLSFDIVASVGQVVGGVGALKFGKVVHSN | |
| | RIKGIGVTLVAHGANNFYESLSPLFFNEYDSGPIRELYRVIAKKMGGDINSADYAYSIVDFSITAYGGY | |
| | SGMKIVPKYNRLIRPSLGNRPGTGRLFHYTSVDFKNKFSLKPTPLKIIQISSTVKKFKVTFYDENTNLR | |
| | IKHGLNNHMPHQLLMNISGTTTGAGIQ | |
| | [SEQ ID NO: 41] | |
| MC017 | TTGCTGTCACTGACACTGATGTCTGCTTTATTATCGCCTTTATCTCTTCAGGCAGCGGATGTCCGGCGT | 1065 |
| | AGCGGAGATGAAGCATTTATCATTCAGCAGCAGCGTCAGGAAGCCCTTGAGCAACAACTGACGCCTTCA | |
| | GCCCCTGATGTTCGCCTTTCTGCACCTGGCTCTTTTGCCCATAAGATTAATTTTCCTGTTGAAACGCCC | |
| | TGTTTTCAGATTAAACAGACGGAACTGAAGGGGGCTGATGCGTTACCACACTGGCTGCCTTTACAAAAA | |
| | ATCGCCAACGGGGCGGTCGGGCATTGCCTGGGGGCGAAAGGAATTAATCTGCTGATGAGTACATTGCAG | |
| | AACCGTCTGGTCGATCATGGTTATGTCACCACCCGTGTTCTGGCACCTTCGCAGGATTTAAAAAGCGGT | |
| | ATCCTCCGGCTGGTTATTATTCCCGGTGTTGTGCGACATGTGCGTCTGACACCGGACAGTGATGACTAT | |
| | ATTCAGTTGTATTCCTCATTCCCGGCACACGAAGGTTCTCTGCTGGATTTACGGGACATTGAGCAGGGG | |
| | GTGGATTTAGGTAACAGCCGGATACAGGGACAACATACTGAGCTGAATGCAACCAGTGGAAATCTGTCT | |
| | ACACAGAATGCGCAACTGAGTGCCGATACGCTTTCCGCCCGGACTGCCGGGCAGTTCAGCAGTAATGGC | |
| | GGTACGATAAATGCCGACACACTGCAGATATCGGCACAAAGCCTGTCAAATCGTAAAGGCAGTCTGATT | |
| | CAGACGGGAACAGGGGATTTTTCGCTGAGTCTGCCGGGAAGCGTGGATAACCGGGAAGGGCTGCTTGCG | |
| | GCAAATGGCGCGGTGCGTCTGGATGCACTGAGCCTTGATAATCGCAAGGGGAAAGTGCAGGCGGAGCAG | |
| | TCACCCTCCCTTCAGAAATCCCCGCCCACGTTTCTGAAACCGTTTGTGGCTGGTGTCTGTGCGGCATTG | |
| | CTGGCGGTCAGCGTGGCTATTCCGGGATGGCAGTTTCTGACACAGCCATCACCGGAGGAGCAGCATTTT | |
| | ACCTGGGGGAATGGTTGTAAAAAGCAGTGA | |
| | [SEQ ID NO: 18] | |
| MC017 | MLSLTLMSALLSPLSLQAADVRRSGDEAFIIQQQRQEALEQQLTPSAPDVRLSAPGSFAHKINFPVETP | 354 |
| prot | CFQIKQTELKGADALPHWLPLQKIANGAVGHCLGAKGINLLMSTLQNRLVDHGYVTTRVLAPSQDLKSG | |
| | ILRLVIIPGVVRHVRLTPDSDDYIQLYSSFPAHEGSLLDLRDIEQGLDLGNSRIQGQHTELNATSGNLS | |
| | TQNAQLSADTLSARTAGQFSSNGGTINADTLQISAQSLSNRKGSLIQTGTGDFSLSLPGSVDNREGLLA | |
| | ANGAVRLDALSLDNRKGKVQAEQSPSLQKSPPTFLKPFVAGVCAALLAVSVAIPGWQFLTQPSPEEQHF | |
| | TWGNGCKKQ | |
| | [SEQ ID NO: 42] | |
| MC018 | ATGAGCGGAAAACCGGCGGCGCGTCAGGGCGACATGACGCAGTATGGCGGTAGCATTGTTCAGGGTTCA | 4200 |
| | GCCGGGGTGCGCATTGGTGCCCCCACCGGCGTGGCCTGTTCGGTGTGCCCCGGCGGAGTGACGTCCGGC | |
| | CATCCGGTCAATCCCCTGCTCGGTGCAAAGGTCCTTCCCGGTGAAACCGACATCGCCCTGCCCGGCCCG | |
| | CTGCCGTTCATCCTCTCCCGCACCTACAGCAGTTACCGGACAAAAACGCCCGCGCCGGTGGGGAGCCTC | |
| | GGCCCCGGCTGGAAAATGCCTGCGGATATCCGCTTACAGCTGCGCGATAACACACTGATACTCAGTGAT | |
| | AACGGCGGCAGAAGCCTGTATTTTGAGCACCTGTTTCCCGGTGAGGACGGTTACAGCCGCAGCGAGTCA | |
| | CTCTGGCTGGTGCGCGGCGGCGTGGCGAAACTGGATGAAGGTCACCGGCTGGCCGCACTCTGGCAGGCG | |
| | CTGCCGGAAGAACTCCGCTTAAGTCCGCATCGTTATCTGGCGACAAACAGTCCGCAGGGGCCGTGGTGG | |
| | CTGCTCGGCTGGTGTGAGCGGGTGCCGGAAGCGGATGAGGTGCTGCCTGCGCCGCTGCGGCCGTACCGG | |
| | GTACTGACCGGGCTGGTGGACCGCTTCGGGCGCACACAGACGTTCCACCGCGAAGCCGCCGGTGAATTC | |
| | AGCGGCGAAATCACCGGCGTGACGGATGGTGCCGGGCGTCACTTCCGGCTGGTACTGACCACGCAGGCG | |
| | CAGCGGGCAGAAGAAGCCCGGCAGCAGGCCATTTCCGGCGGGACGGAACCGTCCGCTTTTCCTGATACC | |
| | CTGCCGGGTTACACCGAATATGGCCGGGACAACGGCATCCGTCTGTCTGCCGTGTGGCTGACGCACGAC | |
| | CCGGAATACCCGGAGAATTTACCTGCCGCGCCGCTGGTGCGCTATGGCTGGACGCCGCGCGGCGAACTG | |
| | GCGGTGGTGTATGACCGTAGTGGCAAACAGGTGCGCAGCTTTACTTACGATGATAAATACCGGGGCCGG | |
| | ATGGTGGCGCACCGTCACACGGGCCGGCCGGAAATCGGTTACCGTTACGACAGCGACGGGCGGGTGACA | |
| | GAACAGCTAAACCCGGCAGGCTTAAGCTACACGTATCAGTATGAGAAAGACCGCATCACCATCACCGAC | |
| | AGCCTGAACCGCCGTGAAGTCCTGCACACGCAGGGTGAAGGCGGGCTGAAGCGGGTGGTGAAAAAGGAA | |
| | CACGCGGACGGCAGCGTCACGCAGAGTCAGTTTGACGCGGTGGGCAGGCTCAGGGCACAGACGGATGCC | |
| | GCAGGCAGGACAACAGAATACAGCCCGGATGTGGTGACGGGCCTCATCACGCGCATCACCACGCCGGAT | |
| | GGCAGGGCATCGGCGTTTTACTATAACCACCACAGCCAGTTAACGTCAGCCACCGGGCCTGACGGGCTG | |
| | GAAATACGCCGGGAATATGATGAATGGGGCCGTCTGATTCAGGAAACTGCCCCTGACGGCGATATCACC | |
| | CGCTACCGTTATGATAATCCACACAGTGACTTACCCTGCGCAACGGAAGATGCCACCGGCAGCCGGAAA | |
| | ACCATGACGTGGAGCCGTTACGGTCAGTTGCTGAGCTTCACCGACTGTTCCGGTTATGTAACCCGTTAT | |
| | GACCATGACCGCTTCGGGCAGATGACGGCGGTGCACCGCGAGGAAGGGCTGAGTCAGTACCGCGCATAC | |
| | GACAGCCGTGGACAGTTAATTGCCGTGAAAGAGACGCAGGGCCATGAAACGCGGTATGAATACAACGCC | |
| | GCCGGTGACCTGACCACCGTCATTGCCCCGGACGGCAGCAGAAACGGGACACAGTACGATGCGTGGGGA | |
| | AAAGCCATCTGTACCACGCAGGGCGGTCTGACGCGCAGTATGGAATACGATGCTGCCGGACGGGTCATC | |
| | CGCCTGACCAGTGAAAACGGCAGCCACACCACCTTCCGTTACGATGTACTCGACCGGCTGATACAGGAA | |
| | ACCGGCTTTGACGGCCGCACACAGCGTTATCACCACGACCTGACCGGCAAACTTATCCGCAGCGAGGAT | |
| | GAGGGGCTGGTCACCCACTGGCACTATGACGAAGGAGACCGCCTCACGCACCGCACCGTGAAGGGTGAA | |
| | ACCGCAGAGCGCTGGCAGTATGACGAACGCGGCTGGCTGACAGACATCAGCCATATCAGCGAAGGGCAC | |
| | CGGGTGACGGTGCATTACGGGTATGATGAGAAAGGCCGGCTGACCGGTGAGCGTCAGACGGTGCATCAC | |
| | CCGCAGACGGAAGCACTGCTCTGGCAGCATGAGACCAGACACGCTTACAACGCGCAGGGGCTGGCGAAC | |
| | CGCTGTATACCGGACAGCCTGCCCGCCGTGGAATGGCTGACCTATGGCAGCGGCTGGCTGGCAGGCATG | |
| | AAGCTCGGCGACACACCGCTGGTGGATTTCACGCGCGACCGCCTGCACCGGAAAACGCTGCGCAGATTC | |
| | GGCCGTTATGAACTCACCACCGCTTATACCCCTGCCGGGCAGTTACAGAGCCAGCACCTGAACAGCCTG | |
| | CAGTATGACCGCGATTACACCTGGAACGACAACGGCGAACTCATCCGCATCAGCAGCCCGCGCCAGACC | |
| | CGGAGTTACAGCTACAGCGACTCCGGCAGGCTGACCGGCGTTCACACCACCGCAGCGAATCTGGATATC | |
| | CGCATCCCGTATGCCACGGACCCGGCAGGTAACCGCCTGCCCGACCCGGAGCTGCACCCGGACAGCACC | |
| | CTCAGCATGTGGCCGGATAACCGTATCGCCCGTGACGCGCACTATCTTTACCGGTATGACCGTCACGGC | |
| | AGGCTGACAGAGAAAACCGACCTCATCCCGGAAGGGGTTATCCGCACGGATGATGAGCGGACTCACCGG | |
| | TACCATTACGACAGTCAGCACCGGCTGGTGCACTACACGCGGACACAATATGAAGAGCCGCTGGTCGAA | |
| | AGCCGCTATCTTTACGACCCGCTGGGCCGCAGGGTGGCAAAACGGGTGTGGCGACGTGAACGGGACCTG | |
| | ACGGGCTGGATGTCGCTGTCACGGAAACCGCAAGTGACCTGGTACGGCTGGGACGGCGACCGGCTGACC | |
| | ACAATACAGAACGACAGAACCCGCATCCAGACGATTTATCAGCCGGGGAGCTTCACGCCACTCATCAGG | |
| | GTTGAAACCGCCACCGGTGAGCTGGCGAAAACGCAGCGCCGCAGCCTGGCGGATGCGCTTCAGCAGTCC | |
| | GGCGGCGAAGACGGTGGCAGTGTGGTGTTCCCGCCGGTGCTGGTGCAGATGCTCGACCGGCTGGAAAGT | |
| | GAAATCCTGGCTGACCGGGTGAGTGAGGAAAGCCGCCGCTGGCTGGCATCGTGCGGCCTGACTGTGGCG | |
| | CAGATGCAAAGCCAGATGGACCCGGTATACACGCGGGCGCGAAAAATCCACCTGTACCACTGCGACCAT | |
| | CGCGGCCTGCCGCTGGCCCTTATCAGTAAGGAAGGGGCAACAGAATGGTGCGCAGAATACGATGAGTGG | |
| | GGCAACCTGCTGAATGAAGAGAACCCGCATCAGCTGCAGCAGCTTATCCGCCTGCCGGGGCAGCAGTAT | |
| | GATGAGGAGTCCGGCCTGTATTACAACCGCCACCGCTATTATGACCCGCTGCAGGGGAGGTATATCACT | |
| | CAGGATCCGATTGGACTGAAGGGGGGATGGAATTTTTATCAGTATCCGTTGAATCCGGTTCAGTATATA | |
| | GATTCAATGGGACTGGCATCAAAATATGGACACTTAAATAATGGCGGATATGGAGCGAGACCGAACAAA | |
| | CCGCCTACGCCCGATCCAAGTAAATTTCCAGACATAGCGAAACAATTAAGACTGCCATATCCTATTGAC | |
| | CAGGCCAGTAGTGCGCCTAATGTTTTCAAAACATTCTTCAGAGCATTAAGCCCTTACGACTACACACTG | |
| | TATTGCAGGAAGTGGGTAAAACCAAATCTGACTTGTACGCCACAGGATGATCCCCAGTATCCAGGGATG | |
| | GATACAAAGACAGCAAGTGATTACCTGCCACAGACAAATTGGCCAACAACTCAATTACCAGCAGGATAT | |
| | ACTTGTGCAGAACCCTATTTATTCCCTGACATTAATAAACCCGATGGGCCAGCAACAGCAGGGATAGAT | |
| | GATTTGGGTGAAATTTTAGCTAAAATGAAACAGAGAACATCGAGAGGAATAAGAAAATGA | |
| | [SEQ ID NO: 19] | |
| MC018 | MSGKPAARQGDMTQYGGSIVQGSAGVRIGAPTGVACSVCPGGVTSGHPVNPLLGAKVLPGETDIALPGP | 1399 |
| prot | LPFILSRTYSSYRTKTPAPVGSLGPGWKMPADIRLQLRDNTLILSDNGGRSLYFEHLFPGEDGYSRSES | |
| | LWLVRGGVAKLDEGHRLAALWQALPEELRLSPHRYLATNSPQGPWWLLGWCERVPEADEVLPAPLPPYR | |
| | VLTGLVDRFGRTQTFHREAAGEFSGEITGVTDGAGRHFRLVLTTQAQRAEEARQQAISGGTEPSAFPDT | |
| | LPGYTEYGRDNGIRLSAVWLTHDPEYPENLPAAPLVRYGWTPRGELAVVYDRSGKQVRSFTYDDKYRGR | |
| | MVAHRHTGRPEIRYRYDSDGRVTEQLNPAGLSYTYQYEKDRITITDSLNRREVLHTQGEGGLKRVVKKE | |
| | HADGSVTQSQFDAVGRLRAQTDAAGRTTEYSPDVVTGLITRITTPDGRASAFYYNHHSQLTSATGPDGL | |
| | EIRREYDEWGRLIQETAPDGDITRYRYDNPHSDLPCATEDATGSRKTMTWSRYGQLLSFTDCSGYVTRY | |
| | DHDRFGQMTAVHREEGLSQYRAYDSRGQLIAVKDTQGHETRYEYNAAGDLTTVIAPDGSRNGTQYDAWG | |
| | KAICTTQGGLTRSMEYDAAGRVIRLTSENGSHTTFRYDVLDRLIQETGFDGRTQRYHHDLTGKLIRSED | |
| | EGLVTHWHYDEADRLTHRTVKGETAERWQYDERGWLTDISHISEGHRVTVHYGYDEKGRLTGERQTVHH | |
| | PQTEALLWQHETRHAYNAQGLANRCIPDSLPAVEWLTYGSGWLAGMKLGDTPLVDFTRDRLHRKTLRRF | |
| | GRYELTTAYTPAGQLQSQHLNSLQYDRDYTWNDNGELIRISSPRQTRSYSYSDSGRLTGVHTTAANLDI | |
| | RIPYATDPAGNRLPDPELHPDSTLSMWPDNRIARDAHYLYRYDRHGRLTEKTDLIPEGVIRTDDERTHR | |
| | YHYDSQHRLVHYTRTQYEEPLVESRYLYDPLGRRVAKRVWRRERDLTGWMSLSRKPQVTWYGWDGDRLT | |
| | TIQNDRTRIQTIYQPGSFTPLIRVETATGELAKTQRRSLADALQQSGGEDGGSVVFPPVLVQMLDRLES | |
| | EILADRVSEESRRWLASCGLTVAQMQSQMDPVYTPARKIHLYHCDHRGLPLALISKEGATEWCAEYDEW | |
| | GNLLNEENPHQLQQLIRLPGQQYDEESGLYYNRHRYYDPLQGRYITQDPIGLKGGWNFYQYPLNPVQYI | |
| | DSMGLASKYGHLNNGGYGARPNKPPTPDPSKFPDIAKQLRLPYPIDQASSAPNVFKTFFRALSPYDYTL | |
| | YCRKWVKPNLTCTPQDDPQYPGMDTKTASDYLPQTNWPTTQLPPGYTCAEPYLFPDINKPDGPATAGID | |
| | DLGEILAKMKQRTSRGIRK | |
| | [SEQ ID NO: 43] | |
| MC019 | ATGGCTGCGGCAGCACAAGGTGTGGTAAACGCCGCAACCCAACAACCAGTTCCTGCACAAATTGCCATT | 7863 |
| | GCAAATGCCAATACGGTGCCCTACACCCTTGGAGCGCTGGAATCGGCCCAAAGCGTTGCCGAACGTTTC | |
| | GGTATTTCGGTGGCTGAGTTACGCAAACTCAACCAGTTTCGTACGTTTGCTCGAGGTTTTGATAATGTC | |
| | CGCCAGGGTGATGAACTGGATGTCCCGGCACAAGTTAGTGAAAATAATTTAACCCCGCCACCGGGTAAT | |
| | AGCAGTGGCAACCTTGAGCAACAGATAGCCAGTAGTTCACAGCAAATCGGGTCTCTGCTCGCCGAGGAT | |
| | ATGAACAGCGAGCAAGCGGCAAATATGGCGCGTGGATGGGCCTCTTCTCAGGCTTCAGGCGCAATGACA | |
| | GACTGGTTAAGCCGCTTCGGTACCGCAAGAATCACGCTGGGCGTGGATGAAGATTTTAGCCTGAAGAAC | |
| | TCCCAGTTCGATTTTCTCCATCCGTGGTATGAAACGCCTGATAATCTCTTTTTCAGTCAGCATACTCTC | |
| | CATCGTACTGACGAGCGTACGCAGATTAACAACGGCTTGGGTTGGCGTCATTTCACTCGCACATGGATG | |
| | TCGGGGATCAACTTCTTTTTCGAGCACGATCTTAGCCGTTACCACTCCCGCGCCGGCATTGGGGCGGAG | |
| | TACTGGCGCGACTATCTAAAATTAAGCAGTAACGGCTATTTGCGACTGACCAACTGGCGCAGCGCACCT | |
| | GAACTGGACAACGATTATGAAGCACGCCGGGCCAATGGCTGGGATGTACGCGCAGAAGGCTGGCTACCC | |
| | GCCTGGCCGCACCTTGGCGGTAAACTGGTCTATGAACAGTATTATGGCGATGAAGTGGCCCTGTTCGAT | |
| | AAAGATGATCGGCAAAGTAATCCTCATGCCATAACCGCTGGACTTAACTATACCCCCTTCCCGCTGATG | |
| | ACCTTCAGCGCGGAGCAACGCGAGGGTAAACAGGGCGAAAATGACACCCGTTTTGCCGTCGATTTTACG | |
| | TGGCAACCTGGAAGCGCGATGCAGAAACAGCTTGACCCGAATGAAGTCGATGCACGGCGTAGCCTTGCA | |
| | GGCAGCCGTTTTGATCTGGTGGATCGCAACAACAACATCGTTCTGGAATATCGCAAAAAAGAACTGGTT | |
| | CGCCTGACCCTGACAGACCCCGTGACAGGGAAGTCAGGAGAAGTGAAATCACTGGTTTCGTCGCTACAA | |
| | ACCAAATATGCCCTGAAAGGCTATAACGTCGAAGCCACCGCTCTGGAAGCTGCCGGTGGTAAAGTGGTT | |
| | ACAACGGGTAAAGATATTCTGGTTACCCTGCCGGCGTACCGGTTCACCAGTACGCCAGAAACCGATAAC | |
| | ACCTGGCCGATTGAAGTCACCGCTGAAGATGTCAAAGGCAATTTTTCGAATCGTGAACAGAGCATGGTA | |
| | GTCGTTCAGGCTCCTACGCTAAGCCAGAAAGATTCCTCGGTATCGTTAAGTAGCCAGACGTTGAGCGCG | |
| | GATTCCCATTCAACCGCCACACTGACTTTTATTGCGCATGATGCAGCAGGTAATCCTGTTATCGGGCTG | |
| | GTGCTCTCGACGCGTCACGAAGGTGTTCAGGACATCACCCTTTCTGACTGGAAAGATAATGGTGACGGA | |
| | AGCTATACCCAGATCCTGACCACAGGAGCGATGTCTGGCACGCTGACGCTGATGCCACAGCTGAACGGT | |
| | GTGGATGCGGCTAAAGCCCCCGCCGTGGTGAATATCATTTCTGTTTCGTCATCCCGGACTCACTCGTCA | |
| | ATTAAGATTGATAAGGACCGTTATCTCTCCGGGAATCCTATCGAGGTGACGGTAGAACTGAGAGATGAA | |
| | AATGACAAACCTGTTAAGGAGCAAAAACAGCAACTGAATACCGCAGTCAGCATCGACAACGTGAAACCT | |
| | GGTGTCACTACAGACTGGAAAGAAACCGCAGATGGCGTCTATAAGGCAACCTATACCGCCTATACCAAA | |
| | GGCAGTGGGCTTACTGCGAAGCTGTTAATGCAAAACTGGAATGAAGATTTGCATACCGCTGGATTTATC | |
| | ATCGACGCCAACCCGCAGTCAGCGAAAATTGCGACATTATCTGCCAGCAATAATGGTGTGCTCGCCAAT | |
| | GAGAATGCAGCAAACACCGTCTCGGTCAATGTCGCTGATGAAGGAAGCAACCCAATCAATGATCATACC | |
| | GTCACGTTTGCGGTATTAAGCGGATCGGCAACTTCCTTTAACAATCAAAACACCGCAAAAACGGATGTT | |
| | AATGGTCTGGCGACTTTTGATCTGAAAAGTAGTAAGCAGGAAGACAACACGGTTGAAGTCACCCTTGAA | |
| | AATGGCGTGAAACAAACGTTAATCGTCAGTTTTGTCGGCGACTCGAGTACCGCGCAGGTTGATCTGCAG | |
| | AAGTCGAAAAATGAAGTGGTCGCTGACGGCAATGACAGTGCCACAATGACCGCGACAGTTCGGGATGCA | |
| | AAAGGCAACCTGCTCAATGACGTCAAGGTCACCTTCAATGTCAATTCAGCAGCAGCGAAACTGAGCCAA | |
| | ACCGAAGTGAATAGCCACGACGGGATCGCCACAGCTACGCTGACCAGTTTGAAAAATGGTGATTATACG | |
| | GTTACGGCCTCTGTGAGCTCTGGTTCTCAGGCTAATCAACAGGTGATTTTTATCGGTGATCAAAGTACT | |
| | GCTGCCCTGACCCTCAGTGTGCCTTCAGGTGATATCACCGTCACCAACACAGCTCCGCTACATATGACT | |
| | GCAACCTTGCAGGATAAAAATGGCAATCCACTAAAAGATAAAGAAATCACCTTCTCTGTGCCAAACGAC | |
| | GTCGCAAGTCGGTTCTCGATTAGCAACAGCGGAAAAGGCATGACGGATAGCAACGGGACTGCAATCGCC | |
| | TCCCTGACCGGCACGTTAGCGGGCACGCATATGATCACGGCTCGTCTGGCTAACAGCAATGTCAGCGAT | |
| | ACACAGCCAATGACGTTTGTGGCGGATAAAGACAGAGCGGTTGTCGTTCTGCAAACATCGAAAGCGGAA | |
| | ATCATTGGGAATGGCGTGGATGAGACGACTCTGACAGCAACAGTTAAAGATCCTTTTGATAACGTGGTT | |
| | AAAAATCTTTCAGTAGTCTTCCGCACCTCCCCCGCAGACACGCAACTGAGTCTGAACGCGCGTAATACT | |
| | AATGAGAACGGTATTGCCGAAGTTACCCTTAAGGGCACGGTTTTGGGTGTTCATACAGCCGAAGCCATA | |
| | CTGCTTAACGGCAACAGAGATACGAAAATCGTCAATATTGCGCCCGATGCCAGCAACGCGCAGGTCACC | |
| | CTGAACATCCCTGCACAACAGGTGGTGACGAATAACAGTGACAGCGTGCAGCTGACGGCGACGGTGAAA | |
| | GACCCGTCGAATCATCCGGTGGCGGGAATAACGGTGAACTTCACCATGCCACAGGACGTGGCGGCAAAC | |
| | TTTACCCTTGAAAATAACGGTATTGCCATCACTCAGGCCAATGGCGAAGCGCATGTCACCCTCAAAGGC | |
| | AAAAAAGCGGGCACGCATACTGTGACCGCCACGCTGGGTAACAATAATGCCAGCGATGCGCAACCAGTC | |
| | ACCTTCGTGGCGGATAAGGACAGCGCGGTTGTCGTTCTGCAAACATCGAAAGCGGAAATCATTGGGAAT | |
| | GGCGTGGATGAGACGACTCTGACGGCAACAGTGAAAGATCCTTTTGATAACGCAGTAAAAGATCTACAG | |
| | GTCACCTTCAGTACCAACCCCGCAGATACTCAACTTAGTCAGAGCAAAAGCAATACTAACGACAGTGGT | |
| | GTGGCCGAAGTTACCTTTAAGGGCACGGTTTTGGGTGTTCATACAGCCGAAGCCACACTGCCTAACGGC | |
| | AACAACGATACGAAGATAGTCAATATTGCGCCCGATGCCAGCAACGCGCAGGTTACGCTGAACATCCCT | |
| | GCTCAACAGGTGGTGACGAATAACAGCGACAGCGTGCAGCTGACGGCGACGGTGAAAGATCCGTCGAAT | |
| | CATCCGGTGGCGGGAATAACGGTGAACTTCACCATGCCACAGGACGTGGCGGCAAACTTTACCCTCGAA | |
| | AATAACGGTATTGCCATCACCCAGGCCAATGGGGAAGCGCATGTCACGCTCAAAGGTAAAAAAGCGGGT | |
| | ACGCATACGGTTACCGCAACGCTGAGTAATAACAATACCAGTGATTCACAGCCGGTAACGTTTGTGGCG | |
| | GACAAAACCTCGGCTCTGGTTGTTCTTCAGATATCAAAAAATGAGATCACAGGTAATGGCGTCGATAGC | |
| | GCAACGCTAACTGCAAGGGTCAAAGATCAGTTCGACAATGAGGTGAACAATCTTCCGGTAACATTCAGC | |
| | ACAGCTTCTTCAGGCCTCACCCTGACCCCAGGGGAAAGTAATACCAATGAGTCTGGCATCGCGCAGGCC | |
| | ACTCTCGCAGGCGTTGGCTTTGGTGAGCAGACGGTCACTGCATCACTGGCTAATAATGGTGCCAGCGAC | |
| | AACAAAACTGTGCATTTTATTGGCGACACAGCGGCGGCAAAAATTATCGAGTTGACGCCTGTCCCAGAC | |
| | AGCATAATCGCAGGTACCCCGCAGAACAGCTCCGGCAGCGTCATCACCGCCACAGTCGTTGATAATAAT | |
| | GGCTTTCCGGTGAAAGGTGTGACTGTGAACTTCACCAGCAACGCAGCGACAGCCGAAATGACGAATGGC | |
| | GGTCAAGCCGTGACGAACGAACAGGGTAAGGCTACCGTCACTTATACCAATACCCGCTCCTCGATAGAA | |
| | TCAGGAGCGAGACCGGATACCGTTGAGGCCAGTCTGGAAAATGGTAGCTCCACGCTTAGCACATCAATT | |
| | AATGTCAACGCTGATGGGTCTACGGCACATCTCACCTTGCTACAGGCACTTTTTGATACAGTCTCCGCA | |
| | GGCGACACTACCAATCrGTATATTGAGGTGAAGGATAATTACGGCAACGGAGTACCCCAGCAGGAGGTA | |
| | ACCCTCAGCGTTTCACGAAGTGAAGGTGTGACCCCCAGTAATAACGCTATATATACGACCAATCACGAC | |
| | GGCAATTTTTACGCAAGCTTTACCGCTACAAAAGCCGGGGTATACCAAGTGACGGCAACCCTCGAAAAT | |
| | GGCGATTCGATGCAACAAACAGTGACCTATGTGCCGAACGTAGCGAATGCTGAAATCTCGCTGGCAGCC | |
| | TCGAAGGATCCGGTAATTGCCAACAATAACGATCTCACGACACTAACAGCAACAGTCGCTGATACAGAG | |
| | GGCAATGCGATAGCCAACAGTGAGGTAACATTTACTCTGCCGGAAGATGTGAGGGCGAACTTCACGCTG | |
| | GGCGATGGCGGTAAAGTGGTTACTGATACTGAAGGCAAAGCGAAAGTCACGCTGAAAGGTACAAAAGCA | |
| | GGCGCTCATACTGTTACAGCATCGATGGCTGGCGGTAAGAGTGAGCAGTTGGTGGTGAACTTTATTGCG | |
| | GATACACTCACTGCGCAGGTTAATCTTAACGTTACCGAGGACAATTTTATCGCTAATAACGTCGGGATG | |
| | ACCAGGCTGCAGGCAACAGTGACTGATGGAAACGGCAACCCGTTASCCAATGAGGCGGTGACATTCACG | |
| | CTACCGGCAGATGTGAGCGCAAGCTTTACTCTCGGACAAGGCGGTTCCGCCATTACTGACATCAACGGC | |
| | AAGGCTGAAGTTACACTGAGCGGTACAAAATCCGGCACCTACCCCoTGACAGTTAGCGTGAACAATTAT | |
| | GGTGTCAGTGATACGAAACAGGTGACTTTGATTGCCGATGCTGGTACCGCAAAACTAGCCTCCTTAACC | |
| | TCTGTATACTCATTCGTCGTCAGCACGACCGAGGGCGCGACCATGACTGCAAGCGTCACTGACGCTAAC | |
| | GGCAACCCGGTAGAAGGTATAAAAGTTAATTTCCGCGGAACTTCCGTCACGCTAAGCAGCACCAGCGTT | |
| | GAAACGGATGATCGGGGTTTCGCTGAAATTCTTGTGACAAGCACCGAGGTCGGACTGAAAACAGTTTCA | |
| | GCCTCTCTGGCAGATAAACCTACTGAAGTCATCTCGCGATTACTGAATGCAAAAGCAGATATTAATTCT | |
| | GCAACGATTACCAGTCTGGAGATACCTGAAGGTCAGGTCATGGTCGCACAAGACGTAGCAGTTAAAGCT | |
| | CACGTCAACGACGAGTTTGGCAATCCGATTCTTAATGAATCTGTAACATTCAGTGCAGAACCACCAGAG | |
| | CACATGACCATCAGCCAAAATATTGTCTCTACTGATACGCATGGTATAGCCGAGGTCACTATGACGCCC | |
| | GAAAGAAACGGTTCGTATATGGTGAAAGCATCCCTGGCGAATGGATCCTCTTATGAGAAGGATCTGGTG | |
| | GTAATCGATCAAAAACTGACACTCTCGGCGTCCAGCCCGCTTATCGGTGTCAATTCCCCAACAGGTGCA | |
| | ACTCTGACGGCAACGCTAACTTCTGCAAATGGCACTCCAGTGGAGGGTCAGGTCATCAACTTTAGCGTA | |
| | ACGCCAGAAGGTGCGACGTTAAGTGGCGGAAAAGTGAGAACCAACTCTTCAGGTCAGGCTCCAGTCGTT | |
| | CTGACCAGCAATAAAGTCGGTACATATACGGTGACTGCATCGTTCCATAACGGCGTAACAATACAGACA | |
| | CAGACAATCGTGAAAGrCACTGGCAACTCAAGCACCGCCCATGTTGCTAGCTTTATCGCTGATCCATCG | |
| | ACTATAGCCGCCACCAACAGTGATTTAAGTACCTTAAAGGCAACGGTTGAGGATGGCAGTGGTAACCTG | |
| | ATCGAAGGTCTCACTGrGTACTTCGCCTTAAAAAGCGGCTCTGCCACATTAACGTCATTAACAGCGGTG | |
| | ACAGATCAAAACGGAATCGCGACAACAAGCGTGAGAGGAGCGATAACGGGGAGCGTCACGGTAAGCGCA | |
| | GTCACGACCGCTGGTGGAATGCAAACAGTAGATATAACGCTGGTGGCAGGCCCGGCAGACGCCTCGCAG | |
| | TCCGTCCTTAAGAACAATCGGTCATCATTGAAAGGAGACTTTACCGATAGTGCTGAGCTACATCTTGTT | |
| | CTGCACGATATATCAGGCAATCCGATCAAAGTTTCTGAAGGGCTGGAATTTGTGCAGTCAGGTACCAAC | |
| | GCGCCCTATGTGCAAGTTAGTGCAATTGACTACAGTAAAAATTTCTCAGGCGAGTACAAAGCCACTGTT | |
| | ACAGGCGGCGGAGAGGGTATCGCAACGCTGATCCCTGTATTGAATGGTGTTCATCAAGCGGGTCTGAGT | |
| | ACCACAATACAATTCACTCGCGCAGAAGACAAAATAATGAGCGGTACAGTGTTAGTCAATGGTGCTAAC | |
| | CTACCGACAACTACATTCCCTTCGCAGGGGTTCACTGGGGCGTATTATCAGTTGAATAATGACAACTTT | |
| | GCCCCAGGAAAAACGGCGGCTGATTATGAGTTTTCAAGCTCTGCCTCCTGGGTTGATGTTGATGCTACC | |
| | GGTAAAGTGACATTTAAAAATGTCGGCAGCAAATGGGAGAGGATTACGGCGACGCCAAAAACAGGCGGC | |
| | CCTAGCTATATATACGAAATCCGAGTGAAGAGTTGGTGGGTGAACGCCGGCGATGCTTTCATGATATAC | |
| | AGCCTTGCTGAAAATTTTTGCAGTAGCAATGGCTACACACTTCCCCTTGGAGACCATTTAAACCATAGT | |
| | CGTTCCCGAGGCATCGGGTCACTGTACAGTGAATGGGGAGATATGGGGCATTACACGACTGAAGCTGGT | |
| | TTTCATTCAAATATGTATTGGTCATCGAGTCCCGCAAACTCAAACGAACAATACGTAGTTTCCCTGGCA | |
| | ACAGGTGATCAAAGCGTATTTGAAAAGCTTGGGTTTGCTTATGCGACATGTTATAAAAACCTCTGA | |
| | [SEQ ID NO: 20] | |
| MC019 | MAAAAQGVVNAATQQPVPAQIAIANANTVPYTLGALESAQSVAERFGISVAELRKLNQFRTFARGFDNV | 2620 |
| prot | RQGDELDVPAQVSENNLTPPPGNSSGNLEQQIASTSQQIGSLLAEDMNSEQAANMARGWASSQASGAMT | |
| | DWLSRFGTARITLGVDEDFSLKNSQFDFLHPWYETPDNLFFSQHTLHRTDERTQINNGLGWRHFTPTWM | |
| | SGINFFFDHDLSRYHSRAGIGAEYWRDYLKLSSNGYLRLTNWRSAPELDNDYEARPANGWDVRAEGWLP | |
| | AWPHLGGKLVYEQYYGDEVALFDKDDRQSNPHAITAGLNYTPFPLMTFSAEQRQGKQGENDTRFAVDFT | |
| | WQPGSAMQKQLDPNEVDARRSLAGSRFDLVDRNNNIVLEYRKKELVRLTLTDPVTGKSGEVKSLVSSLQ | |
| | TKYALKGYNVEATALEAAGGKVVTTGKDILVTLPAYRFTSTPETDNTWPIEVTAEDVKGNFSNREQSMV | |
| | VVQAPTLSQKDSSVSLSSQTLSASSHSTATLTFIAHDAAGNPVIGLVLSTRHEGVQDITLSDWKDNGDG | |
| | SYTQILTTGAMSGTLTLMPQLNGVDAAKAPAVVNILSVSSSRTHSSIKIDKDRYLSGNPIEVTVELRDE | |
| | NDKPVKEQKQQLNTAVSIDNVKPGVTTDWKETADGVYKATYTAYTKGSGLTAKLLMQNWNEDLHTAGFI | |
| | IDANPQSAKIATLSASNNGVLANENAANTVSVNVADEGSNPINDHTVTGAVLSGSATSFNNQNTAKTDV | |
| | NGLATFDLKSSKQEDNTVEVTLENGVKQTLIVSFVGDSSTAQVDLQKSKVEVVADGNDSATMTATVRDA | |
| | KGNLLNDVKVTFNVNSAAAKLSQTEVNSHDGIATATLTSLKNGDYTVTASVSSGSQANQQVIFIGDQST | |
| | AALTLSVPSGDITVTNTAPLHMTATLQDKNGNPLKDKEITFSVPNDVASRFSISNSGKGMTDSNGTAIA | |
| | SLTGTLAGTHMITARLANSNVSDTQPMTFVADKDRAVVVLQTSKAEIIGNGVDETTLTATVKDPFDNVV | |
| | KNLSVVFRTSPADTQLSLNARNTNENGIAEVTLKGTVLGVHTAEAILLNGNRDTKIVNIAPDASNAQVT | |
| | LNIPAQQVVTNNSDSVQLTATVKDPSNHPVAGITVNFTMPQDVAANFTLENNGIAITQANGEAHVTLKG | |
| | KKAGTHTVTATLGNNNASDAQPVTFVADKDSAVVVLQTSKAEIIGNGVDETTLTATVKDPFDNAVKDLQ | |
| | VTFSTNPADTQLSQSKSNTNDSGVAEVTFKGTVLGVHTAEATLPNGNNDTKIVNIAPDASNAQVTLNIP | |
| | AQQVVTNNSDSVQLTATVKDPSNHPVAGITVNFTMPQDVAANFTLENNGIAITQANGEAHVTLKGKKAG | |
| | THTVTATLSNNNTSDSQPVTFVADKTSALVVLQISKNEITGNGVDSATLTATVKDQFDNEVNNLPVTFS | |
| | TASSGLTLTPGESNTNESGIAQATLAGVAFGEQTVTASLANNGASDNKTVHFIGDTAAAKIIELTPVPD | |
| | SIIAGTPQNSSGSVITATVVDNNGFPVKGVTVNFTSNAATAEMTNGGQAVTNEQGKATVTYTNTRSSIE | |
| | SGARPDTVEASLENGSSTLSTSINVNADASTAHLTLLQALFDTVSAGDTTNLYIEVKDNYGNGVPQQEV | |
| | TLSVSPSEGVTPSNNAIYTTNHDGNFYASFTATKAGVYQVTATLENGDSMQQTVTYVPNVANAEISLAA | |
| | SKDPVIANNNDLTTLTATVADTEGNAIANSEVTFTLPEDVRANFTLGDGGKVVTDTEGKAKVTLKGTKA | |
| | GAHTVTASMAGGKSEQLVVNFIADTLTAQVNLNVTEDNFIANNVGMTRLQATVTDGNGNPLANEAVTFT | |
| | LPADVSASFTLGQGGSAITDINGKAEVTLSGTKSGTYPVTVSVNNYGVSDTKQVTLIADAGTAKLASLT | |
| | SVYSFVVSTTEGATMTASVTDANGNPVEGIKVNFRGTSVTLSSTSVETDDRGFAEILVTSTEVGLKTVS | |
| | ASLADKPTEVISRLLNAKADINSATITSLEIPEGQVMVAQDVAVKAHVNDQFGNPILNESVTFSAEPPE | |
| | HMTISQNIVSTDTHGIAEVTMTPERNGSYMVKASLANGSSYEKDLVVIDQKLTLSASSPLIGVNSPTGA | |
| | TLTATLTSANGTPVEGQVINFSVTPEGATLSGGKVRTNSSGQAPVVLTSNKVGTYTVTASFHNGVTIQT | |
| | QTIVKVTGNSSTAHVASFIADPSTIAATNSDLSTLKATVEDGSGNLIEGLTVYFALKSGSATLTSLTAV | |
| | TDQNGIATTSVRGAITGSVTVSAVTTAGGMQTVDITLVAGPADASQSVLKNNRSSLKGDFTDSAELHLV | |
| | LHDISGNPIKVSEGLEFVQSGTNAPYVQVSAIDYSKNFSGEYKATVTGGGEGIATLIPVLNGVHQAGLS | |
| | TTIQFTRAEDKIMSGTVLVNGANLPTTTFPSQGFTGAYYQLNNDNFAPGKTAADYEFSSSASWVDVDAT | |
| | GKVTFKNVGSKWERITATPKTGGPSYIYEIRVKSWWVNAGDAFMIYSLAENFCSSNGYTLPLGDHLNHS | |
| | RSRGIGSLYSEWGDMGHYTTEAGFHSNMYWSSSPANSNEQYVVSLATGDQSVFEKLGFAYATCYKNL | |
| | [SEQ ID NO: 44] | |
| MC021 | GTGCCTTACACGCTTGGTGCGCTGGAATCAGCGCAAAGCGTTGCTAATCGCTTCGGTATTTCACTGGAG | 5037 |
| | GAGCTTCGTCGTCTTAATCAGTTCCGTACTTTTGCTCGCGGCTTTGATAACGTGCGCCAGGGTGAAGAA | |
| | CTGGATGTTCCGGCAACAACCTCGCAGAAAAGCCATGAGCAACAAAATGCCGTACCGCCTGCGAATGGC | |
| | GAAAACACGCTGGAGAATCAAATAGCCAGCACCTCGCAGCGAGTTGGCACTCTGCTTTCACAAGATATG | |
| | AATAGTGAGCAGGCCAGCGGCATGGCGCGTGGTTGGGCGTCTTCAGAAGCCTCAGGCGCGATGACTGAT | |
| | TGGTTAAATAACTTTGGTACTGCGAGAATCTCTCTGGGTGTGGATGAAGATTTTAGCCTGAAAAATTCG | |
| | CAATTCGACTTCCTGCATCCGTGGTATGACACACCTGATTATCTGCTCTTCAGCCAGCATACCCTTCAC | |
| | CGAACAGACGATCGTACCCAGATGAACACCGGTTTGGGCTGGCGTCATTTCACCTCCAGCTGGATGTCA | |
| | GGCATCAACCTTTTTTTTGACCACGACCTGAGCCGCTATCACTCCCGCGCAGGGCTTGGCGCAGAATAC | |
| | TGGCGTGATTATCTGAAGTTGAGCAGCAACGCTTATATCGGCCTGACCGGCTGGCGTAGCGCACCAGAA | |
| | TTGGATAACGACTTCGAAGCCCGCCCGGCCAACGGCTGGGATTTACGCGCGGAAGGCTGGTTACCTGCC | |
| | TGGCCACAACTGGGGGGAAAACTGGTCTATGAACAATACTATGGCGATGAAGTGGCGCTGTTTGACAAG | |
| | AATGATCGTCAAAGTAACCCCCATGCTATTACGGCAGGCCTCAACTATACCCCCTTCCCGCTTCTGACT | |
| | CTCAGTGCGGAACAGCGTCAGGGGAAGCAAGGTGAAAATGACACACGTTTTGCCGTTGATCTGACCTGG | |
| | CAACCCAGCAGTTCAATGCAGAAACAGCTTAATCCGGACGAAGTGGCCGGACGGCGCAGTCTGGCCGGT | |
| | AGTCGTTATGACCTGATTGATCGCAACAACAACATCGTTCTGGAATACCGCAAGAAAGAGCTGATTCGC | |
| | CTGAGTCTGCTGGATCCGGTGAAAGGGAAGTCTGGAGAAATAAAACCGCTGGTTTCCTCGCTACAGACC | |
| | AAATATGCCCTTAAAGGCTATAACATCGAAGCCGCTGCGCTGGAAGCTGCCGGAGGTAAAGTCAGCACG | |
| | TCTGGAAAAGATATCACGGTCACGCTGCCAGGTTACCGCTTCACTAACACCCCAGAAACCGATAATACA | |
| | TGGTCGATAGACGTTACCGCCGAGGATGTAAAAGGTAACCTGTCACGGCATGAACAAAGCATGGTAGTT | |
| | ATTCAGGCTCCGACATTAAGCCAGAAAGATTCTCTGTTATCCGTCAATCCGCTAACCGTGGCTGCAGAT | |
| | AAAAAATCGACGACCACATTGACCGTTACTGCGCACGATTCCGACGGAACTCCGGTGCCGGGGCTGGCG | |
| | CTGCAAACCCGCAGTGAAGGCGTTCAGGATATCACCCTGTCTGACTGGACAGATAACGGTGATGGTAGT | |
| | TACACACAGATACTGACCGCCGGAACGACATCAGGTTCAGTAACACTGACGCCGCAAATTAACGGTGAG | |
| | AGTGCGGTAAAAGAATCCATCGTCGTTAATATCGTCCCTGTTGTCTCATCCCGCGACCATTCATCAATA | |
| | ACAATTGATAACGTATCGTATTATGCCGGAGACGACATCAAGGTTAGGGTGGAACTGAAAGACGATAGC | |
| | AATCAACCGGTTGCATATCAAAAAGAGGAATTGGTAAAAGCCGTTACTGTCGAAAACAGCAAACCTGGC | |
| | GCCACGATTGTCTGGCACGAAGAGCAGCCGGGGGTTTATGCCGCGAATTATCCGGCCTATAAGCAAGGG | |
| | ACTGCACTAAGGGCACAACTTAGCCTTCACAACTGGAATGCTCCACTGCAATCGCATATTTATAACATT | |
| | GAGGCAAACCAGAATAAGGCTCGCGTTGCCACATTATCAGCGACAAATAATGACGTTTACGCCGATAAA | |
| | AAGACATTTAATACCCTCACGATCAACGTCACTGATGAGAGTGATAATCCCCTGACAAATCATCAGGTC | |
| | ACCTTTAAGAATGAAAAAGGAAGCGCGGAGTTTGTCGAACCGCCGCAGCAAAATACGGATGCATATGGT | |
| | GTTGCCACAATAAACATGGTAAGTCAGGTTGCGGAAGAAAATACGATTAGCGCCACGCTGCCAAATGGT | |
| | TTTTCACAACGGATAATTGCGAAATTCGTTAGCGATTCGAGTACGCCAAAATTCAAACAACTGGTTGCC | |
| | GATCCAGATACCATTATTGCTGGCAACAGCCAGGGCAGTACTCTGACCGCCATCATCACAGACTTTCAT | |
| | AACAACCCGTTAAAAGATATGAAAGTGAATTTTGTGGCACCTGGTGGCTCGCAACTGGACAACACGACC | |
| | GCCACAACAGACCAGTCCGGTATTGTGCGGGTGCACCTGACCAGTTCAAAAGCTGGTAGCTATTCCGTC | |
| | GATGCCTCGCTTGAGGTGGATAAAAATATTCACCAGTCGGTCACGATCACCGTGGTCCCAAACAGGGAA | |
| | CAATCGGTAATGACCTTGAATGCCGGGTCGGGCAGTGCGATCGCTAACAATACAAATATCGTTACCCTG | |
| | ACTGCCAGTGTGAAAGATGTTTATGGACACCCGTTGCCGGATGAGGATGTGAAATTTACCTTGCCAGCC | |
| | TCCATGACCGGGAACTTCACGCTAAGTAGTGAAACCGCCCGCACCGATGCAAACGGTGATGCCGTGGTC | |
| | ACATTGCGAGGCACAAAAGCGGGTGAGTTTACAGTTACGGCGACGCTGACCAGAAATAATACCGTTGCT | |
| | TATCAGCAAGTCACTTTTATTGGGGATACAAACAGTGCGCAGCTCCAGCCGCTGACTGCCTCATTAAAT | |
| | TCCATTGTTGCGGGTAACAGTACGGGGAGTACCCTGACGGCAACGATCCTGGACGCTTACCAAAATCCG | |
| | CTTAAAGACCAGTTGGTCACTTTCCAGAGTAACGATGTCACTCTAAGCGAAACAGAAGTCACCACCAAT | |
| | ACGCTGGGTCAGGCGACGGTAACAATGACCAGCAATATTGCCGGACAACATAACGTCGTGGTGAGCCGG | |
| | AAAGCGCAAGCTTCCGATAATAAAACGTTTAGTTTATCAGTGCTACCGGATGAAAGTTCGGCGAAGGTA | |
| | ATAAGTATAACCGGAGCCGAAAAAACGATAACGGTGGGCGAAAACATCACGCTACGGATACTCGTCCAG | |
| | GACGCGTTTAACAATGTAATCGCGGGTCAACGCGTCAGATTAAGTGCGCAGCCAACAACTAACATTACG | |
| | ATAGGCGATACGGCTTACACCGATAATAACGGTTATGCGTACGTTAACCTTCTCAGCACCCAACCTGGG | |
| | GTTTATCAGGTGACGGCAACGCTGGACAATAACAGTAGTAGTAAGGTTGACGTGAATGTGGCAAATGGC | |
| | AAACTCGAGTTAACATCATCGAAACCAGAAACTACGGTCCATAATAGTGAGGGTATTACGGTGACCGCA | |
| | ACGGCGAGAAATGCGCGGGGTGAATTGATGCCAGGGCAAATTATCACCTTTAGCGTAACGCCTGAAGGT | |
| | GCAACGCTAAGCAATACAGGGGAAGTCCTTACTGACCAGTCAGGTCAGGCCAAAGTGACGCTGACCAGT | |
| | GACAAAGTGAATGTCTATACCGTTACGGCCATAATGGGCAAAGATGTTCCCGTTCAGAGCCAGGTAACG | |
| | GTTGCGGTTAAGGCAGATGCTAAAACGGCACATGTTGTGAGCGTCGTGGCTTCTCCTGACACCATCACC | |
| | GCCGACGGCATCGATAGCAGCACCATCACTTGACGAGTAGAAGATGATTACGGATTCCCGGTTGAAGGT | |
| | GTCGATATTAGTCATGGCTTAGACACCAAAGGCAGCCCGGTAGTTAATATTCCAACTACGCGTACCGAT | |
| | CAGTCCGGGCAAGTCACGGCGACAATAACCAGTACATTGGCAGAAACCTTAACAGTCAATGTGCAAGTT | |
| | CCTGGCACAGCCAACCAATCCGCAACCATTACATTGGTTGCCGGCACGGCCGATGAAAGTAAGTCAATT | |
| | TTGAAATCCGATGTTGACACTCTGAAGGCTGACTACCAGCAGAGCGGAAAACTTACGCTAACATTGCAA | |
| | GACAAGTACGGTAACCCGATAGTGACGTCTGATCATCTGGAATTTGTCCAGTCAGGCCCCTTCGTGAAC | |
| | TTTCTCAAGTTGAGCGATATTGATTACAGCCAAAGAAATTATGGCGAGTACACCGTGACTGTCACTGGC | |
| | GGAAAAGAGGGAACAGCGACACTCATTCCCATGCTGAACGGGGTTCATCAGGCAAACTTAAGCATATCG | |
| | CTGAATCTCATCCAATCGATAAAAGAAATGTCCGGTCATGTCACTGCAAACAACCATACCTTCTCCACG | |
| | GCTAAATTCCCGAGCGAAGGCTTTGCAGGAGCGTATTACACACTCAACAATGATAACTTTGAAGCGGGT | |
| | AAAACCGTTGATGATTATATGTTTTCAAGTTCACAGGGTTGGGTGTCTGTCGATGCTTCGGGTAAAGTT | |
| | TCTTTCGCAAATATCGGCGATCAAACGTCAGTCACAATAAGCGCTGTTCCCCGACAAGGAGGTACAACC | |
| | TACCAGACCTTAATTAAGCTGAAAGGCTGGTGGGTGAATAATGGAAATCATACCAATATCTGGCTAGCT | |
| | GCCAATGCGCTCTGTCATGCTAAAAATGATGGATATAATCTTCCTGGCATCACACATTTGACGTCTGGC | |
| | GAAAACAAACGCACGCAGGGATCACTGTATGGTGAATGGGGGAACGTTGGAGCGTTTTCCAGTAATTCG | |
| | GAATTTACACCGGGAGCTTACTGGACAAGTGAATCTGATGATTACAGTCGGCACTACTATGTGCAGATG | |
| | CTAACCGGTATGACCGGAAGCGACGCTGATTCCAGCCCCCAACTGACCGCCTGCCGTAAATCACTTTAA | |
| | [SEQ ID NO: 21] | |
| MC021 | MPYTLGALESAQSVANRFGISLEELRRLNQFRTFARGFDNVRQGEELDVPATTSQKSHEQQNAVPPANG | 1678 |
| prot | ENTLENQIASTSQRVGTLLSQDMNSEQASGMARGWASSEASGAMTDWLNNFGTARISLGVDEDFSLKNS | |
| | QFDFLHPWYDTPDYLLFSQHTLHRTDDRTQINTGLGWRHFTSSWMSGINLFFDHDLSRYHSRAGLGAEY | |
| | WRDYLKLSSNAYIGLTGWRSAPELDNDFEARPANGWDLRAEFWLPAWPQLGGKLVYEQYYGDEVALFDK | |
| | NDRQSNPHAITAGLNYTPFPLLTLSAWQRQGKQGENDTRFAVDLTWQPSSSMQKQLNPDEVAGRRSLAG | |
| | SRYDLIDRNNNIVLEYRKKELIRLSLLDPVKGKSGEIKPLVSSLQTKYALKGYNIEAAALEAAGGKVST | |
| | SGKDITVTLPGYRFTNTPETDNTWSIDVTAEDVKGNLSRHEQSMVVIQAPTLSQKDSLLSVNPLTVAAD | |
| | KKSTTTLTVTAHDSDGTPVPGLALQTRSEGVQDITLSDWTDNGDGSYTQILTAGTTSGSVTLTPQINGE | |
| | SAVKESIVVNIVPVVSSRDHSSITIDNVSYYAGDDIKVRVELKDDSNQPVAYQKEELVKAVTVENSKPG | |
| | ATIVWHEEQPGVYAANYPAYKQGTALRQALSLHNWNAPLQSHIYNIEANQNKARVATLSATNNDVYADK | |
| | KTFNTLTINVTDESDNPLTNHQVTFKNEKGSAEFVEPPQQNTDAYGVATINMVSQVAEENTISATLPNG | |
| | FSQRIIAKFVSDSSTPKFKQLVADPDTIIAGNSQGSTLTAIITDFHNNPLKDMKVNFVAPGGSQLDNTT | |
| | ATTDQSGIVRVHLTSSKAGSYSVDASLEVDKNIHQSVTITVVPNREQSVMTLNAGSGSAIANNTNIVTL | |
| | TASVKDVYGHPLPDEDVKFTLPASMTGNFTLSSETARTDANGDAVVTLRGTKAGEFTVTATLTRNNTVA | |
| | YQQVTFIGDTNSAQLQPLTASLNSIVAGNSTGSTLTATILDAYQNPLKDQLVTFQSNDVTLSETEVTTN | |
| | TLGQATVTMTSNIAGQHNVVVSRKAQASDNKTFSLSVLPDESSAKVISITGAEKTITVGENITLRILVQ | |
| | DAFNNVIAGQRVRLSAQPTTNITIGDTAYTDNNGYAYVNLLSTQPGVYQVTATLDNNSSSKVDVNVANG | |
| | KLELTSSKPETTVHNSEGITLTATARNARGELMPGQIITFSVTPEGATLSNTGEVLTDQSGQAKVTLTS | |
| | DKVNVYTVTAIMGKDVPVQSQVTVAVKADAKTAHVVSVVASPDTITADGIDSSTITSRVEDDYGFPVEG | |
| | VDISHGLDTKGSPVVNIPTTRTDQSGQVTATITSTLAETLTVNVQVPGTANQSATITLVAGTADESKSI | |
| | LKSDVDTLKADYQQSAKLTLTLQDKYGNPIVTSDHLEFVQSGPFVNFLKLSDIDYSQRNYGEYTVTVTG | |
| | GKEGTATLIPMLNGVHQANLSISLNLIQSIKEMSGHVTANNHTFSTAKFPSEGFAGAYYTLNNDNFEAG | |
| | KTVDDYMFSSSQGWVSVDASGKVSFANIGDQTSVTISAVPRQGGTTYQTLIKLKGWWVNNGNHTNIWLA | |
| | ANALCHAKNDGYNLPGITHLTSGENKRTQGSLYGEWGNVGAFSSNSQFTPGAYWTSESDDYSRHYYVQM | |
| | LTGMTGSDADSSPQLTACRKSL | |
| | [SEQ ID NO: 45] | |
| MC022 | ATGGTCGCTAAATTAAAACCAGATGTTTTTGTTAATACTAATCCTTTTTTAGAAGCAATGTATAAGGAG | 2409 |
| | AGGCTTAGTCATAAAGGCTATTCTGATAAAATTGCTTTAAGTATTAATGATAAGAAATATAATGTAAAC | |
| | TCTAAGGATATTGAAAATATTCTTGATGGCAAAGGGGATTTATTTAAAAAACGTACACTGTGGGAGTTT | |
| | GTTCGCGATCTTTTTCCTGGCTCTCATATTAAAGAAGTCAAAGGCTTAATTTACGAATTTGTCACGAAA | |
| | GTTGATAATAAAGCCGAGGTGTTCGATAAGATTAAATCTCTGGCAAAAAAAGAACAACAATGGCGATTT | |
| | AGTACTAAGACAGACTTTACCACAAATGAAAATAATGAAGTTATTGTGAGTAGATCCTTTAATCTCTAC | |
| | ACGGGGGCTACTTCGAATGATAACGAAAAAAAACAGGTTTCTTCAGAGAGATTAACGCTTGATAATTAT | |
| | CTCGATGATTTACATTTTGATAATTCTCCCTTGGTGAGATTAACGTTTGATAATTATTCTGTAAAACTT | |
| | GCGACGCTGATTAAAAATAAAATTCCTATTATTAATACAACAATCAATTTATCATCGTTGTCTAAGGAT | |
| | GTTTTAAATTCATTAAAATATTGTAGTTTTAAGAATGTAATTTTTTCAGGAGTGATAAACAGCCCTAAC | |
| | CTTGAAGGTCCGGTTTTTGAAAATTGCTATTTCGATGATTGTAAATTCAACAACATACAGCTTTATGAG | |
| | ACTAATGATGATACTGTGGAATCAGGAAATAAAAAACCAATAAAAGGTATGTTTAAAGGTTGTTTTATT | |
| | TCTAAATGCAAAATTGAAAATTATAGATGTGAGACGTCTAAAATCTACACCGTTACACAACCTGATAGT | |
| | ATTCAAGAAAAATTAGGTTCTTATCTCTTTATGCAGTCTTTCGTTCAGGATTGTATAATCCAGGGAGGA | |
| | TGTTGTCCTGGCTCGAGTATACTTCTGAGTCATTTTTATAACTGTAATATAATAGGGCTTGATGCTCAT | |
| | GGAATGGATTTTTTGGCAAATTCGTTCAACAAGTCTAATTATGATGAAATCAGGGGTCCGGATACGGGG | |
| | ACTGTGTTTTATAATTGTAATTTGAAGCATATTAAAATAAACGATGGTCTTCGTGAAGATGGTTCAGAT | |
| | AGAACTATGTTTAACCCCAAAGAGTATTTTTCTGATTACATTGAAAGAAGATCGAGAAATGTTAAGTTA | |
| | GATGATAATTTTTCGAAATGCGTAGAGAGTATAAAGAAAGGGACCGATTCTTCATTTAGCAAGAAAAAT | |
| | ACAGAGAACAATAATGTCTTCTTTAAAAATAGTAATTTGATTGGGGCAAATTTAGGCTATTATTACAGT | |
| | GGAGATAGGTGCAAAGACTGTGCTATTGATCCAAATACAAATTATTCAAAAGATGGTCTTACAGCGCAA | |
| | AAATACATGTATATGGATCTGGACGGAGCTATAAGTAAAGAACAAATACCTGATTTCTTAAAAAAGGTA | |
| | AGTGCTATCAATATCGATGTAATAAATTTTTATAATTCCGAGTCAAAGACTAAGGAAAAATATAAAAAC | |
| | GCATTTTTAGAGTTAGAGTCGTTTTTATCAACACTTTATGTTGAAAATAAATCGTACGAGGAAAAATAT | |
| | CATTTTGATAATAGTAGGGTTGAGTTTTTTATTTTTAAAGATATGCAGGAAAATGCACAAAATATCATT | |
| | AACAATATGATAGAAAGTGACCGGATTAAGTTTGTTGAATCTATAATAAATAAAATGATTCCGCCGCCA | |
| | GATGGAACAATACTTACTGAAAATCCAAAAGAATATGTCCAAAAGCAAATAAAGGAGTCTCATAAAGAA | |
| | TCTGCTACAAATGTAACATTTGATTATAAGGAACTTGCACCCATTTTTGAAGGTGTTGAAGAAAAACAA | |
| | ATTAAAGCACTATCAAACCAACTGGAACATATAAAATCGTTTAAGACAGATTACGACTCAAGGCTCAAT | |
| | AAGTATGCTCGCGATTTTTACTATTTTTCCAGTGCATTTGCAATTAACTGGGAGGATTTACTAAAAAAC | |
| | TATCAGGAAATAAGAGCTTCCATTAAAGATTACGATGACTTATTGCGAGAAATTAAAGAGTTAATCATA | |
| | AGTCGTAATAAATCATTAGAGGATAAAAGGACTTTATTTGATAACAAAAGGGACAATTGGAATAGTATA | |
| | GAAGTGCAGGATGAAGTAAATGCATTGAATGCGAAAATTGTCGATTGTGATGATAAAATACGTTCTAAG | |
| | CTTACTATTGTTCGAAATAATAGACTTGAGAATCAATATAAAGACGATAAAAACATCAGTGACGCGATG | |
| | CGTAATATTCTGGATTGGTTCGAACGGTATCCCGATATTGTACAAAATATAACCCAGGCTTAA | |
| | [SEQ ID NO: 22] | |
| MC022 | MVAKLKPDVFVNTNPFLEAMYKERLSHKGYSDKIALSINDKKYNVNSKDIENILDGKGDLFKKRTLWEF | 802 |
| prot | VRDLFPGSHIKEVKGLIYEFVTKVDNKAEVFDKIKSLAKKEQQWRFSTKTDFTTNENNEVIVSRSFNLY | |
| | TGATSNDNEKKQVSSERLTLDNYLDDLHFDNSPLVRLTFDNYSVKLATLIKNKIPIINTTINLSSLSKD | |
| | VLNSLKYCSFKNVIFSGVINSPNLEGPVFENCYFDDCKFNNIQLYETNDDTVESGNKKPIKGMFKGCFI | |
| | SKCKIENYRCETSKIYTVTQPDSIQEKLGSYLFMQSFVQDCIIQGGCCPGSSILLSHFYNCNIIGLDAH | |
| | GMDFLANSFNKSNYDEIRGPDTGTVFYNCNLKHIKINDGLREDGSDRTMFNPKEYFSDYIERRSRNLKL | |
| | DDNFSKCVESIKKGTDSSFSKKNTENNNVFFKNSNLIGANLGYYYSGDRCKDCAIDPNTNYSKDGLTAQ | |
| | KYMYMDLDGAISKEQIPDFLKKVSAINIDVINFYNSESKTKEKYKNAFLELESFLSTLYVENKSYEEKY | |
| | HFDNSRVEFFIFKDMQENAQNIINNMIESDRIKFVESIINKMIPPPDGTILTENPKEYVQKQIKESHKE | |
| | SATNVTFDYKELAPIFEGVEEKQIKALSNQLEHIKSFKTDYDSRLNKYARDFYYFSSAFAINWEDLLKN | |
| | YQEIRASIKDYDDLLREIKELIISRNKSLEDKRTLFDNKRDNWNSIEVQDEVNALNAKIVDCDDKIRSK | |
| | LTIVRNNRLENQYKDDKNISDAMRNILDWFERYPDIVQNITQA | |
| | [SEQ ID NO: 46] | |
| MC023 | ATGTTAAGGACGACACGATGGGTCGCTGCTATTATTTTTTTGTATTCTTTTCCGGGTTATGCTGAAGAA | 2451 |
| | ACTTTTGATACGCACTTTATGATCGGCGGCATGAAAGGAGAAAAAGTTTCCGAATATCATTTCGATAAT | |
| | AAACAACCGCTTCCAGGAAACTACGAACTTGATTTTTATGTAAATAACCAGTGGCGTGGAAAACAAGAT | |
| | ATCACGATCCCAGAGTCACCAGTCAAACCATGTCTGCCTAAGGTGCTACTAACCAAACTGGGGGTAAAG | |
| | ACCGGTAACCTCAATACAGAAGATAATTGTATTTTACTGGATAAAGCGGTTGATGGTGGACAATATCAG | |
| | TGGGATATCAGTGAACACCGACTAAATTTAACAGTACCACAGGCGTATATCAATGAACTTGAAAGAGGT | |
| | TACGTTCCCCCGGAAAGTTGGGATCGCGGCATTGATGCTTTTTATACCTCCTATAATTTAAGTCAGTAT | |
| | CGCTCTTATGACAGCAATAATAACAGTAATACAGCCAGTTATGGCCGATTTAACAGTGGATTAAATTTA | |
| | TTTAGTTGGCAGTTACACTCTGATGCCAGCTATAGCAAACCCGATGATATGAAAGGCACATGGCAAAGT | |
| | AATACTCTTTACCTGGAACATGGCTGGTCGCAGATATTAAGCACAGTTCAGATAGGAGAAAATTACACA | |
| | TCCTCACTCATATTCGACTCTCTGAGATTTAGCGGAATACGCTTATTTCGCGATATGCAAATGCTACCT | |
| | GATTCTATGCAGAGCTTTACACCTTTAGTACAAGGTGTTGCGCAAAGTAATGCCTTGATAACGGTTTCG | |
| | CAAAATGGCTACATCATTTACCAAAAAGAGGTTCCCCCGGGACCATTTACTATCGCAGATTTGCAACTC | |
| | TCGGGCAGCGGCTCTGATCTCGATGTCAGCATAAAGGAAGCAGATGGCAGTGTTCGTTCCTTTTTGGTG | |
| | CCCTACTCTTCCGTGCCTAACATGCTGCAACCTGGTATTTCAAATTTCGATTTTATCGCCGGACGCAGT | |
| | AAGATATATGGTGTAAAGAATCAGGAGGACTTTCTCGAGGCAAATTATATATATGGCCTCAACAATCTT | |
| | TTGACACTTTATGGCGGTACGATTTTATCCGATAACTATAATGCTATAACTCTGGGAAATGGTTGGAAT | |
| | ACACCGCTGGGTGCCATCTCTTTTGACGCTACACGATCGAGTAGTAAGTTAAATAATGATATAACACAC | |
| | GAAGGTACGAGCTATCAGGTCGCCTATAATAAATATCTAGTACAAACGGCCACTCGATTTAGCGTGGCC | |
| | GCCTGGCGTTATGCTTCGCAGGATTACAGGACATTCAGCGACCATCTTTACGAAAATGATAAAATAAAT | |
| | CATCAGAGTGACTATGACGATTTTTATGATATTGGCAGAAAAAATAGCCTTTCTGCCAATATCATGCAA | |
| | CCTTTATCCAATAATCTGGGAAATGTATCATTAAGTGCGCTTTGGCGGAATTACTGGGGGCGAAGTGGA | |
| | AATGCTAAAGATTACCAATTCAGTTATTCCAATAGCTGGCAACGCATCAGTTATACTTTCTCTGCCAGC | |
| | CAATCCTATGATGAAAACGACAAAGAAGAGGAGCGTTTTAATCTGTTTATCTCCATTCCTTTCTACTGG | |
| | GGGGATGATATTGCCAAAACACGTCACCAAATTAACTTATCGAATTCGACCTCATTTTCCAAAGATGGC | |
| | TATTCCTCCAACAATACTGGAATTACTGGCATAGCCGGTGAACATGATCAGTTAAATTATGGTATATAT | |
| | GTTAATCAGCAACAACAAAATAATGATACATCGCTTGGTACGAATTTAAGCTGGAGAACTCCCATCGCC | |
| | ACAATAGATGGCAGCTATAGCCATTCTAAAAACGCCTGGCAAAGTGGTGGAAGTATTAGTAGTGGATTA | |
| | GTTGTCTGGCCCGGTGGTATTAATATCACTAACCAGCTATCCGATACATTTGCAATTCTGGATGCGCCT | |
| | GGGTTAGAAGGCGCGCATATTAATGGACAAAAATACAACCGAACAAACAGCAAAGGCCAGGTTGTTTAC | |
| | GACCTGATGATACCTCATCGTGAAAACCATCTGGTACTTGATACAGCAAACAGTGAAAGTGAAACAGAA | |
| | TTGCAGGGCAATCGTCAAATTATTGCGCCTTACCGTGGAGCAGTTTCTTATGTGCAGTTTACAACTGAC | |
| | CAACGTAAGCCCTGGTATATACAGGCACTGCGTCCCGATGGTTCGCCATTAACTTTTGGCTATGACGTA | |
| | CTGGATCTCCAGGAAAACAATATTGGAGTCGTTGGCCAGGGTAGTCGCCTTTTTATTCGCGTAGATGAA | |
| | ATTCCAACTGGCATAAAAGTTGCTCTCAATGATGAACAGAATTTATTCTGTACTATTACTTTTCAACAC | |
| | GTTATCGATGAAAACAAAACATATATATGCCAGTAA | |
| | [SEQ ID NO: 23] | |
| MC023 | MLRTTRWVAAIFFLYSFPGYAEETFDTHFMIGGMKGEKVSEYHFDNKQPLPGNYELDFYVNNQWRGKQD | 816 |
| prot | ITIPESPVKPCLPKVLLTKLGVKTGNLNTEDNCILLDKAVHGGQYQWDISEHRLNLTVPQAYINELERG | |
| | YVPPESWDRGIDAFYTSYNLSQYRSYDSNNNSNTASYGRFNSGLNLFSWQLHSDASYSKPDDMKGTWQS | |
| | NTLYLEHGWSQILSTVQIGENYTSSLIFDSLRFSGIRLFRDMQMLPDSMQSFTPLVQGVAQSNALITVS | |
| | QNGYIIYQKEVPPGPFTIADLQLSGSGSDLDVSIKEADGSVRSFLVPYSSVPNMLQPGISNFDFIAGRS | |
| | KIYGVKNQEDFLEANYIYGLNNLLTLYGGTILSDNYNAITLGNGWNTPLGAISFDATRSSSKLNNDITH | |
| | EGTSYQVAYNKYLVQTATRFSVAAWRYASQDYRTFSDHLYENDKINHQSDYDDFYDIGRKNSLSANIMQ | |
| | PLSNNLGNVSLSALWRNYWGRSGNAKDYQFSYSNSWQRISYTFSASQSYDENDKEEERFNLFISIPFYW | |
| | GDDIAKTRHQINLSNSTSFSKDGYSSNNTGITGIAGEHDQLNYGIYVNQQQQNNDTSLGTNLSWRTPIA | |
| | TIDGSYSHSKNAWQSGGSISSGLVVWPGGINITNQLSDTFAILDAPGLEGAHINGQKYNRTNSKGQVVY | |
| | DLMIPHRENHLVLDTANSESETELQGNRQIIAPYRGAVSYVQFTTDQRKPWYIQALRPDGSPLTFGYDV | |
| | LDLQENNIGVVGQGSRLFIRVDEIPTGIKVALNDEQNLFCTITFQHVIDENKTYICQ | |
| | [SEQ ID NO: 47] | |
| MC024 | ATGACGGCAGATGAGCGCAACGTGGTTAAAGCGGCAACCGACGATAGTATGGAAGCTGCTTATATGCTA | 2166 |
| | AAAGATAATATCCGCTGGTATTATCATAATGGTGACCTATCATTACCCGCTAACTTCAGCAATAAAAAT | |
| | AAACTGGTGGTGAACGGTAATTTAACGATTTCCGGCGATTATGATGATTATCTGTCAGGTAATGGTCAT | |
| | CTGATTGTATTAGGTAATGTTATTGTCGATAACTTTATTAATCATGATTTCGCGTATGTCAAAGGACAG | |
| | ATGACAGCGAAAGGTTTAGTTTATGCTGATTACAATGATCATAACTTTGAAGTGATGAAGGGAATTAGT | |
| | GCGCGAGGCATTATTGTTTCCGATAAAGCAACGCAATTTGAAGTCATTAAAGCTGAATTTTATATCAAT | |
| | GAAGATGGAAGTGGTGAAGGATATAACTGGGATGAGAATATTCAAAAAGCCTATTCACTGGTGACTGCC | |
| | GATCTTTATGATCACACTGAAATCGAAACCGATAACATTTCCAATGCGTATCCTGACTATGATAGCGTC | |
| | GCAGACAACATCGTTCAGGGTTTACCGCTTTTTCGAGATAAAGCCGCTCCTGAAATTAATGAGAAATTA | |
| | AAATGGATTGAAACGGGGAAACTCGATAATTTCCCAGCGAATAAAATAAAGCATCAAGATCCACTGGTG | |
| | GCACGTTTTCTGACTCATACAGAGAGTTTATCACCCGCCGTTATGCTGCAACTGTTACAGCACCCCGAT | |
| | GACCAGACGCGTGAATCGATGGCGCAGTCCTGGCCCGCTCAACAGATGCATTGGCTTACCGATGAATTA | |
| | ATTAAAGATGAGGCTGTTGCCAGAGGGCTGGTTAAAAACAGTAATATTTCCGCCGATGTGAATAAAAAA | |
| | TTAATGTCTGTACCCGTCGAGAGCGTACAGCTGGAGCAGGCCAGACAGGATAATCTTTCTCCAGATATT | |
| | GTTGCGTCGTTAAGTCATAGCCCCTTTCTCAGTGTACGCAAGACATTACTCAGCCATTACGATTACGCC | |
| | TGGCTGGTTCCGACTGCAGTGGCCGATGAGTTGATAAATAACGAAGACCCGGAGTTACGTGAAAGAATT | |
| | ACCGGAGCAGATTTAACCGCACAACAAGCAGTGATGTTGAGTAAAGATAAATCACTGAAAGTTCGTGAA | |
| | GCATTAGCCCGGACGCTTACTGAATTAAAAATTACCCAACTGTCAGCAACCCTGCGTACAGAGGATATT | |
| | GAACGTATCGCAGAACAAATGTATCTGGATAATAAAGAAAATAAAAATATCGTAAAGGTGTTATTGATA | |
| | GCGTTGCCGGAGATGCGTCAACTGTCTCTGGCAAAAGAAGATGTGCACAATTTGCGCGAAGGTGCGCGT | |
| | TATCTGACATCGAAGGACGTGATTAGTTATCTATTGACTCAGCATGATGTTCCCACCGTCTGGGATGAG | |
| | CTGGCACGCGATAAATTATTGCCGCTGGAATATAAGAAACAGTTATGGCAACGCACATTAAATTTAATG | |
| | ATGAGCAAACGGCAAGAAGATCAAGAGCAAGCCTACGAAGTACAGCTGGCGCTGATTGATAATGGTGTT | |
| | GTTGACGAAGAGATGTTAAATAATGCCATTGATTTACTGGTTGATTTACCCGCTGAATACCGCTATCGA | |
| | ATGCGTAACCAATTATTTGATAATAAAGAATTACCTTCCGGAATTATCAACAAACTCGATCAACAGTAT | |
| | CGCTTCAACTCTGACTGGGCGCTGGCTGTTGTCAGTATGAAAAATAGCACCCGTAGACAAAGTGAAAGA | |
| | GGATTACACCGCTGGAACCATGAGGATAGCGATATTTTTGCGGAACTGGCGACGATAAAAGATAAATCA | |
| | GATGATGAATGGTGGCGTGCATTGCTGGAAAGTCGCAATGATCATTTGCGCCAAACGGCATTACGCAAC | |
| | GCACATACCCCGGCGTCGTTGTTAACGACATTGACTGAGTCTCAGGATCGGTCGCTGGCTATTAATAAT | |
| | CCACAGCTGGCTGCCGATGTGAAAACGGTGTGGCTAAAAGAGGATCCATCATTACTTTTATTTGTCGAT | |
| | AAACCCGATCTTTCGCAGTTACGGGATTTAGTGAAAACCGGGGCAACGCGGAAAATTCGCAATGAAGCG | |
| | CGTCACCGGCTTGAGGAAAAACAATGA | |
| | [SEQ ID NO: 24] | |
| MC024 | MTADERNVVKAATDDSMEAAYMLKDNIRWYYHNGDLSLPANFSNKNKLVVNGNLTISGDYDDYLSGNGH | 721 |
| prot | LIVLGNVIVDNFINHDFAYVKGQMTAKGLVYADYNDHNFEVMKGISARGIIVSDKATQFEVIKAEFYIN | |
| | EDGSGEGYNWDENIQKAYSLVTADLYDHTEIETDNISNAYPDYDSVADNIVQGLPLFRDKAAPEINEKL | |
| | KWIETGKLDNFPANKIKHQDPLVARFLTHTESLSPAVMLQLLQHPDDQTRESMAQSWPAQQMHWLTDEL | |
| | IKDEAVARGLVKNSNISADVNKKLMSVPVESVQLEQARQDNLSPDIVASLSHSPFLSVRKTLLSHYDYA | |
| | WLVPTAVADELINNEDPELRERITGADLTAQQAVMLSKDKSLKVREALARTLTELKITQLSATLRTEDI | |
| | ERIAEQMYLDNKENKNIVKVLLIALPEMRQLSLAKEDVHNLREGARYLTSKDVISYLLTQHDVPTVWDE | |
| | LARDKLLPLEYKKQLWQRTLNLMMSKRQEDQEQAYEVQLALIDNGVVDEEMLNNAIDLLVDLPAEYRYR | |
| | MRNQLFDNKELPSGIINKLDQQYRFNSDWALAVVSMKNSTRRQSERGLHRWNHEDSDIFAELATIKDKS | |
| | DDEWWRALLQSRNDHLRQTALRNAHTPASLLTTLTESQDRSLAINNPQLAADVKTVWLKEDPSLLLFVD | |
| | KPDLSQLRDLVKTGATRKIRNEARHRLEEKQ | |
| | [SEQ ID NO: 48] | |

1. An isolated polypeptide comprising or consisting of:
(a) an amino acid sequence selected from the group consisting of SEQ ID NOs: 25, 26, 27, 28, 29, 30, 31, 32, 33, 34, 35, 36, 37, 38, 39, 40, 41, 42, 43, 44, 45, 46, 47 or 48; or
(b) a variant and/or fragment of (a), for example:
(i) a variant of (a);
(ii) a fragment of (a);
(iii) a variant of a fragment of (a).

2. The isolated polypeptide according to claim 1, wherein (a) is selected from the group consisting of SEQ ID NOs: 25, 26 and 27.

3. The isolated polypeptide according to any preceding claim, wherein (b) exhibits at least 60% sequence identity to an amino acid sequence listed in (a), for example, at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity to an amino acid sequence listed in (a);
(iii) wherein the at least 60% sequence identity is exhibited over at least 60% of the amino acid sequence listed in (a), for example, a contiguous amino acid sequence spanning at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the amino acid sequence listed in (a); or
(iv) wherein the at least 60% sequence identity is exhibited over at least 10 contiguous amino acids of the amino acid sequence listed in (a), for example, at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 350, 400, 450, 500, 550, 600, 650, 700, 750, 800, 850, 900, 950, 1000, 1500, 2000, 2500, 2600, 2601, 2602, 2603, 2604, 2605, 2606, 2607, 2608, 2609, 2610, 2611, 2612, 2613, 2614, 2615, 2616, 2617, 2618 or 2619 contiguous amino acids of the amino acid sequence listed in (a).

4. An isolated nucleic acid molecule comprising or consisting of:
(C) a nucleic acid sequence selected from the group consisting of selected from the group consisting of SEQ ID NO: 1, 2, 3, 4, 5, 6, 7, 8, 9, 10, 11, 12, 13, 14, 15, 16, 17, 18, 19, 20, 21, 22, 23 or 24; or
(D) a fragment, variant and/or fusion of (A).

5. A vector comprising a nucleic acid molecule defined in claim 4.

6. A host cell comprising a nucleic acid molecule defined in claim 4 or a vector as defined in claim 5.

7. A method for producing a polypeptide according to any one of claims 1-3:
comprising or consisting of the steps of culturing a population of host cells according to claim 6 under conditions in which the peptide is expressed, and isolating the peptide therefrom.

8. A vesicle comprising one or more polypeptide defined in claim 1.

9. The vesicle according to claim 8, wherein the vesicle is derived from the membrane of a cell, for example, a Gram positive bacteria membrane vesicle or a Gram negative bacteria outer membrane vesicle (OMV).

10. A binding moiety capable of specifically binding to one or more polypeptide defined in claim 1.

11. A pharmaceutical composition comprising a polypeptide defined in claim 1, a nucleic acid molecule defined in claim 4, a vector as defined in claim 5, a host cell as defined in claim 6, a vesicle as defined in claim 8 and/or a binding moiety as defined in claim 10.

12. A kit comprising or consisting of a polypeptide defined in claim 1, a nucleic acid molecule defined in claim 4, a vector as defined in claim 5, a host cell as defined in claim 6, a vesicle as defined in claim 8, a binding moiety as defined in claim 10 and/or a pharmaceutical composition as defined in claim 11; and (optionally) instructions for use.

13. A polypeptide defined in claim 1, a nucleic acid molecule defined in claim 4, a vector as defined in claim 5, a host cell as defined in claim 6, a vesicle as defined in claim 8, a binding moiety as defined in claim 10 and/or a pharmaceutical composition as defined in claim 11, for use in medicine.

14. A polypeptide defined in claim 1, a nucleic acid molecule defined in claim 4, a vector as defined in claim 5, a host cell as defined in claim 6, a vesicle as defined in claim 8, a binding moiety as defined in claim 10 and/or a pharmaceutical composition as defined in claim 11, for use in preventing or treating bacterial infection and/or symptoms thereof.

15. The use of a nucleic acid molecule defined in claim 4, or a binding moiety as defined in claim 10, for detecting the presence of bacteria.


SEQUENCE LISTING (118 sequences)

SEQ ID NO: 1 - DNA, 960 bp, Escherichia coli
atgaaaaaaagtgtcatcgctggcgtctttattgctctgtcatttaccacgtgttcagctatcgcgaacagccttgcattatcattagcaaatgatgatgcagggaagtttcaaccaatacttaatgatatttatggcaataaacatgaaaacagagatgattactcacaaggcttatttctgggatatagccacgatatctcagactcgagccaattatctctccatattgcgcaagatatttactctccatcaggcagtaataaaagacacaacacagctgtaactggagacagagcttttagtgcatacactcacactggtattgaatggaactcccttgcgaatgactggattcgctatcgattaggtactgacataggtgttgttggccccgacgcaggcggtcagaaagtacaaaataaagctcatgagattattggggcagaaaaatatcatgcatgggatgatcaaatagagaatcgctacggttatactgtaaaagggatgctatccatgacaccaagtatggatattttaggtgctaatgttggattataccctgaagtttctgctgttactggaaacttatttcaatatgtagcatatggcgcaaccattgccattggtaatgataaaaccttcaattcggataatggctttggtctgctggctccccgtggtttaatgcatatgtccgatacaagcggattcaaatacaagatttttgcaggtatggaaagacgagatgtcaatcgcaactatactctcgaaggaaaaacaatacagacgaaacaaacaacagtatcgctaaacaaaactgttgatgaatatcaagttggcgcaacaattgggtatgcacctgtagccttcacactagcatttaataaagtaacatcagaattcaagacaggggatgactattcatttataaatggagcaatcaccttctttttttaa

SEQ ID NO: 2 - DNA, 801 bp, Escherichia coli
atgaattcaaaaaagctttgttgcatatgtgtgttattctcgctgcttgcaggatgtgcctctgaatcttctattgatgaaaagaagaaaaaagctcaagtcacacaaagtaatattaataaaaacactccccagcaactgacagacaaagatttattcggtaatgaaaccactctggccgtatccgaagaggatattcaagcagcgcttgatggagatgagttccgcgttccccttaattctcctgtaatacttgttcaatccggcaaccgcgcaccggaaaccattatgcaggaggagatgcgaaaatattatactgtttccacattctctggtatcccggacaggcaaaagcctctgacttgtaacaaaaacaaagataaaaatgagaacgaggatgttgcccgtgctgagaatatgaactggatgcaggcactgcgttttgtggctgcaaaaggacatcagaaagcgattattgtttaccaggatatgttgcagacaggaaaatatgactctgcgctgaaatcaacagtatggtccgactataaaaatgaaaaactcactgacgctatctccctgcgctacctggtacgtttcacgctggtggatgtggcaacaggtgagtgggctacctggtcgccggtgaattatgaatcgagagtaatttttccgccggctggaataacaaaaacgagcaataaagaactatcaaatgaccatgttacggaagcgcaactgtttaatttgaaacaaaaaacatatgtatcgatggtgaaagacttggtgagtcgattccagtaa

SEQ ID NO: 3 - DNA, 4587 bp, Escherichia coli
atgaacaggaccagtccctattattgtcgtcgctcagtactttccttattgatatctgccttgatatatgccccgcccgggatggctgccttcactcctgatgttattggtgtggtaaacgatgagactgtagatggcagccaacgagtagatgaacgaggtacaacaaataacactcatattatcaaccatggccagcagaatgtttatggcggggtatctaatggaagtcttattgaatctggtggatatcaagatgtaggaaggcataacaattatgtggggcagtctaataataccaccattaacgggggcagacagtcaattcatgacgggggtatttccacaggtacgataatcgagagtggcaatcaggacgtttataaagggggtatcagcaatggaacgacaattaagggcggtgcttcacgcgtagagggagggagtgcgaatggaacactcattgatggtggtagccagatagtaaaagttcaagggcatgctgatggtacaacgataaataagtctggctctcaggacgtagtacaaggaagtctggcaacgaacacaaccataaatggtggtcgacagtatgttgaacagagcacagtagaaacaaccaccatcaaaaatggcggtgagcaaagagtatatgagagccgtgcgctggacacgacgattgaaggcggaactcagtctctgaatagtaagtcaacggcaaaaaatactcagatctattctggtggtacgcaaattattgataacaccagctcctcggatgttattgaagtttattccggtggcgtgcttgatgttagtggtggtacggcaacaaatgttacccagcacgatggtgcaattttaaaaactaacactaacggtacgacggtgagcggtacgaatagtgaaggtgcattctccatccacaatcacgtggcagacaatgtgttgctggaaaacggtggtcatttagacataaacgcatatggttcggcaaacaagacgattattaaagataaaggaacaatgtcagttttaaccaatgctaaagctgatgcgacccgaatagataatggcggggttatggatgttgcaggaaacgcgacaaataccataattaatggtggcacacagaatattaataattatggcatagccacaggcaccaatatcaacagcggaacgcaaaatatcaaaagcggcgggaaagctgacacaacaattatatcctccgggagccggcaggttgttgagaaagatggtacggcaattggcagcaatattagcgccggaggctcgctgattgtctataccggcggtattgcacatggggttaaccaggagacgggcagtgctttagttgccaacacgggtgcagggactgatatcgaaggatacaacaagctctctcacttcactattaccggaggggaggctaattatgttgtgctggaaaataccggcgaactgacggtagtggctaaaacctcggcgaaaaatactaccattgatgctggcggtaagctgattgtccagaaggaggctaaaacagatagcaccagacttaataatggcggcgttctggaggttcaggacggtggtgaggctaagcatgttgagcaacaatccggcggcgcattaattgcttccacgacctccggaacacttatcgaaggaaccaacagttatggtgatgctttctacatcaggaattcagaagctaaaaatgtagtgctggaaaacgctggctcattaacagtcgtcactggttcccgggcagttgacacgattattaatgccaacggcaaaatggatgtttatggaaaagatgttggcactgtactcaatagtgctggcacccaaacaatatatgccagtgccacttctgataaagcaaatatcaaaggtggcaagcaaacggtatatggtttagccactgaagcaaatatcgaaagtggtgaacaaattgttgatggtgggtcaacagagaaaacacacatcaatggtggcacgcaaaccgttcagaattatggtaaggcgatcaataccgatatcgtctctggcctacaacaaattatggcaaacgggacagcggaaggttccattattaatggcggttcacagatagttaatgagggcggtctggctgaaaactcggtgcttaatgatggcggcacactcgatgtgcgggagaaaggcagcgcaacggggatacagcagagtagccagggcgcgttggttgcaaccaccagggcgacgcgggtcacaggaacacgcgcggatggcgtcgcgttcagcatcgagcagggtgcggcgaacaatatcctgctggcaaatggcggagtgttaaccgtggagtcagacacctcttctgacaaaacacaggtcaatacgggcggacgggagatcgtcaaaacaaaagccactgcgacaggcacgacgctcaccggcggtgaacaaattgtcgagggtgtggcgaatgagacaacaattaacgacggcggaatacaaacagtttcagctaacggagaggcaataaaaacaacgatcaatgaaggcggtacgctgacagtcaacgataatggcaaagcgacagatatcgtccagaacagcggtgccgctctccagacgagcacggctaacggtattgaaatcagcggtactcaccagtacggcactttttccatttccggcaatttagcgaccaatatgttgctggaaaatggcggtaatttattggtattagcaggtaccgaagctcgcgactccacggttggcaaggggggggcaatgcaaaaccagggtcaggactccgccacaaaggttaactctggtgggcaatatacccttgggcggtcaaaagatgagtttcaggctctggcccgggcagaagatctccaggttgctggcgggacagcaatcgtctacgcaggtacgctggcggatgcatcggtcagtggcgcgacaggaagcctgtcgttaatgacgccacgggataatgttacgccagttaaactcgaaggggcgatccggattaccgatagcgcgacattaactatcggcaatggcgttgatacgacgcttgccgacctgacggctgccagccggggcagtgtctggcttaacagcaataattcctgtgcaggcaccagcaactgcgagtatagagtaaacagtttgctacttaacgacggtaatgtttatttatcagcacaaacagcagcgcctgccacaactaacggtatatacaatacgctgacaaccaatgaactttccggtagcggtaatttctacctgcataccaacgttgcaggctctcggggcgatcaactggtcgtcaacaacaacgccactggtaattttaaaatctttgttcaggataccggcgtcagtcctcagtctgacgacgcgatgacgctggtgaaaacagggggaggggatgcttcgttttcgctgggcaatactggcggtttcgttgatcttgggacctatgagtatgtcctgaaaagcgatggcaacagcaactggaacctgaccaatgatgtcaaacccaacccggatcccaacccaaatcccaacccaaatccgaagccggatccaaaaccagacccaaaaccggatccgaaaccagacccgactcccgagccaacgccgacacccgttccggagaaacgcatcacgccttctaccgcagccgtactcaatatggcagcaacattaccgttggtatttgatgctgagctaaacagtattcgcgagcggttgaacataatgaaagcgagtccacacaacaataatgtctggggggcgacgtataacacccgtaataatgtcaccaccgatgcgggggccgggtttgagcagacgctgaccggaatgacagtggggatcgacagccctaatgatattcctgaggggattgcgacgctgggcgcttttatgggttattcccattcacatatcggttttgatcgcggaggacatggcagtgtgggcagttattctctgggcggctatgccagttgggaacatgaaagtggtttctatctggacggtgtcgtgaagctgaaccgttttgaaagtaacgtagccggtaaaatgagcagcggtggagccgccaatggcagttaccacagcaacgggctgggcggtcacattgaaaccgggatgcgatttaccgatggtaactggaacctgacgccgtatgcatcgttaacggggttcaccgctgataaccccgaatatcatttatccaatggcatggaatcgaaatcagtcgatacccgcagtatatatcgtgaactgggcgcaacgctgagttacaacatgcgtctggggaacggtatggaaattgagccgtggctgaaggcggctgtgcgcaaagaatttgtcgatgataaccgggtgaaggtgaataatgacggtaatttcgtcaatgatttgtcgggcagacgtggaatataccaggcaggtattaaagcctcattcagcagtacgttaagcgggcatcttggggtggggtatagccatggtgccggtgtggaatccccgtggaacgcggtagctggtgtgaactggtcgttctga

SEQ ID NO: 4 - DNA, 747 bp, Escherichia coli
atggcaggttttttaatattcctgtcttctgctgcttatgctgatatcaatctgtatggtcctggtggcccgcatacagccttgcttgatgcagccaaactttatgccgaaaaaacaggtattatagtgaacgttcattacggcccacagaacaaatggaatgaagatgccaaaaaaaatgcagatatcttgtttggcgcatcagaacaatctgctctggctatcattcgggaccataaagacagcttcagtgaaaaagatattcagcctctttatctgcgaaaaagtattttactggtaaagaaaggtaatcctaaaaatatccggagtattgacgacctgaccagacctgggattggcgtaattgttaatgatggtggtggtaccagtaatacatcaggcactggcgtctgggaagatattgccggacgtaaagggaatatagaaactgtcgccgcaatccgaaaaaatattattttatatgcgcccaatagcggaactgcacgtaaggctcttgagaatcagcctggagcagatgtctggataacctgggctgactgggcagccagtaatccagaaattggtgatgtcgtggaaatagcgccagactacgtgatatggcgtgatatgaacattacagtacgtcaggatgcaaatgatgaaacccgtcgatttgcagaatggctacaaaccgatgaagcggcgcctgcattcaaaaaatatggctggaccaggaaaggcacttga

SEQ ID NO: 5 - DNA, 1101 bp, Escherichia coli
atgaaattaaaaatagttgcggtggttgtaactggtttgttagctgcgaacgtagcacacgctgccgaagtctataacaaggatggtaataaactcgacctttatggcaaggttaccgctctacgttattttactgatgataagcgtgacgatggtgataaaacttatgcccgtctcggctttaaaggagaaacgcaaatcaatgatcaaatgattggttttggtcactgggaatatgattttaaaggctataacgatgaagccaacggctcgcgcgacaacaagacccgtctggcctatgctggtttaaaaattagtgaatttggctctctggactatggccgtaactacggtgtcggctatgacattggttcatggactgatatgttgccagaatttggtggcgatacctggagtcagaaagatgtcttcatgacataccgtaccaccggtgtagcaacctatcgcaactacgatttctttggcttaattgaagggctgaactttgccgcgcaatatcaaggcaaaaatgaacgtactgacaacagtcatctttatggtgctgactacacgcgtgccaacggtgacggtttcggtatctcctcaacttatgtttatgatggctttggtatcggagcggtgtataccaaatccgatcggacaaatgcgcaggaaagagccgctgctaatcctctcaatgcctccggtaagaatgcagaactgtgggctacaggtataaaatatgatgccaacaacatctactttgcagctaattacgctgaaacattaaacatgaccacctatggcgatggttatatttctaacaaagcacaaagttttgaagtggtggcgcaatatcaattcgacttcggcttgcgcccctcactcgcttacctgaaatcgaaaggcatagatctgggccgctacggcgatcaggacatgattgagtatatcgacgttggtgcgacgtatttcttcaacaaaaatatgtcgacctatgttgattataaaatcaacctgattgatgaaagcgactttacccgtgccgtagatattcgcaccgataacatcgtcgctacgggcattacctatcagttctaa

SEQ ID NO: 6 - DNA, 960 bp, Escherichia coli
atgaagtgtatattatttaaatgggtactgtgcctgttactgggtttttcttcggtatcctattcccgggagtttacgatagacttttcgacccaacaaagttatgtctcttcgttaaatagtatacggacagagatatcgacccctcttgaacatatatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggccgggttcgttaatacggcaacaaatactttctaccgtttttcagattttacacatatatcagtgcccggtgtgacaacggtttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgtgtatacgatgacgccgggagacgtggacctcactctgaactgggggcgaatcagcaatgtgcttccggagtatcggggagaggatggtgtcagagtggggagaatatcctttaataatatatcagcgatactggggactgtggccgttatactgaattgccatcatcagggggcgcgttctgttcgcgccgtgaatgaagagagtcaaccagaatgtcagataactggcgacaggcctgttataaaaataaacaatacattatgggaaagtaatacagctgcagcgtttctgaacagaaagtcacagtttttatatacaacgggtaaataa

SEQ ID NO: 7 - DNA, 948 bp, Escherichia coli
atgaaaataattatttttagagtgctaacttttttctttgttatcttttcagttaatgtggttgcgaaggaatttaccttagacttctcgactgcaaagacgtatgtagattcgctgaatgtcattcgctctgcaataggtactccattacagactatttcatcaggaggtacgtctttactgatgattgatagtggcacaggggataatttgtttgcagttgatgtcagagggatagatccagaggaagggcggtttaataatctacggcttattgttgaacgaaataatttatatgtgacaggatttgttaacaggacaaataatgttttttatcgctttgctgatttttcacatgttacctttccaggtacaacagcggttacattgtctggtgacagtagctataccacgttacagcgtgttgcagggatcagtcgtacggggatgcagataaatcgccattcgttgactacttcttatctggatttaatgtcgcatagtggaacctcactgacgcagtctgtggcaagagcgatgttacggtttgttactgtgacagctgaagctttacgttttcggcaaatacagaggggatttcgtacaacactggatgatctcagtgggcgttcttatgtaatgactgctgaagatgttgatcttacattgaactggggaaggttgagtagtgtcctgcctgattatcatggacaagactctgttcgtgtaggaagaatttcttttggaagcattaatgcaattctgggaagcgtggcattaatactgaattgtcatcatcatgcatcgcgagttgccagaatggcatctgatgagtttccttctatgtgtccggcagatggaagagtccgtgggattacgcacaataaaatattgtgggattcatccactctgggggcaattctgatgcgcagaactattagcagttga

SEQ ID NO: 8 - DNA, 1110 bp, Escherichia coli
atgcaaaggaaaggcaataaactgttgattcagttatgcagtgtgatactgctattttttaccacatcctggtatgcattggcgaatgaatgttatatagagagaaatgctgaaggggattatcacatgaagataagctctactcagcttagtctggcgtcacaaatggtcgaggttccgacagaaatagccgaagctacatgggatgtaaatattcaactaagaggcgatgccatagggtgtaaatctcttggggatagtaaggcagttcactttcttaatacagctgacccaagtttaatatccacgtacaccacaacgaatggcgcagcgttattaaaaacaactgttccaggcattgtgtattctgtcgagttattatgccttagttgtggtgccgcagatgaacttgatttatggctacctgcacaaagtggcgcagataacttcataccaagcacccagacgaaatgggcctatgagtacagtgatcaaagttggtatttacgttttcgcttattcataactcctgaatttaaacccaagaatggtgtttccagcggaacaacgatagcaggaaagattgcgtcatggtatataggtaccaatgaccagccgtggatcaacttttacattgacaatgactctttaaagtttttcgtcgatgaaccgacctgtgcaacagttgccctggcacaagatcagggcaacgtcagtggcaatcaggtaacgcttgggaacagctatgtttcggaagtgaaaaatgggcttacgcgggaaatccctttttctatccgtgctgaatactgttatgccagtaaaattacggttaagttgaaagcggcaaataaacccagcgatgccacactggtgggtaaaacgactggctcggcttcaggcgtggctgtaaaagtaaattcaacttatgacaatagcaaagtattgttaaaagcagatggtagcaacacggttgactacaacttcgccgcctggtcaaacaacctgctgtttttaccttttacggcgcagctggtaccggatggtagcggtaatgctgtcggtgttggaacattttcaggtaacgcgaccttctcctttacctacgaataa

SEQ ID NO: 9 - DNA, 1062 bp, Escherichia coli
atgagattcgccaaaggcgtgttgctcgccatctgtctgatttttttgccgctaaaagcggcgctggcgctgaactgctattttggtaccgcaaacggcgctgtagaaaaatcggaagctattatgcccttcgccgttccggccaattccaaacccggcgataaaatctgggaatctgacgatataaaaattccggtctactgtgacaacaacacaaacggtaattttgaaagcgagcacgtctacgcctgggtaaacccttaccccggtattcaggatccctattatcaattgggcgtaacctatgaaggcgtggactatgacgcaagccttgggaaaagccgcatcgacacgaatcagtgcatcgacagtaagaatattgatatttacaccccggagcagatcattgcgatgggctggcaaaataaactttgctccggcgatccaaccgttatgcacaaatcgcggacgtttgtggcgcgtatgcggctttacgttaaagttcgcgccatgccgccccatgattatcagagcaaactcagcgattacatcgtcgtacagtttgatggcgcaggcagcgtgaacgaagaccctaccgcgaagaatctgaaatatcacattaccggtctggaaaatatccgcgtgctggattgcagcgtcaactttgctatctcacctgaaacccaggtcgttgattttggccgatttaacgtgctggatattcgtcgccatacgatgtcgcagcaatttaaaataactacaaccaaatcacaaaacgaccagtgtaccgacggttttaaagtcagctcttcgttttataccgatgaaacccttatcgacgaagataaatcactgctcattggcaatggcttgaagcttcgattgttggatgaaaatgcctcaccctatactttcaacaaatacagtgagtatgccgattttaccagcgacctgttagtgtatgaaaaaacctacacggcagaattgtcatccacgccgggtacgcccatcgacgttggtccctttgatacagtggtacttttcaaaattaactacaactga

SEQ ID NO: 10 - DNA, 1086 bp, Escherichia coli
ttgtgggaatgtgatgcaacagatttgccaaacatctattttctggtcgctaccaatggcgacgaccgcgtggggggcttttacgatgcaggcgggcctgatggtctgagtgatgtctatgccacctggttcgcttttgtcggtctcaagcagaccatggcgggcgtgacgcttggtcgttactggaagaaagtgcccatcaccagttatgccactcagggaactaaaatccagattcgcttacaggatatccctcctcttcatgctgagctttatcgcatcagtacgctacctgatacatcagcaacaacaagttggtgcggtaataataatacagatagtagtggagtcggattcgcaaaaccttccggtacaatctataactgtgttcagcccaatgcctatattcagctttccggtaccagcggcattttatttggtcatgatgagcccggcgaagatagttctgttcattgggatttctggggtgctgataatggttttggttacggaatgcgttcggccaatcgactctacaacaatgccacctgcgttgcccgcagcgccacgccgttagtattgctgccgacaattgcagaagcacaactgaatgcgggcatggaaagtaccggtaattttaatgtccgcgtcgagtgtagtaactcggttcaatcagggattagcgatactcagacagcattaggaatccaggtgtctgaaggtgcatatacagcggcgcaaaaactggggattatcaatagcaacggcggcgtcagcgccctggtctctgataattatgacgcagcagagatggcaaagggcgttgggatctacatttctaacagtgctcaccccgatacggcgatgacgctggttggtcaaccgggcatcgcgaagttaacccccggaggaaatgcagcggggtggtatcctgtatttgaaggggcaacattagaaggtgcgactcaccccggatactccagctatagttactcttttatcgcccggttgaagaaactgccaaatcagacagtcagtgcgggaaaagtgcgggcaacggcttatatattggtgaaaatgcaatga

SEQ ID NO: 11 - DNA, 789 bp, Escherichia coli
atgaatatggctttttacggcaagtggtttgcttgcctgtggctcgctacgtcgtgcgttcaggcggcgagcacagataataaggcgctggagataattcgtcgcgctgacgaaattcgttcgccgaataaaccgtttcgttacacgctgacggtaacggaatataaagcgggcgcaacccagccagagaataaacaggtcctcgatatttccatgcgctttatgaaaccgcagggtaatgaaaaagcggatgcacgctcgctggtgcgttttatttatccgccgcgagataaaggaaaaatcatgctttccgactggtacgatttatggttttacacgccggaactgcgacgtcctatgcctatttcacggcaacaaagattaattgggcaaatctctaatggcgatgtcattgtcactaactttgaatatgcctatgactcgaccctgatgggggaagtcacgtgtgctgaaaagcagtgttacaagctggcgctggtgcgtaaatcggcggatatcacctggccgaaggttatctattacgttgaaaaagacggtgataatcgcccgtggaaggcggcctattattcgcaggatgaccagttaataaaagaagtgttgtatcaggatttccagccggtgttggggaaaacgcgacccatgaaaataaccgttaccgatgttcgtcacggcaataactattcagtgatggagtacagcgatgttcgtctggaatcgctgccggagtttcatttcaccaaagagtatattcagcgaggtgcgaaatga

SEQ ID NO: 12 - DNA, 1389 bp, Escherichia coli
atgaaacgtacgctgaagatctcttcgttgctgtgtgttgcgctgccgttgacggtacaggcggattgcctgtcaggtgacgaggtggcacaaaatagcgatgtgacgctttacgagcaggtgtcttatgtcaataaacaggcttcggcctggcagattgcaggcaaaaatccctacacccgtcacaacggttatcaggaggcggggattggcatcaatagcggttgttcgataatcgacaatacgctggaccttaagctcaatctgtatggaataaacgaatatgcgcttaaacccgcagggaaattcgaaacggatgatagccgcacgcgtgcgttaattaaccgcctcagtctggtctatagcgcctcggacagcgttcaatttgaagcaggcaaatttgccgcgccgtcaggaatgttctttttgcgttcgccttctgatttgcaaacgcattattacaccggattccagtcgacgcgtttgcacgatcccaaaatgacatcggcgtatcaggcgtcatcgtggggcgcaaaaatgagcgtggatacacgcgattatgcgttttcggcgagtgtcataccgaagttagccaccatcgacaaacgctatgtgacgtctggcaactggtcggctaatcagcagggaaacagcgacgaagcctatctgctgagcttcagcgatcatcgctttggtgagcacacgccgacagtcaatgtgcggctcggaccatcgccgtcactcgcgctgtctgacagttatcactacaccccgcagcttacgctgaatgtcgacgcagcctaccaccgctcccagcaatggcggcatctttcccaccgtgagacggcgcaggtcgaggaatatcagtttccggattcgctctatgaaacgaaagatgaaagcggcgtagagctggcgctgggtgggcagtacaccagtgataatttcagcgtgtttggcgttgagtattactttcagagcgagggttattcgcgcgctgaacagcgtcagcagcgcgagttaattgacttcctcaataccacaaccggctatgcgccactggatcaggcatttgattcttacaaatacctgatggcttcagaaatcagcaataccgccaatcaggggatgctgcagggtaagcactatctgaacgcctgggccagtctgccgctggcgggagagtcgacgctgcagccttcgctggtggtgaatctcgttgatggcagcacgttgctgggcctgcattattccacgccgctcagcgctataagtaaccagcttgaagcctatgccggtggctatagcgcgctgggaagtcggtactccgaatttgcgctttttggcgacacgttaggcctttatttgggctttaaatactatctataa

SEQ ID NO: 13 - DNA, 705 bp, Escherichia coli
gtggggatcgacagccgtaatgatattcctgaggggattgcgacgctgggcgcttttatgggttattcccattcacatatcggttttgatcgtggaggacatggcagtgtggacagttattctctgggcggctatgccagttgggaacatgaaagtggtttctatctggacggtgtcgtgaagctgaaccgttttgaaagtaacgtagccggtaaaatgagcagcggtggagccgccaatggcagttaccatagcaacgggctgggcggtcacattgaaaccgggatgcgatttaccgatggtaactggaacctgacgccgtatgcctcgttaacggggttcaccgctgataaccccgaatatcatttatccaatggcatggaatcgaaatcagtcgatacccgcagtatatatcgtgaactgggtgcaacgctgagttacaacatgcgtctggggaacggtatggaagttgagccgtggctgaaggcggctgtgcgcaaagaatttgtcgatgataaccgggtgaaagtgaatagtgacggtaatttcgtcaatgatttgtcgggcagacgtggaatataccaggcaggtattaaagcctcattcagcagtacgttaagcgggcatcttggggtggggtatagcaacggtgctggtatggaatccccgtggaacgcggtggctggtgtgaactggtcgttctga

SEQ ID NO: 14 - DNA, 1260 bp, Escherichia coli
atgaaattcccttcaatatttaacaaaataaaaccacaatccatacagcaacatccagaaaaaaatcaacttaactggatgctcgaattaaataaatggaaagaagaacgtatacttacaggtgaaatccatcgtccggaatgtcgaaacgaagccgctaaaaggataaactgtgcttttttgtcgaaacagaatgacattgatttatcaggacttaatttatctactcaaccaccagggctgcaaaacttcacctctatcaatcttgataataaccaactcacacattttgatgcaaccaactacgatagactcgtaaaacttagtctgaatagtaacactcttgagtcaataaatattcatcaaggcagaaatgtaagcattacacatatatctatgaataataattgtctcagaaatattgatatagataggctttcatcaattacttattttagtgcggcacataataaactagagtttgtgcaattagaatcttgcgaatggctgcaatacctgaatctcagccataatcaattaactgatattgttacaggaaataaagaagaactcttactgctggatctatcccataataaactagcaagtttacacaatgccttatttcccaacttaaatacgttacttatcaacaacaacttgctttctgaaattaaaatgttttatagcaacttctgcaaagttcagacattaaacgctgctaacaatcagttggaaaaaataaaccttcatttcctgacttatctttcatctatcaaaagtttaaggctggacaataataaaataactcgcattgatactgagaacacatccgatattagaagtttattccccataataaagaagagcgaaagcttaaattttttaaatatttctggcgagaacaattgccctactatccagctcatgttatttaatttgttttccccagcacttaagcttaatactggcctggcaattctttcgcctggtgcatttgaagatcactctgacggattagatgtggataacgaattgtttcactatactattaataaagcatataccccatataatatacatacttataaaacagaagaagttgtaaaccagaggaatataaaaattaaaaatatgaccttagatgaaataaacaatacttattgtaataacgattattacaatgaggcaataagagaggaaccgatagactttctggacagatcgttttcctccagctcatggcctttttatcactaa

SEQ ID NO: 15 - DNA, 711 bp, Escherichia coli
gtgaacaaggccccctctttaattgccgctatcgtcctcggactggggattagcgcctgtggttattttgttggcgatggtgttaaacatttaaaaaccaacaaccgttatgtcaatgtgcgcgggctttctgaaaaagaagttcgcgcagatacggcggaattaacaattgcgattaactttaagggtaatgttcccggcgaactgttcccgaaactggaagaagcgcagaaaaaaattgttgccgaactcaatgctcaggggattaacgagaaagagataattctgggccaatggacaagcaagcgaacagattccttttatctgaaagatgatcccacaatgccacgctataacgcagacgggtcagtgacgataaaaacccataatgttgctgctgtcgaaaaagtcgtcgcgaaattaaacgaactgcaagtcgctacggatggcgctatcgctgagagcaaagtcgcctatcgctttaatggtattggcgcactgcgcgctgagatgattgctgccgccaccaaagatgcacgcaacgccgcactacaatttgcaacggactcaggtagccaggtggggtcaattagcgatgcctcacaaggcgtattccagatctttgccagtggcagtgatgaagatgatcctacggcgattaataaaacggtacgcgtagtcacgaccgtaacctatgcgttgcaggattaa

SEQ ID NO: 16 - DNA, 843 bp, Escherichia coli
atgaaaaaatggacgatatttttgacaagtctgatattacttgctctcagtttagaaacaccaaagtgctatgccggtgataaactcatgagtgcaagcttttccagtactaaaatttattatgcgatgaaaaacgttactgcttccggcagtttatatttctatgtcacggtagtgacgccgggagaagtaagctacggacaatacaactccaatgcaagaaaaggcgacaccctcaagttaatatcctggagcggatctggcccggcaccgacactggttttgactgattatagacgtacggacacctcaaattgccctggtatcaatactcgtgtctttgtctgtgcatatatgacttttaacgtcactgtagaatctgataattatggttgtccgtggattgcctcattttattcagtatctgaggcatttggatttggtacgtatacaagcccaacagttcatgattctatttgcccgacaattcccgtcgcttcgtttgatatatcctggagtgataattatgttagccataataaagcgctgcgattgcaatccgatggttcgacaatcacgactacgctttcgacctacctgatggaaagcggcaaactctgcgatggcagtatttttgattccagaggggcatattgtcgtgctgtttctgacttattaacctttacctcgtatggttgtgataatgcaaaggtgacagtcaccccctcacgtcaaccattgacggatagaaagctacacgacattgtggtacaggtgaataccagtagccgtgagccaattgattctacatgccgtttccagtatgtactgaatgaactgtag

SEQ ID NO: 17 - DNA, 912 bp, Escherichia coli
atgggtacagcagctataagtcatttacgatatgatcttaacaaatatgccctttctttacgcaaaaccgcaacacttgcttcaacattttttattgagtccccattagttagattcgagtatctgcaagaaatagaaaatactattaatgatattactcacagattcaattcttcatatgacatcaatgaaaaagcacgacttatcaatgaattaaaaatggagtcagaaacagcaagaaaagaatatcaacttttcagacaaggaaattatgataaatatatcactactgatattttcgaagaacatggtttgataaaatatgtgaacttaagtttcgatattgttgcatctgtaggacaggtcgtaggcggggttggcgcgttgaaatttggtaaggtagtgcattcaaaccgaataaaaggtataggcgtcacattagttgctcatggggcgaataatttttatgaaagcttatcacctttattttttaacgagtatgattcaggtccaatacgtgaactctacagagtaatagcaaaaaaaatgggaggtgatattaattctgctgattatgcatacagcatcgtggacttttcgattactgcatatggtggatattcaggcatgaaaatcgtacctaagtataatagattgattcgtccttcattaggaaatagacctggtacaggacgattatttcattacacatcagttgattttaaaaataaattctcattaaaacctacccctttaaaaatcatacaaattagttctactgtaaaaaaattcaaggttacattttatgatgaaaatacaaatttgaggataaaacatggcctcaataatcatatgccacatcaattattaatgaatatatcaggaactacaactggtgcaggaatacaataa

SEQ ID NO: 18 - DNA, 1065 bp, Escherichia coli
ttgctgtcactgacactgatgtctgctttattatcgcctttatctcttcaggcagcggatgtccggcgtagcggagatgaagcatttatcattcagcagcagcgtcaggaagcccttgagcaacaactgacgccttcagcccctgatgttcgcctttctgcacctggctcttttgcccataagattaattttcctgttgaaacgccctgttttcagattaaacagacggaactgaagggggctgatgcgttaccacactggctgcctttacaaaaaatcgccaacggggcggtcgggcattgcctgggggcgaaaggaattaatctgctgatgagtacattgcagaaccgtctggtcgatcatggttatgtcaccacccgtgttctggcaccttcgcaggatttaaaaagcggtatcctccggctggttattattcccggtgttgtgcgacatgtgcgtctgacaccggacagtgatgactatattcagttgtattcctcattcccggcacacgaaggttctctgctggatttacgggacattgagcaggggctggatttaggtaacagccggatacagggacaacatactgagctgaatgcaaccagtggaaatctgtctacacagaatgcgcaactgagtgccgatacgctttccgcccggactgccgggcagttcagcagtaatggcggtacgataaatgccgacacactgcagatatcggcacaaagcctgtcaaatcgtaaaggcagtctgattcagacgggaacaggggatttttcgctgagtctgccgggaagcgtggataaccgggaagggctgcttgcggcaaatggcgcggtgcgtctggatgcactgagccttgataatcgcaaggggaaagtgcaggcggagcagtcaccctcccttcagaaatccccgcccacgtttctgaaaccgtttgtggctggtgtctgtgcggcattgctggcggtcagcgtggctattccgggatggcagtttctgacacagccatcaccggaggagcagcattttacctgggggaatggttgtaaaaagcagtga

SEQ ID NO: 19 - DNA, 4200 bp, Escherichia coli
atgagcggaaaaccggcggcgcgtcagggcgacatgacgcagtatggcggtagcattgttcagggttcagccggggtgcgcattggtgcccccaccggcgtggcctgttcggtgtgccccggcggagtgacgtccggccatccggtcaatcccctgctcggtgcaaaggtccttcccggtgaaaccgacatcgccctgcccggcccgctgccgttcatcctctcccgcacctacagcagttaccggacaaaaacgcccgcgccggtggggagcctcggccccggctggaaaatgcctgcggatatccgcttacagctgcgcgataacacactgatactcagtgataacggcggcagaagcctgtattttgagcacctgtttcccggtgaggacggttacagccgcagcgagtcactctggctggtgcgcggcggcgtggcgaaactggatgaaggtcaccggctggccgcactctggcaggcgctgccggaagaactccgcttaagtccgcatcgttatctggcgacaaacagtccgcaggggccgtggtggctgctcggctggtgtgagcgggtgccggaagcggatgaggtgctgcctgcgccgctgccgccgtaccgggtactgaccgggctggtggaccgcttcgggcgcacacagacgttccaccgcgaagccgccggtgaattcagcggcgaaatcaccggcgtgacggatggtgccgggcgtcacttccggctggtactgaccacgcaggcgcagcgggcagaagaagcccggcagcaggccatttccggcgggacggaaccgtccgcttttcctgataccctgccgggttacaccgaatatggccgggacaacggcatccgtctgtctgccgtgtggctgacgcacgacccggaatacccggagaatttacctgccgcgccgctggtgcgctatggctggacgccgcgcggcgaactggcggtggtgtatgaccgtagtggcaaacaggtgcgcagctttacttacgatgataaataccggggccggatggtggcgcaccgtcacacgggccggccggaaatccgttaccgttacgacagcgacgggcgggtgacagaacagctaaacccggcaggcttaagctacacgtatcagtatgagaaagaccgcatcaccatcaccgacagcctgaaccgccgtgaagtcctgcacacgcagggtgaaggcgggctgaagcgggtggtgaaaaaggaacacgcggacggcagcgtcacgcagagtcagtttgacgcggtgggcaggctcagggcacagacggatgccgcaggcaggacaacagaatacagcccggatgtggtgacgggcctcatcacgcgcatcaccacgccggatggcagggcatcggcgttttactataaccaccacagccagttaacgtcagccaccgggcctgacgggctggaaatacgccgggaatatgatgaatggggccgtctgattcaggaaactgcccctgacggcgatatcacccgctaccgttatgataatccacacagtgacttaccctgcgcaacggaagatgccaccggcagccggaaaaccatgacgtggagccgttacggtcagttgctgagcttcaccgactgttccggttatgtaacccgttatgaccatgaccgcttcgggcagatgacggcggtgcaccgcgaggaagggctgagtcagtaccgcgcatacgacagccgtggacagttaattgccgtgaaagacacgcagggccatgaaacgcggtatgaatacaacgccgccggtgacctgaccaccgtcattgccccggacggcagcagaaacgggacacagtacgatgcgtggggaaaagccatctgtaccacgcagggcggtctgacgcgcagtatggaatacgatgctgccggacgggtcatccgcctgaccagtgaaaacggcagccacaccaccttccgttacgatgtactcgaccggctgatacaggaaaccggctttgacggccgcacacagcgttatcaccacgacctgaccggcaaacttatccgcagcgaggatgaggggctggtcacccactggcactatgacgaagcagaccgcctcacgcaccgcaccgtgaagggtgaaaccgcagagcgctggcagtatgacgaacgcggctggctgacagacatcagccatatcagcgaagggcaccgggtgacggtgcattacgggtatgatgagaaaggccggctgaccggtgagcgtcagacggtgcatcacccgcagacggaagcactgctctggcagcatgagaccagacacgcttacaacgcgcaggggctggcgaaccgctgtataccggacagcctgcccgccgtggaatggctgacctatggcagcggctggctggcaggcatgaagctcggcgacacaccgctggtggatttcacccgcgaccgcctgcaccggaaaacgctgcgcagattcggccgttatgaactcaccaccgcttatacccctgccgggcagttacagagccagcacctgaacagcctgcagtatgaccgcgattacacctggaacgacaacggcgaactcatccgcatcagcagcccgcgccagacccggagttacagctacagcgactccggcaggctgaccggcgttcacaccaccgcagcgaatctggatatccgcatcccgtatgccacggacccggcaggtaaccgcctgcccgacccggagctgcacccggacagcaccctcagcatgtggccggataaccgtatcgcccgtgacgcgcactatctttaccggtatgaccgtcacggcaggctgacagagaaaaccgacctcatcccggaaggggttatccgcacggatgatgagcggactcaccggtaccattacgacagtcagcaccggctggtgcactacacgcggacacaatatgaagagccgctggtcgaaagccgctatctttacgacccgctgggccgcagggtggcaaaacgggtgtggcgacgtgaacgggacctgacgggctggatgtcgctgtcacggaaaccgcaagtgacctggtacggctgggacggcgaccggctgaccacaatacagaacgacagaacccgcatccagacgatttatcagccggggagcttcacgccactcatcagggttgaaaccgccaccggtgagctggcgaaaacgcagcgccgcagcctggcggatgcgcttcagcagtccggcggcgaagacggtggcagtgtggtgttcccgccggtgctggtgcagatgctcgaccggctggaaagtgaaatcctggctgaccgggtgagtgaggaaagccgccgctggctggcatcgtgcggcctgactgtggcgcagatgcaaagccagatggacccggtatacacgccggcgcgaaaaatccacctgtaccactgcgaccatcgcggcctgccgctggcccttatcagtaaggaaggggcaacagaatggtgcgcagaatacgatgagtggggcaacctgctgaatgaagagaacccgcatcagctgcagcagcttatccgcctgccggggcagcagtatgatgaggagtccggcctgtattacaaccgccaccgctattatgacccgctgcaggggaggtatatcactcaggatccgattggactgaaggggggatggaatttttatcagtatccgttgaatccggttcagtatatagattcaatgggactggcatcaaaatatggacacttaaataatggcggatatggagcgagacccaacaaaccgcctacgcccgatccaagtaaatttccagacatagcgaaacaattaagactgccatatcctattgaccaggccagtagtgcgcctaatgttttcaaaacattcttcagagcattaagcccttacgactacacactgtattgcaggaagtgggtaaaaccaaatctgacttgtacgccacaggatgatccccagtatccagggatggatacaaagacagcaagtgattacctgccacagacaaattggccaacaactcaattaccaccaggatatacttgtgcagaaccctatttattccctgacattaataaacccgatgggccagcaacagcagggatagatgatttgggtgaaattttagctaaaatgaaacagagaacatcgagaggaataagaaaatga

SEQ ID NO: 20 - DNA, 7863 bp, Escherichia coli
atggctgcggcagcacaaggtgtggtaaacgccgcaacccaacaaccagttcctgcacaaattgccattgcaaatgccaatacggtgccctacacccttggagcgctggaatcggcccaaagcgttgccgaacgtttcggtatttcggtggctgagttacgcaaactcaaccagtttcgtacgtttgctcgaggttttgataatgtccgccagggtgatgaactggatgtcccggcacaagttagtgaaaataatttaaccccgccaccgggtaatagcagtggcaaccttgagcaacagatagccagtacttcacagcaaatcgggtctctgctcgccgaggatatgaacagcgagcaagcggcaaatatggcgcgtggatgggcctcttctcaggcttcaggcgcaatgacagactggttaagccgcttcggtaccgcaagaatcacgctgggcgtggatgaagattttagcctgaagaactcccagttcgattttctccatccgtggtatgaaacgcctgataatctctttttcagtcagcatactctccatcgtactgacgagcgtacgcagattaacaacggcttgggttggcgtcatttcactcccacatggatgtcgggcatcaacttctttttcgaccacgatcttagccgttaccactcccgcgccggcattggcgcggagtactggcgcgactatctaaaattaagcagtaacggctatttgcgactgaccaactggcgcagcgcacctgaactggacaacgattatgaagcacgcccggccaatggctgggatgtacgcgcagaaggctggctacccgcctggccgcaccttggcggtaaactggtctatgaacagtattatggcgatgaagtggccctgttcgataaagatgatcggcaaagtaatcctcatgccataaccgctggacttaactatacccccttcccgctgatgaccttcagcgcggagcaacgccagggtaaacagggcgaaaatgacacccgttttgccgtcgattttacctggcaacctggaagcgcgatgcagaaacagcttgacccgaatgaagtcgatgcacggcgtagccttgcaggcagccgttttgatctggtggatcgcaacaacaacatcgttctggaatatcgcaaaaaagaactggttcgcctgaccctgacagaccccgtgacagggaagtcaggagaagtgaaatcactggtttcgtcgctacaaaccaaatatgccctgaaaggctataacgtcgaagccaccgctctggaagctgccggtggtaaagtggttacaacgggtaaagatattctggttaccctgccggcgtaccggttcaccagtacgccagaaaccgataacacctggccgattgaagtcaccgctgaagatgtcaaaggcaatttttcgaatcgtgaacagagcatggtagtcgttcaggctcctacgctaagccagaaagattcctcggtatcgttaagtagccagacgttgagcgcggattcccattcaaccgccacactgacttttattgcgcatgatgcagcaggtaatcctgttatcgggctggtgctctcgacgcgtcacgaaggtgttcaggacatcaccctttctgactggaaagataatggtgacggaagctatacccagatcctgaccacaggagcgatgtctggcacgctgacgctgatgccacagctgaacggtgtggatgcggctaaagcccccgccgtggtgaatatcatttctgtttcgtcatcccggactcactcgtcaattaagattgataaggaccgttatctctccgggaatcctatcgaggtgacggtagaactgagagatgaaaatgacaaacctgttaaggagcaaaaacagcaactgaataccgcagtcagcatcgacaacgtgaaacctggtgtcactacagactggaaagaaaccgcagatggcgtctataaggcaacctataccgcctataccaaaggcagtgggcttactgcgaagctgttaatgcaaaactggaatgaagatttgcataccgctggatttatcatcgacgccaacccgcagtcagcgaaaattgcgacattatctgccagcaataatggtgtgctcgccaatgagaatgcagcaaacaccgtctcggtcaatgtcgctgatgaaggaagcaacccaatcaatgatcataccgtcacgtttgcggtattaagcggatcggcaacttcctttaacaatcaaaacaccgcaaaaacggatgttaatggtctggcgacttttgatctgaaaagtagtaagcaggaagacaacacggttgaagtcacccttgaaaatggcgtgaaacaaacgttaatcgtcagttttgtcggcgactcgagtaccgcgcaggttgatctgcagaagtcgaaaaatgaagtggtcgctgacggcaatgacagtgccacaatgaccgcgacagttcgggatgcaaaaggcaacctgctcaatgacgtcaaggtcaccttcaatgtcaattcagcagcagcgaaactgagccaaaccgaagtgaatagccacgacgggatcgccacagctacgctgaccagtttgaaaaatggtgattatacggttacggcctctgtgagctctggttctcaggctaatcaacaggtgatttttatcggtgatcaaagtactgctgccctgaccctcagtgtgccttcaggtgatatcaccgtcaccaacacagctccgctacatatgactgcaaccttgcaggataaaaatggcaatccactaaaagataaagaaatcaccttctctgtgccaaacgacgtcgcaagtcggttctcgattagcaacagcggaaaaggcatgacggatagcaacgggactgcaatcgcctccctgaccggcacgttagcgggcacgcatatgatcacggctcgtctggctaacagcaatgtcagcgatacacagccaatgacgtttgtggcggataaagacagagcggttgtcgttctgcaaacatcgaaagcggaaatcattgggaatggcgtggatgagacgactctgacagcaacagttaaagatccttttgataacgtggttaaaaatctttcagtagtcttccgcacctcccccgcagacacgcaactgagtctgaacgcgcgtaatactaatgagaacggtattgccgaagttacccttaagggcacggttttgggtgttcatacagccgaagccatactgcttaacggcaacagagatacgaaaatcgtcaatattgcgcccgatgccagcaacgcgcaggtcaccctgaacatccctgcacaacaggtggtgacgaataacagtgacagcgtgcagctgacggcgacggtgaaagacccgtcgaatcatccggtggcgggaataacggtgaacttcaccatgccacaggacgtggcggcaaactttacccttgaaaataacggtattgccatcactcaggccaatggcgaagcgcatgtcaccctcaaaggcaaaaaagcgggcacgcatactgtgaccgccacgctgggtaacaataatgccagcgatgcgcaaccagtcaccttcgtggcggataaggacagcgcggttgtcgttctgcaaacatcgaaagcggaaatcattgggaatggcgtggatgagacgactctgacggcaacagtgaaagatccttttgataacgcagtaaaagatctacaggtcaccttcagtaccaaccccgcagatactcaacttagtcagagcaaaagcaatactaacgacagtggtgtggccgaagttacctttaagggcacggttttgggtgttcatacagccgaagccacactgcctaacggcaacaacgatacgaagatagtcaatattgcgcccgatgccagcaacgcgcaggttacgctgaacatccctgctcaacaggtggtgacgaataacagcgacagcgtgcagctgacggcgacggtgaaagatccgtcgaatcatccggtggcgggaataacggtgaacttcaccatgccacaggacgtggcggcaaactttaccctcgaaaataacggtattgccatcacccaggccaatggggaagcgcatgtcacgctcaaaggtaaaaaagcgggtacgcatacggttaccgcaacgctgagtaataacaataccagtgattcacagccggtaacgtttgtggcggacaaaacctcggctctggttgttcttcagatatcaaaaaatgagatcacaggtaatggcgtcgatagcgcaacgctaactgcaacggtcaaagatcagttcgacaatgaggtgaacaatcttccggtaacattcagcacagcttcttcaggcctcaccctgaccccaggggaaagtaataccaatgagtctggcatcgcgcaggccactctcgcaggcgttgcctttggtgagcagacggtcactgcatcactggctaataatggtgccagcgacaacaaaactgtgcattttattggcgacacagcggcggcaaaaattatcgagttgacgcctgtcccagacagcataatcgcaggtaccccgcagaacagctccggcagcgtcatcaccgccacagtcgttgataataatggctttccggtgaaaggtgtgactgtgaacttcaccagcaacgcagcgacagccgaaatgacgaatggcggtcaagccgtgacgaacgaacagggtaaggctaccgtcacttataccaatacccgctcctcgatagaatcaggagcgagaccggataccgttgaggccagtctggaaaatggtagctccacgcttagcacatcaattaatgtcaacgctgatgcgtctacggcacatctcaccttgctacaggcactttttgatacagtctccgcaggcgacactaccaatctgtatattgaggtgaaggataattacggcaacggagtaccccagcaggaggtaaccctcagcgtttcaccaagtgaaggtgtgacccccagtaataacgctatatatacgaccaatcacgacggcaatttttacgcaagctttaccgctacaaaagccggggtataccaagtgacggcaaccctcgaaaatggcgattcgatgcaacaaacagtgacctatgtgccgaacgtagcgaatgctgaaatctcgctggcagcctcgaaggatccggtaattgccaacaataacgatctcacgacactaacagcaacagtcgctgatacagagggcaatgcgatagccaacagtgaggtaacatttactctgccggaagatgtgagggcgaacttcacgctgggcgatggcggtaaagtggttactgatactgaaggcaaagcgaaagtcacgctgaaaggtacaaaagcaggcgctcatactgttacagcatcgatggctggcggtaagagtgagcagttggtggtgaactttattgcggatacactcactgcgcaggttaatcttaacgttaccgaggacaattttatcgctaataacgtcgggatgaccaggctgcaggcaacagtgactgatggaaacggcaacccgttagccaatgaggcggtgacattcacgctaccggcagatgtgagcgcaagctttactctcggacaaggcggttccgccattactgacatcaacggcaaggctgaagttacactgagcggtacaaaatccggcacctaccccgtgacagttagcgtgaacaattatggtgtcagtgatacgaaacaggtgactttgattgccgatgctggtaccgcaaaactagcctccttaacctctgtatactcattcgtcgtcagcacgaccgagggcgcgaccatgactgcaagcgtcactgacgctaacggcaacccggtagaaggtataaaagttaatttccgcggaacttccgtcacgctaagcagcaccagcgttgaaacggatgatcggggtttcgctgaaattcttgtgacaagcaccgaggtcggactgaaaacagtttcagcctctctggcagataaacctactgaagtcatctcgcgattactgaatgcaaaagcagatattaattctgcaacgattaccagtctggagatacctgaaggtcaggtcatggtcgcacaagacgtagcagttaaagctcacgtcaacgaccagtttggcaatccgattcttaatgaatctgtaacattcagtgcagaaccaccagagcacatgaccatcagccaaaatattgtctctactgatacgcatggtatagccgaggtcactatgacgcccgaaagaaacggttcgtatatggtgaaagcatccctggcgaatggatcctcttatgagaaggatctggtggtaatcgatcaaaaactgacactctcggcgtccagcccgcttatcggtgtcaattccccaacaggtgcaactctgacggcaacgctaacttctgcaaatggcactccagtggagggtcaggtcatcaactttagcgtaacgccagaaggtgcgacgttaagtggcggaaaagtgagaaccaactcttcaggtcaggctccagtcgttctgaccagcaataaagtcggtacatatacggtgactgcatcgttccataacggcgtaacaatacagacacagacaatcgtgaaagtcactggcaactcaagcaccgcccatgttgctagctttatcgctgatccatcgactatagccgccaccaacagtgatttaagtaccttaaaggcaacggttgaggatggcagtggtaacctgatcgaaggtctcactgtgtacttcgccttaaaaagcggctctgccacattaacgtcattaacagcggtgacagatcaaaacggaatcgcgacaacaagcgtgagaggagcgataacggggagcgtcacggtaagcgcagtcacgaccgctggtggaatgcaaacagtagatataacgctggtggcaggcccggcagacgcctcgcagtccgtccttaagaacaatcggtcatcattgaaaggagactttaccgatagtgctgagctacatcttgttctgcacgatatatcaggcaatccgatcaaagtttctgaagggctggaatttgtgcagtcaggtaccaacgcgccctatgtgcaagttagtgcaattgactacagtaaaaatttctcaggcgagtacaaagccactgttacaggcggcggagagggtatcgcaacgctgatccctgtattgaatggtgttcatcaagcgggtctgagtaccacaatacaattcactcgcgcagaagacaaaataatgagcggtacagtgttagtcaatggtgctaacctaccgacaactacattcccttcgcaggggttcactggggcgtattatcagttgaataatgacaactttgccccaggaaaaacggcggctgattatgagttttcaagctctgcctcctgggttgatgttgatgctaccggtaaagtgacatttaaaaatgtcggcagcaaatgggagaggattacggcgacgccaaaaacaggcggccctagctatatatacgaaatccgagtgaagagttggtgggtgaacgccggcgatgctttcatgatatacagccttgctgaaaatttttgcagtagcaatggctacacacttccccttggagaccatttaaaccatagtcgttcccgaggcatcgggtcactgtacagtgaatggggagatatggggcattacacgactgaagctggttttcattcaaatatgtattggtcatcgagtcccgcaaactcaaacgaacaatacgtagtttccctggcaacaggtgatcaaagcgtatttgaaaagcttgggtttgcttatgcgacatgttataaaaacctctga

SEQ ID NO: 21 - DNA, 5037 bp, Escherichia coli
gtgccttacacgcttggtgcgctggaatcagcgcaaagcgttgctaatcgcttcggtatttcactggaggagcttcgtcgtcttaatcagttccgtacttttgctcgcggctttgataacgtgcgccagggtgaagaactggatgttccggcaacaacctcgcagaaaagccatgagcaacaaaatgccgtaccgcctgcgaatggcgaaaacacgctggagaatcaaatagccagcacctcgcagcgagttggcactctgctttcacaagatatgaatagtgagcaggccagcggcatggcgcgtggttgggcgtcttcagaagcctcaggcgcgatgactgattggttaaataactttggtactgcgagaatctctctgggtgtggatgaagattttagcctgaaaaattcgcaattcgacttcctgcatccgtggtatgacacacctgattatctgctcttcagccagcatacccttcaccgaacagacgatcgtacccagatcaacaccggtttgggctggcgtcatttcacctccagctggatgtcaggcatcaaccttttttttgaccacgacctgagccgctatcactcccgcgcagggcttggcgcagaatactggcgtgattatctgaagttgagcagcaacgcttatatcggcctgaccggctggcgtagcgcaccagaattggataacgacttcgaagcccgcccggccaacggctgggatttacgcgcggaaggctggttacctgcctggccacaactggggggaaaactggtctatgaacaatactatggcgatgaagtggcgctgtttgacaagaatgatcgtcaaagtaacccccatgctattaccgcaggcctcaactatacccccttcccgcttctgactctcagtgcggaacagcgtcaggggaagcaaggtgaaaatgacacacgttttgccgttgatctgacctggcaacccagcagttcaatgcagaaacagcttaatccggacgaagtggccggacggcgcagtctggccggtagtcgttatgacctgattgatcgcaacaacaacatcgttctggaataccgcaagaaagagctgattcgcctgagtctgctggatccggtgaaagggaagtctggagaaataaaaccgctggtttcctcgctacagaccaaatatgcccttaaaggctataacatcgaagccgctgcgctggaagctgccggaggtaaagtcagcacgtctggaaaagatatcacggtcacgctgccaggttaccgcttcactaacaccccagaaaccgataatacatggtcgatagacgttaccgccgaggatgtaaaaggtaacctgtcacggcatgaacaaagcatggtagttattcaggctccgacattaagccagaaagattctctgttatccgtcaatccgctaaccgtggctgcagataaaaaatcgacgaccacattgaccgttactgcgcacgattccgacggaactccggtgccggggctggcgctgcaaacccgcagtgaaggcgttcaggatatcaccctgtctgactggacagataacggtgatggtagttacacacagatactgaccgccggaacgacatcaggttcagtaacactgacgccgcaaattaacggtgagagtgcggtaaaagaatccatcgtcgttaatatcgtccctgttgtctcatcccgcgaccattcatcaataacaattgataacgtatcgtattatgccggagacgacatcaaggttagggtggaactgaaagacgatagcaatcaaccggttgcatatcaaaaagaggaattggtaaaagccgttactgtcgaaaacagcaaacctggcgccacgattgtctggcacgaagagcagccgggggtttatgccgcgaattatccggcctataagcaagggactgcactaagggcacaacttagccttcacaactggaatgctccactgcaatcgcatatttataacattgaggcaaaccagaataaggctcgcgttgccacattatcagcgacaaataatgacgtttacgccgataaaaagacatttaataccctcacgatcaacgtcactgatgagagtgataatcccctgacaaatcatcaggtcacctttaagaatgaaaaaggaagcgcggagtttgtcgaaccgccgcagcaaaatacggatgcatatggtgttgccacaataaacatggtaagtcaggttgcggaagaaaatacgattagcgccacgctgccaaatggtttttcacaacggataattgcgaaattcgttagcgattcgagtacgccaaaattcaaacaactggttgccgatccagataccattattgctggcaacagccagggcagtactctgaccgccatcatcacagactttcataacaacccgttaaaagatatgaaagtgaattttgtggcacctggtggctcgcaactggacaacacgaccgccacaacagaccagtccggtattgtgcgggtgcacctgaccagttcaaaagctggtagctattccgtcgatgcctcgcttgaggtggataaaaatattcaccagtcggtcacgatcaccgtggtcccaaacagggaacaatcggtaatgaccttgaatgccgggtcgggcagtgcgatcgctaacaatacaaatatcgttaccctgactgccagtgtgaaagatgtttatggacacccgttgccggatgaggatgtgaaatttaccttgccagcctccatgaccgggaacttcacgctaagtagtgaaaccgcccgcaccgatgcaaacggtgatgccgtggtcacattgcgaggcacaaaagcgggtgagtttacagttacggcgacgctgaccagaaataataccgttgcttatcagcaagtcacttttattggggatacaaacagtgcgcagctccagccgctgactgcctcattaaattccattgttgcgggtaacagtacggggagtaccctgacggcaacgatcctggacgcttaccaaaatccgcttaaagaccagttggtcactttccagagtaacgatgtcactctaagcgaaacagaagtcaccaccaatacgctgggtcaggcgacggtaacaatgaccagcaatattgccggacaacataacgtcgtggtgagccggaaagcgcaagcttccgataataaaacgtttagtttatcagtgctaccggatgaaagttcggcgaaggtaataagtataaccggagccgaaaaaacgataacggtgggcgaaaacatcacgctacggatactcgtccaggacgcgtttaacaatgtaatcgcgggtcaacgcgtcagattaagtgcgcagccaacaactaacattacgataggcgatacggcttacaccgataataacggttatgcgtacgttaaccttctcagcacccaacctggggtttatcaggtgacggcaacgctggacaataacagtagtagtaaggttgacgtgaatgtggcaaatggcaaactcgagttaacatcatcgaaaccagaaactacggtccataatagtgagggtattacgctgaccgcaacggcgagaaatgcgcggggtgaattgatgccagggcaaattatcacctttagcgtaacgcctgaaggtgcaacgctaagcaatacaggggaagtccttactgaccagtcaggtcaggccaaagtgacgctgaccagtgacaaagtgaatgtctataccgttacggccataatgggcaaagatgttcccgttcagagccaggtaacggttgcggttaaggcagatgctaaaacggcacatgttgtgagcgtcgtggcttctcctgacaccatcaccgccgacggcatcgatagcagcaccatcacttcacgagtagaagatgattacggattcccggttgaaggtgtcgatattagtcatggcttagacaccaaaggcagcccggtagttaatattccaactacgcgtaccgatcagtccgggcaagtcacggcgacaataaccagtacattggcagaaaccttaacagtcaatgtgcaagttcctggcacagccaaccaatccgcaaccattacattggttgccggcacggccgatgaaagtaagtcaattttgaaatccgatgttgacactctgaaggctgactaccagcagagcgcaaaacttacgctaacattgcaagacaagtacggtaacccgatagtgacgtctgatcatctggaatttgtccagtcaggccccttcgtgaactttctcaagttgagcgatattgattacagccaaagaaattatggcgagtacaccgtgactgtcactggcggaaaagagggaacagcgacactcattcccatgctgaacggggttcatcaggcaaacttaagcatatcgctgaatctcatccaatcgataaaagaaatgtccggtcatgtcactgcaaacaaccataccttctccacggctaaattcccgagcgaaggctttgcaggagcgtattacacactcaacaatgataactttgaagcgggtaaaaccgttgatgattatatgttttcaagttcacagggttgggtgtctgtcgatgcttcgggtaaagtttctttcgcaaatatcggcgatcaaacgtcagtcacaataagcgctgttccccgacaaggaggtacaacctaccagaccttaattaagctgaaaggctggtgggtgaataatggaaatcataccaatatctggctagctgccaatgcgctctgtcatgctaaaaatgatggatataatcttcctggcatcacacatttgacgtctggcgaaaacaaacgcacgcagggatcactgtatggtgaatgggggaacgttggagcgttttccagtaattcgcaatttacaccgggagcttactggacaagtgaatctgatgattacagtcggcactactatgtgcagatgctaaccggtatgaccggaagcgacgctgattccagcccccaactgaccgcctgccgtaaatcactttaa

SEQ ID NO: 22 - DNA, 2409 bp, Escherichia coli
atggtcgctaaattaaaaccagatgtttttgttaatactaatccttttttagaagcaatgtataaggagaggcttagtcataaaggctattctgataaaattgctttaagtattaatgataagaaatataatgtaaactctaaggatattgaaaatattcttgatggcaaaggggatttatttaaaaaacgtacactgtgggagtttgttcgcgatctttttcctggctctcatattaaagaagtcaaaggcttaatttacgaatttgtcacgaaagttgataataaagccgaggtgttcgataagattaaatctctggcaaaaaaagaacaacaatggcgatttagtactaagacagactttaccacaaatgaaaataatgaagttattgtgagtagatcctttaatctctacacgggggctacttcgaatgataacgaaaaaaaacaggtttcttcagagagattaacgcttgataattatctcgatgatttacattttgataattctcccttggtgagattaacgtttgataattattctgtaaaacttgcgacgctgattaaaaataaaattcctattattaatacaacaatcaatttatcatcgttgtctaaggatgttttaaattcattaaaatattgtagttttaagaatgtaattttttcaggagtgataaacagccctaaccttgaaggtccggtttttgaaaattgctatttcgatgattgtaaattcaacaacatacagctttatgagactaatgatgatactgtggaatcaggaaataaaaaaccaataaaaggtatgtttaaaggttgttttatttctaaatgcaaaattgaaaattatagatgtgagacgtctaaaatctacaccgttacacaacctgatagtattcaagaaaaattaggttcttatctctttatgcagtctttcgttcaggattgtataatccagggaggatgttgtcctggctcgagtatacttctgagtcatttttataactgtaatataatagggcttgatgctcatggaatggattttttggcaaattcgttcaacaagtctaattatgatgaaatcaggggtccggatacggggactgtgttttataattgtaatttgaagcatattaaaataaacgatggtcttcgtgaagatggttcagatagaactatgtttaaccccaaagagtatttttctgattacattgaaagaagatcgagaaatcttaagttagatgataatttttcgaaatgcgtagagagtataaagaaagggaccgattcttcatttagcaagaaaaatacagagaacaataatgtcttctttaaaaatagtaatttgattggggcaaatttaggctattattacagtggagataggtgcaaagactgtgctattgatccaaatacaaattattcaaaagatggtcttacagcgcaaaaatacatgtatatggatctggacggagctataagtaaagaacaaatacctgatttcttaaaaaaggtaagtgctatcaatatcgatgtaataaatttttataattccgagtcaaagactaaggaaaaatataaaaacgcatttttagagttagagtcgtttttatcaacactttatgttgaaaataaatcgtacgaggaaaaatatcattttgataatagtagggttgagttttttatttttaaagatatgcaggaaaatgcacaaaatatcattaacaatatgatagaaagtgaccggattaagtttgttgaatctataataaataaaatgattccgccgccagatggaacaatacttactgaaaatccaaaagaatatgtccaaaagcaaataaaggagtctcataaagaatctgctacaaatgtaacatttgattataaggaacttgcacccatttttgaaggtgttgaagaaaaacaaattaaagcactatcaaaccaactggaacatataaaatcgtttaagacagattacgactcaaggctcaataagtatgctcgcgatttttactatttttccagtgcatttgcaattaactgggaggatttactaaaaaactatcaggaaataagagcttccattaaagattacgatgacttattgcgagaaattaaagagttaatcataagtcgtaataaatcattagaggataaaaggactttatttgataacaaaagggacaattggaatagtatagaagtgcaggatgaagtaaatgcattgaatgcgaaaattgtcgattgtgatgataaaatacgttctaagcttactattgttcgaaataatagacttgagaatcaatataaagacgataaaaacatcagtgacgcgatgcgtaatattctggattggttcgaacggtatcccgatattgtacaaaatataacccaggcttaa

SEQ ID NO: 23 - DNA, 2451 bp, Escherichia coli
atgttaaggacgacacgatgggtcgctgctattatttttttgtattcttttccgggttatgctgaagaaacttttgatacgcactttatgatcggcggcatgaaaggagaaaaagtttccgaatatcatttcgataataaacaaccgcttccaggaaactacgaacttgatttttatgtaaataaccagtggcgtggaaaacaagatatcacgatcccagagtcaccagtcaaaccatgtctgcctaaggtgctactaaccaaactgggggtaaagaccggtaacctcaatacagaagataattgtattttactggataaagcggttcatggtggacaatatcagtgggatatcagtgaacaccgactaaatttaacagtaccacaggcgtatatcaatgaacttgaaagaggttacgttcccccggaaagttgggatcgcggcattgatgctttttatacctcctataatttaagtcagtatcgctcttatgacagcaataataacagtaatacagccagttatggccgatttaacagtggattaaatttatttagttggcagttacactctgatgccagctatagcaaacccgatgatatgaaaggcacatggcaaagtaatactctttacctggaacatggctggtcgcagatattaagcacagttcagataggagaaaattacacatcctcactcatattcgactctctgagatttagcggaatacgcttatttcgcgatatgcaaatgctacctgattctatgcagagctttacacctttagtacaaggtgttgcgcaaagtaatgccttgataacggtttcgcaaaatggctacatcatttaccaaaaagaggttcccccgggaccatttactatcgcagatttgcaactctcgggcagcggctctgatctcgatgtcagcataaaggaagcagatggcagtgttcgttcctttttggtgccctactcttccgtgcctaacatgctgcaacctggtatttcaaatttcgattttatcgccggacgcagtaagatatatggtgtaaagaatcaggaggactttctcgaggcaaattatatatatggcctcaacaatcttttgacactttatggcggtacgattttatccgataactataatgctataactctgggaaatggttggaatacaccgctgggtgccatctcttttgacgctacacgatcgagtagtaagttaaataatgatataacacacgaaggtacgagctatcaggtcgcctataataaatatctagtacaaacggccactcgatttagcgtggccgcctggcgttatgcttcgcaggattacaggacattcagcgaccatctttacgaaaatgataaaataaatcatcagagtgactatgacgatttttatgatattggcagaaaaaatagcctttctgccaatatcatgcaacctttatccaataatctgggaaatgtatcattaagtgcgctttggcggaattactgggggcgaagtggaaatgctaaagattaccaattcagttattccaatagctggcaacgcatcagttatactttctctgccagccaatcctatgatgaaaacgacaaagaagaggagcgttttaatctgtttatctccattcctttctactggggggatgatattgccaaaacacgtcaccaaattaacttatcgaattcgacctcattttccaaagatggctattcctccaacaatactggaattactggcatagccggtgaacatgatcagttaaattatggtatatatgttaatcagcaacaacaaaataatgatacatcgcttggtacgaatttaagctggagaactcccatcgccacaatagatggcagctatagccattctaaaaacgcctggcaaagtggtggaagtattagtagtggattagttgtctggcccggtggtattaatatcactaaccagctatccgatacatttgcaattctggatgcgcctgggttagaaggcgcgcatattaatggacaaaaatacaaccgaacaaacagcaaaggccaggttgtttacgacctgatgatacctcatcgtgaaaaccatctggtacttgatacagcaaacagtgaaagtgaaacagaattgcagggcaatcgtcaaattattgcgccttaccgtggagcagtttcttatgtgcagtttacaactgaccaacgtaagccctggtatatacaggcactgcgtcccgatggttcgccattaacttttggctatgacgtactggatctccaggaaaacaatattggagtcgttggccagggtagtcgcctttttattcgcgtagatgaaattccaactggcataaaagttgctctcaatgatgaacagaatttattctgtactattacttttcaacacgttatcgatgaaaacaaaacatatatatgccagtaa

SEQ ID NO: 24 - DNA, 2166 bp, Escherichia coli
atgacggcagatgagcgcaacgtggttaaagcggcaaccgacgatagtatggaagctgcttatatgctaaaagataatatccgctggtattatcataatggtgacctatcattacccgctaacttcagcaataaaaataaactggtggtgaacggtaatttaacgatttccggcgattatgatgattatctgtcaggtaatggtcatctgattgtattaggtaatgttattgtcgataactttattaatcatgatttcgcgtatgtcaaaggacagatgacagcgaaaggtttagtttatgctgattacaatgatcataactttgaagtgatgaagggaattagtgcgcgaggcattattgtttccgataaagcaacgcaatttgaagtcattaaagctgaattttatatcaatgaagatggaagtggtgaaggatataactgggatgagaatattcaaaaagcctattcactggtgactgccgatctttatgatcacactgaaatcgaaaccgataacatttccaatgcgtatcctgactatgatagcgtcgcagacaacatcgttcagggtttaccgctttttcgagataaagccgctcctgaaattaatgagaaattaaaatggattgaaacggggaaactcgataatttcccagcgaataaaataaagcatcaagatccactggtggcacgttttctgactcatacagagagtttatcacccgccgttatgctgcaactgttacagcaccccgatgaccagacgcgtgaatcgatggcgcagtcctggcccgctcaacagatgcattggcttaccgatgaattaattaaagatgaggctgttgccagagggctggttaaaaacagtaatatttccgccgatgtgaataaaaaattaatgtctgtacccgtcgagagcgtacagctggagcaggccagacaggataatctttctccagatattgttgcgtcgttaagtcatagcccctttctcagtgtacgcaagacattactcagccattacgattacgcctggctggttccgactgcagtggccgatgagttgataaataacgaagacccggagttacgtgaaagaattaccggagcagatttaaccgcacaacaagcagtgatgttgagtaaagataaatcactgaaagttcgtgaagcattagcccggacgcttactgaattaaaaattacccaactgtcagcaaccctgcgtacagaggatattgaacgtatcgcagaacaaatgtatctggataataaagaaaataaaaatatcgtaaaggtgttattgatagcgttgccggagatgcgtcaactgtctctggcaaaagaagatgtgcacaatttgcgcgaaggtgcgcgttatctgacatcgaaggacgtgattagttatctattgactcagcatgatgttcccaccgtctgggatgagctggcacgcgataaattattgccgctggaatataagaaacagttatggcaacgcacattaaatttaatgatgagcaaacggcaagaagatcaagagcaagcctacgaagtacagctggcgctgattgataatggtgttgttgacgaagagatgttaaataatgccattgatttactggttgatttacccgctgaataccgctatcgaatgcgtaaccaattatttgataataaagaattaccttccggaattatcaacaaactcgatcaacagtatcgcttcaactctgactgggcgctggctgttgtcagtatgaaaaatagcacccgtagacaaagtgaaagaggattacaccgctggaaccatgaggatagcgatatttttgcggaactggcgacgataaaagataaatcagatgatgaatggtggcgtgcattgctgcaaagtcgcaatgatcatttgcgccaaacggcattacgcaacgcacataccccggcgtcgttgttaacgacattgactgagtctcaggatcggtcgctggctattaataatccacagctggctgccgatgtgaaaacggtgtggctaaaagaggatccatcattacttttatttgtcgataaacccgatctttcgcagttacgggatttagtgaaaaccggggcaacgcggaaaattcgcaatgaagcgcgtcaccggcttgaggaaaaacaatga

SEQ ID NO: 25 - protein, 319 aa, Escherichia coli
MKKSVIAGVFIALSFTTCSAIANSLALSLANDDAGKFQPILNDIYGNKHENRDDYSQGLFLGYSHDISDSSQLSLHIAQDIYSPSGSNKRHNTAVTGDRAFSAYTHTGIEWNSLANDWIRYRLGTDIGVVGPDAGGQKVQNKAHEIIGAEKYHAWDDQIENRYGYTVKGMLSMTPSMDILGANVGLYPEVSAVTGNLFQYVAYGATIAIGNDKTFNSDNGFGLLAPRGLMHMSDTSGFKYKIFAGMERRDVNRNYTLEGKTIQTKQTTVSLNKTVDEYQVGATIGYAPVAFTLAFNKVTSEFKTGDDYSFINGAITFFF

SEQ ID NO: 26 - protein, 266 aa, Escherichia coli
MNSKKLCCICVLFSLLAGCASESSIDEKKKKAQVTQSNINKNTPQQLTDKDLFGNETTLAVSEEDIQAALDGDEFRVPLNSPVILVQSGNRAPETIMQEEMRKYYTVSTFSGIPDRQKPLTCNKNKDKNENEDVARAENMNWMQALRFVAAKGHQKAIIVYQDMLQTGKYDSALKSTVWSDYKNEKLTDAISLRYLVRFTLVDVATGEWATWSPVNYESRVIFPPAGITKTSNKELSNDHVTEAQLFNLKQKTYVSMVKDLVSRFQ

SEQ ID NO: 27 - protein, 1528 aa, Escherichia coli
MNRTSPYYCRRSVLSLLISALIYAPPGMAAFTPDVIGVVNDETVDGSQRVDERGTTNNTHIINHGQQNVYGGVSNGSLIESGGYQDVGRHNNYVGQSNNTTINGGRQSIHDGGISTGTIIESGNQDVYKGGISNGTTIKGGASRVEGGSANGTLIDGGSQIVKVQGHADGTTINKSGSQDVVQGSLATNTTINGGRQYVEQSTVETTTIKNGGEQRVYESRALDTTIEGGTQSLNSKSTAKNTQIYSGGTQIIDNTSSSDVIEVYSGGVLDVSGGTATNVTQHDGAILKTNTNGTTVSGTNSEGAFSIHNHVADNVLLENGGHLDINAYGSANKTIIKDKGTMSVLTNAKADATRIDNGGVMDVAGNATNTIINGGTQNINNYGIATGTNINSGTQNIKSGGKADTTIISSGSRQVVEKDGTAIGSNISAGGSLIVYTGGIAHGVNQETGSALVANTGAGTDIEGYNKLSHFTITGGEANYVVLENTGELTVVAKTSAKNTTIDAGGKLIVQKEAKTDSTRLNNGGVLEVQDGGEAKHVEQQSGGALIASTTSGTLIEGTNSYGDAFYIRNSEAKNVVLENAGSLTVVTGSRAVDTIINANGKMDVYGKDVGTVLNSAGTQTIYASATSDKANIKGGKQTVYGLATEANIESGEQIVDGGSTEKTHINGGTQTVQNYGKAINTDIVSGLQQIMANGTAEGSIINGGSQIVNEGGLAENSVLNDGGTLDVREKGSATGIQQSSQGALVATTRATRVTGTRADGVAFSIEQGAANNILLANGGVLTVESDTSSDKTQVNTGGREIVKTKATATGTTLTGGEQIVEGVANETTINDGGIQTVSANGEAIKTTINEGGTLTVNDNGKATDIVQNSGAALQTSTANGIEISGTHQYGTFSISGNLATNMLLENGGNLLVLAGTEARDSTVGKGGAMQNQGQDSATKVNSGGQYTLGRSKDEFQALARAEDLQVAGGTAIVYAGTLADASVSGATGSLSLMTPRDNVTPVKLEGAIRITDSATLTIGNGVDTTLADLTAASRGSVWLNSNNSCAGTSNCEYRVNSLLLNDGNVYLSAQTAAPATTNGIYNTLTTNELSGSGNFYLHTNVAGSRGDQLVVNNNATGNFKIFVQDTGVSPQSDDAMTLVKTGGGDASFSLGNTGGFVDLGTYEYVLKSDGNSNWNLTNDVKPNPDPNPNPNPNPKPDPKPDPKPDPKPDPTPEPTPTPVPEKRITPSTAAVLNMAATLPLVFDAELNSIRERLNIMKASPHNNNVWGATYNTRNNVTTDAGAGFEQTLTGMTVGIDSPNDIPEGIATLGAFMGYSHSHIGFDRGGHGSVGSYSLGGYASWEHESGFYLDGVVKLNRFESNVAGKMSSGGAANGSYHSNGLGGHIETGMRFTDGNWNLTPYASLTGFTADNPEYHLSNGMESKSVDTRSIYRELGATLSYNMRLGNGMEIEPWLKAAVRKEFVDDNRVKVNNDGNFVNDLSGRRGIYQAGIKASFSSTLSGHLGVGYSHGAGVESPWNAVAGVNWSF

SEQ ID NO: 28 - protein, 248 aa, Escherichia coli
MAGFLIFLSSAAYADINLYGPGGPHTALLDAAKLYAEKTGIIVNVHYGPQNKWNEDAKKNADILFGASEQSALAIIRDHKDSFSEKDIQPLYLRKSILLVKKGNPKNIRSIDDLTRPGIGVIVNDGGGTSNTSGTGVWEDIAGRKGNIETVAAIRKNIILYAPNSGTARKALENQPGADVWITWADWAASNPEIGDVVEIAPDYVIWRDMNITVRQDANDETRRFAEWLQTDEAAPAFKKYGWTRKGT

SEQ ID NO: 29 - protein, 366 aa, Escherichia coli
MKLKIVAVVVTGLLAANVAHAAEVYNKDGNKLDLYGKVTALRYFTDDKRDDGDKTYARLGFKGETQINDQMIGFGHWEYDFKGYNDEANGSRDNKTRLAYAGLKISEFGSLDYGRNYGVGYDIGSWTDMLPEFGGDTWSQKDVFMTYRTTGVATYRNYDFFGLIEGLNFAAQYQGKNERTDNSHLYGADYTRANGDGFGISSTYVYDGFGIGAVYTKSDRTNAQERAAANPLNASGKNAELWATGIKYDANNIYFAANYAETLNMTTYGDGYISNKAQSFEVVAQYQFDFGLRPSLAYLKSKGIDLGRYGDQDMIEYIDVGATYFFNKNMSTYVDYKINLIDESDFTRAVDIRTDNIVATGITYQF

SEQ ID NO: 30 - protein, 319 aa, Escherichia coli
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPECQITGDRPVIKINNTLWESNTAAAFLNRKSQFLYTTGK

SEQ ID NO: 31 - protein, 315 aa, Escherichia coli [sequence omitted]

SEQ ID NO: 32 - protein, 369 aa, Escherichia coli
MQRKGNKLLIQLCSVILLFFTTSWYALANECYIERNAEGDYHMKISSTQLSLASQMVEVPTEIAEATWDVNIQLRGDAIGCKSLGDSKAVHFLNTADPSLISTYTTTNGAALLKTTVPGIVYSVELLCLSCGAADELDLWLPAQSGADNFIPSTQTKWAYEYSDQSWYLRFRLFITPEFKPKNGVSSGTTIAGKIASWYIGTNDQPWINFYIDNDSLKFFVDEPTCATVALAQDQGNVSGNQVTLGNSYVSEVKNGLTREIPFSIRAEYCYASKITVKLKAANKPSDATLVGKTTGSASGVAVKVNSTYDNSKVLLKADGSNTVDYNFAAWSNNLLFLPFTAQLVPDGSGNAVGVGTFSGNATFSFTYE

SEQ ID NO: 33 - protein, 353 aa, Escherichia coli
MRFAKGVLLAICLIFLPLKAALALNCYFGTANGAVEKSEAIMPFAVPANSKPGDKIWESDDIKIPVYCDNNTNGNFESEHVYAWVNPYPGIQDPYYQLGVTYEGVDYDASLGKSRIDTNQCIDSKNIDIYTPEQIIAMGWQNKLCSGDPTVMHKSRTFVARMRLYVKVRAMPPHDYQSKLSDYIVVQFDGAGSVNEDPTAKNLKYHITGLENIRVLDCSVNFAISPETQVVDFGRFNVLDIRRHTMSQQFKITTTKSQNDQCTDGFKVSSSFYTDETLIDEDKSLLIGNGLKLRLLDENASPYTFNKYSEYADFTSDLLVYEKTYTAELSSTPGTPIDVGPFDTVVLFKINYN

SEQ ID NO: 34 - protein, 361 aa, Escherichia coli
MWECDATDLPNIYFLVATNGDDRVGGFYDAGGPDGLSDVYATWFAFVGLKQTMAGVTLGRYWKKVPITSYATQGTKIQIRLQDIPPLHAELYRISTLPDTSATTSWCGNNNTDSSGVGFAKPSGTIYNCVQPNAYIQLSGTSGILFGHDEPGEDSSVHWDFWGADNGFGYGMRSANRLYNNATCVARSATPLVLLPTIAEAQLNAGMESTGNFNVRVECSNSVQSGISDTQTALGIQVSEGAYTAAQKLGIINSNGGVSALVSDNYDAAEMAKGVGIYISNSAHPDTAMTLVGQPGIAKLTPGGNAAGWYPVFEGATLEGATHPGYSSYSYSFIARLKKLPNQTVSAGKVRATAYILVKMQ

SEQ ID NO: 35 - protein, 262 aa, Escherichia coli
MNMAFYGKWFACLWLATSCVQAASTDNKALEIIRRADEIRSPNKPFRYTLTVTEYKAGATQPENKQVLDISMRFMKPQGNEKADARSLVRFIYPPRDKGKIMLSDWYDLWFYTPELRRPMPISRQQRLIGQISNGDVIVTNFEYAYDSTLMGEVTCAEKQCYKLALVRKSADITWPKVIYYVEKDGDNRPWKAAYYSQDDQLIKEVLYQDFQPVLGKTRPMKITVTDVRHGNNYSVMEYSDVRLESLPEFHFTKEYIQRGAK

SEQ ID NO: 36 - protein, 462 aa, Escherichia coli
MKRTLKISSLLCVALPLTVQADCLSGDEVAQNSDVTLYEQVSYVNKQASAWQIAGKNPYTRHNGYQEAGIGINSGCSIIDNTLDLKLNLYGINEYALKPAGKFETDDSRTRALINRLSLVYSASDSVQFEAGKFAAPSGMFFLRSPSDLQTHYYTGFQSTRLHDPKMTSAYQASSWGAKMSVDTRDYAFSASVIPKLATIDKRYVTSGNWSANQQGNSDEAYLLSFSDHRFGEHTPTVNVRLGPSPSLALSDSYHYTPQLTLNVDAAYHRSQQWRHLSHRETAQVEEYQFPDSLYETKDESGVELALGGQYTSDNFSVFGVEYYFQSEGYSRAEQRQQRELIDFLNTTTGYAPLDQAFDSYKYLMASEISNTANQGMLQGKHYLNAWASLPLAGESTLQPSLVVNLVDGSTLLGLHYSTPLSAISNQLEAYAGGYSALGSRYSEFALFGDTLGLYLGFKYYL

SEQ ID NO: 37 - protein, 234 aa, Escherichia coli
MGIDSRNDIPEGIATLGAFMGYSHSHIGFDRGGHGSVDSYSLGGYASWEHESGFYLDGVVKLNRFESNVAGKMSSGGAANGSYHSNGLGGHIETGMRFTDGNWNLTPYASLTGFTADNPEYHLSNGMESKSVDTRSIYRELGATLSYNMRLGNGMEVEPWLKAAVRKEFVDDNRVKVNSDGNFVNDLSGRRGIYQAGIKASFSSTLSGHLGVGYSNGAGMESPWNAVAGVNWSF

SEQ ID NO: 38 - protein, 419 aa, Escherichia coli
MKFPSIFNKIKPQSIQQHPEKNQLNWMLELNKWKEERILTGEIHRPECRNEAAKRINCAFLSKQNDIDLSGLNLSTQPPGLQNFTSINLDNNQLTHFDATNYDRLVKLSLNSNTLESINIHQGRNVSITHISMNNNCLRNIDIDRLSSITYFSAAHNKLEFVQLESCEWLQYLNLSHNQLTDIVTGNKEELLLLDLSHNKLASLHNALFPNLNTLLINNNLLSEIKMFYSNFCKVQTLNAANNQLEKINLHFLTYLSSIKSLRLDNNKITRIDTENTSDIRSLFPIIKKSESLNFLNISGENNCPTIQLMLFNLFSPALKLNTGLAILSPGAFEDHSDGLDVDNELFHYTINKAYTPYNIHTYKTEEVVNQRNIKIKNMTLDEINNTYCNNDYYNEAIREEPIDFLDRSFSSSSWPFYH

SEQ ID NO: 39 - protein, 236 aa, Escherichia coli
MNKAPSLIAAIVLGLGISACGYFVGDGVKHLKTNNRYVNVRGLSEKEVRADTAELTIAINFKGNVPGELFPKLEEAQKKIVAELNAQGINEKEIILGQWTSKRTDSFYLKDDPTMPRYNADGSVTIKTHNVAAVEKVVAKLNELQVATDGAIAESKVAYRFNGIGALRAEMIAAATKDARNAALQFATDSGSQVGSISDASQGVFQIFASGSDEDDPTAINKTVRVVTTVTYALQD

SEQ ID NO: 40 - protein, 280 aa, Escherichia coli
MKKWTIFLTSLILLALSLETPKCYAGDKLMSASFSSTKIYYAMKNVTASGSLYFYVTVVTPGEVSYGQYNSNARKGDTLKLISWSGSGPAPTLVLTDYRRTDTSNCPGINTRVFVCAYMTFNVTVESDNYGCPWIASFYSVSEAFGFGTYTSPTVHDSICPTIPVASFDISWSDNYVSHNKALRLQSDGSTITTTLSTYLMESGKLCDGSIFDSRGAYCRAVSDLLTFTSYGCDNAKVTVTPSRQPLTDRKLHDIVVQVNTSSREPIDSTCRFQYVLNEL

SEQ ID NO: 41 - protein, 303 aa, Escherichia coli
MGTAAISHLRYDLNKYALSLRKTATLASTFFIESPLVRFEYLQEIENTINDITHRFNSSYDINEKARLINELKMESETARKEYQLFRQGNYDKYITTDIFEEHGLIKYVNLSFDIVASVGQVVGGVGALKFGKVVHSNRIKGIGVTLVAHGANNFYESLSPLFFNEYDSGPIRELYRVIAKKMGGDINSADYAYSIVDFSITAYGGYSGMKIVPKYNRLIRPSLGNRPGTGRLFHYTSVDFKNKFSLKPTPLKIIQISSTVKKFKVTFYDENTNLRIKHGLNNHMPHQLLMNISGTTTGAGIQ

SEQ ID NO: 42 - protein, 354 aa, Escherichia coli
MLSLTLMSALLSPLSLQAADVRRSGDEAFIIQQQRQEALEQQLTPSAPDVRLSAPGSFAHKINFPVETPCFQIKQTELKGADALPHWLPLQKIANGAVGHCLGAKGINLLMSTLQNRLVDHGYVTTRVLAPSQDLKSGILRLVIIPGVVRHVRLTPDSDDYIQLYSSFPAHEGSLLDLRDIEQGLDLGNSRIQGQHTELNATSGNLSTQNAQLSADTLSARTAGQFSSNGGTINADTLQISAQSLSNRKGSLIQTGTGDFSLSLPGSVDNREGLLAANGAVRLDALSLDNRKGKVQAEQSPSLQKSPPTFLKPFVAGVCAALLAVSVAIPGWQFLTQPSPEEQHFTWGNGCKKQ

SEQ ID NO: 43 - protein, 1399 aa, Escherichia coli
MSGKPAARQGDMTQYGGSIVQGSAGVRIGAPTGVACSVCPGGVTSGHPVNPLLGAKVLPGETDIALPGPLPFILSRTYSSYRTKTPAPVGSLGPGWKMPADIRLQLRDNTLILSDNGGRSLYFEHLFPGEDGYSRSESLWLVRGGVAKLDEGHRLAALWQALPEELRLSPHRYLATNSPQGPWWLLGWCERVPEADEVLPAPLPPYRVLTGLVDRFGRTQTFHREAAGEFSGEITGVTDGAGRHFRLVLTTQAQRAEEARQQAISGGTEPSAFPDTLPGYTEYGRDNGIRLSAVWLTHDPEYPENLPAAPLVRYGWTPRGELAVVYDRSGKQVRSFTYDDKYRGRMVAHRHTGRPEIRYRYDSDGRVTEQLNPAGLSYTYQYEKDRITITDSLNRREVLHTQGEGGLKRVVKKEHADGSVTQSQFDAVGRLRAQTDAAGRTTEYSPDVVTGLITRITTPDGRASAFYYNHHSQLTSATGPDGLEIRREYDEWGRLIQETAPDGDITRYRYDNPHSDLPCATEDATGSRKTMTWSRYGQLLSFTDCSGYVTRYDHDRFGQMTAVHREEGLSQYRAYDSRGQLIAVKDTQGHETRYEYNAAGDLTTVIAPDGSRNGTQYDAWGKAICTTQGGLTRSMEYDAAGRVIRLTSENGSHTTFRYDVLDRLIQETGFDGRTQRYHHDLTGKLIRSEDEGLVTHWHYDEADRLTHRTVKGETAERWQYDERGWLTDISHISEGHRVTVHYGYDEKGRLTGERQTVHHPQTEALLWQHETRHAYNAQGLANRCIPDSLPAVEWLTYGSGWLAGMKLGDTPLVDFTRDRLHRKTLRRFGRYELTTAYTPAGQLQSQHLNSLQYDRDYTWNDNGELIRISSPRQTRSYSYSDSGRLTGVHTTAANLDIRIPYATDPAGNRLPDPELHPDSTLSMWPDNRIARDAHYLYRYDRHGRLTEKTDLIPEGVIRTDDERTHRYHYDSQHRLVHYTRTQYEEPLVESRYLYDPLGRRVAKRVWRRERDLTGWMSLSRKPQVTWYGWDGDRLTTIQNDRTRIQTIYQPGSFTPLIRVETATGELAKTQRRSLADALQQSGGEDGGSVVFPPVLVQMLDRLESEILADRVSEESRRWLASCGLTVAQMQSQMDPVYTPARKIHLYHCDHRGLPLALISKEGATEWCAEYDEWGNLLNEENPHQLQQLIRLPGQQYDEESGLYYNRHRYYDPLQGRYITQDPIGLKGGWNFYQYPLNPVQYIDSMGLASKYGHLNNGGYGARPNKPPTPDPSKFPDIAKQLRLPYPIDQASSAPNVFKTFFRALSPYDYTLYCRKWVKPNLTCTPQDDPQYPGMDTKTASDYLPQTNWPTTQLPPGYTCAEPYLFPDINKPDGPATAGIDDLGEILAKMKQRTSRGIRK

SEQ ID NO: 44 - protein, 2620 aa, Escherichia coli
MAAAAQGVVNAATQQPVPAQIAIANANTVPYTLGALESAQSVAERFGISVAELRKLNQFRTFARGFDNVRQGDELDVPAQVSENNLTPPPGNSSGNLEQQIASTSQQIGSLLAEDMNSEQAANMARGWASSQASGAMTDWLSRFGTARITLGVDEDFSLKNSQFDFLHPWYETPDNLFFSQHTLHRTDERTQINNGLGWRHFTPTWMSGINFFFDHDLSRYHSRAGIGAEYWRDYLKLSSNGYLRLTNWRSAPELDNDYEARPANGWDVRAEGWLPAWPHLGGKLVYEQYYGDEVALFDKDDRQSNPHAITAGLNYTPFPLMTFSAEQRQGKQGENDTRFAVDFTWQPGSAMQKQLDPNEVDARRSLAGSRFDLVDRNNNIVLEYRKKELVRLTLTDPVTGKSGEVKSLVSSLQTKYALKGYNVEATALEAAGGKVVTTGKDILVTLPAYRFTSTPETDNTWPIEVTAEDVKGNFSNREQSMVVVQAPTLSQKDSSVSLSSQTLSADSHSTATLTFIAHDAAGNPVIGLVLSTRHEGVQDITLSDWKDNGDGSYTQILTTGAMSGTLTLMPQLNGVDAAKAPAVVNIISVSSSRTHSSIKIDKDRYLSGNPIEVTVELRDENDKPVKEQKQQLNTAVSIDNVKPGVTTDWKETADGVYKATYTAYTKGSGLTAKLLMQNWNEDLHTAGFIIDANPQSAKIATLSASNNGVLANENAANTVSVNVADEGSNPINDHTVTFAVLSGSATSFNNQNTAKTDVNGLATFDLKSSKQEDNTVEVTLENGVKQTLIVSFVGDSSTAQVDLQKSKNEVVADGNDSATMTATVRDAKGNLLNDVKVTFNVNSAAAKLSQTEVNSHDGIATATLTSLKNGDYTVTASVSSGSQANQQVIFIGDQSTAALTLSVPSGDITVTNTAPLHMTATLQDKNGNPLKDKEITFSVPNDVASRFSISNSGKGMTDSNGTAIASLTGTLAGTHMITARLANSNVSDTQPMTFVADKDRAVVVLQTSKAEIIGNGVDETTLTATVKDPFDNVVKNLSVVFRTSPADTQLSLNARNTNENGIAEVTLKGTVLGVHTAEAILLNGNRDTKIVNIAPDASNAQVTLNIPAQQVVTNNSDSVQLTATVKDPSNHPVAGITVNFTMPQDVAANFTLENNGIAITQANGEAHVTLKGKKAGTHTVTATLGNNNASDAQPVTFVADKDSAVVVLQTSKAEIIGNGVDETTLTATVKDPFDNAVKDLQVTFSTNPADTQLSQSKSNTNDSGVAEVTFKGTVLGVHTAEATLPNGNNDTKIVNIAPDASNAQVTLNIPAQQVVTNNSDSVQLTATVKDPSNHPVAGITVNFTMPQDVAANFTLENNGIAITQANGEAHVTLKGKKAGTHTVTATLSNNNTSDSQPVTFVADKTSALVVLQISKNEITGNGVDSATLTATVKDQFDNEVNNLPVTFSTASSGLTLTPGESNTNESGIAQATLAGVAFGEQTVTASLANNGASDNKTVHFIGDTAAAKIIELTPVPDSIIAGTPQNSSGSVITATVVDNNGFPVKGVTVNFTSNAATAEMTNGGQAVTNEQGKATVTYTNTRSSIESGARPDTVEASLENGSSTLSTSINVNADASTAHLTLLQALFDTVSAGDTTNLYIEVKDNYGNGVPQQEVTLSVSPSEGVTPSNNAIYTTNHDGNFYASFTATKAGVYQVTATLENGDSMQQTVTYVPNVANAEISLAASKDPVIANNNDLTTLTATVADTEGNAIANSEVTFTLPEDVRANFTLGDGGKVVTDTEGKAKVTLKGTKAGAHTVTASMAGGKSEQLVVNFIADTLTAQVNLNVTEDNFIANNVGMTRLQATVTDGNGNPLANEAVTFTLPADVSASFTLGQGGSAITDINGKAEVTLSGTKSGTYPVTVSVNNYGVSDTKQVTLIADAGTAKLASLTSVYSFVVSTTEGATMTASVTDANGNPVEGIKVNFRGTSVTLSSTSVETDDRGFAEILVTSTEVGLKTVSASLADKPTEVISRLLNAKADINSATITSLEIPEGQVMVAQDVAVKAHVNDQFGNPILNESVTFSAEPPEHMTISQNIVSTDTHGIAEVTMTPERNGSYMVKASLANGSSYEKDLVVIDQKLTLSASSPLIGVNSPTGATLTATLTSANGTPVEGQVINFSVTPEGATLSGGKVRTNSSGQAPVVLTSNKVGTYTVTASFHNGVTIQTQTIVKVTGNSSTAHVASFIADPSTIAATNSDLSTLKATVEDGSGNLIEGLTVYFALKSGSATLTSLTAVTDQNGIATTSVRGAITGSVTVSAVTTAGGMQTVDITLVAGPADASQSVLKNNRSSLKGDFTDSAELHLVLHDISGNPIKVSEGLEFVQSGTNAPYVQVSAIDYSKNFSGEYKATVTGGGEGIATLIPVLNGVHQAGLSTTIQFTRAEDKIMSGTVLVNGANLPTTTFPSQGFTGAYYQLNNDNFAPGKTAADYEFSSSASWVDVDATGKVTFKNVGSKWERITATPKTGGPSYIYEIRVKSWWVNAGDAFMIYSLAENFCSSNGYTLPLGDHLNHSRSRGIGSLYSEWGDMGHYTTEAGFHSNMYWSSSPANSNEQYVVSLATGDQSVFEKLGFAYATCYKNL

SEQ ID NO: 45 - protein, 1678 aa, Escherichia coli
MPYTLGALESAQSVANRFGISLEELRRLNQFRTFARGFDNVRQGEELDVPATTSQKSHEQQNAVPPANGENTLENQIASTSQRVGTLLSQDMNSEQASGMARGWASSEASGAMTDWLNNFGTARISLGVDEDFSLKNSQFDFLHPWYDTPDYLLFSQHTLHRTDDRTQINTGLGWRHFTSSWMSGINLFFDHDLSRYHSRAGLGAEYWRDYLKLSSNAYIGLTGWRSAPELDNDFEARPANGWDLRAEGWLPAWPQLGGKLVYEQYYGDEVALFDKNDRQSNPHAITAGLNYTPFPLLTLSAEQRQGKQGENDTRFAVDLTWQPSSSMQKQLNPDEVAGRRSLAGSRYDLIDRNNNIVLEYRKKELIRLSLLDPVKGKSGEIKPLVSSLQTKYALKGYNIEAAALEAAGGKVSTSGKDITVTLPGYRFTNTPETDNTWSIDVTAEDVKGNLSRHEQSMVVIQAPTLSQKDSLLSVNPLTVAADKKSTTTLTVTAHDSDGTPVPGLALQTRSEGVQDITLSDWTDNGDGSYTQILTAGTTSGSVTLTPQINGESAVKESIVVNIVPVVSSRDHSSITIDNVSYYAGDDIKVRVELKDDSNQPVAYQKEELVKAVTVENSKPGATIVWHEEQPGVYAANYPAYKQGTALRAQLSLHNWNAPLQSHIYNIEANQNKARVATLSATNNDVYADKKTFNTLTINVTDESDNPLTNHQVTFKNEKGSAEFVEPPQQNTDAYGVATINMVSQVAEENTISATLPNGFSQRIIAKFVSDSSTPKFKQLVADPDTIIAGNSQGSTLTAIITDFHNNPLKDMKVNFVAPGGSQLDNTTATTDQSGIVRVHLTSSKAGSYSVDASLEVDKNIHQSVTITVVPNREQSVMTLNAGSGSAIANNTNIVTLTASVKDVYGHPLPDEDVKFTLPASMTGNFTLSSETARTDANGDAVVTLRGTKAGEFTVTATLTRNNTVAYQQVTFIGDTNSAQLQPLTASLNSIVAGNSTGSTLTATILDAYQNPLKDQLVTFQSNDVTLSETEVTTNTLGQATVTMTSNIAGQHNVVVSRKAQASDNKTFSLSVLPDESSAKVISITGAEKTITVGENITLRILVQDAFNNVIAGQRVRLSAQPTTNITIGDTAYTDNNGYAYVNLLSTQPGVYQVTATLDNNSSSKVDVNVANGKLELTSSKPETTVHNSEGITLTATARNARGELMPGQIITFSVTPEGATLSNTGEVLTDQSGQAKVTLTSDKVNVYTVTAIMGKDVPVQSQVTVAVKADAKTAHVVSVVASPDTITADGIDSSTITSRVEDDYGFPVEGVDISHGLDTKGSPVVNIPTTRTDQSGQVTATITSTLAETLTVNVQVPGTANQSATITLVAGTADESKSILKSDVDTLKADYQQSAKLTLTLQDKYGNPIVTSDHLEFVQSGPFVNFLKLSDIDYSQRNYGEYTVTVTGGKEGTATLIPMLNGVHQANLSISLNLIQSIKEMSGHVTANNHTFSTAKFPSEGFAGAYYTLNNDNFEAGKTVDDYMFSSSQGWVSVDASGKVSFANIGDQTSVTISAVPRQGGTTYQTLIKLKGWWVNNGNHTNIWLAANALCHAKNDGYNLPGITHLTSGENKRTQGSLYGEWGNVGAFSSNSQFTPGAYWTSESDDYSRHYYVQMLTGMTGSDADSSPQLTACRKSL

SEQ ID NO: 46 - protein, 802 aa, Escherichia coli
MVAKLKPDVFVNTNPFLEAMYKERLSHKGYSDKIALSINDKKYNVNSKDIENILDGKGDLFKKRTLWEFVRDLFPGSHIKEVKGLIYEFVTKVDNKAEVFDKIKSLAKKEQQWRFSTKTDFTTNENNEVIVSRSFNLYTGATSNDNEKKQVSSERLTLDNYLDDLHFDNSPLVRLTFDNYSVKLATLIKNKIPIINTTINLSSLSKDVLNSLKYCSFKNVIFSGVINSPNLEGPVFENCYFDDCKFNNIQLYETNDDTVESGNKKPIKGMFKGCFISKCKIENYRCETSKIYTVTQPDSIQEKLGSYLFMQSFVQDCIIQGGCCPGSSILLSHFYNCNIIGLDAHGMDFLANSFNKSNYDEIRGPDTGTVFYNCNLKHIKINDGLREDGSDRTMFNPKEYFSDYIERRSRNLKLDDNFSKCVESIKKGTDSSFSKKNTENNNVFFKNSNLIGANLGYYYSGDRCKDCAIDPNTNYSKDGLTAQKYMYMDLDGAISKEQIPDFLKKVSAINIDVINFYNSESKTKEKYKNAFLELESFLSTLYVENKSYEEKYHFDNSRVEFFIFKDMQENAQNIINNMIESDRIKFVESIINKMIPPPDGTILTENPKEYVQKQIKESHKESATNVTFDYKELAPIFEGVEEKQIKALSNQLEHIKSFKTDYDSRLNKYARDFYYFSSAFAINWEDLLKNYQEIRASIKDYDDLLREIKELIISRNKSLEDKRTLFDNKRDNWNSIEVQDEVNALNAKIVDCDDKIRSKLTIVRNNRLENQYKDDKNISDAMRNILDWFERYPDIVQNITQA

SEQ ID NO: 47 - protein, 816 aa, Escherichia coli
MLRTTRWVAAIIFLYSFPGYAEETFDTHFMIGGMKGEKVSEYHFDNKQPLPGNYELDFYVNNQWRGKQDITIPESPVKPCLPKVLLTKLGVKTGNLNTEDNCILLDKAVHGGQYQWDISEHRLNLTVPQAYINELERGYVPPESWDRGIDAFYTSYNLSQYRSYDSNNNSNTASYGRFNSGLNLFSWQLHSDASYSKPDDMKGTWQSNTLYLEHGWSQILSTVQIGENYTSSLIFDSLRFSGIRLFRDMQMLPDSMQSFTPLVQGVAQSNALITVSQNGYIIYQKEVPPGPFTIADLQLSGSGSDLDVSIKEADGSVRSFLVPYSSVPNMLQPGISNFDFIAGRSKIYGVKNQEDFLEANYIYGLNNLLTLYGGTILSDNYNAITLGNGWNTPLGAISFDATRSSSKLNNDITHEGTSYQVAYNKYLVQTATRFSVAAWRYASQDYRTFSDHLYENDKINHQSDYDDFYDIGRKNSLSANIMQPLSNNLGNVSLSALWRNYWGRSGNAKDYQFSYSNSWQRISYTFSASQSYDENDKEEERFNLFISIPFYWGDDIAKTRHQINLSNSTSFSKDGYSSNNTGITGIAGEHDQLNYGIYVNQQQQNNDTSLGTNLSWRTPIATIDGSYSHSKNAWQSGGSISSGLVVWPGGINITNQLSDTFAILDAPGLEGAHINGQKYNRTNSKGQVVYDLMIPHRENHLVLDTANSESETELQGNRQIIAPYRGAVSYVQFTTDQRKPWYIQALRPDGSPLTFGYDVLDLQENNIGVVGQGSRLFIRVDEIPTGIKVALNDEQNLFCTITFQHVIDENKTYICQ

SEQ ID NO: 48 - protein, 721 aa, Escherichia coli
MTADERNVVKAATDDSMEAAYMLKDNIRWYYHNGDLSLPANFSNKNKLVVNGNLTISGDYDDYLSGNGHLIVLGNVIVDNFINHDFAYVKGQMTAKGLVYADYNDHNFEVMKGISARGIIVSDKATQFEVIKAEFYINEDGSGEGYNWDENIQKAYSLVTADLYDHTEIETDNISNAYPDYDSVADNIVQGLPLFRDKAAPEINEKLKWIETGKLDNFPANKIKHQDPLVARFLTHTESLSPAVMLQLLQHPDDQTRESMAQSWPAQQMHWLTDELIKDEAVARGLVKNSNISADVNKKLMSVPVESVQLEQARQDNLSPDIVASLSHSPFLSVRKTLLSHYDYAWLVPTAVADELINNEDPELRERITGADLTAQQAVMLSKDKSLKVREALARTLTELKITQLSATLRTEDIERIAEQMYLDNKENKNIVKVLLIALPEMRQLSLAKEDVHNLREGARYLTSKDVISYLLTQHDVPTVWDELARDKLLPLEYKKQLWQRTLNLMMSKRQEDQEQAYEVQLALIDNGVVDEEMLNNAIDLLVDLPAEYRYRMRNQLFDNKELPSGIINKLDQQYRFNSDWALAVVSMKNSTRRQSERGLHRWNHEDSDIFAELATIKDKSDDEWWRALLQSRNDHLRQTALRNAHTPASLLTTLTESQDRSLAINNPQLAADVKTVWLKEDPSLLLFVDKPDLSQLRDLVKTGATRKIRNEARHRLEEKQ

SEQ ID NO: 49 - DNA, 36 bp, Artificial Sequence
  Description of Artificial Sequence Synthetic oligonucleotide
  Primer MCRL-34Fw
ctgtacttccagggctttcaaccaatacttaatgat

SEQ ID NO: 50 - DNA, 33 bp, Artificial Sequence
  Description of Artificial Sequence Synthetic oligonucleotide
  Primer MCRL-34Rv
aattaagtcgcgttaaaaaaagaaggtgattgc

SEQ ID NO: 51 - DNA, 35 bp, Artificial Sequence
  Description of Artificial Sequence Synthetic oligonucleotide
  Primer MCRL-24Fw
ctgtacttccagggcgatatcaatctgtatggtcc

SEQ ID NO: 52 - DNA, 30 bp, Artificial Sequence
  Description of Artificial Sequence Synthetic oligonucleotide
  Primer MCRL-24Rv
aattaagtcgcgttaagtgcctttcctggt

SEQ ID NO: 53 - DNA, 31 bp, Artificial Sequence
  Description of Artificial Sequence Synthetic oligonucleotide
  Primer MCRL-40Fw
ctgtacttccagggcgacctttatggcaagg

SEQ ID NO: 54 - DNA, 33 bp, Artificial Sequence
  Description of Artificial Sequence Synthetic oligonucleotide
  Primer MCRL-40Rv
aattaagtcgcgttagaactgataggtaatgcc

SEQ ID NO: 55 - DNA, 36 bp, Artificial Sequence
  Description of Artificial Sequence Synthetic oligonucleotide
  Primer MCRL-35Fw
ctgtacttccagggcgagtttacgatagacttttcg

SEQ ID NO: 56 - DNA, 37 bp, Artificial Sequence
  Description of Artificial Sequence Synthetic oligonucleotide
  Primer MCRL-35Rv
aattaagtcgcgttatttacccgttgtatataaaaac

SEQ ID NO: 57 - DNA, 33 bp, Artificial Sequence
  Description of Artificial Sequence Synthetic oligonucleotide
  Primer MCRL-32Fw
ctgtacttccagggcacgtatgtagattcgctg

SEQ ID NO: 58 - DNA, 33 bp, Artificial Sequence
  Description of Artificial Sequence Synthetic oligonucleotide
  Primer MCRL-32Rv
aattaagtcgcgttaactgctaatagttctgcg

SEQ ID NO: 59 - DNA, 33 bp, Artificial Sequence
  Description of Artificial Sequence Synthetic oligonucleotide
  Primer MCRL-41Fw
ctgtacttccagggcatgaagataagctctact

SEQ ID NO: 60 - DNA, 30 bp, Artificial Sequence
  Description of Artificial Sequence Synthetic oligonucleotide
  Primer MCRL-41Rv
aattaagtcgcgttattcgtaggtaaagga

SEQ ID NO: 61 - DNA, 32 bp, Artificial Sequence
  Description of Artificial Sequence Synthetic oligonucleotide
  Primer MCRL-28Fw
ctgtacttccagggccagcaactgacagacaa

SEQ ID NO: 62 - DNA, 31 bp, Artificial Sequence
  Description of Artificial Sequence Synthetic oligonucleotide
  Primer MCRL-28Rv
aattaagtcgcgttactggaatcgactcacc

SEQ ID NO: 63 - DNA, 33 bp, Artificial Sequence
  Description of Artificial Sequence Synthetic oligonucleotide
  Primer MCRL-36Fw
ctgtacttccagggcaactgctattttggtacc

SEQ ID NO: 64 - DNA, 36 bp, Artificial Sequence
  Description of Artificial Sequence Synthetic oligonucleotide
  Primer MCRL-36Rv
aattaagtcgcgttagttgtagttaattttgaaaag

SEQ ID NO: 65 - DNA, 31 bp, Artificial Sequence
  Description of Artificial Sequence Synthetic oligonucleotide
  Primer MCRL-38Fw
ctgtacttccagggcatgtgggaatgtgatg

SEQ ID NO: 66 - DNA, 30 bp, Artificial Sequence
  Description of Artificial Sequence Synthetic oligonucleotide
  Primer MCRL-38Rv
aattaagtcgcgttattgcattttcaccaa

SEQ ID NO: 67 - DNA, 31 bp, Artificial Sequence
  Description of Artificial Sequence Synthetic oligonucleotide
  Primer MCRL-26Fw
ctgtacttccagggcacgctgacggtaacgg

SEQ ID NO: 68 - DNA, 30 bp, Artificial Sequence
  Description of Artificial Sequence Synthetic oligonucleotide
  Primer MCRL-26Rv
aattaagtcgcgttatttcgcacctcgctg

SEQ ID NO: 69 - DNA, 30 bp, Artificial Sequence
  Description of Artificial Sequence Synthetic oligonucleotide
  Primer MCRL-43Fw
ctgtacttccagggcacgctttacgagcag

SEQ ID NO: 70 - DNA, 35 bp, Artificial Sequence
  Description of Artificial Sequence Synthetic oligonucleotide
  Primer MCRL-43Rv
aattaagtcgcgttatagatagtatttaaagccgt

SEQ ID NO: 71 - DNA, 30 bp, Artificial Sequence
  Description of Artificial Sequence Synthetic oligonucleotide
  Primer MCRL-22Fw
ctgtacttccagggcgtggggatcgacagc

SEQ ID NO: 72 - DNA, 32 bp, Artificial Sequence
  Description of Artificial Sequence Synthetic oligonucleotide
  Primer MCRL-22Rv
aattaagtcgcgttagaacgaccagttcacac

SEQ ID NO: 73 - DNA, 30 bp, Artificial Sequence
  Description of Artificial Sequence Synthetic oligonucleotide
  Primer MCRL-42Fw
ctgtacttccagggcatgaaattcccttca

SEQ ID NO: 74 - DNA, 30 bp, Artificial Sequence
  Description of Artificial Sequence Synthetic oligonucleotide
  Primer MCRL-42Rv
aattaagtcgcgttagtgataaaaaggcca

SEQ ID NO: 75 - DNA, 31 bp, Artificial Sequence
  Description of Artificial Sequence Synthetic oligonucleotide
  Primer MCRL-23Fw
ctgtacttccagggcggttattttgttggcg

SEQ ID NO: 76 - DNA, 30 bp, Artificial Sequence
  Description of Artificial Sequence Synthetic oligonucleotide
  Primer MCRL-23Rv
aattaagtcgcgttaatcctgcaacgcata

SEQ ID NO: 77 - DNA, 33 bp, Artificial Sequence
  Description of Artificial Sequence Synthetic oligonucleotide
  Primer MCRL-29Fw
ctgtacttccagggcatttattatgcgatgaaa

SEQ ID NO: 78 - DNA, 36 bp, Artificial Sequence
  Description of Artificial Sequence Synthetic oligonucleotide
  Primer MCRL-29Rv
aattaagtcgcgttacagttcattcagtacatactg

SEQ ID NO: 79 - DNA, 33 bp, Artificial Sequence
  Description of Artificial Sequence Synthetic oligonucleotide
  Primer MCRL-30Fw
ctgtacttccagggcatgggtacagcagctata

SEQ ID NO: 80 - DNA, 31 bp, Artificial Sequence
  Description of Artificial Sequence Synthetic oligonucleotide
  Primer
aattaagtcgcgttattgtattcctgcacca

SEQ ID NO: 81 - DNA, 30 bp, Artificial Sequence
  Description of Artificial Sequence Synthetic oligonucleotide
  Primer MCRL-37Fw
ctgtacttccagggcgatgtccggcgtagc

SEQ ID NO: 82 - DNA, 34 bp, Artificial Sequence
  Description of Artificial Sequence Synthetic oligonucleotide
  Primer MCRL-37Rv
aattaagtcgcgttactgctttttacaaccattc

SEQ ID NO: 83 - DNA, 30 bp, Artificial Sequence
  Description of Artificial Sequence Synthetic oligonucleotide
  Primer MCRL-48Fw
ctgtacttccagggcatgagcggaaaaccg

SEQ ID NO: 84 - DNA, 35 bp, Artificial Sequence
  Description of Artificial Sequence Synthetic oligonucleotide
  Primer MCRL-48Rv
aattaagtcgcgttattttcttattcctctcgatg

SEQ ID NO: 85 - DNA, 30 bp, Artificial Sequence
  Description of Artificial Sequence Synthetic oligonucleotide
  Primer MCRL-52Fw
ctgtacttccagggcatggctgcggcagca

SEQ ID NO: 86 - DNA, 30 bp, Artificial Sequence
  Description of Artificial Sequence Synthetic oligonucleotide
  Primer MCRL-52Rv
aattaagtcgcgttacgcaatattgacgat

SEQ ID NO: 87 - DNA, 30 bp, Artificial Sequence
  Description of Artificial Sequence Synthetic oligonucleotide
  Primer MCRL-49Fw
ctgtacttccagggcgtggggcagtctaat

SEQ ID NO: 88 - DNA, 33 bp, Artificial Sequence
  Description of Artificial Sequence Synthetic oligonucleotide
  Primer MCRL-49Rv
aattaagtcgcgttaactcgctttcattatgtt

SEQ ID NO: 89 - DNA, 33 bp, Artificial Sequence
  Description of Artificial Sequence Synthetic oligonucleotide
  Primer MCRL-51Fw
ctgtacttccagggcgtgccttacacgcttggt

SEQ ID NO: 90 - DNA, 33 bp, Artificial Sequence
  Description of Artificial Sequence Synthetic oligonucleotide
  Primer MCRL-51Rv
aattaagtcgcgttaaagtgatttacggcaggc

SEQ ID NO: 91 - DNA, 34 bp, Artificial Sequence
  Description of Artificial Sequence Synthetic oligonucleotide
  Primer MCRL-45Fw
ctgtacttccagggcatggtcgctaaattaaaac

SEQ ID NO: 92 - DNA, 33 bp, Artificial Sequence
  Description of Artificial Sequence Synthetic oligonucleotide
  Primer MCRL-45Rv
aattaagtcgcgttaagcctgggttatattaac

SEQ ID NO: 93 - DNA, 34 bp, Artificial Sequence; identical to SEQ ID NO: 91
  Description of Artificial Sequence Synthetic oligonucleotide
  Primer MCRL-45Fw

SEQ ID NO: 94 - DNA, 33 bp, Artificial Sequence; identical to SEQ ID NO: 92
  Description of Artificial Sequence Synthetic oligonucleotide
  Primer MCRL-45Rv

SEQ ID NO: 95 - DNA, 30 bp, Artificial Sequence
  Description of Artificial Sequence Synthetic oligonucleotide
  Primer MCRL-46Fw
ctgtacttccagggcagttatggccgattt

SEQ ID NO: 96 - DNA, 33 bp, Artificial Sequence
  Description of Artificial Sequence Synthetic oligonucleotide
  Primer MCRL-46Rv
aattaagtcgcgttatgtaaactgcacataaga

SEQ ID NO: 97 - DNA, 26 bp, Artificial Sequence
  Description of Artificial Sequence Synthetic oligonucleotide
  Primer pET-TEV-Fw
taacgcgacttaattctagcataacc

SEQ ID NO: 98 - DNA, 21 bp, Artificial Sequence
  Description of Artificial Sequence Synthetic oligonucleotide
  Primer pET-TEV-Rv
gccctggaagtacaggttttc

SEQ ID NO: 99 - DNA, 103 bp, Artificial Sequence
  Description of Artificial Sequence Synthetic polynucleotide
  Primer TolR-Cat-Fw
tggaggtcgatttgccagacgctactgaatcacaggcggtgagcagtaacgataatccgccagtgattgttgacatatgaatatcctccttagttcctattcc

SEQ ID NO: 100 - DNA, 96 bp, Artificial Sequence
  Description of Artificial Sequence Synthetic oligonucleotide
  Primer TolR-Cat-Rv
ccttgaaacggctggacacttccgccaccacctgctctggtggtaaacgctccaggcgatctttctcaaccagtgtaggctggagctgcttcgaag

SEQ ID NO: 101 - DNA, 39 bp, Artificial Sequence
  Description of Artificial Sequence Synthetic oligonucleotide
  Primer GMMA-MC001Fw
caggaggaattaaccatgaaaaaaagtgtcatcgctggc

SEQ ID NO: 102 - DNA, 39 bp, Artificial Sequence
  Description of Artificial Sequence Synthetic oligonucleotide
  Primer GMMA-MC001Rv-NONhis
atgatgatgatgatgatgaaaaaagaaggtgattgctcc

SEQ ID NO: 103 - DNA, 38 bp, Artificial Sequence
  Description of Artificial Sequence Synthetic oligonucleotide
  Primer GMMA-MC007Fw
caggaggaattaaccatggggcagtctaataataccac

SEQ ID NO: 104 - DNA, 39 bp, Artificial Sequence
  Description of Artificial Sequence Synthetic oligonucleotide
  Primer GMMA-MC007Rv-NONhis
atgatgatgatgatgatggaacgaccagttcacaccagc

SEQ ID NO: 105 - DNA, 39 bp, Artificial Sequence
  Description of Artificial Sequence Synthetic oligonucleotide
  Primer GMMA-MC020Fw
caggaggaattaaccatggggcagtctaataataccacc

SEQ ID NO: 106 - DNA, 38 bp, Artificial Sequence
  Description of Artificial Sequence Synthetic oligonucleotide
  Primer GMMA-MC020Rv-NONhis
atgatgatgatgatgatggaacgaccagttcacaccag

SEQ ID NO: 107 - DNA, 68 bp, Artificial Sequence
  Description of Artificial Sequence Synthetic oligonucleotide
  Primer MC001tagFw
gattacaaagacgatgatgacaaggattacaaagacgatgatgacaagaacagccttgcattatcatt

SEQ ID NO: 108 - DNA, 68 bp, Artificial Sequence
  Description of Artificial Sequence Synthetic oligonucleotide
  Primer MC001tagRv
cttgtcatcatcgtctttgtaatccttgtcatcatcgtctttgtaatccgcgatagctgaacacgtgg

SEQ ID NO: 109 - DNA, 68 bp, Artificial Sequence
  Description of Artificial Sequence Synthetic oligonucleotide
  Primer MC007tagFw
gattacaaagacgatgatgacaaggattacaaagacgatgatgacaagtctgaatcttctattgatga

SEQ ID NO: 110 - DNA, 68 bp, Artificial Sequence
  Description of Artificial Sequence Synthetic oligonucleotide
  Primer MC007tagRv
cttgtcatcatcgtctttgtaatccttgtcatcatcgtctttgtaatcggcacatcctgcaagcagcg

SEQ ID NO: 111 - DNA, 68 bp, Artificial Sequence
  Description of Artificial Sequence Synthetic oligonucleotide
  Primer MC020tagFw
gattacaaagacgatgatgacaaggattacaaagacgatgatgacaaggccttcactcctgatgttat

SEQ ID NO: 112 - DNA, 68 bp, Artificial Sequence
  Description of Artificial Sequence Synthetic oligonucleotide
  Primer MC020tagRv
cttgtcatcatcgtctttgtaatccttgtcatcatcgtctttgtaatcagccatcccgggcggggcat

SEQ ID NO: 113 - DNA, 25 bp, Artificial Sequence
  Description of Artificial Sequence Synthetic oligonucleotide
  Primer pBADRv
catggttaattcctcctgttagccc

SEQ ID NO: 114 - DNA, 25 bp, Artificial Sequence
  Description of Artificial Sequence Synthetic oligonucleotide
  Primer pBADFwNonHIS
tgagtttaaacggtctccagcttgg

SEQ ID NO: 115 - protein, 296 aa, Salmonella enterica
SSLAISVANDDAGIFQPSLNALYGHPAADRGDYTAGLFLGYSHDLTDASQLSFHIAQDIYSPSGANKRKPEAVKGDRAFSAFLHTGLEWNSLATNWLRYRLGTDIGVIGPDAGGQEVQNRAHRIIGAEKYPAWQDQIENRYGYTAKGMVSLTPAIDILGVNVGFYPEVSAVGGNLFQYLGYGATVALGNDKTFNSDNGFGLLSRRGLIHTQKEGLIYKVFAGVERREVDKNYTLQGKTLQTKMETVDINKTVDEYRVGATIGYSPVAFSLSLNKVTSEFRTGDDYSYINGDITFFF

SEQ ID NO: 116 - protein, 8 aa, Artificial Sequence
  Description of Artificial Sequence Synthetic peptide
DYKDDDDK

SEQ ID NO: 117 - protein, 296 aa, Salmonella enterica
SSLAISVANDDAGIFQPSLNALYGHPAADRGDYTAGLFLGYSHDLTDASQLSFHIAQDIYSPSGANKRKPEAVKGDRAFSAFLHTGLEWNSLATNWLRYRLGTDIGVIGPDAGGQEVQNRAHRIIGAEKYPAWQDQIENRYGYTAKGMVSLTPAIDILGVNVGFYPEVSAVGGNLFQYLGYGATVALGNDKTFNSDNGFGLLSRRGLIHTQKEGLIYKVFAGVERREVDKNYTLQGKTLQTKMETVDINKTVDEYRVGATIGYSPVAESLSLNKVTSEFRTGDDYSYINGDITFFF

SEQ ID NO: 118 - protein, 319 aa, Unknown
  Description of Unknown MC001 sequence
MKKSVIAGVFIALSFTTCSAIANSLALSLANDDAGKFQPILNDIYGNKHENRDDYSQGLFLGYSHDISDSSQLSLHIAQDIYSPSGSNKRHNTAVTGDRAFSAYTHTGIEWNSLANDWIRYRLGTDIGVVGPDAGGQKVQNKAHEIIGAEKYHAWDDQIENRYGYTVKGMLSMTPSMDILGANVGLYPEVSAVTGNLFQYVAYGATIAIGNDKTFNSDNGFGLLAPRGLMHMSDTSGFKYKIFAGMERRDVNRNYTLEGKTIQTKQTTVSLNKTVDEYQVGATIGYAPVAFTLAFNKVTSEEKTGDDYSFINGAITFFF